SEQUENCE LISTING (281 sequences)

SEQ ID NO: 1 - DNA, 23 bp, Artificial Sequence
  Description of Artificial Sequence Synthetic primer
cccactggagatgaacagtcttc

SEQ ID NO: 2 - DNA, 22 bp, Artificial Sequence
  Description of Artificial Sequence Synthetic primer
tggcaaagttcttccagaaagg

SEQ ID NO: 3 - DNA, 21 bp, Artificial Sequence
  Description of Artificial Sequence Synthetic probe
tgtttagaaaaccagctacct

SEQ ID NO: 4 - DNA, 30 bp, Artificial Sequence
  Description of Artificial Sequence Synthetic primer
ccaagataaaggagaagaagaattacagaa

SEQ ID NO: 5 - DNA, 21 bp, Artificial Sequence
  Description of Artificial Sequence Synthetic primer
agcaacgagaaacgcattttg

SEQ ID NO: 6 - DNA, 17 bp, Artificial Sequence
  Description of Artificial Sequence Synthetic probe
catccaggagagccaag

SEQ ID NO: 7 - DNA, 21 bp, Artificial Sequence
  Description of Artificial Sequence Synthetic primer
gagggagcggctgacattatt

SEQ ID NO: 8 - DNA, 24 bp, Artificial Sequence
  Description of Artificial Sequence Synthetic primer
acaccagggtttactggagtcatt

SEQ ID NO: 9 - DNA, 22 bp, Artificial Sequence
  Description of Artificial Sequence Synthetic probe
tcggacacttatgtatcagaca

SEQ ID NO: 10 - DNA, 21 bp, Artificial Sequence
  Description of Artificial Sequence Synthetic primer
cccctcttcctccgactagtg

SEQ ID NO: 11 - DNA, 19 bp, Artificial Sequence
  Description of Artificial Sequence Synthetic primer
cgtctgggtctggcttggt

SEQ ID NO: 12 - DNA, 18 bp, Artificial Sequence
  Description of Artificial Sequence Synthetic probe
catgggaatgttgctgaa

SEQ ID NO: 13 - DNA, 21 bp, Artificial Sequence
  Description of Artificial Sequence Synthetic primer
tgactgaacgctgctcagatg

SEQ ID NO: 14 - DNA, 22 bp, Artificial Sequence
  Description of Artificial Sequence Synthetic primer
cccctttaaaaaacagcatggt

SEQ ID NO: 15 - DNA, 17 bp, Artificial Sequence
  Description of Artificial Sequence Synthetic probe
ctggagctttgatgcta

SEQ ID NO: 16 - DNA, 21 bp, Artificial Sequence
  Description of Artificial Sequence Synthetic primer
caccgtggagaatgtcaagct

SEQ ID NO: 17 - DNA, 20 bp, Artificial Sequence
  Description of Artificial Sequence Synthetic primer
ccgtagccaagtcccagaac

SEQ ID NO: 18 - DNA, 19 bp, Artificial Sequence
  Description of Artificial Sequence Synthetic probe
ctcttcttccaaggtgacc

SEQ ID NO: 19 - DNA, 24 bp, Artificial Sequence
  Description of Artificial Sequence Synthetic primer
cacaaatccaaatggaacataacc

SEQ ID NO: 20 - DNA, 23 bp, Artificial Sequence
  Description of Artificial Sequence Synthetic primer
ggtcaggaaaatggcatactcat

SEQ ID NO: 21 - DNA, 18 bp, Artificial Sequence
  Description of Artificial Sequence Synthetic probe
tggagtcctatgtggtcc

SEQ ID NO: 22 - DNA, 21 bp, Artificial Sequence
  Description of Artificial Sequence Synthetic primer
tccctgaggactccatcttca

SEQ ID NO: 23 - DNA, 22 bp, Artificial Sequence
  Description of Artificial Sequence Synthetic primer
gggattaagatgggctctggtt

SEQ ID NO: 24 - DNA, 17 bp, Artificial Sequence
  Description of Artificial Sequence Synthetic probe
ctgaccgaggtgaatgt

SEQ ID NO: 25 - DNA, 20 bp, Artificial Sequence
  Description of Artificial Sequence Synthetic primer
tgtggcatttggacatgctt

SEQ ID NO: 26 - DNA, 23 bp, Artificial Sequence
  Description of Artificial Sequence Synthetic primer
atgggagaattccttgcagactt

SEQ ID NO: 27 - DNA, 15 bp, Artificial Sequence
  Description of Artificial Sequence Synthetic probe
agagaggccgggatt

SEQ ID NO: 28 - DNA, 25 bp, Artificial Sequence
  Description of Artificial Sequence Synthetic primer
tgttccataaactcacctcctcttt

SEQ ID NO: 29 - DNA, 26 bp, Artificial Sequence
  Description of Artificial Sequence Synthetic primer
tgcttcaggactacaggatattcttc

SEQ ID NO: 30 - DNA, 20 bp, Artificial Sequence
  Description of Artificial Sequence Synthetic probe
tgtgattcagagattgatgc

SEQ ID NO: 31 - DNA, 21 bp, Artificial Sequence
  Description of Artificial Sequence Synthetic primer
tccaattttcagctggaggaa

SEQ ID NO: 32 - DNA, 28 bp, Artificial Sequence
  Description of Artificial Sequence Synthetic primer
cagacactgtaaactccacataggtaga

SEQ ID NO: 33 - DNA, 16 bp, Artificial Sequence
  Description of Artificial Sequence Synthetic probe
tcagcttgtgcccctc

SEQ ID NO: 34 - DNA, 21 bp, Artificial Sequence
  Description of Artificial Sequence Synthetic primer
gctggcagaaaagcaatatgg

SEQ ID NO: 35 - DNA, 18 bp, Artificial Sequence
  Description of Artificial Sequence Synthetic primer
caacctctgccccaccaa

SEQ ID NO: 36 - DNA, 18 bp, Artificial Sequence
  Description of Artificial Sequence Synthetic probe
aggcaacactcagtgaga

SEQ ID NO: 37 - DNA, 20 bp, Artificial Sequence
  Description of Artificial Sequence Synthetic primer
ggctacaggcaggtccaaga

SEQ ID NO: 38 - DNA, 22 bp, Artificial Sequence
  Description of Artificial Sequence Synthetic primer
cgagaggtgactttgcagttga

SEQ ID NO: 39 - DNA, 14 bp, Artificial Sequence
  Description of Artificial Sequence Synthetic probe
cagcgagaagcgac

SEQ ID NO: 40 - DNA, 19 bp, Artificial Sequence
  Description of Artificial Sequence Synthetic primer
aggattctccgcctttgga

SEQ ID NO: 41 - DNA, 24 bp, Artificial Sequence
  Description of Artificial Sequence Synthetic primer
tggagctggaatgagtaggagaag

SEQ ID NO: 42 - DNA, 17 bp, Artificial Sequence
  Description of Artificial Sequence Synthetic probe
ccagaaggacgttcgtg

SEQ ID NO: 43 - DNA, 21 bp, Artificial Sequence
  Description of Artificial Sequence Synthetic primer
cggaattccatgcagtgtctt

SEQ ID NO: 44 - DNA, 22 bp, Artificial Sequence
  Description of Artificial Sequence Synthetic primer
tgtcgttggaaatctgctgttg

SEQ ID NO: 45 - DNA, 16 bp, Artificial Sequence
  Description of Artificial Sequence Synthetic probe
cagatgtggaagatct

SEQ ID NO: 46 - DNA, 26 bp, Artificial Sequence
  Description of Artificial Sequence Synthetic primer
gatatgcttacaaaaatccacattgg

SEQ ID NO: 47 - DNA, 21 bp, Artificial Sequence
  Description of Artificial Sequence Synthetic primer
gcatttttggctgcttgttct

SEQ ID NO: 48 - DNA, 23 bp, Artificial Sequence
  Description of Artificial Sequence Synthetic probe
cactctatgttttaaaggtttct

SEQ ID NO: 49 - DNA, 24 bp, Artificial Sequence
  Description of Artificial Sequence Synthetic primer
tgaatatcccaacacgatcagttt

SEQ ID NO: 50 - DNA, 18 bp, Artificial Sequence
  Description of Artificial Sequence Synthetic primer
ggcagaatcagcgccatt

SEQ ID NO: 51 - DNA, 20 bp, Artificial Sequence
  Description of Artificial Sequence Synthetic probe
tcttgtaacactgggtttta

SEQ ID NO: 52 - DNA, 21 bp, Artificial Sequence
  Description of Artificial Sequence Synthetic primer
ggcactatcgaagtccccaaa

SEQ ID NO: 53 - DNA, 23 bp, Artificial Sequence
  Description of Artificial Sequence Synthetic primer
gatgcatcagttttccaaaaagc

SEQ ID NO: 54 - DNA, 18 bp, Artificial Sequence
  Description of Artificial Sequence Synthetic probe
cttcaaggaacacagttc

SEQ ID NO: 55 - DNA, 18 bp, Artificial Sequence
  Description of Artificial Sequence Synthetic primer
cagccccgggtactcctt

SEQ ID NO: 56 - DNA, 20 bp, Artificial Sequence
  Description of Artificial Sequence Synthetic primer
ttggtggcgtgcttcatgta

SEQ ID NO: 57 - DNA, 14 bp, Artificial Sequence
  Description of Artificial Sequence Synthetic probe
ctctgcccgagctt

SEQ ID NO: 58 - DNA, 23 bp, Artificial Sequence
  Description of Artificial Sequence Synthetic primer
agaggaaatgctggaaaatgtca

SEQ ID NO: 59 - DNA, 18 bp, Artificial Sequence
  Description of Artificial Sequence Synthetic primer
ccggaggtgcttgaatcg

SEQ ID NO: 60 - DNA, 17 bp, Artificial Sequence
  Description of Artificial Sequence Synthetic probe
tctgtccaaaagatgcg

SEQ ID NO: 61 - DNA, 21 bp, Artificial Sequence
  Description of Artificial Sequence Synthetic primer
tccacagtttccaagagggtg

SEQ ID NO: 62 - DNA, 22 bp, Artificial Sequence
  Description of Artificial Sequence Synthetic primer
gcctgtgttccattcaaattca

SEQ ID NO: 63 - DNA, 19 bp, Artificial Sequence
  Description of Artificial Sequence Synthetic probe
ggcattatgatgaagagaa

SEQ ID NO: 64 - DNA, 19 bp, Artificial Sequence
  Description of Artificial Sequence Synthetic primer
ccaagctgggtgcctgtaa

SEQ ID NO: 65 - DNA, 24 bp, Artificial Sequence
  Description of Artificial Sequence Synthetic primer
gtttggcaaagaagaagtggatct

SEQ ID NO: 66 - DNA, 20 bp, Artificial Sequence
  Description of Artificial Sequence Synthetic probe
tgatggaggtatttaagttt

SEQ ID NO: 67 - DNA, 20 bp, Artificial Sequence
  Description of Artificial Sequence Synthetic primer
tggaagaggctggaggtgaa

SEQ ID NO: 68 - DNA, 22 bp, Artificial Sequence
  Description of Artificial Sequence Synthetic primer
agacgacaggtttccaaagctg

SEQ ID NO: 69 - DNA, 17 bp, Artificial Sequence
  Description of Artificial Sequence Synthetic probe
cagactcctcgttgttt

SEQ ID NO: 70 - DNA, 20 bp, Artificial Sequence
  Description of Artificial Sequence Synthetic primer
gctgcatgtggatcctgaga

SEQ ID NO: 71 - DNA, 25 bp, Artificial Sequence
  Description of Artificial Sequence Synthetic primer
tgagtagccagaataatcaccatca

SEQ ID NO: 72 - DNA, 18 bp, Artificial Sequence
  Description of Artificial Sequence Synthetic probe
cttcaagctcctgggtaa

SEQ ID NO: 73 - DNA, 22 bp, Artificial Sequence
  Description of Artificial Sequence Synthetic primer
ctagcctgtggagcaagatgaa

SEQ ID NO: 74 - DNA, 22 bp, Artificial Sequence
  Description of Artificial Sequence Synthetic primer
gacaggtttccaaagctgtcaa

SEQ ID NO: 75 - DNA, 16 bp, Artificial Sequence
  Description of Artificial Sequence Synthetic probe
aggctggaggtgaagc

SEQ ID NO: 76 - DNA, 20 bp, Artificial Sequence
  Description of Artificial Sequence Synthetic primer
caaggcctggccaactatgt

SEQ ID NO: 77 - DNA, 16 bp, Artificial Sequence
  Description of Artificial Sequence Synthetic primer
cgcacgggagcacaca

SEQ ID NO: 78 - DNA, 18 bp, Artificial Sequence
  Description of Artificial Sequence Synthetic probe
atcagtacgatgggaaag

SEQ ID NO: 79 - DNA, 22 bp, Artificial Sequence
  Description of Artificial Sequence Synthetic primer
gacccggtgctacctttttacc

SEQ ID NO: 80 - DNA, 20 bp, Artificial Sequence
  Description of Artificial Sequence Synthetic primer
cactgcttctcgccattgaa

SEQ ID NO: 81 - DNA, 17 bp, Artificial Sequence
  Description of Artificial Sequence Synthetic probe
ttaagtgacgcaaaatg

SEQ ID NO: 82 - DNA, 24 bp, Artificial Sequence
  Description of Artificial Sequence Synthetic primer
ggtgctgagacaaatattccatgt

SEQ ID NO: 83 - DNA, 25 bp, Artificial Sequence
  Description of Artificial Sequence Synthetic primer
tgcggtctgagactcataattgtaa

SEQ ID NO: 84 - DNA, 16 bp, Artificial Sequence
  Description of Artificial Sequence Synthetic probe
tgcaaatggacatggc

SEQ ID NO: 85 - DNA, 24 bp, Artificial Sequence
  Description of Artificial Sequence Synthetic primer
gaaggcatgaacaaagagaccttt

SEQ ID NO: 86 - DNA, 27 bp, Artificial Sequence
  Description of Artificial Sequence Synthetic primer
cctcaacacaagtctgaataccaatag

SEQ ID NO: 87 - DNA, 19 bp, Artificial Sequence
  Description of Artificial Sequence Synthetic probe
cttgaaggaagtcctgaat

SEQ ID NO: 88 - DNA, 17 bp, Artificial Sequence
  Description of Artificial Sequence Synthetic primer
gcaccagctggccattg

SEQ ID NO: 89 - DNA, 25 bp, Artificial Sequence
  Description of Artificial Sequence Synthetic primer
tgaatacttctggtcctttgggata

SEQ ID NO: 90 - DNA, 18 bp, Artificial Sequence
  Description of Artificial Sequence Synthetic probe
aggagtttgaagaaacct

SEQ ID NO: 91 - DNA, 20 bp, Artificial Sequence
  Description of Artificial Sequence Synthetic primer
caccatctgtgccggctact

SEQ ID NO: 92 - DNA, 18 bp, Artificial Sequence
  Description of Artificial Sequence Synthetic primer
gcgcacatcgcggtagtt

SEQ ID NO: 93 - DNA, 14 bp, Artificial Sequence
  Description of Artificial Sequence Synthetic probe
cccaccatgacccg

SEQ ID NO: 94 - DNA, 19 bp, Artificial Sequence
  Description of Artificial Sequence Synthetic primer
tctgtgccacccactttgg

SEQ ID NO: 95 - DNA, 20 bp, Artificial Sequence
  Description of Artificial Sequence Synthetic primer
aggaggcccagggattctag

SEQ ID NO: 96 - DNA, 15 bp, Artificial Sequence
  Description of Artificial Sequence Synthetic probe
acccacaggccagca

SEQ ID NO: 97 - DNA, 16 bp, Artificial Sequence
  Description of Artificial Sequence Synthetic primer
ccggctcacctgcgaa

SEQ ID NO: 98 - DNA, 17 bp, Artificial Sequence
  Description of Artificial Sequence Synthetic primer
cggcagccgcatgttag

SEQ ID NO: 99 - DNA, 15 bp, Artificial Sequence
  Description of Artificial Sequence Synthetic probe
ctgggaagcgagtgc

SEQ ID NO: 100 - DNA, 21 bp, Artificial Sequence
  Description of Artificial Sequence Synthetic primer
tgcacatcggtcactgatctc

SEQ ID NO: 101 - DNA, 20 bp, Artificial Sequence
  Description of Artificial Sequence Synthetic primer
gggtcagtttggccgataaa

SEQ ID NO: 102 - DNA, 16 bp, Artificial Sequence
  Description of Artificial Sequence Synthetic probe
cctactggcacagacg

SEQ ID NO: 103 - DNA, 24 bp, Artificial Sequence
  Description of Artificial Sequence Synthetic primer
gaagacataccacgtaggagaaca

SEQ ID NO: 104 - DNA, 18 bp, Artificial Sequence
  Description of Artificial Sequence Synthetic primer
aggtctgcggcagttgtc

SEQ ID NO: 105 - DNA, 26 bp, Artificial Sequence
  Description of Artificial Sequence Synthetic primer
gctcacagcatcacttttaaacttct

SEQ ID NO: 106 - DNA, 21 bp, Artificial Sequence
  Description of Artificial Sequence Synthetic primer
ctgggcttcaatcgtgacttc

SEQ ID NO: 107 - DNA, 13 bp, Artificial Sequence
  Description of Artificial Sequence Synthetic probe
cccgcccaccact

SEQ ID NO: 108 - DNA, 18 bp, Artificial Sequence
  Description of Artificial Sequence Synthetic primer
tggtggcctccgcagtaa

SEQ ID NO: 109 - DNA, 28 bp, Artificial Sequence
  Description of Artificial Sequence Synthetic primer
ggtaataggtgaatgaagggtaaattct

SEQ ID NO: 110 - DNA, 22 bp, Artificial Sequence
  Description of Artificial Sequence Synthetic probe
ctaaatgtcctctatggtccag

SEQ ID NO: 111 - DNA, 19 bp, Artificial Sequence
  Description of Artificial Sequence Synthetic primer
tcgagtgcattccattccg

SEQ ID NO: 112 - DNA, 21 bp, Artificial Sequence
  Description of Artificial Sequence Synthetic primer
atggaatggcatcaaacggaa

SEQ ID NO: 113 - DNA, 15 bp, Artificial Sequence
  Description of Artificial Sequence Synthetic probe
tggctgtccattcca

SEQ ID NO: 114 - DNA, 19 bp, Artificial Sequence
  Description of Artificial Sequence Synthetic primer
tcgagtgcattccattccg

SEQ ID NO: 115 - DNA, 21 bp, Artificial Sequence
  Description of Artificial Sequence Synthetic primer
atggaatggcatcaaacggaa

SEQ ID NO: 116 - DNA, 15 bp, Artificial Sequence
  Description of Artificial Sequence Synthetic probe
tggctgtccattcca

SEQ ID NO: 117 - DNA, 65 bp, Artificial Sequence
  Description of Artificial Sequence Synthetic oligonucleotide
atgcagcaaggcacagactaarcaaggagasgcaaaattttcrtaggggagagaaatgggtcatt

SEQ ID NO: 118 - DNA, 22 bp, Artificial Sequence
  Description of Artificial Sequence Synthetic primer
atgcagcaaggcacagactacg

SEQ ID NO: 119 - DNA, 23 bp, Artificial Sequence
  Description of Artificial Sequence Synthetic primer
agaggggagagaaatgggtcatt

SEQ ID NO: 120 - DNA, 25 bp, Artificial Sequence
  Description of Artificial Sequence Synthetic primer
caaggcacagactaagcaaggagag

SEQ ID NO: 121 - DNA, 35 bp, Artificial Sequence
  Description of Artificial Sequence Synthetic primer
ggcaaaattttcataggggagagaaatgggtcatt

SEQ ID NO: 122 - DNA, 20 bp, Artificial Sequence
  Description of Artificial Sequence Synthetic primer
acagaagtctgggatgtgga

SEQ ID NO: 123 - DNA, 20 bp, Artificial Sequence
  Description of Artificial Sequence Synthetic primer
gcccaaaaagacagacagaa

SEQ ID NO: 124 - DNA, 20 bp, Artificial Sequence
  Description of Artificial Sequence Synthetic primer
acctgttgtatggcagcagt

SEQ ID NO: 125 - DNA, 21 bp, Artificial Sequence
  Description of Artificial Sequence Synthetic primer
agttgactctttccccaacta

SEQ ID NO: 126 - DNA, 22 bp, Artificial Sequence
  Description of Artificial Sequence Synthetic primer
tttggtaagaaaaacatctccc

SEQ ID NO: 127 - DNA, 20 bp, Artificial Sequence
  Description of Artificial Sequence Synthetic primer
ggctgcagttagctgtcatt

SEQ ID NO: 128 - DNA, 19 bp, Artificial Sequence
  Description of Artificial Sequence Synthetic primer
cctgggcaacaagagcaaa

SEQ ID NO: 129 - DNA, 20 bp, Artificial Sequence
  Description of Artificial Sequence Synthetic primer
agcagagagacataattgtg

SEQ ID NO: 130 - DNA, 20 bp, Artificial Sequence
  Description of Artificial Sequence Synthetic primer
acctgccaaattttaccagg

SEQ ID NO: 131 - DNA, 21 bp, Artificial Sequence
  Description of Artificial Sequence Synthetic primer
gacagagagagggaataaacc

SEQ ID NO: 132 - DNA, 21 bp, Artificial Sequence
  Description of Artificial Sequence Synthetic primer
atatatgcacatccatccatg

SEQ ID NO: 133 - DNA, 23 bp, Artificial Sequence
  Description of Artificial Sequence Synthetic primer
ggccaaagatagatagcaaggta

SEQ ID NO: 134 - DNA, 20 bp, Artificial Sequence
  Description of Artificial Sequence Synthetic primer
acatcgctccttaccccatc

SEQ ID NO: 135 - DNA, 21 bp, Artificial Sequence
  Description of Artificial Sequence Synthetic primer
tgtacccattaaccatcccca

SEQ ID NO: 136 - DNA, 20 bp, Artificial Sequence
  Description of Artificial Sequence Synthetic primer
acagaagtctgggatgtgga

SEQ ID NO: 137 - DNA, 20 bp, Artificial Sequence
  Description of Artificial Sequence Synthetic primer
gcccaaaaagacagacagaa

SEQ ID NO: 138 - DNA, 24 bp, Artificial Sequence
  Description of Artificial Sequence Synthetic primer
agagtgagattctgtctcaattaa

SEQ ID NO: 139 - DNA, 20 bp, Artificial Sequence
  Description of Artificial Sequence Synthetic primer
ggccctgtgtagaagctgta

SEQ ID NO: 140 - DNA, 20 bp, Artificial Sequence
  Description of Artificial Sequence Synthetic primer
atcaggattccaggaggaaa

SEQ ID NO: 141 - DNA, 18 bp, Artificial Sequence
  Description of Artificial Sequence Synthetic primer
acctgggaggcggagctc

SEQ ID NO: 142 - DNA, 23 bp, Artificial Sequence
  Description of Artificial Sequence Synthetic primer
ggcataaaaatagtacagcaagc

SEQ ID NO: 143 - DNA, 20 bp, Artificial Sequence
  Description of Artificial Sequence Synthetic primer
atttgaacagaggcatgtac

SEQ ID NO: 144 - DNA, 20 bp, Artificial Sequence
  Description of Artificial Sequence Synthetic primer
catggatgcagaattcacag

SEQ ID NO: 145 - DNA, 20 bp, Artificial Sequence
  Description of Artificial Sequence Synthetic primer
tcatctccctgtttggtagc

SEQ ID NO: 146 - DNA, 21 bp, Artificial Sequence
  Description of Artificial Sequence Synthetic primer
agggatcttcagagaaacagg

SEQ ID NO: 147 - DNA, 21 bp, Artificial Sequence
  Description of Artificial Sequence Synthetic primer
tgacactatcagctctctggc

SEQ ID NO: 148 - DNA, 23 bp, Artificial Sequence
  Description of Artificial Sequence Synthetic primer
ggtgcttgctgtaaatataattg

SEQ ID NO: 149 - DNA, 20 bp, Artificial Sequence
  Description of Artificial Sequence Synthetic primer
cactacagcagattgcacca

SEQ ID NO: 150 - DNA, 20 bp, Artificial Sequence
  Description of Artificial Sequence Synthetic primer
caaacccgactaccagcaac

SEQ ID NO: 151 - DNA, 20 bp, Artificial Sequence
  Description of Artificial Sequence Synthetic primer
gagccatgttcatgccactg

SEQ ID NO: 152 - DNA, 26 bp, Artificial Sequence
  Description of Artificial Sequence Synthetic primer
caaagagtgaatgctgtacaaacagc

SEQ ID NO: 153 - DNA, 26 bp, Artificial Sequence
  Description of Artificial Sequence Synthetic primer
caagatgtgagtgtgcttttcaggag

SEQ ID NO: 154 - DNA, 20 bp, Artificial Sequence
  Description of Artificial Sequence Synthetic primer
atcccacaggatgcctattt

SEQ ID NO: 155 - DNA, 20 bp, Artificial Sequence
  Description of Artificial Sequence Synthetic primer
acgggagcttttgagaagtt

SEQ ID NO: 156 - DNA, 23 bp, Artificial Sequence
  Description of Artificial Sequence Synthetic primer
tcaaatttttaagtctcaccagg

SEQ ID NO: 157 - DNA, 20 bp, Artificial Sequence
  Description of Artificial Sequence Synthetic primer
gcctgtagaaagcaacaacc

SEQ ID NO: 158 - DNA, 20 bp, Artificial Sequence
  Description of Artificial Sequence Synthetic primer
ggtgacagagcaagaccttg

SEQ ID NO: 159 - DNA, 20 bp, Artificial Sequence
  Description of Artificial Sequence Synthetic primer
gcctcttgtcatcccaagta

SEQ ID NO: 160 - DNA, 20 bp, Artificial Sequence
  Description of Artificial Sequence Synthetic primer
ctctcttcatccaccattgg

SEQ ID NO: 161 - DNA, 20 bp, Artificial Sequence
  Description of Artificial Sequence Synthetic primer
gctgtaagagacctgtgttg

SEQ ID NO: 162 - DNA, 20 bp, Artificial Sequence
  Description of Artificial Sequence Synthetic primer
tggaaccacttcattcttgg

SEQ ID NO: 163 - DNA, 19 bp, Artificial Sequence
  Description of Artificial Sequence Synthetic primer
atttcagaccaagataggc

SEQ ID NO: 164 - DNA, 21 bp, Artificial Sequence
  Description of Artificial Sequence Synthetic primer
cctatttaagtttctgtaagg

SEQ ID NO: 165 - DNA, 20 bp, Artificial Sequence
  Description of Artificial Sequence Synthetic primer
atggtgtagaccctgtggaa

SEQ ID NO: 166 - DNA, 22 bp, Artificial Sequence
  Description of Artificial Sequence Synthetic primer
ctgcacaacatagtgagacctg

SEQ ID NO: 167 - DNA, 24 bp, Artificial Sequence
  Description of Artificial Sequence Synthetic primer
agattacccagaaatgagatcagc

SEQ ID NO: 168 - DNA, 20 bp, Artificial Sequence
  Description of Artificial Sequence Synthetic primer
tcatgtgacaaaagccacac

SEQ ID NO: 169 - DNA, 25 bp, Artificial Sequence
  Description of Artificial Sequence Synthetic primer
agacagaaatatagatgagaatgca

SEQ ID NO: 170 - DNA, 20 bp, Artificial Sequence
  Description of Artificial Sequence Synthetic primer
tcatgtgacaaaagccacac

SEQ ID NO: 171 - DNA, 25 bp, Artificial Sequence
  Description of Artificial Sequence Synthetic primer
agacagaaatatagatgagaatgca

SEQ ID NO: 172 - DNA, 20 bp, Artificial Sequence
  Description of Artificial Sequence Synthetic primer
tttccagtggaaaccaaact

SEQ ID NO: 173 - DNA, 20 bp, Artificial Sequence
  Description of Artificial Sequence Synthetic primer
tccagcaacaacaagagaca

SEQ ID NO: 174 - DNA, 21 bp, Artificial Sequence
  Description of Artificial Sequence Synthetic primer
gtcctcatcctgtaaaacggg

SEQ ID NO: 175 - DNA, 24 bp, Artificial Sequence
  Description of Artificial Sequence Synthetic primer
ccactaactagtttgtgactttgg

SEQ ID NO: 176 - DNA, 22 bp, Artificial Sequence
  Description of Artificial Sequence Synthetic primer
ctgtcctctaggctcatttagc

SEQ ID NO: 177 - DNA, 20 bp, Artificial Sequence
  Description of Artificial Sequence Synthetic primer
ttatgaagcagtgatgccaa

SEQ ID NO: 178 - DNA, 20 bp, Artificial Sequence
  Description of Artificial Sequence Synthetic primer
agctggagagggatagcatt

SEQ ID NO: 179 - DNA, 20 bp, Artificial Sequence
  Description of Artificial Sequence Synthetic primer
tgcattgcatgaaagtagga

SEQ ID NO: 180 - DNA, 21 bp, Artificial Sequence
  Description of Artificial Sequence Synthetic primer
gatgatagagatggcacatga

SEQ ID NO: 181 - DNA, 23 bp, Artificial Sequence
  Description of Artificial Sequence Synthetic primer
tcttcatacatgctttatcatgc

SEQ ID NO: 182 - DNA, 18 bp, Artificial Sequence
  Description of Artificial Sequence Synthetic primer
gtgagtcaattccccaag

SEQ ID NO: 183 - DNA, 22 bp, Artificial Sequence
  Description of Artificial Sequence Synthetic primer
gttgtattagtcaatgttctcc

SEQ ID NO: 184 - DNA, 22 bp, Artificial Sequence
  Description of Artificial Sequence Synthetic primer
atgatgaatgcatagatggatg

SEQ ID NO: 185 - DNA, 19 bp, Artificial Sequence
  Description of Artificial Sequence Synthetic primer
aatgtgtgtccttccaggc

SEQ ID NO: 186 - DNA, 19 bp, Artificial Sequence
  Description of Artificial Sequence Synthetic primer
ttgcagggaaaccacagtt

SEQ ID NO: 187 - DNA, 20 bp, Artificial Sequence
  Description of Artificial Sequence Synthetic primer
tccttggaataaattcccgg

SEQ ID NO: 188 - DNA, 24 bp, Artificial Sequence
  Description of Artificial Sequence Synthetic primer
cttagagggacagaactaataggc

SEQ ID NO: 189 - DNA, 20 bp, Artificial Sequence
  Description of Artificial Sequence Synthetic primer
agcctattgtgggtttgtga

SEQ ID NO: 190 - DNA, 21 bp, Artificial Sequence
  Description of Artificial Sequence Synthetic primer
atgtacatgtgtctgggaagg

SEQ ID NO: 191 - DNA, 24 bp, Artificial Sequence
  Description of Artificial Sequence Synthetic primer
ttctctacatatttactgccaaca

SEQ ID NO: 192 - DNA, 24 bp, Artificial Sequence
  Description of Artificial Sequence Synthetic primer
gagtttgaaaataaagtgttctgc

SEQ ID NO: 193 - DNA, 20 bp, Artificial Sequence
  Description of Artificial Sequence Synthetic primer
ccccaccccttttagtttta

SEQ ID NO: 194 - DNA, 25 bp, Artificial Sequence
  Description of Artificial Sequence Synthetic primer
atgtacgatacgtaatacttgacaa

SEQ ID NO: 195 - DNA, 18 bp, Artificial Sequence
  Description of Artificial Sequence Synthetic primer
gtcccaaaggacctgctc

SEQ ID NO: 196 - DNA, 21 bp, Artificial Sequence
  Description of Artificial Sequence Synthetic primer
gcacccctttatacttgggtg

SEQ ID NO: 197 - DNA, 25 bp, Artificial Sequence
  Description of Artificial Sequence Synthetic primer
tagtactctaccatccatctatccc

SEQ ID NO: 198 - DNA, 24 bp, Artificial Sequence
  Description of Artificial Sequence Synthetic primer
aacagaaccaataggctatctatc

SEQ ID NO: 199 - DNA, 24 bp, Artificial Sequence
  Description of Artificial Sequence Synthetic primer
tacagtaaatcacttggtaggaga

SEQ ID NO: 200 - DNA, 17 bp, Artificial Sequence
  Description of Artificial Sequence Synthetic primer
tccgcgaagtgaagaac

SEQ ID NO: 201 - DNA, 20 bp, Artificial Sequence
  Description of Artificial Sequence Synthetic primer
cttggggagaaccatcctca

SEQ ID NO: 202 - DNA, 20 bp, Artificial Sequence
  Description of Artificial Sequence Synthetic primer
ccggctacaagtgatgtcta

SEQ ID NO: 203 - DNA, 24 bp, Artificial Sequence
  Description of Artificial Sequence Synthetic primer
cctaggtaacatagtgagaccttg

SEQ ID NO: 204 - DNA, 20 bp, Artificial Sequence
  Description of Artificial Sequence Synthetic primer
cctctaaagcatagggtcca

SEQ ID NO: 205 - DNA, 19 bp, Artificial Sequence
  Description of Artificial Sequence Synthetic primer
cccatctgagaacacgctg

SEQ ID NO: 206 - DNA, 23 bp, Artificial Sequence
  Description of Artificial Sequence Synthetic primer
agtttaggagattatcaagctgg

SEQ ID NO: 207 - DNA, 23 bp, Artificial Sequence
  Description of Artificial Sequence Synthetic primer
gttcccataatagatgtatccag

SEQ ID NO: 208 - DNA, 24 bp, Artificial Sequence
  Description of Artificial Sequence Synthetic primer
ttggtacttaataaaccctctttt

SEQ ID NO: 209 - DNA, 22 bp, Artificial Sequence
  Description of Artificial Sequence Synthetic primer
ctagagggacagaaccaatagg

SEQ ID NO: 210 - DNA, 22 bp, Artificial Sequence
  Description of Artificial Sequence Synthetic primer
tgtctgctaatgaatgatttgg

SEQ ID NO: 211 - DNA, 20 bp, Artificial Sequence
  Description of Artificial Sequence Synthetic primer
ccatcccctaaacctctcat

SEQ ID NO: 212 - DNA, 20 bp, Artificial Sequence
  Description of Artificial Sequence Synthetic primer
gtgggaccttgtgattgtgt

SEQ ID NO: 213 - DNA, 20 bp, Artificial Sequence
  Description of Artificial Sequence Synthetic primer
ctggctgacacttagggaaa

SEQ ID NO: 214 - DNA, 20 bp, Artificial Sequence
  Description of Artificial Sequence Synthetic primer
acagaaaaccttttgggacc

SEQ ID NO: 215 - DNA, 20 bp, Artificial Sequence
  Description of Artificial Sequence Synthetic primer
cccagccctgaatattatca

SEQ ID NO: 216 - DNA, 22 bp, Artificial Sequence
  Description of Artificial Sequence Synthetic primer
tgcacttaatatctggtgatgg

SEQ ID NO: 217 - DNA, 20 bp, Artificial Sequence
  Description of Artificial Sequence Synthetic primer
atttctttccctctgcaacc

SEQ ID NO: 218 - DNA, 22 bp, Artificial Sequence
  Description of Artificial Sequence Synthetic primer
agcccattttcataataaatcc

SEQ ID NO: 219 - DNA, 24 bp, Artificial Sequence
  Description of Artificial Sequence Synthetic primer
aatcagtgctttctgtactattgg

SEQ ID NO: 220 - DNA, 20 bp, Artificial Sequence
  Description of Artificial Sequence Synthetic primer
cacttcatggcttaccacag

SEQ ID NO: 221 - DNA, 20 bp, Artificial Sequence
  Description of Artificial Sequence Synthetic primer
gacctttggaaagctagtgt

SEQ ID NO: 222 - DNA, 20 bp, Artificial Sequence
  Description of Artificial Sequence Synthetic primer
tgtcacgtttaccctggaac

SEQ ID NO: 223 - DNA, 23 bp, Artificial Sequence
  Description of Artificial Sequence Synthetic primer
tattcttctatccaaccaacagc

SEQ ID NO: 224 - DNA, 18 bp, Artificial Sequence
  Description of Artificial Sequence Synthetic primer
ttgggtggggacacagag

SEQ ID NO: 225 - DNA, 20 bp, Artificial Sequence
  Description of Artificial Sequence Synthetic primer
cctggctcaaggaattacaa

SEQ ID NO: 226 - DNA, 20 bp, Artificial Sequence
  Description of Artificial Sequence Synthetic primer
ccagcctggctgttagagta

SEQ ID NO: 227 - DNA, 25 bp, Artificial Sequence
  Description of Artificial Sequence Synthetic primer
atattcttatattccatatggcaca

SEQ ID NO: 228 - DNA, 20 bp, Artificial Sequence
  Description of Artificial Sequence Synthetic primer
tggagtctctgggtgaagag

SEQ ID NO: 229 - DNA, 20 bp, Artificial Sequence
  Description of Artificial Sequence Synthetic primer
caggagtatgggatcaccag

SEQ ID NO: 230 - DNA, 18 bp, Artificial Sequence
  Description of Artificial Sequence Synthetic primer
cagcaatttttcaaaggc

SEQ ID NO: 231 - DNA, 25 bp, Artificial Sequence
  Description of Artificial Sequence Synthetic primer
agatcattcatataacctcaaaaga

SEQ ID NO: 232 - DNA, 879 bp, Homo sapiens
gcagggagagagaactggccagggtataaaaagggcccacaagagaccggctctaggatcccaaggcccaactccccgaaccactcagggtcctgtggacagctcacctagtggcaatggctccaggctcccggacgtccctgctcctggcttttgccctgctctgcctgccctggcttcaagaggctggtgccgtccaaaccgttccgttatccaggctttttgaccacgctatgctccaagcccatcgcgcgcaccagctggccattgacacctaccaggagtttgaagaaacctatatcccaaaggaccagaagtattcattcctgcatgactcccagacctccttctgcttctcagactctattccgacaccctccaacatggaggaaacgcaacagaaatccaatctagagctgctccgcatctccctgctgctcatcgagtcgtggctggagcccgtgcggttcctcaggagtatgttcgccaacaacctggtgtatgacacctcggacagcgatgactatcacctcctaaaggacctagaggaaggcatccaaacgctgatggggaggctggaagacggcagccgccggactgggcagatcctcaagcagacctacagcaagtttgacacaaactcgcacaaccatgacgcactgctcaagaactacgggctgctctactgcttcaggaaggacatggacaaggtcgagacattcctgcgcatggtgcagtgccgctctgtggagggcagctgtggcttctaggtgcccgagtagcatcctgtgacccctccccagtgcctctcctggccctgaaggtgccactccagtgcccaccagccttgtcctaataaaattaagttgtatcatttca

SEQ ID NO: 233 - DNA, 731 bp, Homo sapiens
gagaactcttgagaccgggagcccagctgcccaccctctggacattcacccagccaggtggtctcgtcacctcagaggctccgccagactcctgcccaggccaggactgaggcaagcctcaaggcacttctaggacctgcctcttctcaccaagatgaactcactggtttcttggcagctactgcttttcctctgtgccacccactttggggagccattagaaaaggtggcctctgtggggaattctagacccacaggccagcagctagaatccctgggcctcctggcccccggggagcagagcctgccgtgcaccgagaggaagccagctgctactgccaggctgagccgtcgggggacctcgctgtccccgccccccgagagctccgggagcccccagcagccgggcctgtccgccccccacagccgccagatccccgcaccccagggcgcggtgctggtgcagcgggagaaggacctgccgaactacaactggaactccttcggcctgcgcttcggcaagcgggaggcggcaccagggaaccacggcagaagcgctgggcggggctgagggcgcaggtgcggggcagtgaacttcagaccccaaaggagtcagagcatgcggggcgggggcggggggcggggacgtagggctaagggagggggcgctggagcttccaacccgaggcaataaaagaaatgttgcgtaactcaaaaaaaaaaaaaaaaaa

SEQ ID NO: 234 - DNA, 1279 bp, Homo sapiens
agaaactcagagaccaagtccattgagagactgaggggaaagagaggagagaaagaaaaagagagtgggaacagtaaagagaaaggaagacaacctccagagaaagcccccggagacgtctctctgcagagaggcggcagcacccggctcacctgcgaagcgcctgggaagcgagtgcccctaacatgcggctgccgctgcttgtgtccgcgggagtcctgctggtggctctcctgccctgcccgccatgcagggcgctcctgagccgcgggccggtcccgggagctcggcaggcgccgcagcaccctcagcccttggatttcttccagccgccgccgcagtccgagcagccccagcagccgcaggctcggccggtcctgctccgcatgggagaggagtacttcctccgcctggggaacctcaacaagagcccggccgctcccctttcgcccgcctcctcgctcctcgccggaggcagcggcagccgcccttcgccggaacaggcgaccgccaactttttccgcgtgttgctgcagcagctgctgctgcctcggcgctcgctcgacagccccgcggctctcgcggagcgcggcgctaggaatgccctcggcggccaccaggaggcaccggagagagaaaggcggtccgaggagcctcccatctccctggatctcaccttccacctcctccgggaagtcttggaaatggccagggccgagcagttagcacagcaagctcacagcaacaggaaactcatggagattattgggaaataaaacggtgcgtttggccaaaaagaatctgcatttagcacaaaaaaaatttaaaaaaatacagtattctgtaccatagcgctgctcttatgccatttgtttatttttatatagcttgaaacatagagggagagagggagagagcctataccccttacttagcatgcacaaagtgtattcacgtgcagcagcaacacaatgttattcgttttgtctacgtttagtttccgtttccaggtgtttatagtggtgttttaaagagaatgtagacctgtgagaaaacgttttgtttgaaaaagcagacagaagtcactcaattgtttttgttgtggtctgagccaaagagaatgccattctcttgggtgggtaagactaaatctgtaagctctttgaaacaactttctcttgtaaacgtttcagtaataaaacatctttccagtccttggtcagtttggttgtgtaagagaatgttgaatacttatatttttaataaaagttgcaaaggt

SEQ ID NO: 235 - DNA, 879 bp, Homo sapiens
tccagcacctttctcgggtcacggcctcctcctggctcccaggaccccaccataggcagaggcaggccttcctacaccctactccctgtgcctccagcctcgactagtccctagcactcgacgactgagtctctgaggtcacttcaccgtggtctccgcctcacccttggcgctggaccagtgagaggagagggctggggcgctccgctgagccactcctgcgcccccctggccttgtctacctcttgccccccgaggggttagtgtcgagctcaccccagcatcctatcacctcctggtggccttgccgcccccacaaccccgaggtataaagccaggtacacgaggcaggggacgcaccaaggatggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagcccatcccgactcccggggccctcggacaccccgatcctcccacaataaaggcttctcaatccgc

SEQ ID NO: 236 - DNA, 2067 bp, Homo sapiens
cgctttttttttttttttttatgaataaattgtatgtgtttctagctgaaactactcaccaatatgcatgttaaattcaatcctctcttacctattctttgatataactccttgaagttttcccattctcctacatcatctcccccacccccactgagtcatttctataatacaaacatattatctttttattaaattaaaaatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccaaaaaaaaaaaaaaa

SEQ ID NO: 237 - DNA, 1706 bp, Homo sapiens
acagaaggaggaaggacagcacagctgacagccgtgctcagacagcttctggatcccaggctcatctccacagaggagaacacacaggcagcagagaccatggggcccctcccagccccttcctgcacacagcgcatcacctggaaggggctcctgctcacagcatcacttttaaacttctggaacccgcccaccactgccgaagtcacgattgaagcccagccacccaaagtttctgaggggaaggatgttcttctacttgtccacaatttgccccagaatcttcctggctacttctggtacaaaggggaaatgacggacctctaccattacattatatcgtatatagttgatggtaaaataattatatatgggcctgcatacagtggaagagaaacagtatattccaacgcatccctgctgatccagaatgtcacccggaaggatgcaggaacctacaccttacacatcataaagcgaggtgatgagactagagaagaaattcgacatttcaccttcaccttatacttggagactcccaagccctacatctccagcagcaacttaaaccccagggaggccatggaggctgtgcgcttaatctgtgatcctgagactctggacgcaagctacctatggtggatgaatggtcagagcctccctgtgactcacaggttgcagctgtccaaaaccaacaggaccctctatctatttggtgtcacaaagtatattgcaggaccctatgaatgtgaaatacggaacccagtgagtgccagtcgcagtgacccagtcaccctgaatctcctcccgaagctgcccatcccctacatcaccatcaacaacttaaaccccagggagaataaggatgtcttagccttcacctgtgaacctaagagtgagaactacacctacatttggtggctaaacggtcagagcctccccgtcagtcccggggtaaagcgacccattgaaaacaggatactcattctacccagtgtcacgagaaatgaaacaggaccctatcaatgtgaaatacgggaccgatatggtggcctccgcagtaacccagtcatcctaaatgtcctctatggtccagacctccccagaatttacccttcattcacctattaccgttcaggagaaaacctcgacttgtcctgcttcacggaatctaacccaccggcagagtatttttggacaattaatgggaagtttcagcaatcaggacaaaagctctttatcccccaaattactagaaatcatagcgggctctatgcttgctctgttcataactcagccactggcaaggaaatctccaaatccatgacagtcaaagtctctggtccctgccatggagacctgacagagtctcagtcatgactgcaacaactgagacactgagaaaaagaacaggctgataccttcatgaaattcaagacaaagaagaaaaaaactcaatgttattggactaaataatcaaaaggataatgttttcataattttttattggaaaatgtgctgattctttgaatgttttattctccagatttatgaactttttttcttcagcaattggtaaagtatacttttgtaaacaaaaattgaaatatttgcttttgctgtctatctgaatgccccagaattgtgaaactattcatgagtattcataggtttatggtaataaagttatttgcacatgttccgta

SEQ ID NO: 238 - DNA, 883 bp, Homo sapiens
cagggagagagaactggccagggtataaaaagggcccacaagagaccggctctaggatcccaaggcccaactccccgaaccactcagggtcctgtggacagctcacctagcggcaatggctgcaggctcccggacgtccctgctcctggcttttgccctgctctgcctgccctggcttcaagaggctggtgccgtccaaaccgttccgttatccaggctttttgaccacgctatgctccaagcccatcgcgcgcaccagctggccattgacacctaccaggagtttgaagaaacctatatcccaaaggaccagaagtattcattcctgcatgactcccagacctccttctgcttctcagactctattccgacaccctccaacatggaggaaacgcaacagaaatccaatctagagctgctccgcatctccctgctgctcatcgagtcgtggctggagcccgtgcggttcctcaggagtatgttcgccaacaacctggtgtatgacacctcggacagcgatgactatcacctcctaaaggacctagaggaaggcatccaaacgctgatggggaggctggaagacggcagccgccggactgggcagatcctcaagcagacctacagcaagtttgacacaaactcacacaaccatgacgcactgctcaagaactacgggctgctctactgcttcaggaaggacatggacaaggtcgagacattcctgcgcatggtgcagtgccgctctgtagagggtagctgtggcttctaggtgcccgcgtggcatcctgtgaccgacccctccccagtgcctctcctggccctggaaggtgccactccagtgcccatcagccttgtcctaataaaattaagttgtatcatttca

SEQ ID NO: 239 - DNA, 992 bp, Homo sapiens
agcgtttaaacttaagcttggagttatttccaccatgcccgggcaagaactcaggacgctgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcctgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgaactagtactaagccgaattctgcagatatcc

SEQ ID NO: 240 - DNA, 979 bp, Homo sapiens
ggacgccttgcccagcgggccgcccgaccccctgcaccatggaccccgctcgccccctggggctgtcgattctgctgcttttcctgacggaggctgcactgggcgatgctgctcaggagccaacaggaaataacgcggagatctgtctcctgcccctagactacggaccctgccgggccctacttctccgttactactacgacaggtacacgcagagctgccgccagttcctgtacgggggctgcgagggcaacgccaacaatttctacacctgggaggcttgcgacgatgcttgctggaggatagaaaaagttcccaaagtttgccggctgcaagtgagtgtggacgaccagtgtgaggggtccacagaaaagtatttctttaatctaagttccatgacatgtgaaaaattcttttccggtgggtgtcaccggaaccggattgagaacaggtttccagatgaagctacttgtatgggcttctgcgcaccaaagaaaattccatcattttgctacagtccaaaagatgagggactgtgctctgccaatgtgactcgctattattttaatccaagatacagaacctgtgatgctttcacctatactggctgtggagggaatgacaataactttgttagcagggaggattgcaaacgtgcatgtgcaaaagctttgaaaaagaaaaagaagatgccaaagcttcgctttgccagtagaatccggaaaattcggaagaagcaattttaaacattcttaatatgtcatcttgtttgtctttatggcttatttgcctttatggttgtatctgaagaataatatgacagcatgaggaaacaaatcattggtgatttattcaccagtttttattaatacaagtcactttttcaaaaatttggatttttttatatataactagctgctattcaaatgtgagtctaccatttttaatttatggttcaactgtttgtgagactgaattc

SEQ ID NO: 241 - DNA, 2779 bp, Homo sapiens
ctaggcctcagtctgtctgcatccaggtgcttattaaaacagtgtgttgctccacaccgcctcgtgttgtctgttggcgcgctctccgggttccaaccaatgcaagagccttggggctggccctgaaacctgcgaggggcttccgtccacgtccccagtggacctaccacccctccatctgggaaagcaggccacagcagccggacaaaggaagctcctcagcctctagtcgcctctctgtgcatgcacatcggtcactgatctcgcctactggcacagacgtgtttatcggccaaactgaccctcacaaaaagctaccaccgaagtggacaggcccctacactgtgatactcagcacaccaactgcagtgagagtccgaggactccccaactggatccatcgcaccagggtcaagctcacccccaaggcagcttcttcctccaaaacattaacagctaagtgtttgtctgggccaatttctcctaccaagtttaaattaaccaacatttttttcttaaaaccaaaacacaaggaagactaaccacgtgcttccaggaatggcctgtatctacccaaccactttctatacctctcttccaaccaaaagtcttaatatgggaatatccctcaccacgatcctaatactgtcagtagctgtcctgctgtccacagcagcccctccgagctgccgtgagtgttatcagtctttgcactacagaggggagatgcaacaatactttacttaccatactcatatagaaagatcctgttatggaaacttaatcgaggaatgtgttgaatcaggaaagagttattataaagtaaagaatctaggagtatgtggcagtcgtaatggggctatttgccccagagggaagcagtggctttgcttcaccaaaattggacaatggggagtaaacactcaggtgcttgaggacataaagagagaacagattatagccaaagccaaagcctcaaaaccaacaactccccctgaaaatcgcccgcggcatttccattcctttatacaaaaactataagcagatgcatcccttcctaagccaggaaaaaatctgtttgtagatctaggagaaccattgtgcttaccatgaatgtgtccaattgttgggtatgcgggggagctttatgagtgaacagtggctgtgggacgggatagacattcccccttacttacaggcatcccaaaaccccagactcactttcactcctcaggaatgcccgcagtcctggacacttaccaacccagtatgagggacggtgtgcatatcccgcaagtggactgataaaacccatcgcgccgtaggtgaaaacccgtcaccaaaccctaacagtcaatgcctccatagctgagtggtggccaaggttaccccctggagcctggtctccttctaacttaagctacctcaattgtgtcttgtcaaaaaaggcctggtactgtacgaacaccactaacccttatgccgcatacctccgcctaagtgtactatgcgacaatcctaggaacaccagctgacaatggactgccactgacggattcctgtggatatggggaacccaggcttactcacagctaccttatcactggcaaggtacttgcttcctaggcacaattcaacctggattctttttacttccgaagcaggcgggcaacaccctcagagtccctgtgtatgataaccagagaaaaatgatccttggaggtaggagggagccaaagattgtgagaggacgagtggcctctgcaacggatcattgaatactatggtcctgccacttgggcagaggatggttcatggggttatcgcactcccatatatatgccaaatagagcgattagactacaagctgttctagagataatcactaaccaaactgcctcagccctagaaatgctcgcgcaacaacaaaaccaaatgcgcgcggcaatttatcaaaacaggctggccctagactacttattagcagaagagggtgcgggctgtggtaagtttaacatctccaattgctgtcttaacataggcaataatggagaagaggttctggaaatcgcttcaaacatcagaaaagtagcccgtgtaccagtccaaacctgggagggatgggacccagcaaaccttctaggagggtggttctctaatttaggaggatttaaaatgctggtggggacagtcattttcatcactggggtcctcctgtttctcccctgtggtatcccattaaaactcttgttgaaactacagttaacctcctgacaatccagatgatgctcctgctacagcggcacgatggataccaacccgtctctcaagaataccccaaaaattaagtttttctttttccaaggtgcccacgccacccctatgtcacgcctgaagtagttattgagaaagtcgtccctttccccttttctataaccaaatagacaggaatggaagattctcctcggggcctgaaagcttgcgggatgaataactcctcctcctcaggcccagtcccaaggtacaaacttgcaccagcagcaagatagcagaggcaggaagagagctggctggaagacacgtaccccctgaagatcaagagggaggtcgccctggtactacatagcagtcacgttaggctgggacaattcctgtttacagaggactataaaacccctgccccatcctcacttggggctgatgccattttaggcctcagcctgtctgcatgcaggcgctcattaaaacagcatgttgctccaaaaaaaaaaaaaaaaaa

SEQ ID NO: 242 - DNA, 5411 bp, Homo sapiens
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 243 - DNA, 2306 bp, Homo sapiens
gagcttgagaattgctcctgccctgggaagaggctcagcacagaaagaggaaggacagcacagctgacagccgtgctcagagagtttctggatcctaggcttatctccacagaggagaacacacaagcagcagagaccatgggaaccctctcagcccctccctgcacacagcgcatcaaatggaaggggctcctgctcacagcatcacttttaaacttctggaacctgcccaccactgcccaagtcacgattgaagccgagccaaccaaagtttccgaggggaaggatgttcttctacttgtccacaatttgccccagaatcttaccggctacatctggtacaaagggcaaatgagggacctctaccattacattacatcatatgtagtagacggtgaaataattatatatgggcctgcatatagtggacgagaaacagcatattccaatgcatccctgctgatccagaatgtcacccgggaggacgcaggatcctacaccttacacatcataaagggagatgatgggactagaggagtaactggacgtttcaccttcaccttacacctggagactcctaagccctccatctccagcagcaacttaaatcccagggagaccatggaggctgtgagcttaacctgtgaccctgagactccagacgcaagctacctgtggtggatgaatggtcagagcctccctatgactcacagcttgaagctgtccgaaaccaacaggaccctctttctattgggtgtcacaaagtatactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttaaacttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacgggaccgatatggtggcatccgcagtgacccagtcaccctgaatgtcctctatggtccagacctccccagaatttacccttcattcacctattaccgttcaggagaagtcctctacttgtcctgttctgcggactctaacccaccggcacagtattcttggacaattaatgaaaagtttcagctaccaggacaaaagctctttatccgccatattactacaaagcatagcgggctctatgtttgctctgttcgtaactcagccactggcaaggaaagctccaaatccatgacagtcgaagtctctggtaagtggatcccagcatcgttggcaatagggttttaggtggagtctatctggcattcagagaagagtcaggaaaacaattgtattcccagcctgtgtcccatgggcacaagcaaatcccaaattctcctcctgaaccctccaaatttgtctaagaacttcgaaaactttaacaaacaggctgatatcttcataatattcccagcctagaccaagcaggaagaacattgatttcattgaaataattgataataatgaagataatgtttttatgatttttatttgaaaatttgctgattctttaaatggtttgttttctacattgatggaatttttctcttttaatctatctacagcttatagcagttcaataaactatacttctgggaaccgtaattgaaacatttacttttgctttctacctgactgccccagaattgggcaactattcatgagaattgatatgtttatggtaatacacatatttgcacaagtacagtaacaatctgctttctttgtaacatgacacatttgaaatcattggttatattaccaatgctttgattcggatgttatattaaaaacatagatagaatgaaccaatatgaactgcaggcaaagtctgaagtcagccttggtttggcttcctattctcaagaggtttgtgaagatttaatctcagattccttataaaaacttagagaaaagaaaattttagaagacagcctacatggtccattgctactcttgctgcacttatgtaaacaatcagaccacatttgaagaaactccacctattttgcaaacaaacttattctactgaaattatcattggtaaaagtagagatgcccatagagggaaaaattatgtggaaaataaaaactgtagtatacctaaaaaaaaaaaaaaaaaaaaaaaaaaaaaa

SEQ ID NO: 244 - DNA, 816 bp, Homo sapiens
caacaaaaaagagcctcaggatccagcacacattatcacaaacttagtgtccatccatcactgctgaccctctccggacctgactccacccctgagggacacaggtcagccttgaccaatgacttttaagtaccatggagaacagggggccagaacttcggcagtaaagaataaaaggccagacagagaggcagcagcacatatctgcttccgacacagctgcaatcactagcaagctctcaggcctggcatcatggtgcattttactgctgaggagaaggctgccgtcactagcctgtggagcaagatgaatgtggaagaggctggaggtgaagccttgggcagactcctcgttgtttacccctggacccagagattttttgacagctttggaaacctgtcgtctccctctgccatcctgggcaaccccaaggtcaaggcccatggcaagaaggtgctgacttcctttggagatgctattaaaaacatggacaacctcaagcccgcctttgctaagctgagtgagctgcactgtgacaagctgcatgtggatcctgagaacttcaagctcctgggtaacgtgatggtgattattctggctactcactttggcaaggagttcacccctgaagtgcaggctgcctggcagaagctggtgtctgctgtcgccattgccctggcccataagtaccactgagttctcttccagtttgcaggtgttcctgtgaccctgacaccctccttctgcacatggggactgggcttggccttgagagaaagccttctgtttaataaagtacattttcttcagtaatc

SEQ ID NO: 245 - DNA, 1801 bp, Homo sapiens
tttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaa

SEQ ID NO: 246 - DNA, 1559 bp, Homo sapiens
aaagaaccagttttcaggcggattgcctcagatcacactatctccacttgcccagccctgtggaagattagcggccatgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtagagaaggaactcaccccagaggtgctgcaagagtggctggatgaattggaggagatgatgctggtggtccacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcctttcctggtttttataagagaagttcctctgaacactattatcttcatgggcagagtagccaacccttgtgttaagtaaaatgttcttattctttgcacctcttcctatttttggtttgtgaacagaagtaaaaataaatacaaactacttccatctcacattaaaa

SEQ ID NO: 247 - DNA, 1594 bp, Homo sapiens
tacctttcccagcagagcacctgggttggtcccgaagcctccaaccacctgcacgcctgccagggcctctctggggcagccatgaagtccctcgtcctgctcctttgtcttgctcagctctggggctgccactcagccccacatggcccagggctgatttatagacaaccgaactgcgatgatccagaaactgaggaagcagctctggtggctatagactacatcaatcaaaaccttccttggggatacaaacacaccttgaaccagattgatgaagtaaaggtgtggcctcagcagccctccggagagctgtttgagattgaaatagacaccctggaaaccacctgccatgtgctggaccccacccctgtggcaagatgcagcgtgaggcagctgaaggagcatgctgtcgaaggagactgtgatttccagctgttgaaactagatggcaagttttccgtggtatacgcaaaatgtgattccagtccagactcagccgaggacgtgcgcaaggtgtgccaagactgccccctgctggccccgctgaacgacaccagggtggtgcacgccgcgaaagctgccctggccgccttcaacgctcagaacaacggctccaattttcagctggaggaaatttcccgggctcagcttgtgcccctcccaccttctacctatgtggagtttacagtgtctggcactgactgtgttgctaaagaggccacagaggcagccaagtgtaacctgctggcagaaaagcaatatggcttttgtaaggcaacactcagtgagaagcttggtggggcagaggttgcagtgacctgcatggtgttccaaacacagcccgtgagctcacagccccaaccagaaggtgccaatgaagcagtccccacacccgtggtggacccagatgcacctccgtcccctccacttggcgcacctggactccctccagctggctcacccccagactcccatgtgttactggcagctcctccaggacaccagttgcaccgggcgcactacgacctgcgccacaccttcatgggtgtggtctcattggggtcaccctcaggagaagtgtcgcacccccggaaaacacgcacagtggtgcagcctagtgttggtgctgctgctgggccagtggttcctccatgtccggggaggatcagacacttcaaggtctaggctagacatggcagagatgaggaggtttggcacagaaaacatagccaccattttgtccaagcctgggcatgggtggggggccttgtctgctggccacgcaagtgtcacatgcgatctacattaatatcaagtcttgactccctacttcccgtcattcctcacaggacagaagcagagtgggtggtggttatgtttgacagaaggcattaggttgacaacttgtcatgattttgacggtaagccaccatgattgtgttctctgcctctggttgaccttacaaaaaccattggaactgtgactttgaaaggtgctcttgctaagcttatatgtgcctgttaatgaaagtgcctgaaagaccttccttaataaagaaggttctaagctgaaaaaaaaaaaaaaaaaaaaaaa

SEQ ID NO: 248 - DNA, 1766 bp, Homo sapiens
gttatgcaatcaatgatctgggtttttctcttcagagaaatttgttgtacagaaaattgctgttgggatgaagctttgcagccttgcagtccttgtacccattgttctcttctgtgagcagcatgtcttcgcgtttcagagtggccaagttctagctgctcttcctagaacctctaggcaagttcaagttctacagaatcttactacaacatatgagattgttctctggcagccggtaacagctgaccttattgtgaagaaaaaacaagtccatttttttgtaaatgcatctgatgtcgacaatgtgaaagcccatttaaatgtgagcggaattccatgcagtgtcttgctggcagatgtggaagatcttattcaacagcagatttccaacgacacagtcagcccccgagcctccgcatcgtactatgaacagtatcactcactaaatgaaatctattcttggatagaatttataactgagaggcatcctgatatgcttacaaaaatccacattggatcctcatttgagaagtacccactctatgttttaaaggtttctggaaaagaacaagcagccaaaaatgccatatggattgactgtggaatccatgccagagaatggatctctcctgctttctgcttgtggttcataggccatataactcaattctatgggataatagggcaatataccaatctcctgaggcttgtggatttctatgttatgccagtggttaatgtggatggttatgactactcatggaaaaagaatcgaatgtggagaaagaaccgttctttctatgcgaacaatcattgcatcggaacagacctgaataggaactttgcttccaaacactggtgtgaggaaggtgcatccagttcctcatgctcggaaacctactgtggactttatcctgagtcagaaccagaagtgaaggcagtggctagtttcttgagaagaaatatcaaccagattaaagcatacatcagcatgcattcatactcccagcatatagtgtttccatattcctatacacgaagtaaaagcaaagaccatgaggaactgtctctagtagccagtgaagcagttcgtgctattgagaaaactagtaaaaataccaggtatacacatggccatggctcagaaaccttatacctagctcctggaggtggggacgattggatctatgatttgggcatcaaatattcgtttacaattgaacttcgagatacgggcacatacggattcttgctgccggagcgttacatcaaacccacctgtagagaagcttttgccgctgtctctaaaatagcttggcatgtcattaggaatgtttaatgcccctgattttatcattctgcttccgtattttaatttactgattccagcaagaccaaatcattgtatcaaattatttttaagttttatccgtagttttgataaaagattttcctattccttggttctgtcagagaacctaataagtgctactttgccattaaggcagactagggttcatgtctttttaccctttaaaaaaaattgtaaaagtctagttacctactttttctttgattttcgacgtttgactagccatctcaagcaagtttcgacgtttgactagccatctcaagcaagtttaatcaatgatcatctcacgctgatcattggatcctactcaacaaaaggaagggtggtcagaagtacattaaagatttctgctccaaattttcaataaatttctgcttgtgcctttagaaataca

SEQ ID NO: 249 - DNA, 1216 bp, Homo sapiens
ggctctgtctttttagcagacgaaaaccactttggtagtgccagtgtgactcatccacaatgatttctccagtgctcatcttgttctcgagttttctctgccatgttgctattgcaggacggacctgtcccaagccagatgatttaccattttccacagtggtcccgttaaaaacattctatgagccaggagaagagattacgtattcctgcaagccgggctatgtgtcccgaggagggatgagaaagtttatctgccctctcacaggactgtggcccatcaacactctgaaatgtacacccagagtatgtccttttgctggaatcttagaaaatggagccgtacgctatacgacttttgaatatcccaacacgatcagtttttcttgtaacactgggttttatctgaatggcgctgattctgccaagtgcactgaggaaggaaaatggagcccggagcttcctgtctgtgctcccatcatctgccctccaccatccatacctacgtttgcaacacttcgtgtttataagccatcagctggaaacaattccctctatcgggacacagcagtttttgaatgtttgccacaacatgcgatgtttggaaatgatacaattacctgcacgacacatggaaattggactaaattaccagaatgcagggaagtaaaatgcccattcccatcaagaccagacaatggatttgtgaactatcctgcaaaaccaacactttattacaaggataaagccacatttggctgccatgatggatattctctggatggcccggaagaaatagaatgtaccaaactgggaaactggtctgccatgccaagttgtaaagcatcttgtaaagtacctgtgaaaaaagccactgtggtgtaccaaggagagagagtaaagattcaggaaaaatttaagaatggaatgctacatggtgataaagtttctttcttctgcaaaaataaggaaaagaagtgtagctatacagaggatgctcagtgtatagatggcactatcgaagtccccaaatgcttcaaggaacacagttctctggctttttggaaaactgatgcatccgatgtaaagccatgctaaggtggttttcagattccacacaaaatgtcacacttgtttcttgttcatccaaggaacctaattgaaatttaaaaataaagctactgaatttattgccgcacccaaaaaaaaaaaaaaaa

SEQ ID NO: 250 - DNA, 2032 bp, Homo sapiens
tccatattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaatg

SEQ ID NO: 251 - DNA, 533 bp, Homo sapiens
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 252 - DNA, 14121 bp, Homo sapiens
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaagaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgacgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagaccctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaacaaccacaaaagcccaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacatttgcacaccgtgacttcagtgcagaatatgaagaagatggcaaatatgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaagaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcagtgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 253 - DNA, 1389 bp, Homo sapiens
atggctagggtactgggagcacccgttgcactggggttgtggagcctatgctggtctctggccattgccacccctcttcctccgactagtgcccatgggaatgttgctgaaggcgagaccaagccagacccagacgtgactgaacgctgctcagatggctggagctttgatgctaccaccctggatgacaatggaaccatgctgttttttaaaggggagtttgtgtggaagagtcacaaatgggaccgggagttaatctcagagagatggaagaatttccccagccctgtggatgctgcattccgtcaaggtcacaacagtgtctttctgatcaagggggacaaagtctgggtataccctcctgaaaagaaggagaaaggatacccaaagttgctccaagatgaatttcctggaatcccatccccactggatgcagctgtggaatgtcaccgtggagaatgtcaagctgaaggcgtcctcttcttccaaggtgaccgcgagtggttctgggacttggctacgggaaccatgaaggagcgttcctggccagctgttgggaactgctcctctgccctgagatggctgggccgctactactgcttccagggtaaccaattcctgcgcttcgaccctgtcaggggagaggtgcctcccaggtacccgcgggatgtccgagactacttcatgccctgccctggcagaggccatggacacaggaatgggactggccatgggaacagtacccaccatggccctgagtatatgcgctgtagcccacatctagtcttgtctgcactgacgtctgacaaccatggtgccacctatgccttcagtgggacccactactggcgtctggacaccagccgggatggctggcatagctggcccattgctcatcagtggccccagggtccttcagcagtggatgctgccttttcctgggaagaaaaactctatctggtccagggcacccaggtatatgtcttcctgacaaagggaggctataccctagtaagcggttatccgaagcggctggagaaggaagtcgggacccctcatgggattatcctggactctgtggatgcggcctttatctgccctgggtcttctcggctccatatcatggcaggacggcggctgtggtggctggacctgaagtcaggagcccaagccacgtggacagagcttccttggccccatgagaaggtagacggagccttgtgtatggaaaagtcccttggccctaactcatgttccgccaatggtcccggcttgtacctcatccatggtcccaatttgtactgctacagtgatgtggagaaactgaatgcagccaaggcccttccgcaaccccagaatgtgaccagtctcctgggctgcactcactga

SEQ ID NO: 254 - DNA, 2910 bp, Homo sapiens
acagggcagcaggagccttagagcatggacggtgccatggggcctcgggggctgctgttgtgcatgtacctggtatctctcctcatcctgcaggccatgcctgccctgggctcggctacaggcaggtccaagagcagcgagaagcgacaggctgtggacaccgctgtcgatggcgtgttcatccggagtttgaaagtcaactgcaaagtcacctctcgcttcgcccactatgttgtcaccagccaagtggtcaacactgccaatgaagccagggaagtggccttcgacctggaaatccccaagacagcattcatcagtgactttgccgttacagcagatggaaacgcatttatcggagacataaaggacaaggtgactgcatggaagcagtaccggaaagcagctatctcaggagagaatgccggccttgtcagggcctcggggagaactatggagcaattcaccatccacctcaccgtcaatccccagagcaaggtcacgtttcagctgacttatgaggaagtgctgaagagaaaccatatgcagtatgaaattgtcatcaaagtcaagcccaagcagctggtgcatcattttgagattgatgtggacatcttcgagccccaggggatcagcaagctggatgcccaggcctctttcctgccgaaggaactggcagcccaaactatcaagaagtccttctcaggaaaaaagggtcatgtgctgttccgtcccaccgtgagccagcagcagtcctgccccacatgctctacatccttactgaacgggcacttcaaggtgacctacgatgtcagtcgagacaagatctgcgacctcctggtggccaataaccactttgcccacttctttgccccccaaaacctgacaaacatgaacaagaacgtggtttttgtgattgacatcagtggctccatgagaggccagaaagtgaagcagaccaaggaggcactccttaaaattctgggggacatgcagccaggggactactttgacctggttctttttgggactcgagtacaatcgtggaagggctcgctggtgcaagcatctgaggccaacctacaagcagctcaagactttgtgcggggcttttccctggatgaggccacaaacctgaatggaggtttgctccggggaattgagatcttgaaccaagttcaggaaagcctcccagaactcagcaaccatgcctcaatactcatcatgttgacagatggcgatcccacagagggggtgacggaccgttcccaaatcctcaagaacgtccgcaacgccatccggggcaggttcccgctctacaacctgggtttcggccacaatgtggactttaactttctggaggtcatgtccatggagaacaacggacgggcccagagaatctacgaggaccatgatgccacccagcagctgcagggtttctacagccaggtagccaaacccctgctggtggatgtggatttgcagtacccccaggatgctgtcttggccctgacccagaaccaccataaacagtactacgaaggctcagagattgtggtggccgggcgcattgctgacaacaaacagagcagcttcaaggctgatgtgcaggcccatggggagggacaagaattcagtataacctgcctagtggatgaggaggagatgaagaaactgctccgagagcgtggccacatgctggagaaccacgtcgagcgcctctgggcctacctcaccatccaggagctgctggccaagcggatgaaggtggacagggaggagagggccaacctgtcatcccaggccctgcagatgtcgctggactatgggtttgtgaccccactgacctccatgagcatcaggggcatggcggaccaggacggcctgaagcccaccatcgacaagccctcagaggattctccgcctttggagatgctgggacccagaaggacgttcgtgctgtcagccttgcagccttctcctactcattccagctccaatacccagcggctgccagaccgagtgaccggcgtggacacagaccctcacttcatcatccacgtgccccagaaagaggacaccctgtgcttcaacatcaatgaggagcctggtgttatcctgagcctggtacaggaccccaacacaggcttctcagtgaatggacagctcattggcaacaaggccaggagccctgggcagcatgacggcacgtacttcgggcggctgggaatcgcaaaccctgccacggactttcagttggaagtgactcctcagaacattacgctgaaccccggctttggtgggcctgtgttttcctggagggaccaagctgtgctgcggcaggacggggtggtggtgaccatcaacaagaagaggaacctggtggtgtctgtggacgacggtggcacctttgaggttgttttgcaccgagtgtggaaggggagctcggtccaccaggacttcctgggcttctatgtgctggacagtcatcggatgtcagcccggacgcacgggctgctggggcaatttttccaccccatcggttttgaagtgtctgacatccacccaggctctgaccccacaaagccagatgccacgatggtggtgaggaaccgccggctcacggtcaccaggggtttgcaaaaagactacagcaaggacccgtggcatggggccgaggtgtcctgctggttcattcacaacaatggggctggactcatcgatggtgcctacactgattatatcgtccccgacatcttctgagccctctggccagcacgcctgtcctcccccggggccaaggcagaggaggaggacgacatcctgacctgctgctgaggctgtacctccttgactaagctggttccttgtgtcaaagcacctcatgccttccattaaagagaggccgtgtcc

SEQ ID NO: 255 - DNA, 1450 bp, Homo sapiens
attgtgcttggccaatgcctcttctgaagcagccatcccggcctcttggtactgctgaccccagccaggctacagggatcgattggagctgtccttggggctgtaattggccccagctgagcagggcaaacactgaggtcaactacaagccacaggccccttccccagcctcagttcacagctgccctgttgcagggaggcggtggcccttctgttgctagaccgagcctgtgggatataccaaggcagaggagcccatagccatgaggagcctcggggccctgctcttgctgctgagcgcctgcctggcggtgagcgctggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaagtttctctatcacaaatccaaatggaacataaccatggagtcctatgtggtccacaccaactatgatgagtatgccattttcctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgagagtccggagggctgtgctaccccaagaagaggaaggatcagggggtgggcaactggtaactgaagtcaccaagaaagaagattcctgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtggcggcctgcaatctccccatagtccggggcccctgccgagccttcatccagctctgggcatttgatgctgtcaaggggaagtgcgtcctcttcccctacgggggctgccagggcaacgggaacaagttctactcagagaaggagtgcagagagtactgcggtgtccctggtgatggtgatgaggagctgctgcgcttctccaactgacaactggccggtctgcaagtcagaggatggccagtgtctgtcccggggtcctgtggcaggcagcgccaagcaacctgggtccaaataaaaactaaattgtaaactcctgaaaaaaaaaaaaaaaaaaa

SEQ ID NO: 256 - DNA, 2243 bp, Homo sapiens
gggacactggggctgggggggtttgttgggctggtgagttttcgaggtgactgtgctgtgctcggtgaggggacgcccagagagccctgacccggggtcactccgtcgccgttctcctcttgtctacgtgctggacccggtgctacctttttacccacacttaagtgacgcaaaatgcccttcaatggcgagaagcagtgtgtgggagaggaccagccaagcgattctgattcttcccggttttccgaaagcatggcttcgctcagtgactatgaatgctccaggcagagctttgcaagtgactcctccagcaaatccagctctcctgcttcaacaagccctccaagggttgtaacatttgatgaagtgatggctacagcaaggaacttatcaaacttgactcttgctcatgagattgctgtaaatgagaactttcaattgaaacaagaggctctcccagaaaagagtttggctggtcgagtgaagcacattgttcaccaggccttctgggacgtcttggattcagaactaaatgctgaccctcctgagtttgaacatgccatcaaactgtttgaagaaatcagagagattcttctctcttttctcactcccggtggcaaccggcttcgcaaccaaatctgtgaagttttggacacagacctcattaggcagcaggctgagcacagtgctgttgacatccaaggcctggccaactatgtcatcagtacgatgggaaagctgtgtgctcccgtgcgagataatgatatcagagagttaaaggctactggcaacatcgtggaggtgctgagacaaatattccatgtcctggacctcatgcaaatggacatggccaattttacaattatgagtctcagaccgcaccttcaacgccagttggtggaatatgagagaaccaagttccaggaaattttggaagaaactccaagtgctcttgatcagactacagaatggataaaagaatctgtaaatgaagaattattttctctttctgagagtgctttaactcctggggccgaaaatacctccaagccaagcctgagccctactttggtgctaaataatagttacttgaaactgttacagtgggattatcagaaaaaagaattaccagagacacttatgacagatggagcacgtcttcaggaactaacagaaaagctgaatcaattgaaaattattgcctgcctgtccctaattaccaacaacatggtgggtgctattacaggaggcctgcctgagcttgcaagcaggttaacaaggatttcagctgttctacttgaaggcatgaacaaagagacctttaacttgaaggaagtcctgaattctattggtattcagacttgtgttgaggttaacaagaccctgatggaaagaggtttacccactttaaatgctgagattcaagctaatcttataggtcaattttcaagcattgaagaggaggacaatcctatctggtccttgattgataaacgaattaagctttacatgagaaggctactttgtcttccaagccctcaaaaatgcatgcctcctatgccaggaggcctagctgtcattcagcaggagctagaagccctaggctctcaatatgcaaacattgtgaatctcaacaaacaagtgtatggaccattttatgcaaatatacttcgaaagctgctcttcaatgaggaagccatggggaaggtagatgcttcacctcctactaactaaagaagaactgacattggacgagagattggaaatccagtactttggtatccagtccacttccattgatggcattagagatccagcacattctcagtactgtggtgcagtattagcccaaatctgtgtaatgggtaatattagcattacagaagacacacacatcacatagaccctcagaagacgtaaacatcacatagaccctatttgtgcatcattttcaagtttaaaacagatatttgtaatgaacagaaaacaatttgtaattaattatattacctatataatacttgtaaatgttttcttaaccatttatatttggcttatgacatttaacccctaaggagttgtttttctcacttgttattatcaaacctaatggtttttaattttggtacaactccttaaagggttgaaggttgtgacaataactgagggaactgatgttctgaataaatgatgtgaagtaaacacaattgtatttgaaaaaaaaaaaaaaaaaaaaaaaaaaa

SEQ ID NO: 257 - protein, 217 aa, Homo sapiens
MAPGSRTSLLLAFALLCLPWLQEAGAVQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 258 - protein, 138 aa, Homo sapiens
MNSLVSWQLLLFLCATHFGEPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 259 - protein, 196 aa, Homo sapiens
MRLPLLVSAGVLLVALLPCPPCRALLSRGPVPGARQAPQHPQPLDFFQPPPQSEQPQQPQARPVLLRMGEEYFLRLGNLNKSPAAPLSPASSLLAGGSGSRPSPEQATANFFRVLLQQLLLPRRSLDSPAALAERGARNALGGHQEAPERERRSEEPPISLDLTFHLLREVLEMARAEQLAQQAHSNRKLMEIIGK

SEQ ID NO: 260 - protein, 165 aa, Homo sapiens
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 261 - protein, 163 aa, Homo sapiens
MSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 262 - protein, 426 aa, Homo sapiens
MGPLPAPSCTQRITWKGLLLTASLLNFWNPPTTAEVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYLETPKPYISSSNLNPREAMEAVRLICDPETLDASYLWWMNGQSLPVTHRLQLSKTNRTLYLFGVTKYIAGPYECEIRNPVSASRSDPVTLNLLPKLPIPYITINNLNPRENKDVLAFTCEPKSENYTYIWWLNGQSLPVSPGVKRPIENRILILPSVTRNETGPYQCEIRDRYGGLRSNPVILNVLYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 263 - protein, 217 aa, Homo sapiens
MAAGSRTSLLLAFALLCLPWLQEAGAVQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 264 - protein, 308 aa, Homo sapiens
MPGQELRTLNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 265 - protein, 235 aa, Homo sapiens
MDPARPLGLSILLLFLTEAALGDAAQEPTGNNAEICLLPLDYGPCRALLLRYYYDRYTQSCRQFLYGGCEGNANNFYTWEACDDACWRIEKVPKVCRLQVSVDDQCEGSTEKYFFNLSSMTCEKFFSGGCHRNRIENRFPDEATCMGFCAPKKIPSFCYSPKDEGLCSANVTRYYFNPRYRTCDAFTYTGCGGNDNNFVSREDCKRACAKALKKKKKMPKLRFASRIRKIRKKQF

SEQ ID NO: 266 - protein, 179 aa, Homo sapiens
RPQSVCIQVLIKTVCCSTPPRVVCWRALRVPTNARALGLALKPARGFRPRPQWTYHPSIWESRPQQPDKGSSSASSRLSVHAHRSLISPTGTDVFIGQTDPHKKLPPKWTGPYTVILSTPTAVRVRGLPNWIHRTRVKLTPKAASSSKTLTAKCLSGPISPTKFKLTNIFFLKPKHKED

SEQ ID NO: 267 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 268 - protein, 426 aa, Homo sapiens
MGTLSAPPCTQRIKWKGLLLTASLLNFWNLPTTAQVTIEAEPTKVSEGKDVLLLVHNLPQNLTGYIWYKGQMRDLYHYITSYVVDGEIIIYGPAYSGRETAYSNASLLIQNVTREDAGSYTLHIIKGDDGTRGVTGRFTFTLHLETPKPSISSSNLNPRETMEAVSLTCDPETPDASYLWWMNGQSLPMTHSLKLSETNRTLFLLGVTKYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGEVLYLSCSADSNPPAQYSWTINEKFQLPGQKLFIRHITTKHSGLYVCSVRNSATGKESSKSMTVEVSGKWIPASLAIGF

SEQ ID NO: 269 - protein, 147 aa, Homo sapiens
MVHFTAEEKAAVTSLWSKMNVEEAGGEALGRLLVVYPWTQRFFDSFGNLSSPSAILGNPKVKAHGKKVLTSFGDAIKNMDNLKPAFAKLSELHCDKLHVDPENFKLLGNVMVIILATHFGKEFTPEVQAAWQKLVSAVAIALAHKYH

SEQ ID NO: 270 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 271 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 272 - protein, 367 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 273 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV

SEQ ID NO: 274 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 275 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 276 - protein, 99 aa, Homo sapiens
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 277 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 278 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 279 - protein, 911 aa, Homo sapiens
MDGAMGPRGLLLCMYLVSLLILQAMPALGSATGRSKSSEKRQAVDTAVDGVFIRSLKVNCKVTSRFAHYVVTSQVVNTANEAREVAFDLEIPKTAFISDFAVTADGNAFIGDIKDKVTAWKQYRKAAISGENAGLVRASGRTMEQFTIHLTVNPQSKVTFQLTYEEVLKRNHMQYEIVIKVKPKQLVHHFEIDVDIFEPQGISKLDAQASFLPKELAAQTIKKSFSGKKGHVLFRPTVSQQQSCPTCSTSLLNGHFKVTYDVSRDKICDLLVANNHFAHFFAPQNLTNMNKNVVFVIDISGSMRGQKVKQTKEALLKILGDMQPGDYFDLVLFGTRVQSWKGSLVQASEANLQAAQDFVRGFSLDEATNLNGGLLRGIEILNQVQESLPELSNHASILIMLTDGDPTEGVTDRSQILKNVRNAIRGRFPLYNLGFGHNVDFNFLEVMSMENNGRAQRIYEDHDATQQLQGFYSQVAKPLLVDVDLQYPQDAVLALTQNHHKQYYEGSEIVVAGRIADNKQSSFKADVQAHGEGQEFSITCLVDEEEMKKLLRERGHMLENHVERLWAYLTIQELLAKRMKVDREERANLSSQALQMSLDYGFVTPLTSMSIRGMADQDGLKPTIDKPSEDSPPLEMLGPRRTFVLSALQPSPTHSSSNTQRLPDRVTGVDTDPHFIIHVPQKEDTLCFNINEEPGVILSLVQDPNTGFSVNGQLIGNKARSPGQHDGTYFGRLGIANPATDFQLEVTPQNITLNPGFGGPVFSWRDQAVLRQDGVVVTINKKRNLVVSVDDGGTFEVVLHRVWKGSSVHQDFLGFYVLDSHRMSARTHGLLGQFFHPIGFEVSDIHPGSDPTKPDATMVVRNRRLTVTRGLQKDYSKDPWHGAEVSCWFIHNNGAGLIDGAYTDYIVPDIF

SEQ ID NO: 280 - protein, 352 aa, Homo sapiens
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 281 - protein, 519 aa, Homo sapiens
MPFNGEKQCVGEDQPSDSDSSRFSESMASLSDYECSRQSFASDSSSKSSSPASTSPPRVVTFDEVMATARNLSNLTLAHEIAVNENFQLKQEALPEKSLAGRVKHIVHQAFWDVLDSELNADPPEFEHAIKLFEEIREILLSFLTPGGNRLRNQICEVLDTDLIRQQAEHSAVDIQGLANYVISTMGKLCAPVRDNDIRELKATGNIVEVLRQIFHVLDLMQMDMANFTIMSLRPHLQRQLVEYERTKFQEILEETPSALDQTTEWIKESVNEELFSLSESALTPGAENTSKPSLSPTLVLNNSYLKLLQWDYQKKELPETLMTDGARLQELTEKLNQLKIIACLSLITNNMVGAITGGLPELASRLTRISAVLLEGMNKETFNLKEVLNSIGIQTCVEVNKTLMERGLPTLNAEIQANLIGQFSSIEEEDNPIWSLIDKRIKLYMRRLLCLPSPQKCMPPMPGGLAVIQQELEALGSQYANIVNLNKQVYGPFYANILRKLLFNEEAMGKVDASPPTN

(19) United States
(12) Patent Application Publication
(10) Pub. No.: US 2010/0304978 A1
(43) Pub. Date: Dec. 2, 2010

(54) Methods and compositions for identifying a fetal cell

(76) Inventors: David Xingfei Deng, Mountain View, CA (US); Yun Bao, Fremont, CA (US); Yue-Jen Chuu, Cupertino, CA (US); Daniel Shoemaker, San Diego, CA (US); David L. Robbins, Temecula, CA (US)

Correspondence Address: WILSON, SONSINI, GOODRICH & ROSATI, 650 PAGE MILL ROAD, PALO ALTO, CA, 94304-1050, US

(21) Appl. No.: 12/657,723

(22) Filed: Jan. 26, 2010

Related U.S. Application Data
(60) Provisional application No. 61/147,456, filed on Jan. 26, 2009.

(51) Int. Cl. C40B 30/00; C12Q 1/68

(52) U.S. Cl. 506/7; 435/6

(57) ABSTRACT

The present invention provides methods and compositions for specifically identifying a fetal cell. An initial screening of approximately 400 candidate genes by digital PCR in different fetal and adult tissues identified a subset of 24 gene markers specific for fetal nucleated RBC and trophoblasts. The specific expression of those genes was further evaluated and verified in more defined tissues and isolated cells through quantitative RT-PCR using custom Taqman probes specific for each gene. A subset of fetal cell specific markers (FCM) was tested and validated by RNA fluorescent in situ hybridization (FISH) in blood samples from non-pregnant women, and pre-termination and post-termination pregnant women. Applications of these gene markers include, but are not limited to, distinguishing a fetal cell from a maternal cell for fetal cell identification and genetic diagnosis, identifying circulating fetal cell types in maternal blood, purifying or enriching one or more fetal cells, and enumerating one or more fetal cells during fetal cell enrichment.

### CROSS-REFERENCE

This application claims the benefit of U.S. Patent Application Ser. No. 61/147,456, filed Jan. 26, 2009, which is incorporated herein by reference in its' entirety.

### BACKGROUND OF THE INVENTION

Circulating fetal cells (CFCs) are present in maternal blood during pregnancy. Successful isolation and enrichment of one or more CFCs from maternal peripheral blood can be used to perform noninvasive genetic diagnosis of fetal well being. However, the number of CFCs in circulating maternal blood is relatively low, with approximately one fetal cell per one ml of whole blood. Owing to their low numbers, it is technically challenging to enrich and purify a fetal cell from maternal blood samples.

Fetal call identification (FCID) using fetal cell-type specific markers (FCMs) can play a role in fetal cell enrichment, enumeration, and genetic analysis. FCID markers can be DNA, RNA or proteins. DNA markers, such as loci on the Y-chromosome or other chromosomes, can be used to distinguish a maternal and fetal cell. A fetal cell can be identified using techniques such as by RNA fluorescent in situ hybridization (FISH) or immunocytochemical (ICC) staining for one or more protein markers. Cell surface protein markers can also be used for both cell selection and identification.

A gene expression panel that can be used to identify a circulating fetal cell such as a fetal nucleated red blood cell (fnRBC) or a trophoblast would be useful for the enrichment, enumeration, purification or analysis of these cells. Currently, available fetal cell markers have some drawbacks and are not specific for the various fetal cell types present in maternal samples in the first and second trimesters.

Specific FCMs are useful in identification, enrichment, purification, and enumeration of a fetal cell. Identification of one or more genes whose expression is specific for a fetal cell can be used to identify a fetal cell, such as through RNA fluorescent in situ hybridization (FISH), and/or isolate a target fetal cell to high purity such as by immunocytometry. The corresponding protein markers of these genes can also be used in ICC for FCID.

### SUMMARY OF THE INVENTION

In one aspect, a method for identifying a fnRBC comprising detecting transcript or protein expression of a HBE, AFP, AHSG, or J42-4-d gene is provided. In one embodiment, said detecting comprises using at least two primers and at least one probe that anneals to a cDNA generated from a transcript expressed by said HBE, AFP, AHSG, or J42-4-d gene.

In another aspect, a method for identifying a trophoblast comprising detecting transcript or protein expression of a KISS1, LOC90625, AFP, hPL, beta-hCG, or FN1 gene is provided. In one embodiment, said detecting comprises using at least two primers and at least one probe that anneals to a cDNA generated from a transcript expressed by said KISS1, LOC90625, AFP, hPL, beta-hCG, or FN1 gene.

In another aspect, a method for identifying a fetal cell in a maternal sample is provided comprising detecting transcript or protein expression by a cell of one or more of the KISS1, LOC90625, FN1, or AHSG genes to distinguish said fetal cell from a maternal cell.

In another aspect, a method for identifying a fetal cell in a maternal sample is provided comprising detecting transcript or protein expression by a cell of three or more of the hPL, KISS1, LOC90625, FN1, PSG9, HBE, AFP, beta-hCG, AHSG or J42-4-d genes to distinguish said fetal cell from a maternal cell.

In one embodiment, the maternal sample is a maternal blood sample, amniocentesis sample, or cervical swab. In another embodiment, said fetal cell is a fetal nucleated RBC or a placental cell. In another embodiment, said sample is taken in the 1^{st} or early 2^{nd} trimester. In another embodiment, said sample is taken in the 2^{nd} trimester. In another embodiment, said fetal cell is a fetal nucleated red blood cell and said gene is AHSG. In another embodiment, said fetal cell is a trophoblast and said gene is FN1. In another embodiment, said detecting comprises RNA FISH, RNA-FISH with a molecule beacon probe, RT-PCR, Q-PCR, digital mRNA profiling, Northern blotting, ribonuclease protection assay, or RNA expression profiling using microarrays. In another embodiment, said detecting comprises binding a protein with one or more binding moieties. In another embodiment, said one or more binding moieties is an antibody, Fab fragment, Fc fragment, scFv fragment, peptidomimetic, or peptoid.

In another aspect, a method for identifying a fetal cell in a maternal sample is provided comprising: enriching a fetal cell and detecting protein or transcript expression of one or more genes by said fetal cell, wherein said expression of said one or more genes distinguishes said fetal cell from a maternal cell, wherein said one or more genes is hPL, CHS2, KISS1, GDF15, CRH, TFP12, CGB, LOC90625, FN1, COL1A2, PSG9, PSG1, AFP, APOC3, SERPINC1, AMBP, CPB2, ITIH1, APOH, HPX, beta-hCG, AHSG, APOB, or J42-4-d. In one embodiment, the step of enriching a fetal cell comprises one or more steps of density centrifugation, size based separation, affinity separation, magnetic separation, microfluidic fluorescent cell sorting, dielectrophoretic enrichment, or antibody separation. In another embodiment, the sample is a maternal blood sample, amniocentesis sample, or cervical swab. In another embodiment, said cell is a fetal nucleated RBC or a placental cell. In another embodiment, the method further comprises enriching a fetal nucleated RBC by magnetic enrichment. In another embodiment, the method further comprises enriching one or more fetal nucleated RBCs by anti-CD71 or anti-GLA selection. In another embodiment, the method further comprises enriching one or more trophoblasts by anti-HLA-G or anti-EGFR selection. In another embodiment, said cell is a fetal nucleated RBC and said one or more genes is AFP, AHSG, or J42-4-d. In another embodiment, said cell is a trophoblast and said one or more genes is KISS1, LOC90625, AFP, hPL, beta-hCG, or FN1. In another embodiment, said detecting is by RNA FISH, RNA-FISH with a molecule beacon probe, RT-PCR, Q-PCR, digital mRNA profiling, Northern blotting, ribonuclease protection assay, or RNA expression profiling using microarrays. In another embodiment, said fetal cell is from a maternal sample obtained in the 1^{st} trimester or 2^{nd} trimester of pregnancy. In another embodiment, said detecting protein expression comprises binding a protein with a binding moiety. In another embodiment, said binding moiety is an antibody, Fab fragment, Fc fragment, scFv fragment, peptidomimetic, or peptoid.

In another aspect, a method for identifying a fetal cell specific transcript is provided comprising isolating a transcript from a sample containing a fetal cell and a transcript from a sample lacking fetal cells; producing cDNAs of said transcripts; performing quantitative PCR on said cDNAs; and comparing results of said quantitative PCR between samples to identify a marker transcript with higher expression in a fetal cell relative to a non-fetal cell. In one embodiment, said fetal cell is first enriched from a maternal sample by size based separation. In another embodiment, the method further comprises a verifying step comprising detecting a marker transcript by quantitative PCR.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D illustrate embodiments of a size-based separation module.

FIG. 2A illustrates cells flowing through an array of obstacles.

FIG. 2B illustrates antibody coated posts.

FIG. 2C illustrates one embodiment of an affinity separation module.

FIG. 3 illustrates one embodiment of a magnetic separation module.

FIG. 4 illustrates one embodiment of a multiplex enrichment module of the present invention.

FIG. 5 illustrates exemplary genes that can be analyzed from enriched cells, such as epithelial cells, endothelial cells, circulating tumor cells, progenitor cells, etc.

FIG. 6 illustrates one embodiment for genotyping rare cell(s) or rare DNA using, e.g., Affymetrix DNA microarrays.

FIG. 7 illustrates one embodiment for genotyping rare cell(s) or rare DNA using, e.g., Illumina bead arrays.

FIG. 8 illustrates one embodiment for determining gene expression of rare cell(s) or rare DNA using, e.g., Affymetrix expression chips.

FIG. 9 illustrates one embodiment for determining gene expression of rare cell(s) or rare DNA using, e.g., Illumina bead arrays.

FIG. 10 illustrates one embodiment for high-throughput sequencing of rare cell(s) or rare DNA using, e.g., single molecule sequence by synthesis methods (e.g., Helicos BioSciences Corporation).

FIG. 11 illustrates one embodiment for high-throughput sequencing of rare cell(s) or rare DNA using, e.g., amplification of nucleic acid molecules on a bead (e.g., 454 Lifesciences).

FIG. 12 illustrates one embodiment for high-throughput sequencing of rare cell(s) or rare DNA using, e.g., clonal single molecule arrays technology (e.g., Solexa, Inc.).

FIG. 13 illustrates one embodiment for high-throughput sequencing of rare cell(s) or rare DNA using, e.g., single base polymerization using enhanced nucleotide fluorescence (e.g., Genovoxx GmbH).

FIG. 14 illustrates methods of fetal diagnostic assays. A fetal cell is isolated by CSM-HE enrichment of target cells from blood. The designation of a cell as a fetal cell can be confirmed using techniques comprising FISH staining (using slides or membranes and optionally an automated detector), FACS, and/or binning Binning can comprise distribution of enriched cells across wells in a plate (such as a 96 or 384 well plate), microencapsulation of cells in droplets that are separated in an emulsion, or by introduction of cells into microarrays of nanofluidic bins. A fetal cell is then identified using methods that can comprise the use of biomarkers (such as fetal (gamma) hemoglobin), allele-specific SNP panels that could detect fetal genome DNA, detection of differentially expressed maternal and fetal transcripts (such as Affymetrix chips), or primers and probes directed to fetal specific loci (such as the multi-repeat DYZ locus on the Y-chromosome). Binning sites that contain a fetal cell are then be analyzed for aneuploidy and/or other genetic defects using a technique such as CGH array detection, ultra deep sequencing (such as Solexa, 454, or mass spectrometry), STR analysis, or SNP detection.

FIG. 15 illustrates methods of fetal diagnostic assays, further comprising the step of whole genome amplification prior to analysis of aneuploidy and/or other genetic defects.

FIGS. 16A-D illustrate various embodiments of a size-based separation module.

FIGS. 17A and B illustrate cell smears of the product and waste fractions.

FIG. 18 illustrates an initial screening strategy for identifying fetal cell markers.

FIG. 19 illustrates an experimental setup for identification of fetal specific RNAs.

FIG. 20 illustrates a strategy for screening for fetal specific markers with a Fluidigm Chip.

FIG. 21 illustrates a strategy for verifying fetal specific markers.

FIG. 22 depicts an experimental protocol for verifying fetal specific markers.

FIG. 23 illustrates RNA FISH using cDNA probes.

FIG. 24 illustrates validation of gene labeling specificity by single cell analysis.

FIG. 25 illustrates a summary of a fetal cell marker screening.

FIG. 26A depicts 12 placental (trophoblast) specific markers.

FIG. 26B depicts 12 fetal liver (fnRBC) specific markers.

FIG. 27A depicts 13 fnRBC markers selected for further verification by RT-PCR.

FIG. 27B depicts 7 trophoblast markers selected for further verification by RT-PCR.

FIG. 28 displays the expression levels of gene markers for fnRBC in different tissues and isolated cells.

FIG. 29 displays the expression levels of gene markers for trophoblasts in different tissues and isolated cells.

FIG. 30 displays relative gene expression results and cell type specificity for RNA markers.

FIG. 31 illustrates RNA FISH in cultured cell-lines.

FIG. 32 illustrates RNA FISH in cord blood and non-pregnant samples.

FIG. 33 illustrates RNA FISH staining of fnRBC in pre-termination pregnant blood samples.

FIG. 34 illustrates preliminary results of RNA FISH staining in pre-term and post-term blood samples.

FIG. 35 illustrates detection of AFP expression in LCM isolated fnRBCs.

FIG. 36 illustrates that AFP is expressed in HBE antibody-stained positive cells, but not in negative cells.

FIG. 37 illustrates a strategy for enriching a fetal cell from maternal blood.

FIG. 38 illustrates a strategy for direct gene expression profiling from fetal cell enriched products.

FIG. 39 illustrates results that 35 HBE positive cell counts (one count/well).

FIG. 40 illustrates fetal trophoblast cell type and count.

FIG. 41 shows a comparison between fetal cell marker results with Y chromosome genotyping results using 10 ml whole blood.

FIG. 42 lists sequences of transcripts that can be fetal cell markers.

FIG. 43 lists sequences of proteins that can be fetal cell markers

FIG. 44 illustrates an overview for diagnosing, prognosing, or monitoring a prenatal condition in a fetus.

FIG. 45A-C illustrates one embodiment of a sample splitting apparatus.

FIG. 46 illustrates the detection of single copies of a fetal cell genome by qPCR.

FIG. 47 illustrates detection of single fetal cells in binned samples by SNP analysis.

FIG. 48 illustrates fetal cell enumeration by PCR analysis.

FIG. 49 illustrates a method for fetal cell identification and verification.

FIG. 50 illustrates expression of hPL, Beta-hCG and AFP in fetal trophoblasts.

FIGS. 51A-F illustrate isolated fetal cells confirmed by the reliable presence of male Y chromosome.

FIG. 52 illustrates trisomy 21 pathology in an isolated fetal nucleated red blood cell.

### DETAILED DESCRIPTION OF THE INVENTION

In general, methods and compositions for identifying a fetal cell by detecting expression of one or more genes are provided. Detection of expression of fetal cell-specific markers can be used to distinguish a fetal cell from a reference cell (e.g., maternal cell), distinguish between types of fetal cells, purify and/or enrich a fetal cell, and enumerate a fetal cell.

I. Sample Collection/Preparation

Sample Type

Samples containing one or more rare cells (e.g., one or more fetal cells) can be obtained from any animal in need of a diagnosis or prognosis or from an animal pregnant with a fetus in need of a diagnosis or prognosis. In one embodiment, a sample can be obtained from an animal suspected of being pregnant, pregnant, or that has been pregnant to detect the presence of a fetus or fetal abnormality. When the animal is a human, the sample can be taken during the first trimester (about the first three months of pregnancy), the 2nd trimester (about months 4-6 of pregnancy), or the third trimester (about months 7-9 of pregnancy). An animal of the present invention can be a human or a domesticated animal such as a cow, chicken, pig, horse, rabbit, dogs, cat, or goat. Samples derived from an animal, e.g., a human, can include, e.g., whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid. The sample can include. a sample of amniotic fluid (via amniocentesis), a biopsy of the placenta (e.g., by chorionic villi sampling, CVS), a maternal blood sample, an umbilical cord blood sample, or cervical swab.

Samples, including reference samples, can be collected for the purpose of identifying fetal cell-specific markers. Samples can include cord blood, peripheral blood cells from a non-pregnant woman (NP-PBC), adult bone marrow (ABM), fetal liver, or placenta. Fetal liver contains fnRBCs, and placenta contains trophoblasts and connective tissue. When the sample is taken from a pregnant woman, or a woman suspected of being pregnant, the sample can be taken in the 1^{st}, 2^{nd}, or 3^{rd} trimester.

To obtain a blood sample, a device known in the art can be used, e.g., a syringe or other vacuum suction device.

A maternal sample can contain one or more different types of fetal cells. A fetal cell can be any cell derived from a zygote, blastocyst, or embryo. A fetal cell can include, for example, T cells, B cells, natural-killer (NK) cells, antigen-presenting cells, erythroblasts, nucleated erythrocytes, leukocytes, pregnancy-associated progenitor cells (PAPCs), fetal mesenchymal stem cells, CD34+ cells (hematopoietic stem cells; HSCs); CD34+CD38+ cells, epithelial cells, endometrial cells, and placental cells. Placental cells can include trophoblasts, e.g., syncytiotrophoblasts (cells of the outer syncytial layer of the trophoblast) and cytotrophoblasts (cells of the inner layer of the trophoblast).

When obtaining a sample from an animal (e.g., blood sample), the amount of sample can vary depending upon animal size, its gestation period, and the condition being screened. In one embodiment, up to 50, 40, 30, 20, 10, 9, 8, 7, 6, 5, 4, 3, 2, or 1 mL of a sample is obtained. In one embodiment, 1-50, 2-40, 3-30, or 4-20 mL of sample is obtained. In one embodiment, more than 5, 10, 15, 20, 25, 30, 35, 40, 45, 50, 55, 60, 65, 70, 75, 80, 85, 90, 95, or 100 mL of a sample is obtained. In one embodiment between about 10-20 ml of a peripheral blood sample is obtained from a pregnant female.

To detect one or more fetal abnormalities, a blood sample can be obtained from a pregnant animal or human within 36, 24, 22, 20, 18, 16, 14, 12, 10, 8, 6, or 4 weeks of conception or even after a pregnancy has terminated.

In one embodiment, the sample is a maternal blood sample taken in the 1^{st} trimester or 2^{nd} trimester.

Pre-Treatment of a Sample

A blood sample can be optionally pre-treated or processed prior to enrichment. In one embodiment a pre-treatment step includes the addition of one or more reagents including, but not limited to, a membrane stabilizer, a preservative, a fixative, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent. In one embodiment the fixative used is formaldehyde, paraformaldehyde, glutaraldehyde, acrolein, glyoxal, malonaldehyde, diacetyl, polyaldehydes, carbodiimides, diisocyanates, diazonium compounds, diimido esters, diethylpyrocarbonate, maleimides, benzoquinone, and metallic ions, Dinitrobenzaldehyde, Dinitrobenzene sulfonic acids, or Dinitrobenzoic acids. In another embodiment the fixative is a Dinitrophenols, 3,5-Dinitrosalicylic acid, 2,4-Dinitrobenzoic acid, 5-Sulfosalicylic acid, 2,5-Dihydroxy-1,4-benzene disulfonic acid, 3,5-Dinitrobenzoic acid, 8-Hydroxyquinoline-5-sulfonic acid, 4-Nitrophenol, 3,5-Dinitrosalicylaldehyde, 3,5-Dinitroaniline, Paratoluene sulfonic acid, 2-Mesitylene sulfonic acid, 2-(Trifluoromethyl)benzoic acid, 3,5-Dinitrobenzonitrile, and 2,4-Dinitrobenzene sulfonic acid, 3,5-Dinitrobenzoic acid, 2,4-Dinitrobenzoic acid, 2,4-Dinitrobenzene sulfonic acid, 2,6-Dinitrobenzene sulfonic acid, 3,5-Dinitrobenzene sulfonic acid, or 2,4-Dinitrophenol. Fixatives are described in U.S. Pat. No. 5,422,277, issued Jun. 6, 1995, which is herein incorporated by reference. In one embodiment the cell membrane stabilizer used is potassium dichromate, a monosaccaride (e.g., glucose, fructose), a sugar alcohol (e.g., sorbitol, inositol), a disaccharide (e.g., sucrose, trehalose, lactose, maltose), a trisaccharide (e.g., raffinose), a oligosaccharide (e.g., cycloinulohexaose), a polysaccharide (e.g., ficoll, or dextran), or a polymer (e.g., poly-vinyl-pyrrolidone, polyethyleneglycol). In one embodiment the molecule that can change the magnetic property of, e.g., red blood cells' hemoglobin, is CO₂, N₂, or NaNO₂.

When a blood sample is obtained, a preservative such an anti-coagulation agent and/or a stabilizer can be added to the sample prior to enrichment. This addition allows for an extended time for analysis/detection. Thus, a sample, such as a blood sample, can be enriched and/or analyzed under any of the methods and systems herein within 1 week, 6 days, 5 days, 4 days, 3 days, 2 days, 1 day, 12 hrs, 6 hrs, 3 hrs, 2 hrs, or 1 hr from the time the sample is obtained.

II. Enrichment/Purification

Concentration

A sample (e.g., blood sample) can be enriched for one or more rare analytes or rare cells (e.g. one or more fetal cells or epithelial cells) using one or more any methods known in the art (e.g. Guetta, E M et al. Stem Cells Dev, 13(1):93-9 (2004), which is herein incorporated by reference in its entirety) or described herein. The enrichment increases the concentration of one or more rare cells or the ratio of one or more rare cells to non-rare cells in the sample. For example, enrichment can increase the concentration of an analyte of interest such as a fetal cell or epithelial cell by a factor of at least 2, 4, 6, 8, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000, 2,000,000,000, or 5,000,000,000 fold over its concentration in the original sample. In particular, when enriching one or more fetal cells from a maternal peripheral venous blood sample, the initial concentration of the one or more fetal cells in a sample can be about 1:50,000,000 and it can be increased to at least 1:5,000 or 1:500. Rare cells can also be enriched in a sample by the removal of fluid. A fluid sample (e.g., a blood sample) of greater than 10, 15, 20, 50, or 100 mL total volume can comprise rare components of interest, and it can be concentrated such that the rare component of interest is concentrated into a concentrated solution of less than 0.5, 1, 2, 3, 5, or mL total volume.

Density Gradient Centrifugation

Density gradient centrifugation is a method of separating cells based on the different densities of cell types in a mixture. The method can be used in a single step to separate cells into two compartments which contain cells that are either lighter or heavier than a specific density of the gradient material used. Density gradient centrifugation can be carried out through repetitive steps based on a series of different density gradients or in combination with affinity separation, cell panning, cell sorting, and the like. Alternatively, density gradient centrifugation can be performed using multiple layers of the different gradient densities. This method allows cells of different densities to form zones or bands at their corresponding densities after centrifugation. The cells in the different zones are then collected by placing a pipette at the appropriate location. Methods for enriching specific cell-types by density gradient centrifugation are described in U.S. Pat. No. 5,840,502, which is herein incorporated by reference in its entirety.

Methods of identifying fetal cells in a specimen using density gradient centrifugation utilize density gradient medium. The density gradient medium can be colloidal polyvinylpyrrolidone-coated silica (e.g. PercolD, Nycodenz, a nonionic polysucrose (Ficoll) either alone or with sodium diatrizoate (e.g. Ficoll-Paque or Histopaque), or mixtures thereof. The density of the reagent employed is selected to separate the fetal cells of interest from other blood components.

Enrichment can occur using one or more types of separation modules. Several different modules are described herein, all of which can be fluidly coupled with one another in series for enhanced performance.

Enrichment by Lysis

In one embodiment, enrichment occurs by selective lysis. In one embodiment, a blood sample can be combined with an agent that selectively lyses one or more cells or components in a blood sample. For example, one or more fetal cells can be selectively lysed and their nuclei released when a blood sample including one or more fetal cells is combined with deionized water. Such selective lysis allows for the subsequent enrichment of fetal nuclei using, e.g., size or affinity based separation. In another example platelets and/or enucleated red blood cells are selectively lysed to generate a sample enriched in nucleated cells, such as fetal nucleated red blood cells (fnRBC's), maternal nucleated blood cells (mnBC), or epithelial cells. fnRBCs can be subsequently separated from mnBC's using, e.g., antigen-i affinity or differences in. hemoglobin.

Size-Based Enrichment

In one embodiment, enrichment of rare cells occurs using one or more size-based separation modules. Examples of size-based separation modules include filtration modules, sieves, matrixes, etc. Examples of size-based separation modules contemplated by the present invention include those disclosed in International Publication No. WO 2004/113877, which is herein incorporated by reference in its entirety. Other size based separation modules are disclosed in International Publication No. WO 2004/0144651 and U.S. Patent Application Publication Nos. US20080138809A1 and US20080220422A1, which are herein incorporated by reference in their entirety.

In one embodiment, a size-based separation module comprises one or more arrays of obstacles forming a network of gaps. The obstacles are configured to direct particles as they flow through the array/network of gaps into different directions or outlets based on the particle's hydrodynamic size. For example, as a blood sample flows through an array of obstacles, nucleated cells or cells having a hydrodynamic size larger than a predetermined size, e.g., 8 microns, are directed to a first outlet located on the opposite side of the array of obstacles from the fluid flow inlet, while the enucleated cells or cells having a hydrodynamic size smaller than a predetermined size, e.g., 8 microns, are directed to a second outlet also located on the opposite side of the array of obstacles from the fluid flow inlet.

An array can be configured to separate cells smaller or larger than a predetermined size by adjusting the size of the gaps, obstacles, and offset in the period between each successive row of obstacles. For example, in one embodiment, obstacles or gaps between obstacles can be up to 10, 20, 50, 70, 100, 120, 150, 170, or 200 microns in length or about 2, 4, 6, 8 or 10 microns in length. In one embodiment, an array for size-based separation includes more than 100, 500, 1,000, 5,000, 10,000, 50,000 or 100,000 obstacles that are arranged into more than 10, 20, 50, 100, 200, 500, or 1000 rows. In one embodiment, obstacles in a first row of obstacles are offset from a previous (upstream) row of obstacles by up to 50% the period of the previous row of obstacles. In one embodiment, obstacles in a first row of obstacles are offset from a previous row of obstacles by up to 45, 40, 35, 30, 25, 20, 15 or 10% the period of the previous row of obstacles. Furthermore, the distance between a first row of obstacles and a second row of obstacles can be up to 10, 20, 50, 70, 100, 120, 150, 170 or 200 microns. A particular offset can be continuous (repeating for multiple rows) or non-continuous. In one embodiment, a separation module includes multiple discrete arrays of obstacles fluidly coupled such that they are in series with one another. Each array of obstacles has a continuous offset. But each subsequent (downstream) array of obstacles has an offset that is different from the previous (upstream) offset. In one embodiment, each subsequent array of obstacles has a smaller offset that the previous array of obstacles. This arrangement allows for a refinement in the separation process as cells migrate through the array of obstacles. Thus, a plurality of arrays can be fluidly coupled in series or in parallel, (e.g., more than 2, 4, 6, 8, 10, 20, 30, 40, 50). Fluidly coupling separation modules (e.g., arrays) in parallel allows for high-throughput analysis of the sample, such that at least 1, 2, 5, 10, 20, 50, 100, 200, or 500 mL per hour flows through the enrichment modules or at least 1, 5, 10, or 50 million cells per hour are sorted or flow through the device.

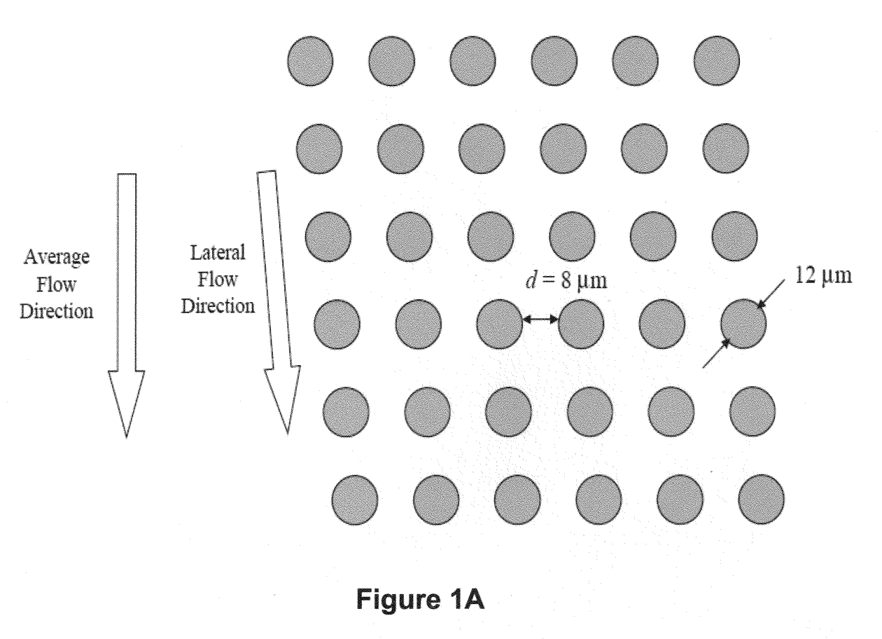

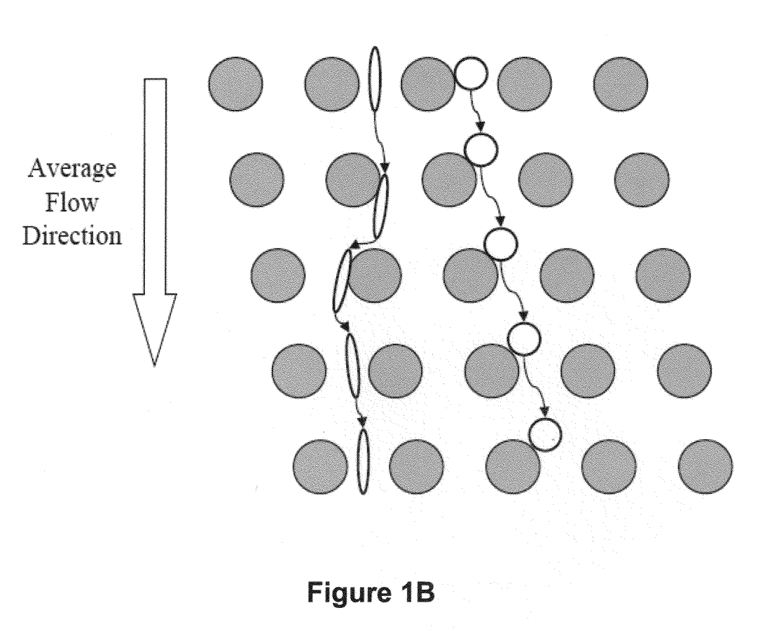

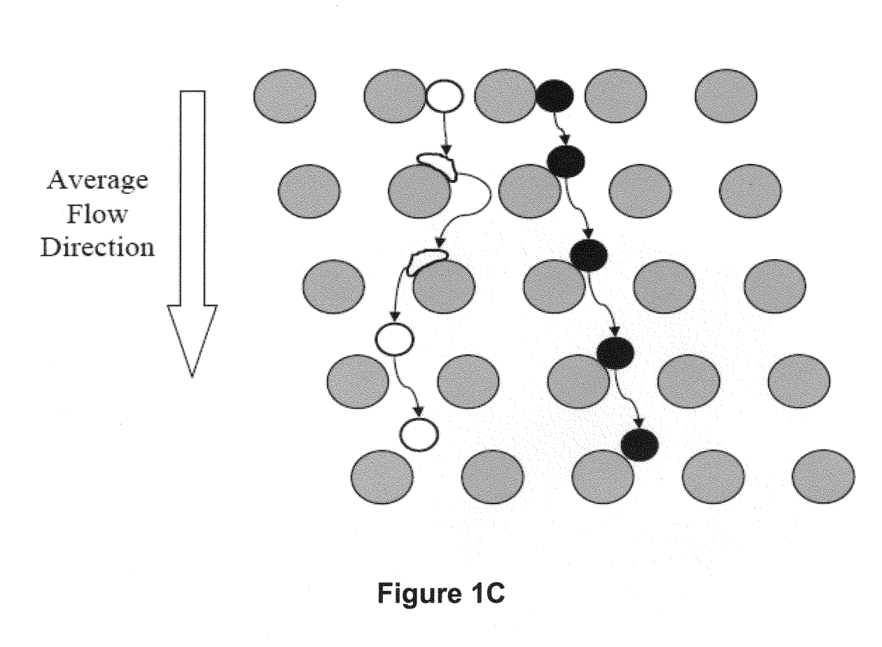

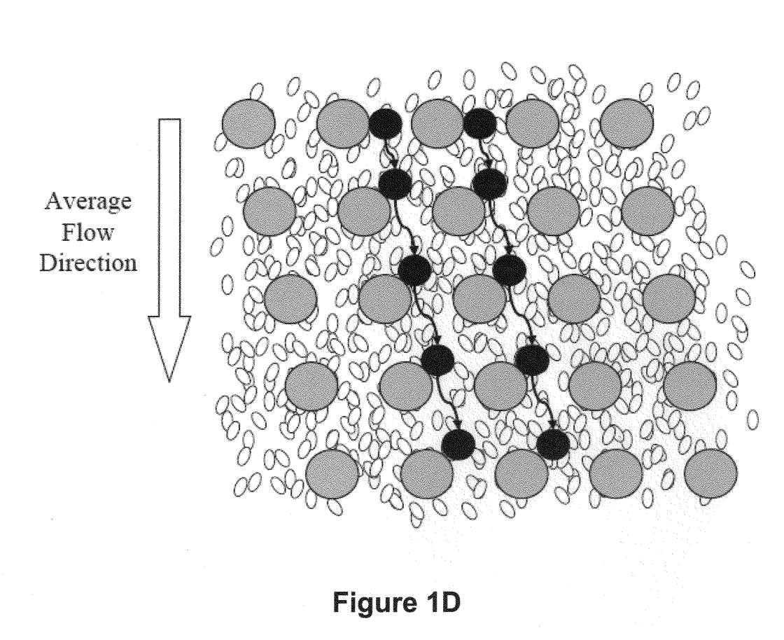

FIG. 1A illustrates an example of a size-based separation module. In one embodiment, a fetal cell can be labeled by (which can be of any shape) are coupled to a flat substrate to form an array of gaps. A transparent cover or lid can be used to cover the array. The obstacles form a two-dimensional array with each successive row shifted horizontally with respect to the previous row of obstacles, where the array of obstacles directs one or more components having a hydrodynamic size smaller than a predetermined size in a first direction and one or more components having a hydrodynamic size larger that a predetermined size in a second direction. For enriching epithelial cells from enucleated cells, the predetermined size of gaps in an array of obstacles can be 6-12 μm or 6-8 μm. For enriching one or more fetal cells from a mixed sample (e.g., maternal blood sample) the predetermined size of gaps in an array of obstacles can be between 4-10 μm or 6-8 μm. The flow of sample into the array of obstacles can be aligned at a small angle (flow angle) with respect to a line-of-sight of the array. Optionally, the array is coupled to an infusion pump to perfuse the sample through the obstacles. The flow conditions of the size-based separation module described herein are such that cells are sorted by the array with minimal damage. This allows for downstream analysis of intact cells and intact nuclei to be more efficient and reliable.

In one embodiment, a size-based separation module comprises an array of obstacles configured to direct cells larger than a predetermined size to migrate along a line-of-sight within the array (e.g., towards a first outlet or bypass channel leading to a first outlet), while directing cells and analytes smaller than a predetermined size to migrate through the array of obstacles in a different direction than the larger cells (e.g., towards a second outlet). Such embodiments are illustrated in part in FIGS. 1B-1D.

A variety of enrichment protocols can be utilized. In one embodiment the cells are handled gently to reduce mechanical damage to the cells or their DNA. This gentle handling can serve to preserve the small number of one or more fetal cells in the sample. Integrity of the nucleic acid being evaluated is an important feature to permit the distinction between the genomic material from the one or more fetal cells and other cells in the sample. In particular, the enrichment and separation of one or more fetal cells using the arrays of obstacles provides gentle treatment which minimizes cellular damage. Moreover, this gentle treatment maximizes nucleic acid integrity, permits exceptional levels of separation, and allows for the ability to subsequently utilize various formats to analyze the genome of the cells.

Affinity-Based Enrichment

In one embodiment, enrichment of one or more rare cells (e.g., one or more fetal cells or epithelial cells) occurs using one or more capture modules that selectively inhibit the mobility of one or more cells of interest. In one embodiment, a capture module is fluidly coupled downstream to a size-based separation module. Capture modules can include a substrate having multiple obstacles that restrict the movement of cells or analytes greater than a predetermined size. Examples of capture modules that inhibit the migration of cells based on size are disclosed in U.S. Pat. Nos. 5,837,115 and 6,692,952, which are herein incorporated by reference in their entirety.

In one embodiment, a capture module includes a two dimensional array of obstacles that selectively filters or captures cells or analytes having a hydrodynamic size greater than a particular gap size (predetermined size), International Publication No: WO 2004/113877, which is herein incorporated by reference in its entirety.

In one embodiment a capture module captures analytes (e.g., cells of interest or not of interest) based on their affinity for a binding moiety. For example, an affinity-based separation module that can capture cells or analytes can include an array of obstacles adapted for permitting sample flow through, but for the fact that the obstacles are covered with binding moieties that selectively bind one or more analytes (e.g., cell populations) of interest (e.g., one or more red blood cells, fetal cells, epithelial cells or nucleated cells) or analytes not-of-interest (e.g., white blood cells). Arrays of obstacles adapted for separation by capture can include obstacles having one or more shapes and can be arranged in a uniform or non-uniform order. In one embodiment, a two-dimensional array of obstacles is staggered such that each subsequent row of obstacles is offset from the previous row of obstacles to increase the number of interactions between the analytes being sorted (separated) and the obstacles. Other types of binding modules can be used.

Binding moieties coupled to the obstacles can include e.g., proteins (e.g., ligands/receptors), nucleic acids having complementary counterparts in retained analytes, antibodies, etc. In one embodiment, an affinity-based separation module comprises a two-dimensional array of obstacles covered with one or more antibodies that are: anti-CD71, anti-CD235a, anti-CD36, anti-carbohydrates, anti-selectin, anti-CD45, anti-GPA, anti-antigen-i, anti-EpCAM, anti-E-cadherin, anti-Muc-1, anti-hPL, anti-CHS2, anti-KISS1, anti-GDF15, anti-CRH, anti-TFP12, anti-CGB, anti-LOC90625, anti-FN1, anti-COL1A2, anti-PSG9, anti-PSG1, anti-HBE, anti-AFP, anti-APOC3, anti-SERPINC1, anti-AMBP, anti-CPB2, anti-ITIH1, anti-APOH, anti-HPX, anti-beta-hCG, anti-AHSG, anti-APOB, or anti-J42-4-d.

In one embodiment, a fnRBC is enriched using anti-CD71 or anti-GLA selection. In another embodiment, a trophoblast is enriched using anti-HLA-G or anti-EGFR selection. In another embodiment, a fnRBC is enriched using one or more antibodies or antibody fragments that can bind a protein expressed from the genes HBE, AFP, APOC3, SERPINC1, AMBP, CPB2, ITIH1, APOH, HPX, beta-hCG, AHSG, APOB, or J42-4-d. In another embodiment, a trophoblast is enriched using one or more antibodies or antibody fragments that can bind a protein expressed from the genes hPL, CHS2, KISS1, GDF15, CRH, TFP12, CGB, LOC90625, FN1, COL1A2, PSG9, or PSG1.

The binding moiety can be a single moiety, e.g., a polypeptide or protein, or it can include two or more moieties, e.g., a pair of polypeptides such as a pair of single chain antibody domains. Methods of generating antibodies are well know to those skilled in the art, e.g., by immunization strategies for the generation of monoclonal or polyclonal antibodies or in vitro methods for generating alternative binding members. Polyclonal antibodies can include, e.g., sheep, goat, rabbit, or rat polyclonal antibody. In addition any suitable molecule capable of high affinity binding can be used including antibody fragments such as single chain antibodies (scFv), Fab and scFv antibodies which can be obtained by phage-display or single domain antibodies (VHH) or chimeric antibodies. The binding moiety can be derived from a naturally occurring protein or polypeptide; it can be designed de novo, or it can be selected from a library. For example, the binding moiety can be or be derived from an antibody, a single chain antibody (scFv), a single domain antibody (VHH), a lipocalin, a single chain MHC molecule, an Anticalin™ (Pieris), an Affibody™, a nanobody (Ablynx) or a Trinectin™ (Phylos). Methods of generating binding members of various types are well known in the art.

Antibodies

A binding member can according to the invention be an antibody, such as any suitable antibody known in the art including other immunologically active fragments of antibodies or single chain antibodies. Antibody molecules are typically Y-shaped molecules whose basic unit consist of four polypeptides, two identical heavy chains and two identical light chains, which are covalently linked together by disulfide bonds. Each of these chains is folded in discrete domains. The C-terminal regions of both heavy and light chains are conserved in sequence and are called the constant regions, also known as C-domains. The N-terminal regions, also known as V-domains, are variable in sequence and are responsible for the antibody specificity. The antibody specifically recognizes and binds to an antigen mainly through six ‘short complementarity-determining regions located in their V-domains.

Antibody Fragments

In one embodiment of the invention the binding member is a fragment of an antibody, e.g., an antigen binding fragment or a variable region. Examples of antibody fragments useful with the present invention include Fab, Fab′, F(ab′) 2 and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′) 2 fragment that has two antigen binding fragments which are capable of cross-linking antigen, and a residual other fragment (which is termed pFc′).

Additional fragments can include diabodies, linear antibodies, single-chain antibody molecules, and multispecific antibodies formed from antibody fragments.

The antibody fragments Fab, Fv and scFv differ from whole antibodies in that the antibody fragments carry only a single antigen-binding site. Recombinant fragments with two binding sites have been made in several ways, for example, by chemical cross-linking of cysteine residues introduced at the C-terminus of the VH of an Fv (Cumber et al., 1992 which is herein incorporated by reference in its entirety), or at the C-terminus of the VL of an scFv (Pack and Pluckthun, 1992, which is herein incorporated by reference in its entirety), or through the hinge cysteine residues of Fab's (Carter et al., 1992, which is herein incorporated by reference in its entirety).

Antibody fragments retain some or essentially all the ability of an antibody to selectively bind with its antigen or receptor. Examples of antibody fragments include the following:

Fab is the fragment that contains a monovalent antigen-binding fragment of an antibody molecule. A Fab fragment can be produced by digestion of whole antibody with the enzyme papain to yield an intact light chain and a portion of one heavy chain.

Fab′ is the fragment of an antibody molecule and can be obtained by treating whole antibody with pepsin, followed by reduction, to yield an intact light chain and a portion of the heavy chain. Two Fab′ fragments are obtained per antibody molecule. Fab 1 fragments differ from Fab fragments by the addition of a few residues at the carboxyl terminus of the heavy chain CH 1 domain including one or more cysteines from the antibody hinge region.

(Fab′)₂ is the fragment of an antibody that can be obtained by treating whole antibody with the enzyme pepsin without subsequent reduction. F(ab′)2 is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (VH-V L dimer). It is in this configuration that the three CDRs of each variable domain interact to define an antigen binding site on the surface of the VH-V L dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The antibody can be a single chain antibody (“SCA”), defined as a genetically engineered molecule containing the variable region of the light chain, the variable region of the heavy chain, linked by a suitable polypeptide linker as a genetically fused single chain molecule. Such single chain anti-bodies are also referred to as “single-chain Fv” or “sFv” antibody fragments. Generally, the Fv polypeptide further comprises a polypeptide linker between the VH and VL domains that enables the sFv to form the desired structure for antigen binding.

The antibody fragments according to the invention can be produced in any suitable manner known to the person skilled in the art. Several microbial expression systems have already been developed for producing active antibody fragments, e.g., the production of Fab in various hosts, such as E. coli, yeast, and the filamentous fungus Trichoderma reesei are known in the art. The recombinant protein yields in these alternative systems can be relatively high (1-2 g/l for Fab secreted to the periplasmic space of E. coli in high cell density fermentation or at a lower level, e.g. about 0.1 mg/l for Fab in yeast in fermenters, and 150 mg/l for a fusion protein CBHI-Fab and 1 mg/l for Fab in Trichoderma in fermenters and such production is very cheap compared to whole antibody production in mammalian cells (hybridoma, myeloma, CHO).

The fragments can be produced as Fab's or as Fv's, but additionally it has been shown that a VH and a VL can be genetically linked in either order by a flexible polypeptide linker, which combination is known as an scFv.

Natural Single Domain Antibodies

Heavy-chain antibodies (HCAbs) are naturally produced by camelids (camels, dromedaries and llamas). HCAbs are homodimers of heavy chains only, devoid of light chains and the first constant domain (Hamers-Casterman et al., 1993, which is herein incorporated by reference in its entirety). The possibility to immunize these animals allows for the cloning, selection and production of an antigen binding unit consisting of a single-domain only. Furthermore these minimal-sized antigen binding fragments are well expressed in bacteria, interact with the antigen with high affinity and are very stable.

New or Nurse Shark Antigen Receptor (NAR) protein exists as a dimer of two heavy chains with no associated light chains. Each chain is composed of one variable (V) and five constant domains. The NAR proteins constitute a single immunoglobulin variable-like domain (Greenberg et al) which is much lighter than an antibody molecule.

According to the invention natural single domain antibodies can be considered an antibody fragment. The proteins can be produced and purified by any suitable method know by a person skilled in the art as described above.

In a further embodiment the binding member is active fragments of antibodies selected from Fab, Fab', F(ab)2, Fv, HCAbs and NARs.

In one embodiment of the methods and compositions of the provided invention, one or more antibodies are used that can bind one or more proteins expressed by a fetal cell from a hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d gene.

In one embodiment of the methods and compositions of the provided invention, one or more antibodies are used to bind one or more proteins expressed by an fnRBC from a HBE, AFP, AHSG, or J42-4-d gen.

In one embodiment of the methods and composition of the provided invention, one or more antibodies are used to bind one or more proteins expressed by a trophoblast from an hPL, beta-hCG, FN1, KISS1, or LOC90625 gene.

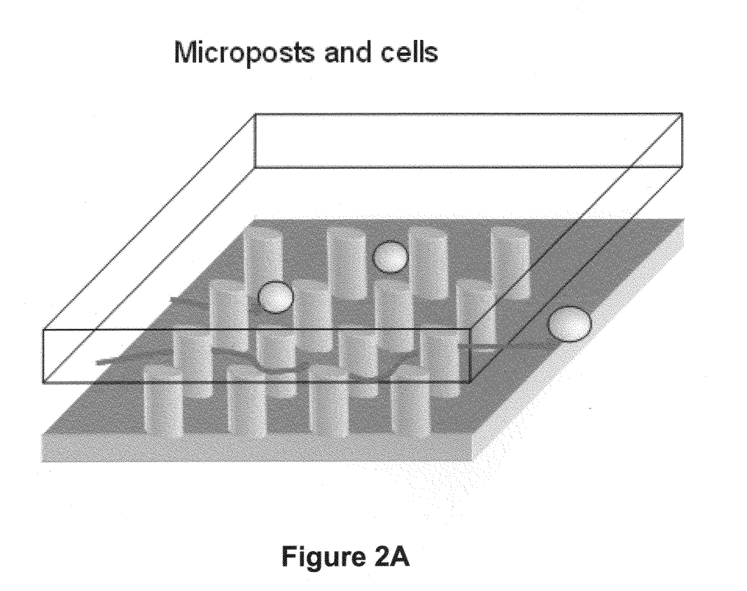

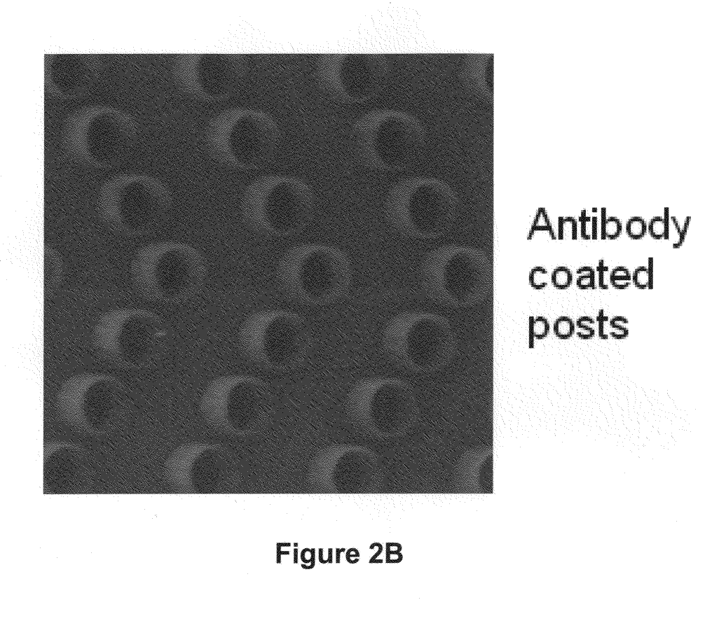

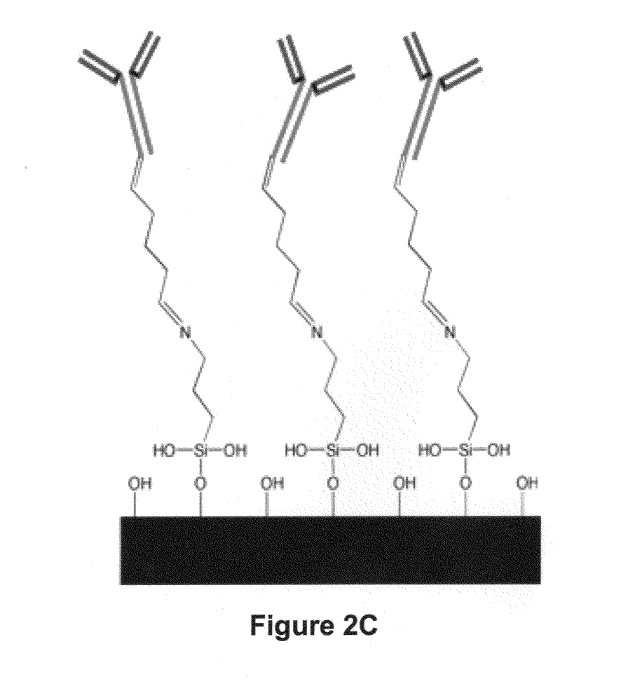

FIG. 2A illustrates a path of a first analyte through an array of posts wherein an analyte that does not specifically bind to a post continues to migrate through the array, while an analyte that does bind a post is captured by the array. FIG. 2B is a picture of antibody coated posts. FIG. 2C illustrates an embodiment of antibodies coupled to a substrate (e.g., obstacles, side walls, etc.) as contemplated by the present invention. Examples of such affinity-based separation modules are described in International Publication No. WO 2004/029221, which is herein incorporated by reference in its entirety.

Magnetic-Based Enrichment

In one embodiment, a capture module utilizes a magnetic field to separate and/or enrich one or more analytes (cells) based on a magnetic property or magnetic potential in such analyte of interest or an analyte not of interest. For example, red blood cells which are slightly diamagnetic (repelled by magnetic field) in physiological conditions can be made paramagnetic (attributed by magnetic field) by deoxygenation of the hemoglobin into methemoglobin. This magnetic property can be achieved through physical or chemical treatment of the red blood cells. Thus, a sample containing one or more red blood cells and one or more white blood cells can be enriched for the red blood cells by first inducing a magnetic property in the red blood cells and then separating the red blood cells from the white blood cells by flowing the sample through a magnetic field (uniform or non-uniform).

For example, a maternal blood sample can flow first through a size-based separation module to remove enucleated cells and cellular components (e.g., analytes having a hydrodynamic size less than 6 gins) based on size. Subsequently, the enriched nucleated cells (e.g., analytes having a hydrodynamic size greater than 6 μms) white blood cells and nucleated red blood cells are treated with a reagent, such as CO₂, N₂, or NaNO₂, that changes the magnetic property of the red blood cells' hemoglobin. Other means of rendering cells magnetic include by adsorption of magnetic cations. Paramagnetic cations include, for example, Cr⁺³, Co⁺², Mn⁺², Ni⁺², Fe⁺³, Fe⁺², La⁺³, Cu⁺², GD⁺³, Ce⁺³, Tb⁺³, Pr⁺³, Dy⁺³, Nd⁺³, Ho⁺³, Pm⁺³, Er⁺³, Sm⁺³, Tm⁺³, Eu⁺³, Yb⁺³, and Lu⁺³ (U.S. Patent Application Publication No. 20060078502, which is herein incorporated by reference in its entirety). For example, red blood cells can be rendered paramagnetic with chromium by contacting cells with an aqueous solution of chromate ions (Eisenberg et al. U.S. Pat. No. 4,669,481, which is herein incorporated by reference in its entirety).

The treated sample then flows through a magnetic field (e.g., a column coupled to an external magnet), such that the paramagnetic analytes (e.g., red blood cells) will be captured by the magnetic field while the white blood cells and any other non-red blood cells will flow through the device to result in a sample enriched in nucleated red blood cells (including fetal nucleated red blood cells or fnRBC's). Additional examples of magnetic separation modules are described in U.S. application Ser. No. 11/323,971, filed Dec. 29, 2005, entitled “Devices and Methods for Magnetic Enrichment of Cells and Other Particles” and U.S. application Ser. No. 11/227,904, filed Sep. 15, 2005, entitled “Devices and Methods for Enrichment and Alteration of Cells and Other Particles”, which are herein incorporated by reference in their entirety.

In one embodiment, where the analyte desired to be separated (e.g., red blood cells nucleated red blood cells, placental cells (e.g., trophoblasts) or white blood cells) can be coupled to a magnetic particle (e.g., a bead) or compound (e.g., Fe³⁺) to give the analyte a magnetic property. In one embodiment, a bead can be coupled to an antibody that selectively binds to an analyte of interest, such as a fetal cell. In one embodiment the bead is couple to an antibody or fragment of an antibody that is an anti CD71, anti-CD75, anti-hPL, anti-CHS2, anti-KISS1, anti-GDF15, anti-CRH, anti-TFP12, anti-CGB, anti-LOC90625, anti-FN1, anti-COL1A2, anti-PSG9, anti-PSG1, anti-HBE, anti-AFP, anti-APOC3, anti-SERPINC1, anti-AMBP, anti-CPB2, anti-ITIH1, anti-APOH, anti-HPX, anti-beta-hCG, anti-AHSG, anti-APOB, or anti-J42-4-d antibody or fragment of an antibody. In one embodiment a magnetic compound, such as Fe³⁺, can be coupled to an antibody such as those described above. The magnetic particles or magnetic antibodies herein can be coupled to any one or more of the devices herein prior to contact with a sample or can be mixed with the sample prior to delivery of the sample to the device(s). Magnetic particles can also be used to decorate one or more analytes (cells of interest or not of interest) to increase the size prior to performing size-based separation.

A magnetic field used to separate analytes/cells in any of the embodiments herein can be uniform or non-uniform as well as external or internal to the device(s) herein. An external magnetic field is one whose source is outside a device herein (e.g., container, channel, obstacles). An internal magnetic field is one whose source is within a device contemplated herein. An example of an internal magnetic field is one where magnetic particles can be attached to obstacles present in the device (or manipulated to create obstacles) to increase surface area for analytes to interact with to increase the likelihood of binding. Analytes captured by a magnetic field can be released by demagnetizing the magnetic regions retaining the magnetic particles. For selective release of analytes from regions, the demagnetization can be limited to selected obstacles or regions. For example, the magnetic field can be designed to be electromagnetic, enabling turn-on and turn-off off the magnetic fields for each individual region or obstacle at will.

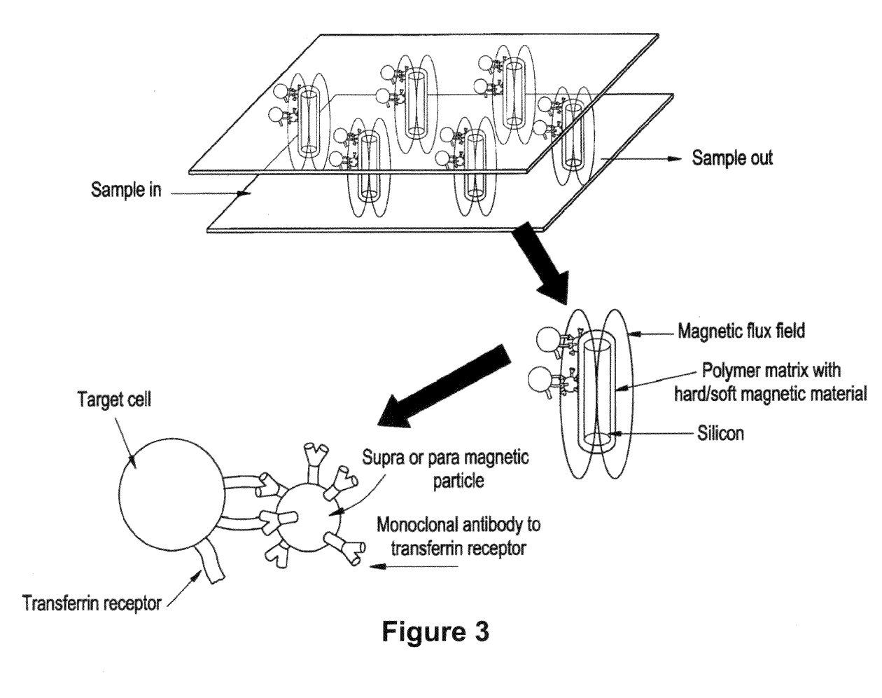

FIG. 3 illustrates an embodiment of a device configured for capture and isolation of cells expressing the transferrin receptor from a complex mixture. Monoclonal antibodies to CD71 receptor can be covalently coupled to magnetic materials, such as a particle including but not limited to ferrous doped polystyrene, ferroparticles or ferro-colloids (e.g., from Miltenyi and Dynal). In one embodiment the anti CD71 bound to magnetic particles is flowed into the device. The antibody coated particles are drawn to the floor, walls or obstacles (e.g., posts) and are retained by the strength of the magnetic field interaction between the particles and the magnetic field. In one embodiment loosely retained particles can be removed by a wash solution.

Enrichment by Flow Cytometry

In one embodiment, one or more rare cells (e.g., one or more fnRBCs, placental cells, etc.) can be enriched or purified using flow cytometry, fluorescent activated cell sorting (FACS) or microfluidic fluorescent cell sorting (e.g. the Cellula platform). In one embodiment one or more molecules (e.g., nucleic acids, proteins) in a rare cell of interest (e.g., fnRBC, placental cell, etc.) can be fluorescently labeled. For binding proteins, a fluorescent molecule can be attached a binding moiety, e.g., an antibody or antibody-based fragment. For enriching cells based on binding nucleic acids, a fluorescent label can be attached to a nucleic acid, e.g., a DNA or RNA probe. Techniques can include RNA-FISH. Expression products (e.g. transcripts or proteins) of any of the genes hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d can be bound with any of the probes mentioned above and used to enrich or purify a cell by flow cytometry (e.g., FACS).

The probe can be a molecular beacon probe, in which the probe can anneal to form a hairpin that juxtaposes a fluorescent molecule attached to one end of the probe with a quenching moiety attached to the other end of the probe. In the hairpin formation, the probe is unable to fluoresce. In the presence of the target molecule for the probe, the probe hybridizes to the target, forcing the fluorescent molecule and the quenching moiety apart, and allowing fluorescence. A molecular beacon probe can be 25 nucleotides long. The five nucleotides at the 5′ and 3′ ends of the probe can be complementary to each other but not anneal to the target DNA, and the internal 15 nucleotides can anneal to the target DNA. One or more molecular beacon probes can designed to hybridize to one or more transcripts expressed from the genes hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d. These probes can be used, for example, to identify, enrich, purify, or enumerate one or more fetal cells.

Subsequent enrichment steps can be used to separate the rare cells (e.g., fnRBC's or placental cells) from non-rare cells, e.g., maternal nucleated red blood cells. In one embodiment, a sample enriched by size-based separation followed by affinity/magnetic separation is further enriched for rare cells using fluorescence activated cell sorting (FACS) or selective lysis of a subset of the cells.

Dielectrophoretic Enrichment

In one embodiment an electric field exert forces on a neutral but polarisable particle, such as cell, suspended in a liquid. According to this particular electrokinetic principle, which is called dielectrophoresis (DEP), a neutral particle, when subject to non-uniform electric fields, experiences a net force directed towards locations with increasing (positive dielectrophoresis—pDEP) or decreasing (negative dielectrophoresis—nDEP) field intensities. More specifically, a particle can be subject to pDEP or nDEP according to the (frequency-dependent) electrical properties of the particle and its suspending medium, the particle dimension and the gradient of the electric field. In one embodiment, the electric field is generated by a silicon chip directly interfaced to a microchamber containing living or non-living particles in liquid suspension. The microchamber is confined between the chip surface and a conductive transparent lid spaced tens of microns apart. The chip surface implements a two dimensional array of microlocations, each consisting of a surface electrode, embedded sensors and logic. The electrodes induce suitable closed nDEP cages in the spatial region above selected microsites, within which single particles may be trapped and levitated individually. Step by step, DEP potential cages can be moved around the device plane concurrently and independently, thus grabbing and dragging single cells and/or microbeads to or from any microchamber location. Separation of heterogeneous populations can be performed by either exploiting DEP spectrum characterisation (i.e. using the frequency-dependent DEP force changing from positive to negative or vice versa) or by using labelling techniques based on functionalised microbeads or fluorescent dyes.

In another embodiment an apparatus can be used to enrich a particle such as a fetal cell by establishing closed dielectrophoretic potential cages and precise displacement thereof. The apparatus can comprise a first array of selectively addressable electrodes, lying on a substantially planar substrate and facing toward a second array comprising one electrode. The arrays define the upper and lower bounds of a micro-chamber where particles are placed in liquid suspension. By applying in-phase and counter-phase periodic signals to electrodes, one or more independent potential cages can be established which cause particles to be attracted to or repelled from cages according to signal frequency and the dielectric characteristics of the particles and suspending medium. By properly applying voltage signal patterns into arrays, cages may trap one or more particles, thus permitting them to levitate steadily and/or move. In one embodiment, an array can be integrated on a semiconductor substrate, displacement of particles can be monitored by embedded sensors.

Enrichment by Apoptosis

In one embodiment, enrichment involves detection and/or isolation of one or more rare cells or rare DNA (e.g. one or more fetal cells or fetal DNA) by selectively initiating apoptosis in the one or more rare cells. This enrichment can be accomplished, for example, by subjecting a sample that includes rare cells (e.g. a mixed sample) to hyperbaric pressure (increased levels of CO₂; e.g. 4% CO₂). This process will selectively initiate condensation and/or apoptosis in the one or more rare or fragile cells in the sample (e.g., one or more fetal cells). Once the one or more rare cells (e.g., one or more fetal cells) begin apoptosis, their nuclei will condense and optionally be ejected from the rare cells. At that point, the one or more rare cells or nuclei can be detected using any technique known in the art to detect condensed nuclei, including DNA gel electrophoresis, in situ labeling fluorescence labeling, and in situ labeling of DNA nicks using terminal deoxynucleotidyl transferase (TdT)-mediated dUTP in situ nick labeling (TUNEL) (Gavrieli, Y., et al. J. Cell Biol. 119:493-501 (1992), which is herein incorporated by reference in its entirety), and ligation of DNA strand breaks having one or two-base 3′ overhangs (Taq polymerase-based in situ ligation; Didenko V., et al. J. Cell Biol. 135:1369-76 (1996), which is herein incorporated by reference in its entirety).

In one embodiment ejected nuclei can further be detected using a size based separation module adapted to selectively enrich nuclei and other analytes smaller than a predetermined size (e.g. 6 microns) and isolate them from cells and analytes having a hydrodynamic diameter larger than 6 microns. Thus, in one embodiment, the present invention contemplated detecting one or more fetal cells/fetal DNA and optionally using such fetal DNA to diagnose or prognose a condition in a fetus. Such detection and diagnosis can occur by obtaining a blood sample from the female pregnant with the fetus, enriching the sample for cells and analytes larger than 8 microns using, for example, an array of obstacles adapted for size-base separation where the predetermined size of the separation is 8 microns (e.g. the gap between obstacles is up to 8 microns). Then, the enriched product is further enriched for red blood cells (RBC's) by oxidizing the sample to make the hemoglobin paramagnetic and flowing the sample through one or more magnetic regions. This selectively captures the RBC's and removes other cells (e.g. white blood cells) from the sample. Subsequently, the fnRBC's can be enriched from mnRBC's in the second enriched product by subjecting the second enriched product to hyperbaric or hypobaric pressure or other stimulus that selectively causes the one or more fetal cells to begin apoptosis and condense/eject their nuclei. Such condensed nuclei are then identified/isolated using, e.g., laser capture microdissection or a size based separation module that separates components smaller than 3, 4, 5 or 6 microns from a sample. Such fetal nuclei can then by analyzed using any method known in the art or described herein.

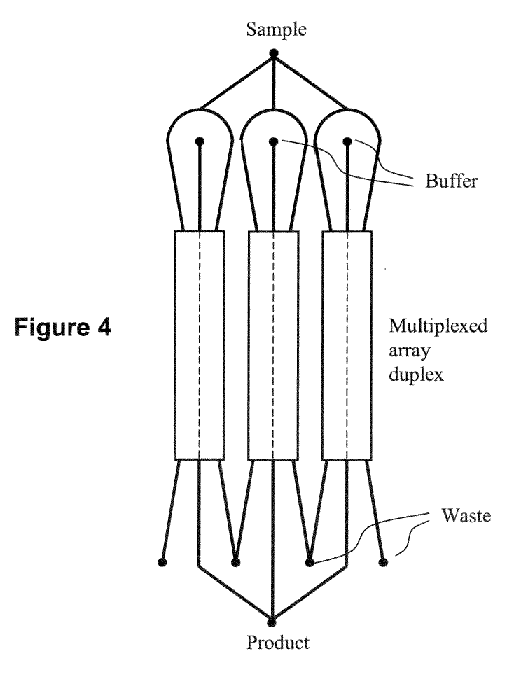

In one embodiment, a fluid sample such as a blood sample is first flowed through one or more size-base separation module. Such modules can be fluidly connected in series and/or in parallel. FIG. 4 illustrates one embodiment of three size-based enrichment modules that are fluidly coupled in parallel. The waste (e.g., cells having hydrodynamic size less than 4 microns) are directed into a first outlet and the product (e.g., cells having hydrodynamic size greater than 4 microns) are directed to a second outlet. The product is subsequently enriched using the inherent magnetic property of hemoglobin. The product is modified (e.g., by addition of one or more reagents) such that the hemoglobin in the red blood cells becomes paramagnetic. Subsequently, the product is flowed through one or more magnetic fields. The cells that are trapped by the magnetic field are subsequently analyzed using the one or more methods herein.

One or more of the enrichment modules herein (e.g., size-based separation module(s) and capture module(s)) can be fluidly coupled in series or in parallel with one another. For example a first outlet from a separation module can be fluidly coupled to a capture module. In one embodiment, the separation module and capture module are integrated such that a plurality of obstacles acts both to deflect certain analytes according to size and direct them in a path different than the direction of analyte(s) of interest, and also as a capture module to capture, retain, or bind certain analytes based on size, affinity, magnetism or other physical property.

Efficiency of Enrichment

In any of the embodiments herein, the enrichment steps performed have a specificity and/or sensitivity greater than 50, 60, 70, 80, 90, 95, 96, 97, 98, 99, 99.1, 99.2, 99.3, 99.4, 99.5, 99.6, 99.7, 99.8, 99.9, or 99.95% The retention rate of the enrichment module(s) herein is such that 60, 70, 80, 90, 91, 92, 93, 94, 95, 96, 97, 98, 99, or 99.9% of the analytes or cells of interest (e.g., nucleated cells or nucleated red blood cells or nucleated from red blood cells) are retained. Simultaneously, the enrichment modules are configured to remove ≧50, 60, 70, 80, 85, 90, 91, 92, 93, 94, 95, 96, 97, 98, 99, or 99.9% of all unwanted analytes (e.g., red blood-platelet enriched cells) from a sample.

For example, in one embodiment the analytes of interest are retained in an enriched solution that is less than 50, 40, 30, 20, 10, 9.0, 8.0, 7.0, 6.0, 5.0, 4.5, 4.0, 3.5, 3.0, 2.5, 2.0, 1.5, 1.0, or 0.5 fold diluted from the original sample. In one embodiment, any or all of the enrichment steps increase the concentration of the analyte of interest (fetal cell), for example, by transferring them from the fluid sample to an enriched fluid sample (sometimes in a new fluid medium, such as a buffer).

III. Fetal Biomarkers

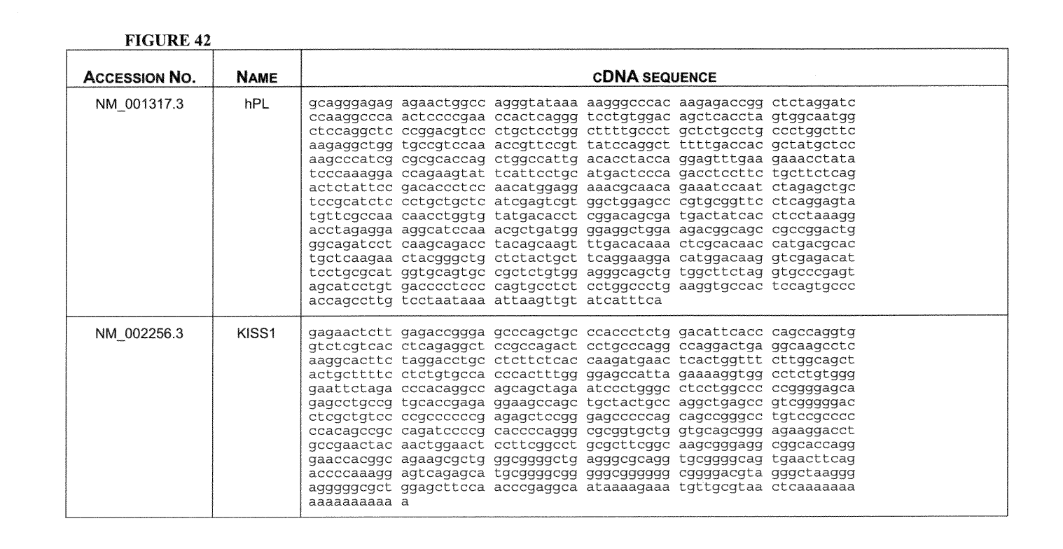

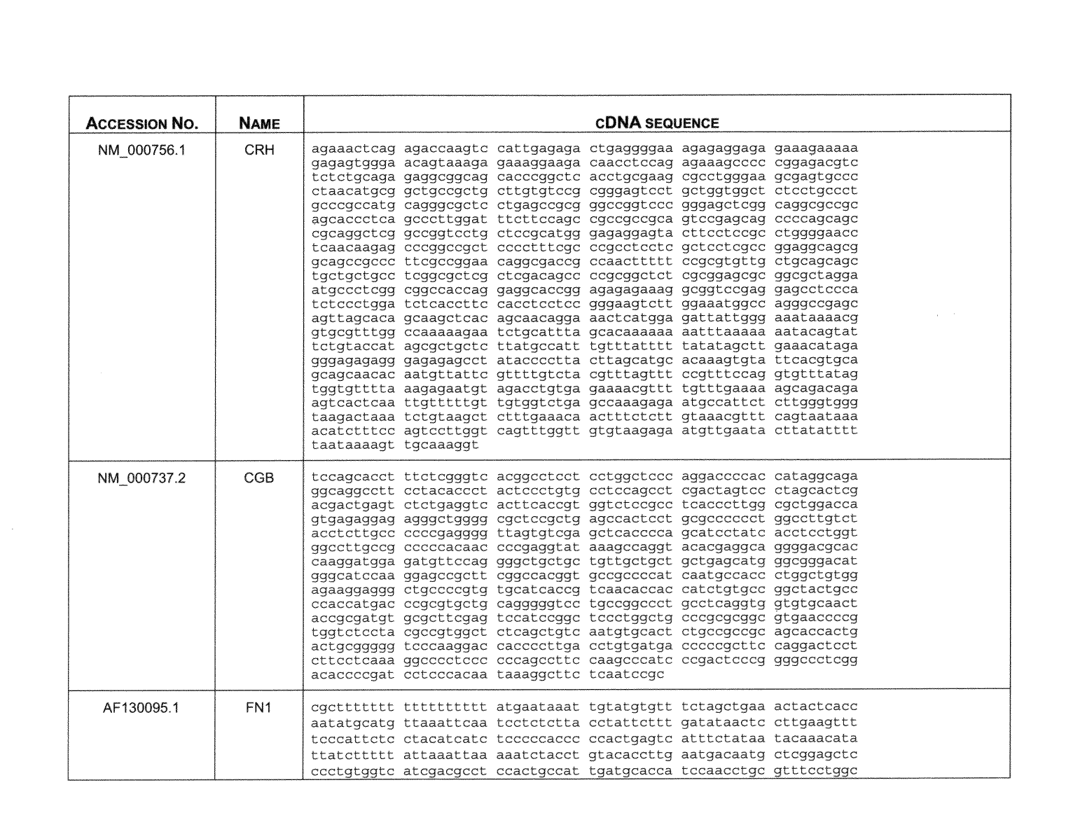

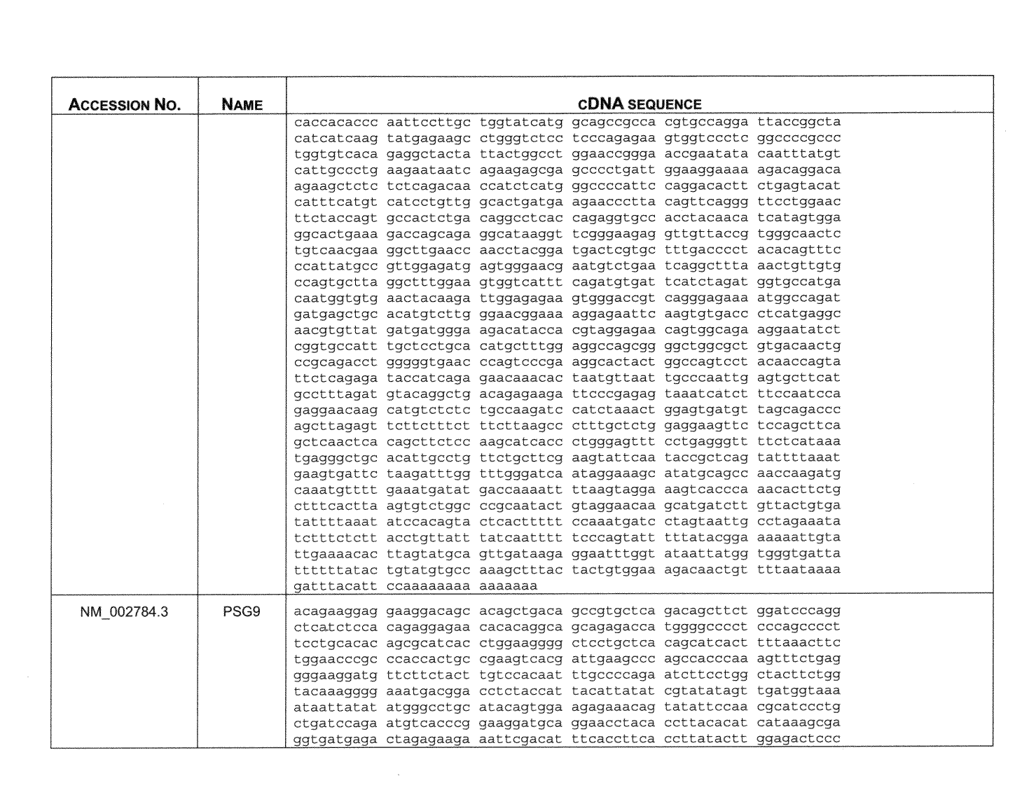

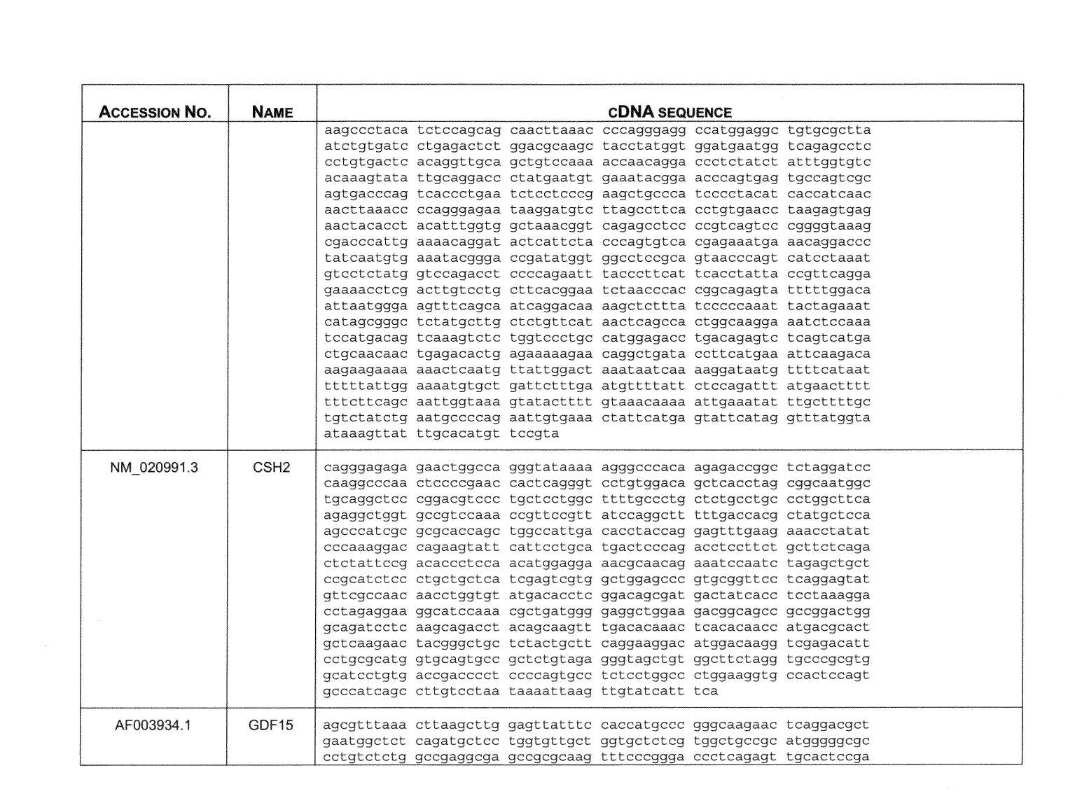

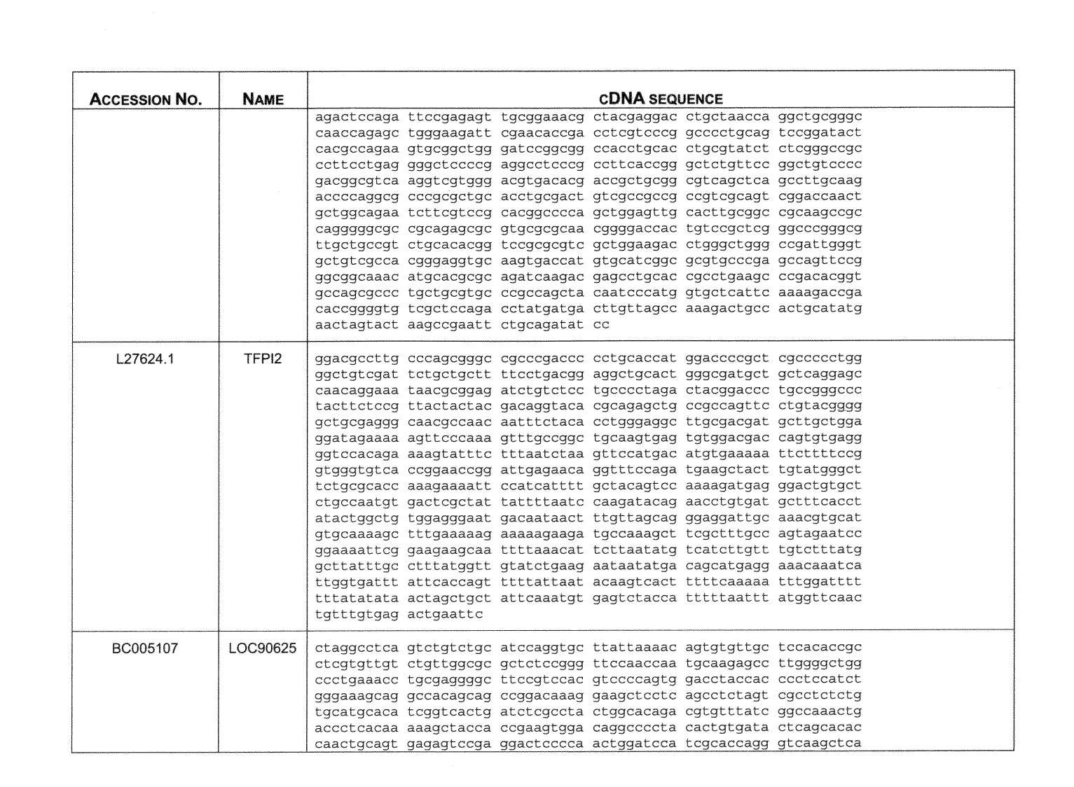

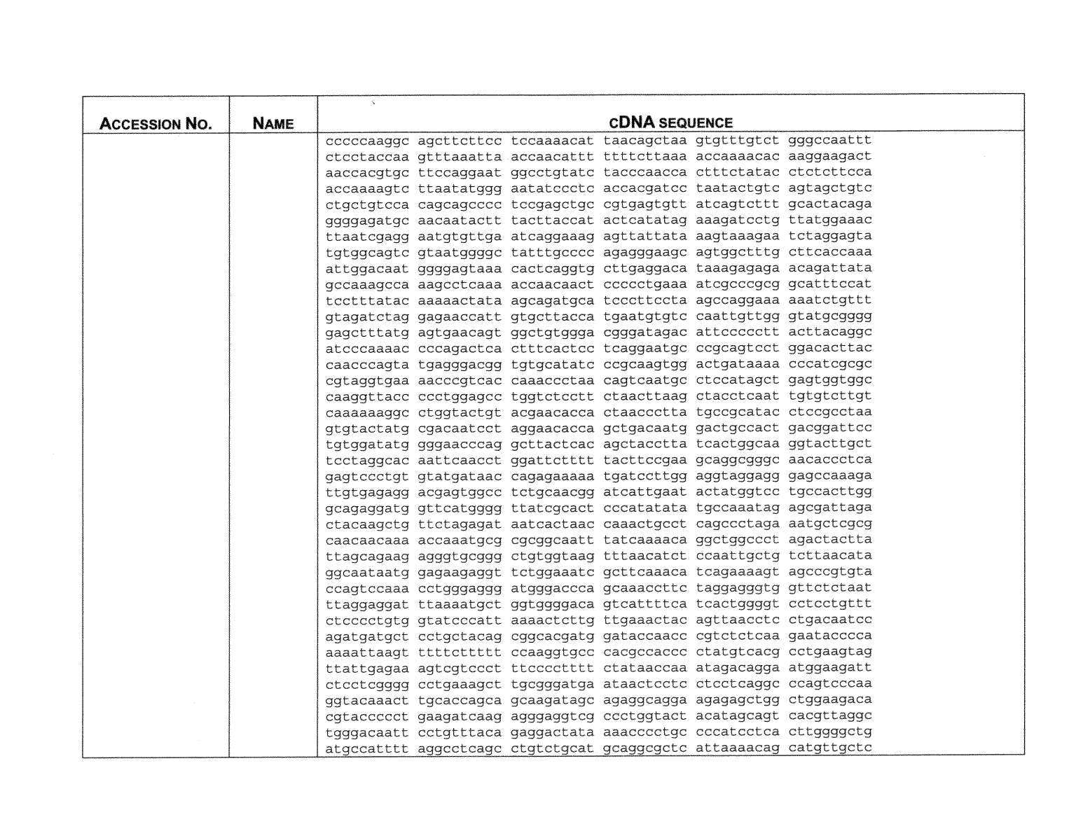

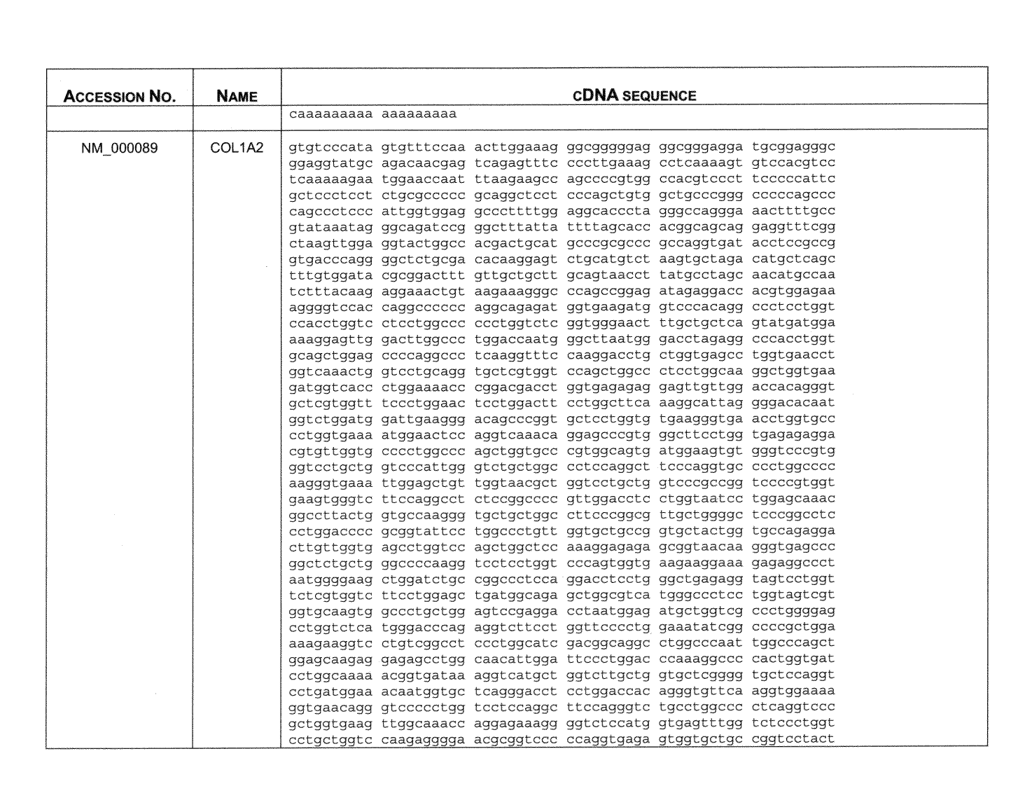

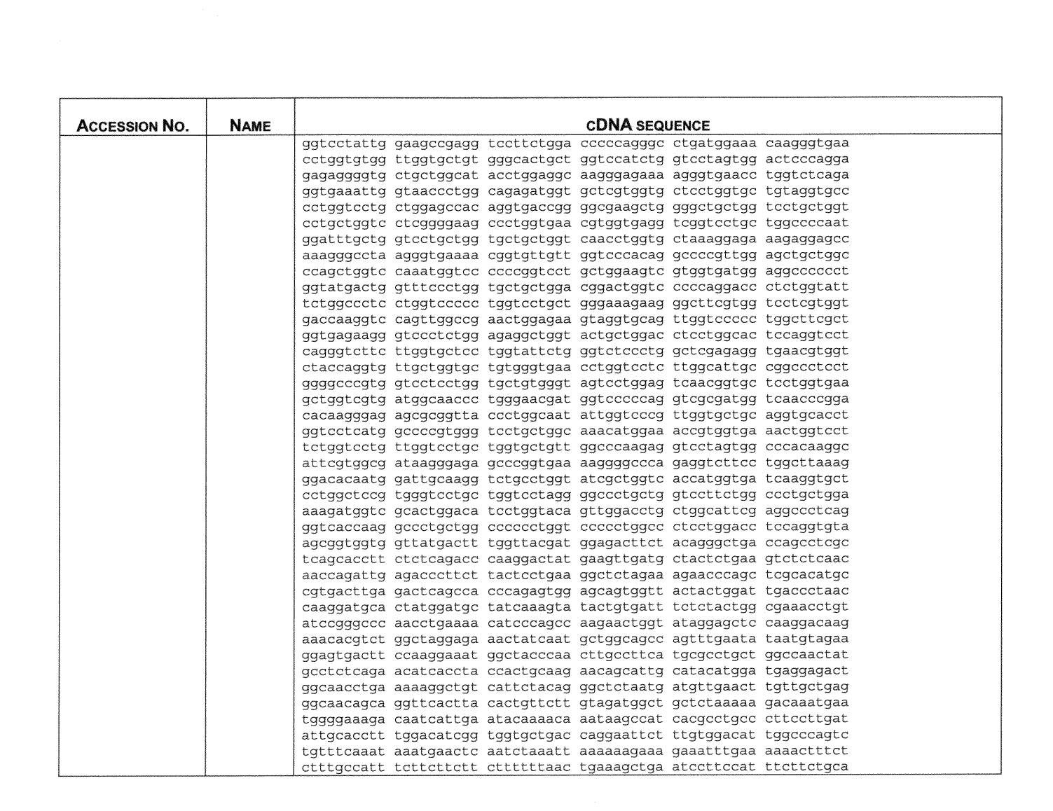

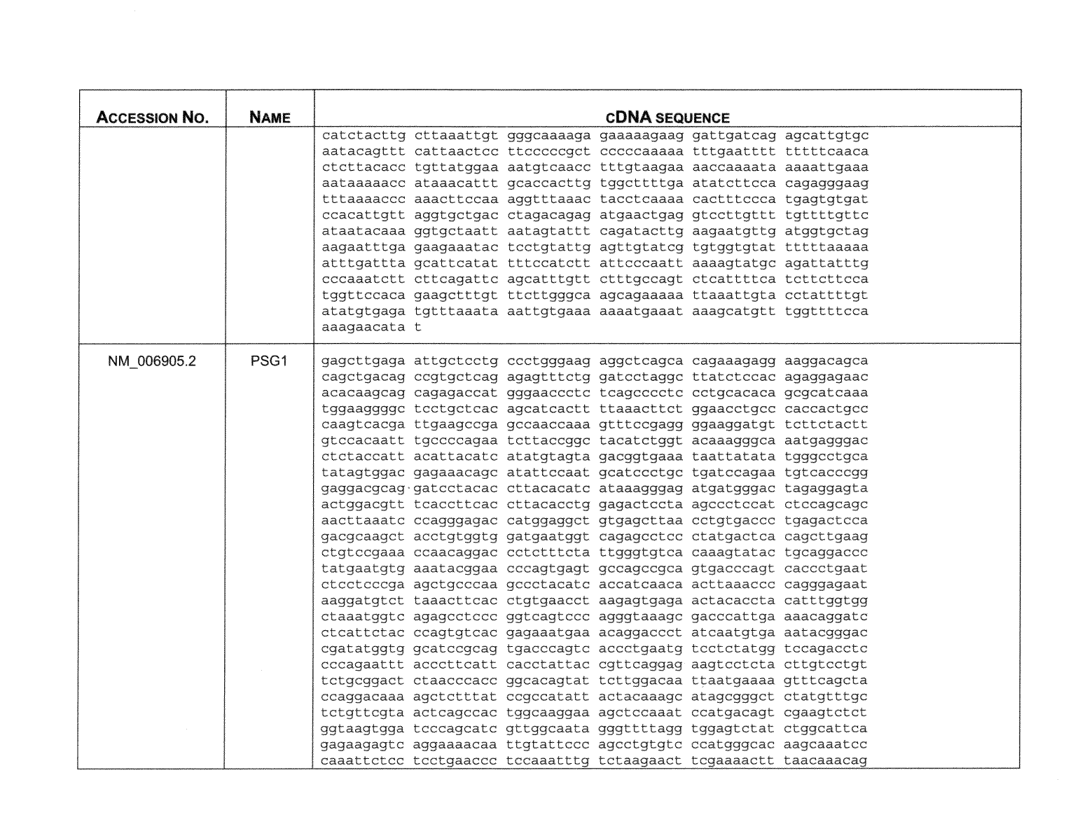

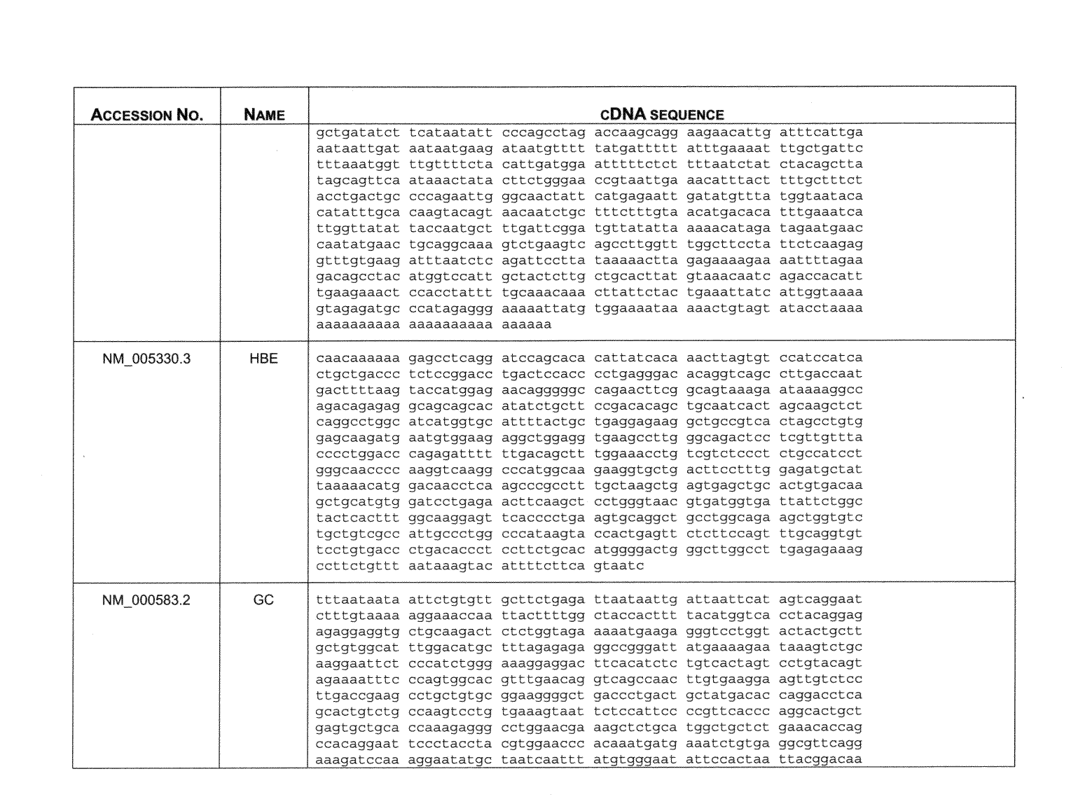

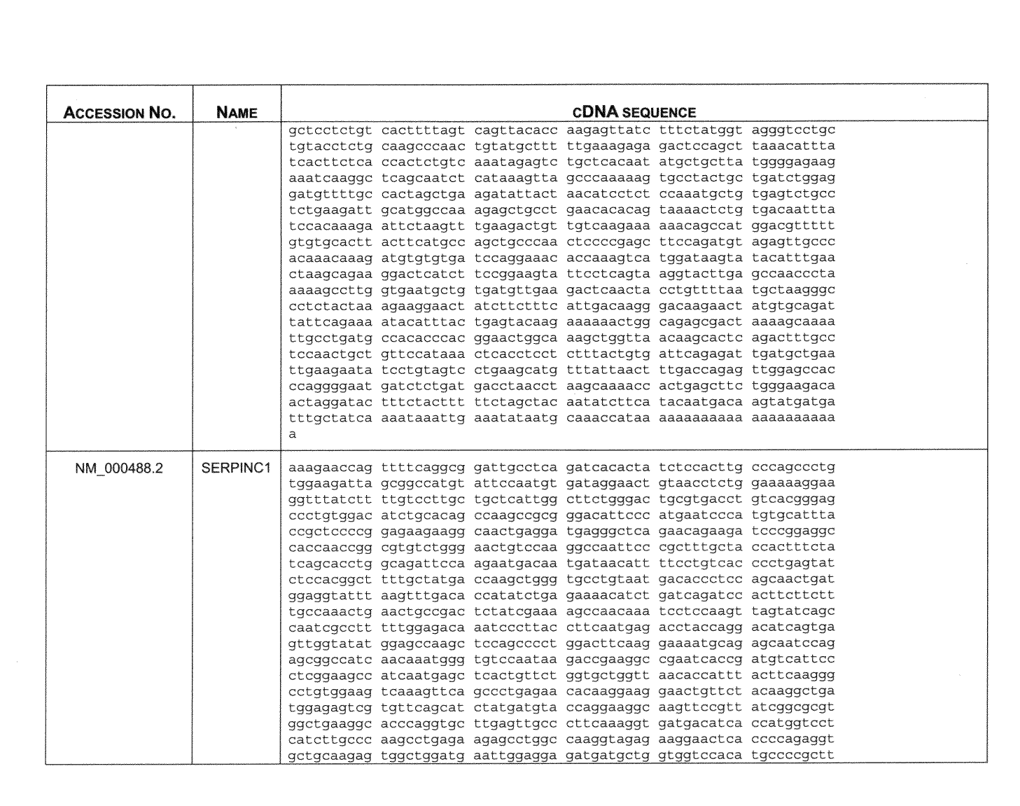

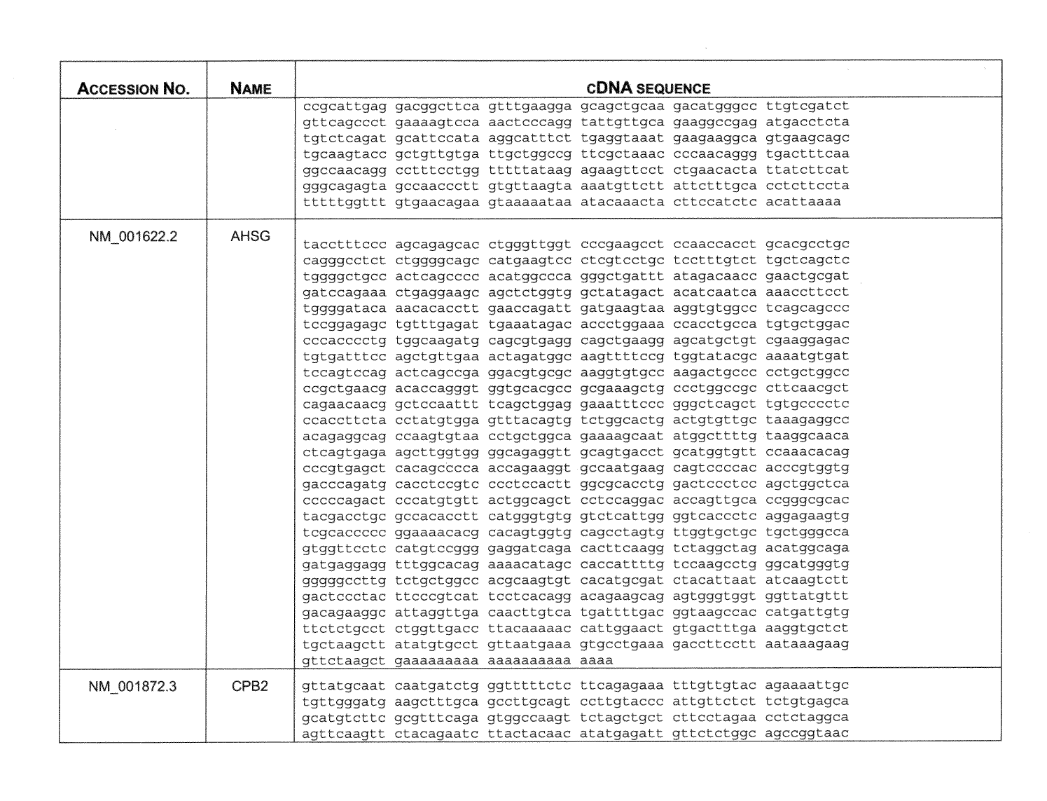

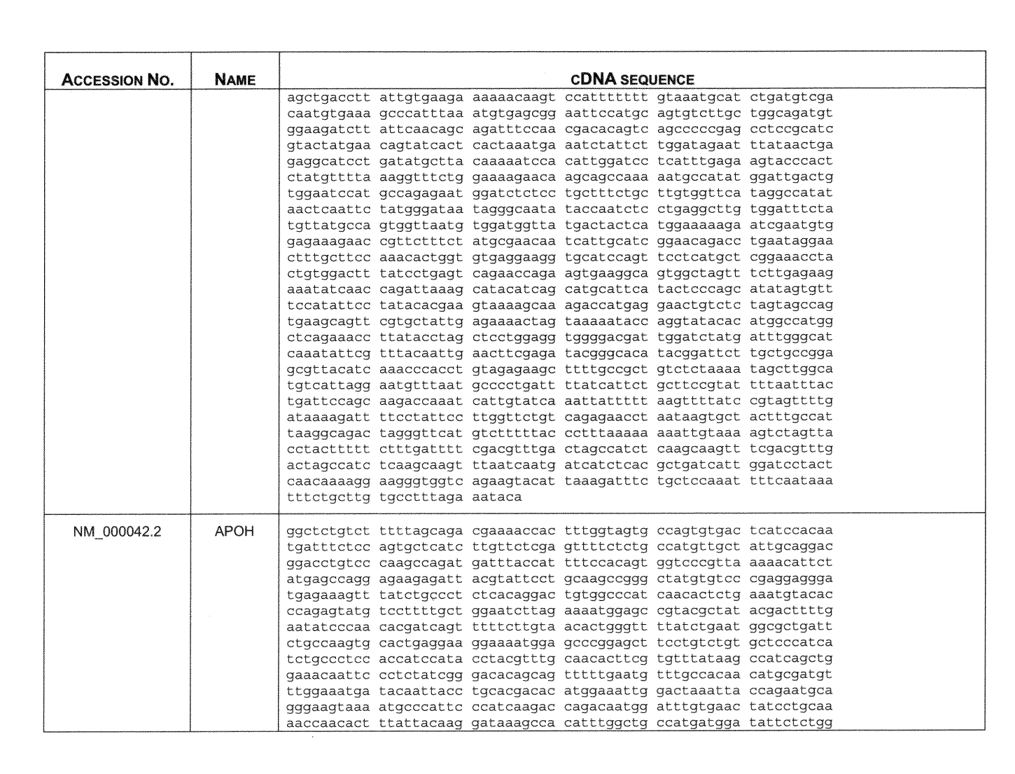

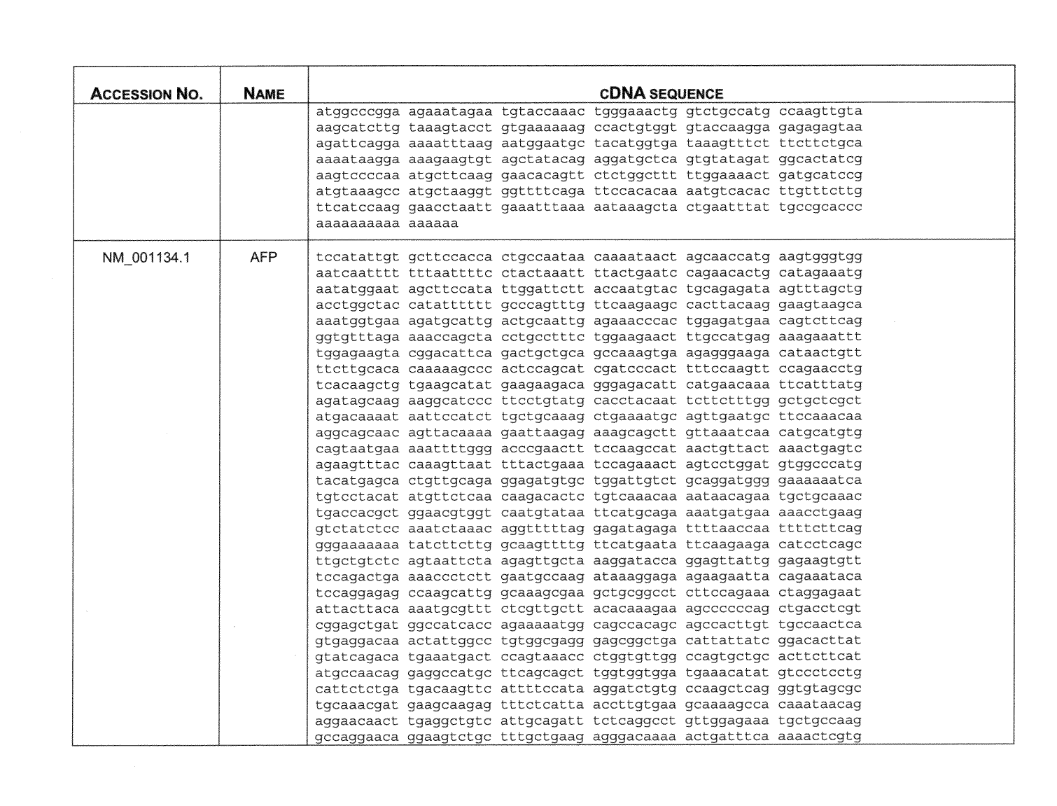

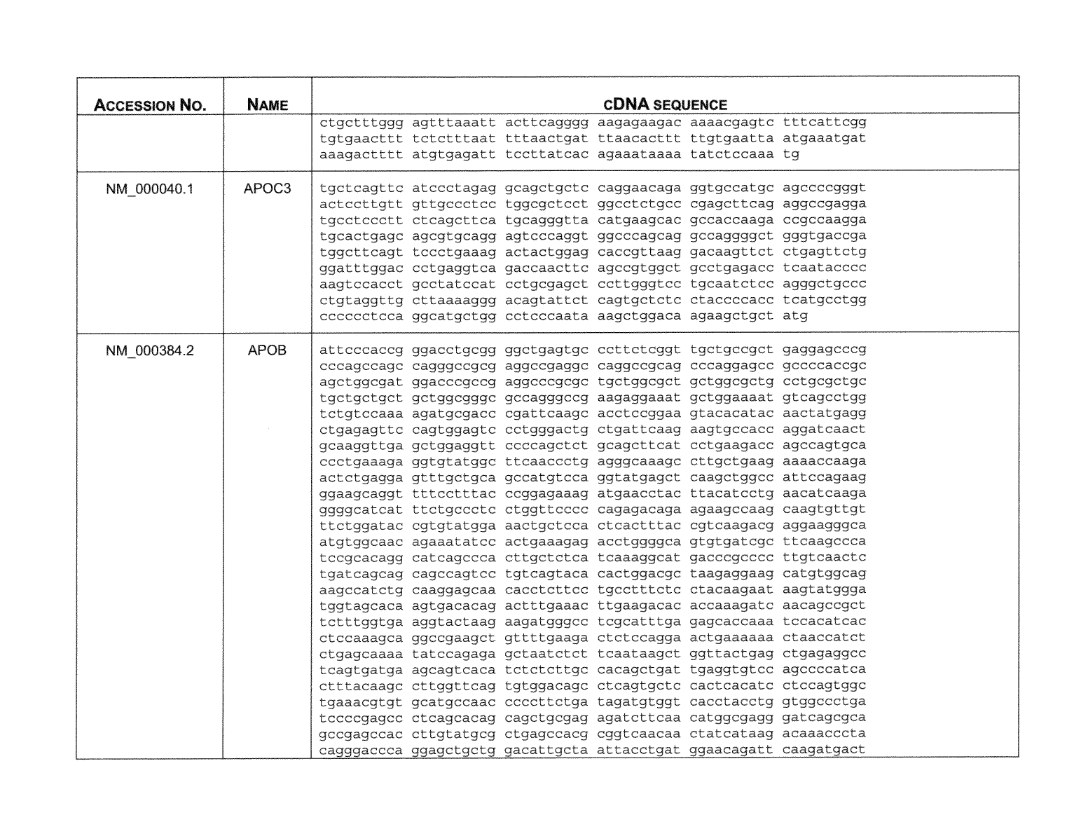

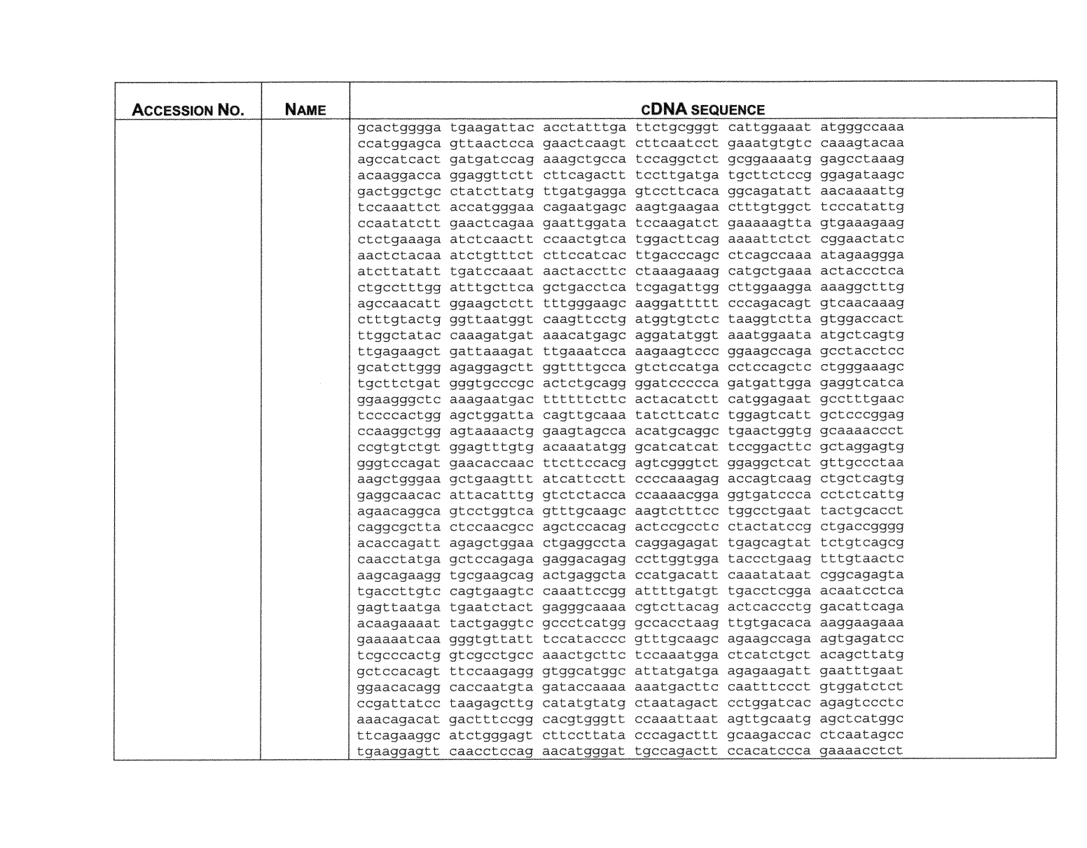

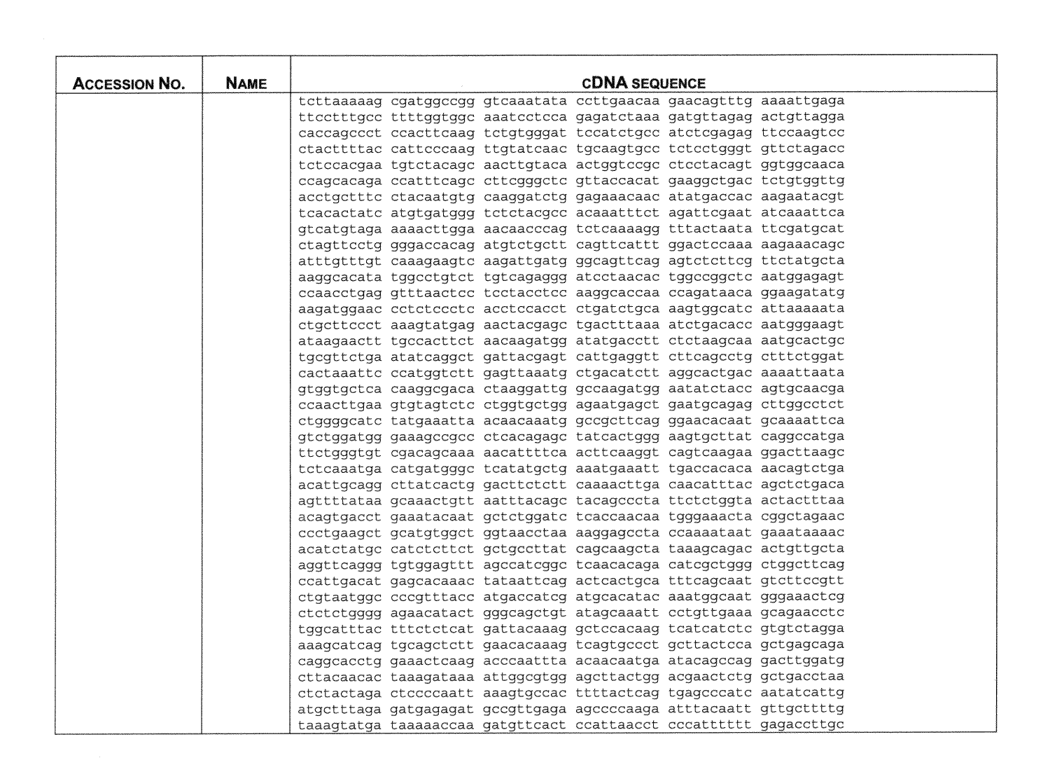

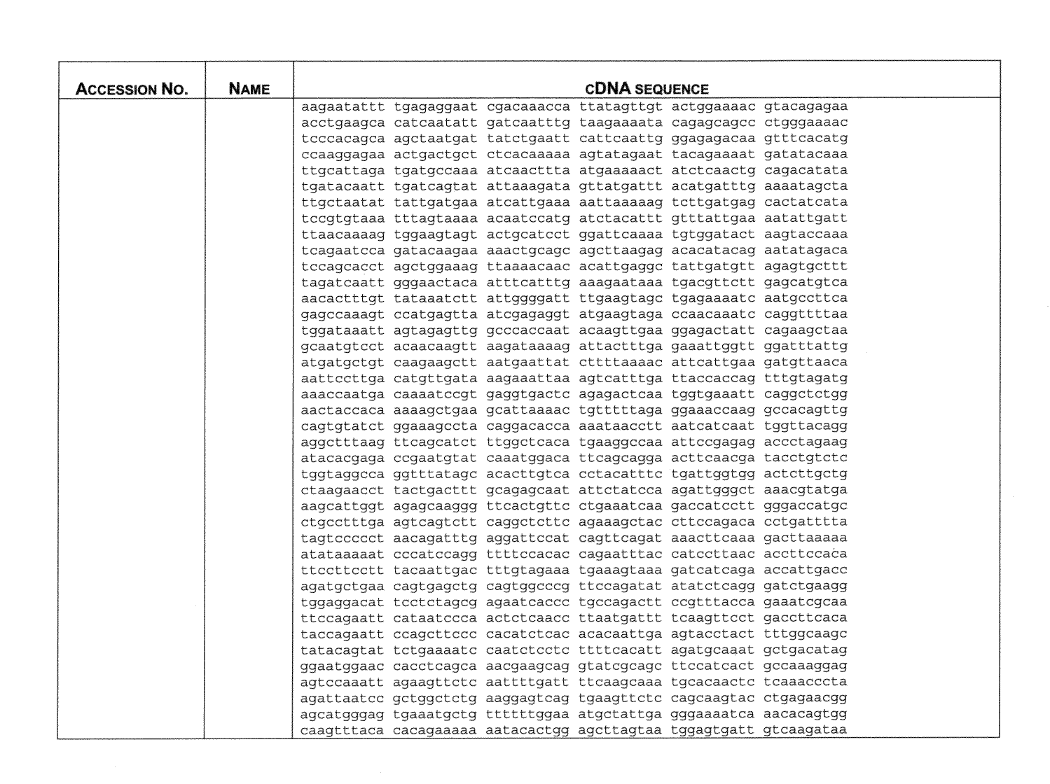

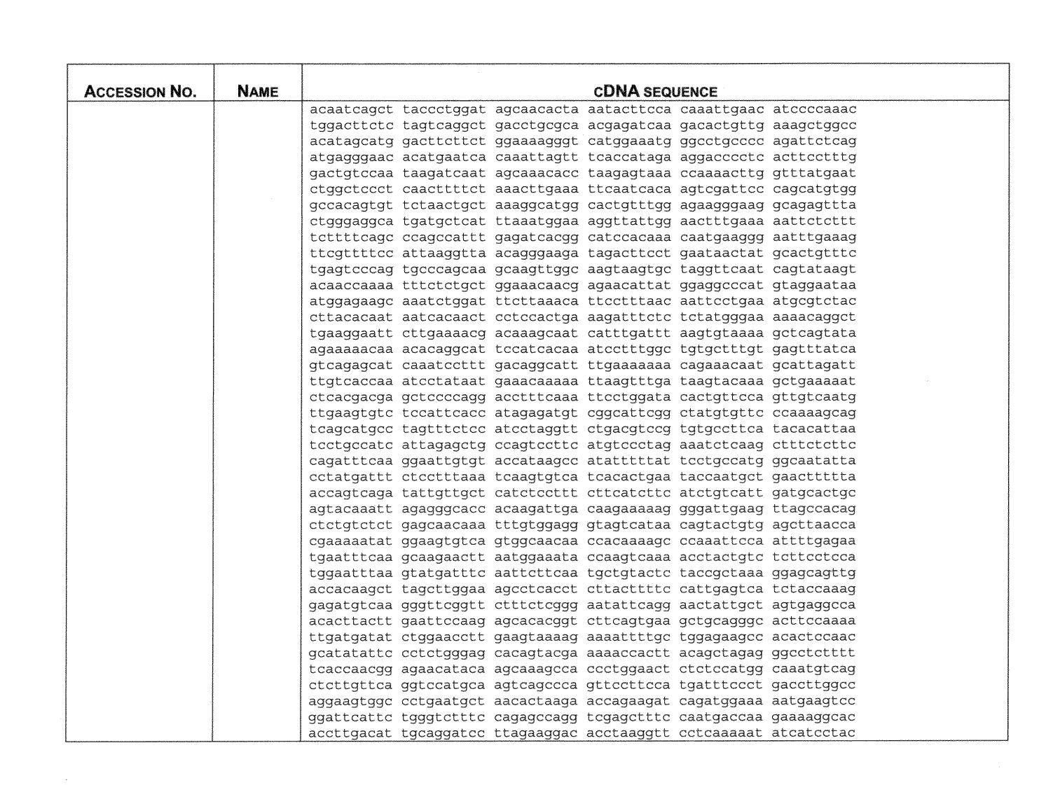

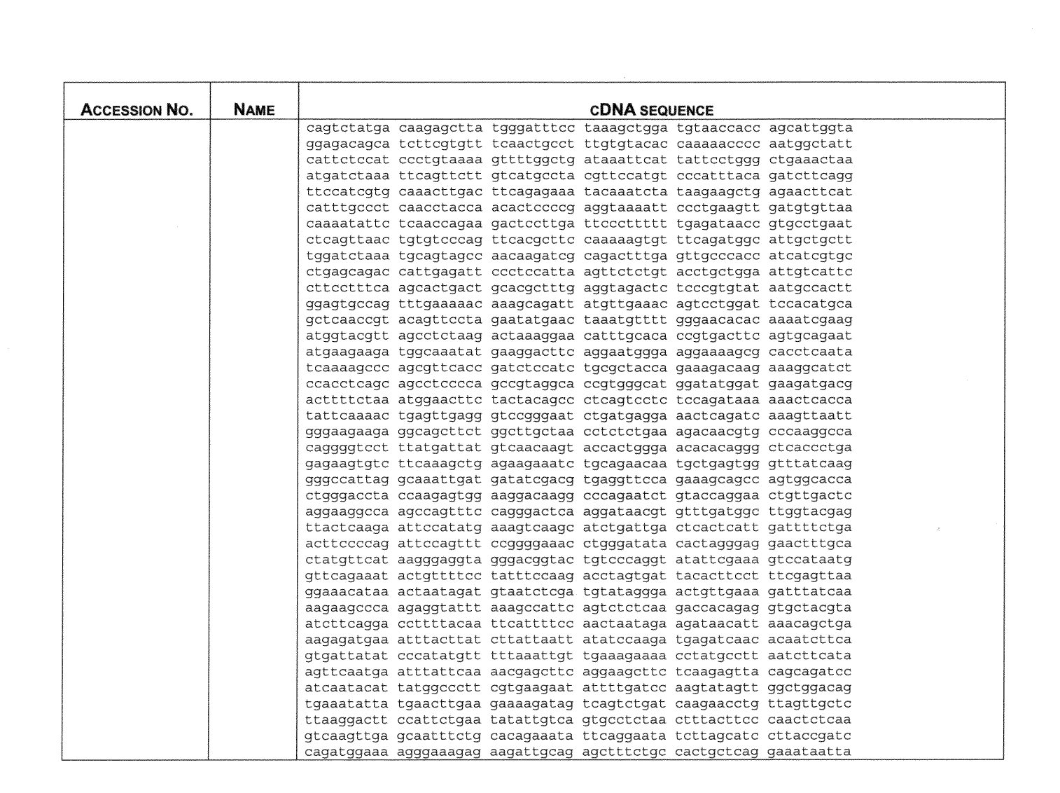

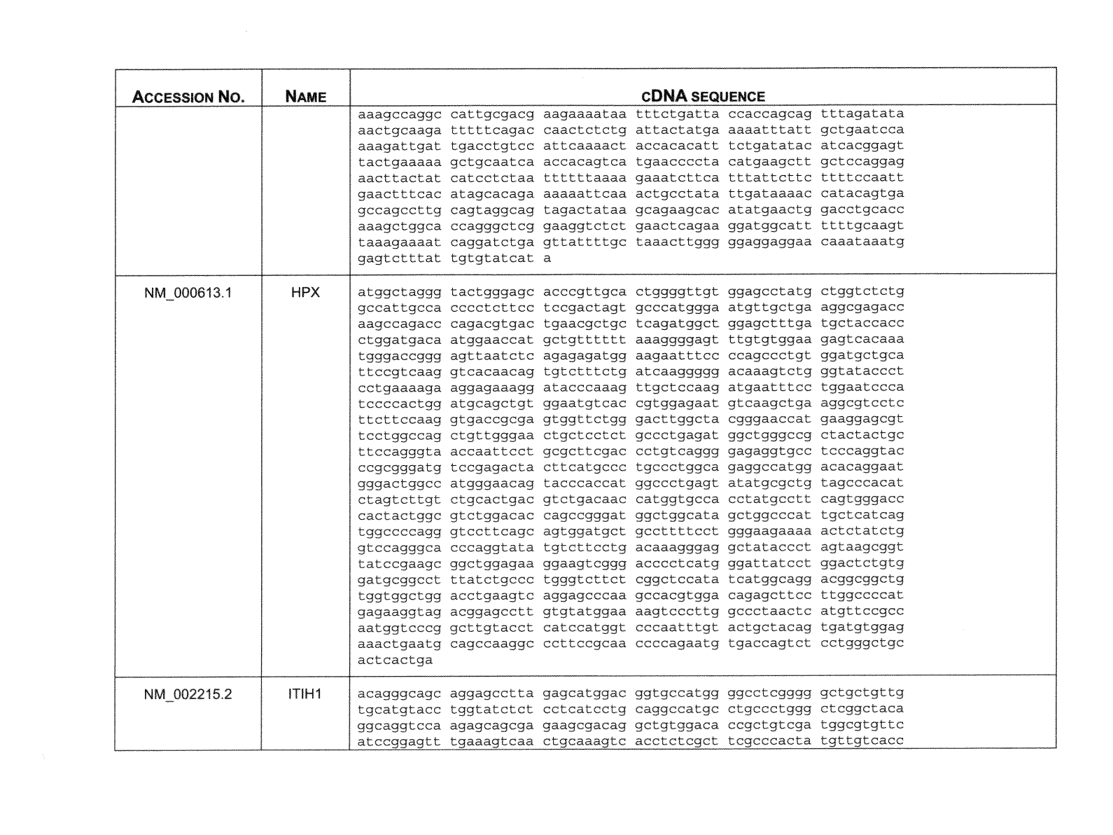

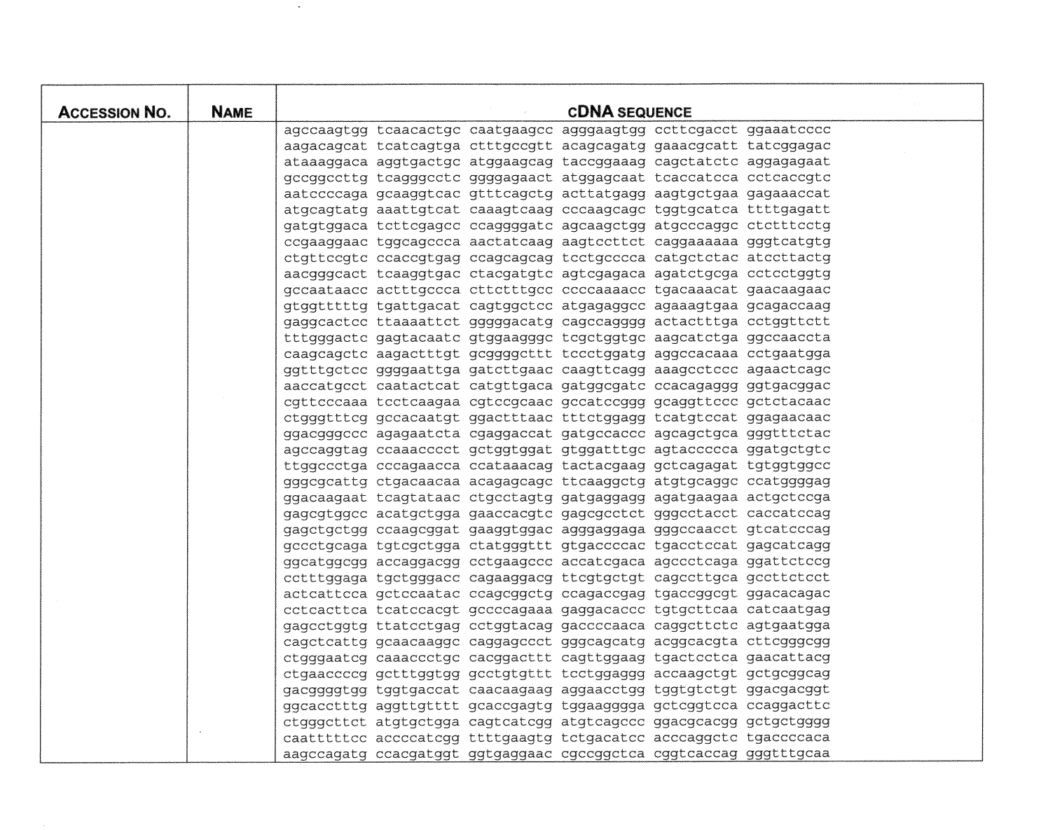

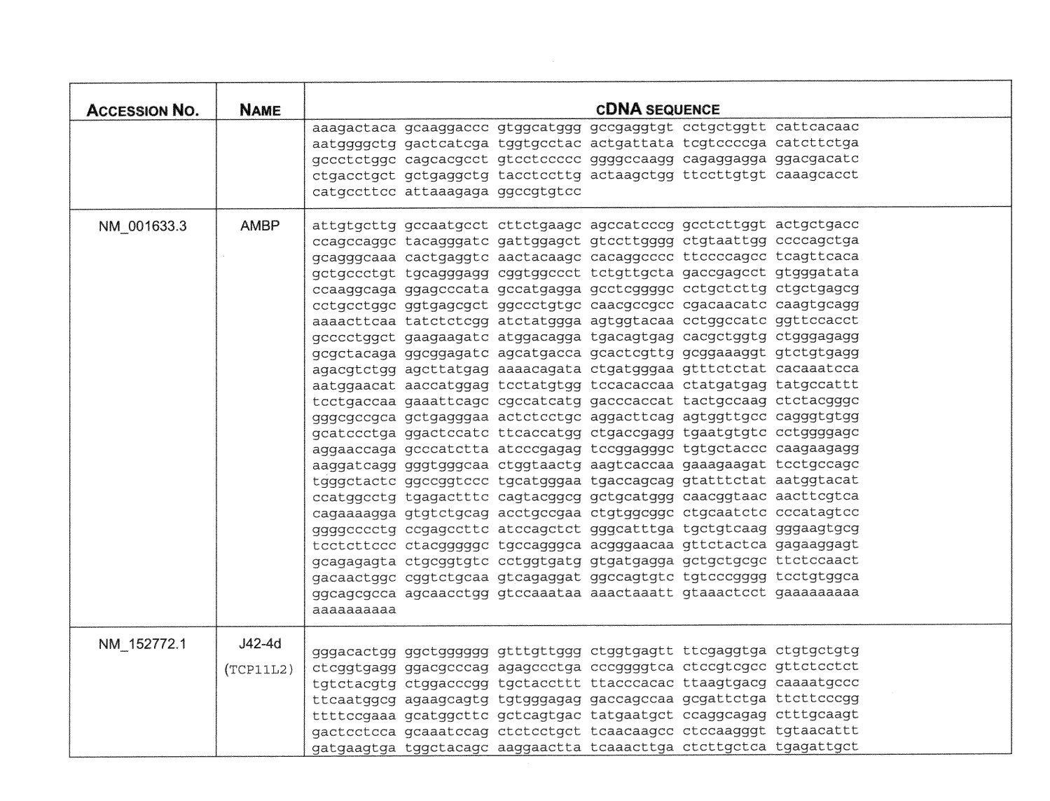

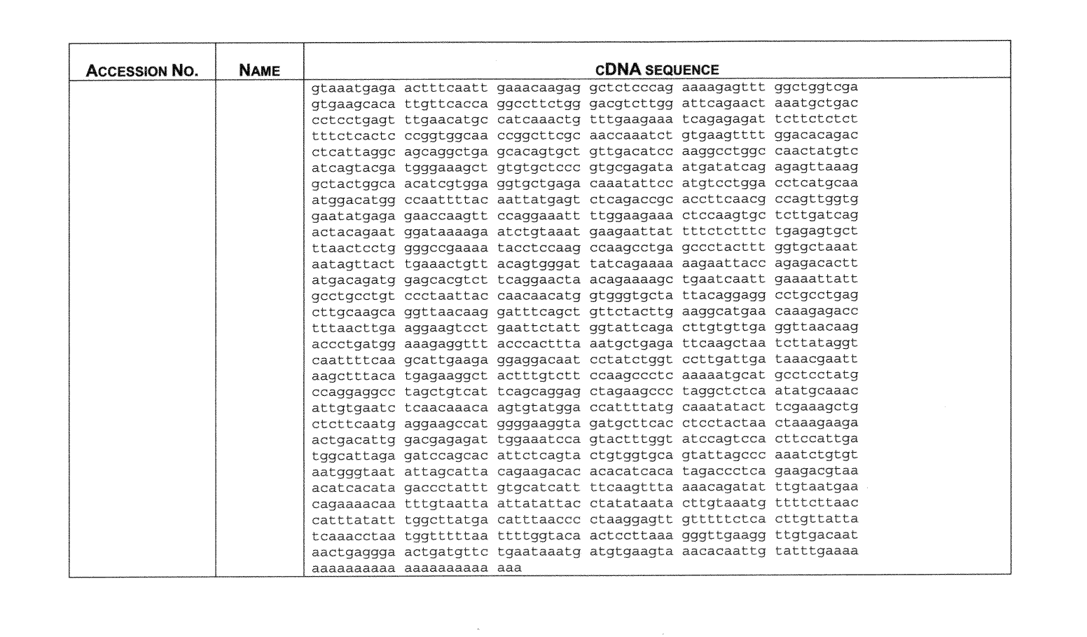

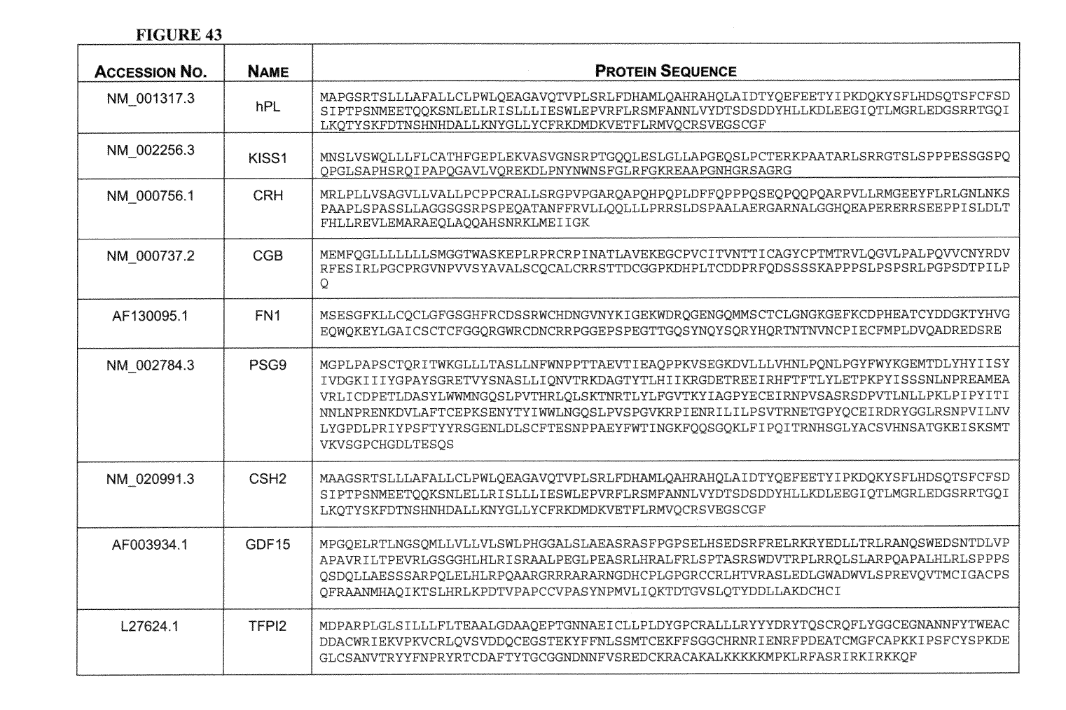

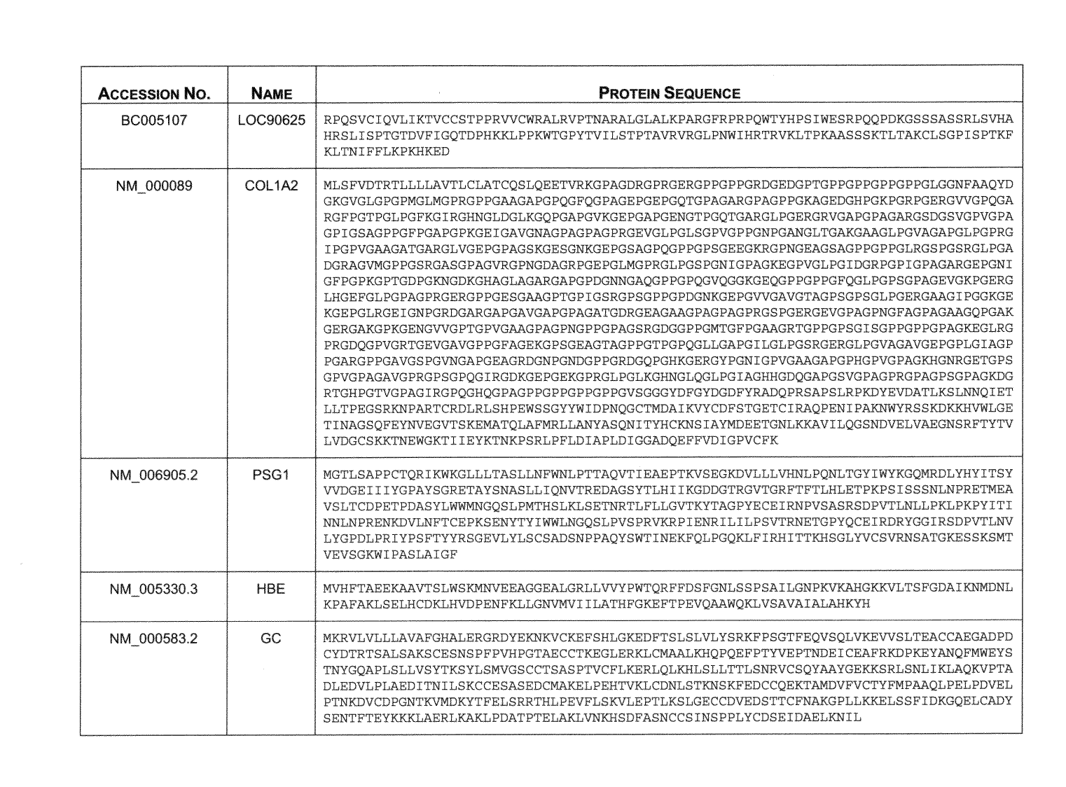

In one embodiment fetal biomarkers can be used to detect and/or isolate one or more fetal cells. For example, this can be performed by distinguishing between fetal and maternal nRBCs based on relative expression of a gene (e.g., DYS1, DYZ, CD-71, ∈- and ζ-globin) that is differentially expressed during fetal development. In one embodiment of the provided invention, detection of transcript or protein expression of one or more genes including, hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d, is used to enrich, purify, enumerate, identify detect or distinguish a fetal cell. The expression can include a transcript expressed from these genes (FIG. 42) or a protein (FIG. 43). In one embodiment of the provided invention, expression of one or more genes including HBE, AFP, AHSG, or J42-4-d is used to identify, purify, enrich, or enumerate an fnRBC. In another embodiment of the provided invention, transcript or protein expression of one or more genes including hPL, beta-hCG, FN1, KISS1, or LOC90625 is used to identify, purify, enrich, or enumerate a trophoblast.

In one embodiment samples can be taken at different times during the pregnancy of a mother (e.g., trimester, early 2^{nd} trimester, 2^{nd} trimester, or 3^{rd} trimester) and expression of genes (e.g., transcript or protein) in cells from the samples can be used to detect, distinguish, identify, purify, enrich, or enumerate a fetal cell (e.g., a fnRBC or trophoblast). In one embodiment, a maternal sample is taken in the 1^{st} or early 2^{nd} trimester, and expression of HBE is used to detect, distinguish, identify, purify, enrich, or enumerate a fnRBC. In another embodiment, a maternal sample is taken in the early 2^{nd} trimester, and detection of transcript or protein expression of AFP, AHSG, or J42-4-d is used to detect, distinguish, identify, purify, enrich, or enumerate an fnRBC. In another embodiment, a maternal sample is taken in the 1^{st} or early 2^{nd} trimester, and detection of transcript or protein expression of hPL, beta-hCG, or FN1 is used to detect, distinguish, identify, purify, enrich, or enumerate a trophoblast.

Genes

hPL (also known as CH1; CSA; CSMT; and FLJ75407) encodes a protein that is a member of the somatotropin/prolactin family of hormones. This protein plays a role in growth control. The gene is located at the growth hormone locus on chromosome 17 along with four other related genes in the same transcriptional orientation. Although the five genes share a remarkably high degree of sequence identity, they are expressed selectively in different tissues. Alternative splicing generates additional isoforms of each of the five growth hormones, leading to further diversity and potential for specialization. This particular family member is expressed mainly in the placenta and utilizes multiple transcription initiation sites. Expression of the identical mature proteins for chorionic somatomammotropin hormones 1 and 2 is up regulated during development, although the ratio of 1 to 2 increases by term. Mutations in this gene result in placental lactogen deficiency and Silver-Russell syndrome.

CSH2 (also know as CSB; CS-2; and hCS-B) encodes a protein that is a member of the somatotropin/prolactin family of hormones and plays a role in growth control. The gene is located at the growth hormone locus on chromosome 17 along with four other related genes in the same transcriptional orientation; an arrangement which is thought to have evolved by a series of gene duplications. Although the five genes share a remarkably high degree of sequence identity, they are expressed selectively in different tissues. Alternative splicing generates additional isoforms of each of the five growth hormones. This particular family member is expressed mainly in the placenta and utilizes multiple transcription initiation sites. Expression of the identical mature proteins for chorionic somatomammotropin hormones 1 and 2 is up regulated during development, while the ratio of 1 to 2 increases by term. Structural and expression differences provide avenues for developmental regulation and tissue specificity.

KISS1 (also known as KiSS-1; METASTIN; and MGC39258) is a metastasis suppressor gene that suppresses metastases of melanomas and breast carcinomas without affecting tumorigenicity. The encoded protein may function to inhibit chemotaxis and invasion, attenuating metastasis in malignant melanomas. Studies suggest a putative role in the regulation of events downstream of cell-matrix adhesion, perhaps involving cytoskeletal reorganization. A polymorphism in the terminal exon of this mRNA results in two protein isoforms. An adenosine present at the polymorphic site represents the third position in a stop codon. When the adenosine is absent, a downstream stop codon is utilized and the encoded protein extends for an additional seven amino acid residues.

GDF15 (also known as PDF; MIC1; PLAB; MIC-1; NAG-1; PTGFB; and GDF-15) is a member of the transforming growth factor-beta superfamily and regulates tissue differentiation and maintenance. It is synthesized as a precursor molecule that is processed at a dibasic cleavage site to release a C-terminal domain containing a characteristic motif of 7 conserved cysteines in the mature protein.

CRH (also known as Corticotropin-releasing hormone; and CRF) is a 41-amino acid peptide derived from a 191-amino acid preprohormone. CRH is secreted by the paraventricular nucleus (PVN) of the hypothalamus in response to stress. Marked reduction in CRH has been observed in association with Alzheimer disease and autosomal recessive hypothalamic corticotropin deficiency has multiple and potentially fatal metabolic consequences including hypoglycemia and hepatitis. In addition to production in the hypothalamus, CRH is also synthesized in peripheral tissues, such as T lymphocytes and is highly expressed in the placenta. In the placenta CRH is a marker that determines the length of gestation and the timing of parturition and delivery. A rapid increase in circulating levels of CRH occurs at the onset of parturition, suggesting that, in addition to its metabolic functions, CRH may act as a trigger for parturition.

TFPI2 (also known as tissue factor pathway inhibitor 2; PPS; REF1; TFPI-2; and FLJ21164) weakly inhibits the coagulation proteins factor Xa and factor VIIa/TF complex. Targets of TFPI-2 include serine proteases, e.g., kallikrein, trypsin, chymotrypsin, and plasmin. TFPI-2 expressed by endothelial cells of various origins localizes within the ECM. TFPI-2 can limit the enzymatic activity of matrix metalloproteinases (MMPs).

Beta-hCG (also know as b-hCG, HCG, CGB, CGB3 and hCGB) is a member of the glycoprotein hormone beta chain family and encodes the beta 3 subunit of chorionic gonadotropin (CG). Glycoprotein hormones are heterodimers consisting of a common alpha subunit and an unique beta subunit which confers biological specificity. CG is produced by the trophoblastic cells of the placenta and stimulates the ovaries to synthesize the steroids that are essential for the maintenance of pregnancy. The beta subunit of CG is encoded by 6 genes which are arranged in tandem and inverted pairs on chromosome 19q13.3 and contiguous with the luteinizing hormone beta subunit gene.

LOC90625 (also known as chromosome 21 open reading frame 105; C21orf105) is expressed in the placenta and is overexpressed in trisomy 21 placentas.

FN1 (also known as FN; CIG; FNZ; MSF; ED-B; FINC; GFND; LETS; GFND2; DKFZp686H0342; DKFZp6861I1370; DKFZp686F10164; and DKFZp686O13149) encodes fibronectin, a glycoprotein present in a soluble dimeric form in plasma, and in a dimeric or multimeric form at the cell surface and in extracellular matrix. Fibronectin is involved in cell adhesion and migration processes including embryogenesis, wound healing, blood coagulation, host defense, and metastasis. The gene has three regions subject to alternative splicing, with the potential to produce 20 different transcript variants.

COL1A2 (also known as collagen, type I, alpha 2; OI4) encodes the pro-alpha2 chain of type I collagen whose triple helix comprises two alpha1 chains and one alpha2 chain. Type I is a fibril-forming collagen found in most connective tissues and is abundant in bone, cornea, dermis and tendon. Mutations in this gene are associated with osteogenesis imperfecta types I-IV, Ehlers-Danlos syndrome type VIIB, recessive Ehlers-Danlos syndrome Classical type, idiopathic osteoporosis, and atypical Marfan syndrome. Symptoms associated with mutations in this gene, however, tend to be less severe than mutations in the gene for the alpha1 chain of type I collagen (COL1A21) reflecting the different role of alpha2 chains in matrix integrity. Three transcripts, resulting from the use of alternate polyadenylation signals, have been identified for this gene.

PSG9 (also known as pregnancy specific beta-1-glycoprotein 9; PSG11; and PSGII) is a member of the carcinoembryonic antigen (CEA)/PSG family. PSG9 is produced at high levels during pregnancy, mainly by syncytiotrophoblasts.

PSG1 (also known as pregnancy specific beta-1-glycoprotein 1; SP1; B1G1; PBG1; CD66f; PSBG1; PSGGA; DHFRP2; PSGIIA; FLJ90598; and FLJ90654) is a pregnancy associated protein produced by the human placenta. PSG1 shares sequence similarity with carcinoembryonic antigen (CEA) family members, and is structurally similar to immunoglobulins (Igs).

HBE (also know as hemoglobin, epsilon 1, HBE1) is normally expressed in the embryonic yolk sac: two epsilon chains together with two zeta chains (an alpha-like globin) constitute the embryonic hemoglobin Hb Gower I; two epsilon chains together with two alpha chains form the embryonic Hb Gower II. Both of these embryonic hemoglobins are normally supplanted by fetal, and later, adult hemoglobin. The five beta-like globin genes are found within a 45 kb cluster on chromosome 11 in the following order: 5′-epsilon-G-gamma-A-gamma-delta-beta-3′.

AFP (also known as alpha-fetoprotein; FETA; and HPAFP) encodes alpha-fetoprotein, a major plasma protein produced by the yolk sac and the liver during fetal life. Alpha-fetoprotein expression in adults is often associated with hepatoma or teratoma. However, hereditary persistance of alpha-fetoprotein may also be found in individuals with no obvious pathology. The protein is thought to be the fetal counterpart of serum albumin, and the alpha-fetoprotein and albumin genes are present in tandem in the same transcriptional orientation on chromosome 4. Alpha-fetoprotein is found in monomeric as well as dimeric and trimeric forms, and binds copper, nickel, fatty acids and bilirubin. The level of alpha-fetoprotein in amniotic fluid is used to measure renal loss of protein to screen for spina bifida and anencephaly.

GC (also known as group-specific component (vitamin D binding protein); DBP; VDBG; VDBP; and DBP/GC) encodes a protein that belongs to the albumin gene family. It is a multifunctional protein found in plasma, ascitic fluid, or cerebrospinal fluid and on the surface of many cell types. It binds to vitamin D and its plasma metabolites and transports them to target tissues.

APOC3 (also known as apolipoprotein C-III; APOCIII; and MGC150353) encodes a very low density lipoprotein (VLDL) protein. APOC3 inhibits lipoprotein lipase and hepatic lipase; it is thought to delay catabolism of triglyceride-rich particles. The APOA1, APOC3 and APOA4 genes are closely linked in both rat and human genomes. The A-I and A-IV genes are transcribed from the same strand, while the A-1 and C-III genes are convergently transcribed. An increase in apoC-III levels induces the development of hypertriglyceridemia.

SERPINC1 (also known as serpin peptidase inhibitor, Glade C (antithrombin), member 1; AT3; ATIII; and MGC22579) is a glycoprotein that can inactivate several enzymes of the coagulation system. SERPINC1 produced by the liver and consists of 432 amino acids. It contains three disulfide bonds and four possible glycosylation sites. The dominant form of antithrombin found in blood plasma is α-antithrombin. α-antithrombin has an oligosaccharide occupying each of its four glycosylation sites. In the minor form of antithrombin, β-antithrombin, a single glycosylation site remains consistently un-occupied.

APOB (also known as apolipoprotein B (including Ag(x) antigen) and FLDB) is the main apolipoprotein of chylomicrons and low density lipoproteins. It occurs in plasma as two main isoforms, apoB-48 and apoB-100: the former is synthesized exclusively in the gut and the latter in the liver. The intestinal and the hepatic forms of apoB are encoded by a single gene from a single, very long mRNA. The two isoforms share a common N-terminal sequence. The shorter apoB-48 protein is produced after RNA editing of the apoB-100 transcript at residue 2180 (CAA->UAA), resulting in the creation of a stop codon, and early translation termination. Mutations in this gene or its regulatory region cause hypobetalipoproteinemia, normotriglyceridemic hypobetalipoproteinemia, and hypercholesterolemia due to ligand-defective apoB, diseases affecting plasma cholesterol and apoB levels.

AHSG (also known as alpha-2-HS-glycoprotein; AHS; A2HS; HSGA; and FETUA) is a glycoprotein present in the serum and can be synthesized by hepatocytes. The AHSG molecule consists of two polypeptide chains, which are both cleaved from a proprotein encoded from a single mRNA. It is involved in several functions, such as endocytosis, brain development and the formation of bone tissue. The protein is commonly present in the cortical plate of the immature cerebral cortex and bone marrow hemopoietic matrix, and it has therefore been postulated that it participates in the development of the tissues.

HPX (also known as hemopexin) can bind heme. It can protect the body from the oxidative damage that can be caused by free heme by scavenging the heme released or lost by the turnover of heme proteins such as hemoglobin. To preserve the body's iron, upon interacting with a specific receptor situated on the surface of liver cells, hemopexin can release its bound ligand for internalisation.

CPB2 (also known as carboxypeptidase B2 (plasma); CPU; PCPB; and TAFI) is an enzyme that can hydrolyze C-terminal peptide bonds. The carboxypeptidase family includes metallo-, serine, and cysteine carboxypeptidases. According to their substrate specificity, these enzymes are referred to as carboxypeptidase A (cleaving aliphatic residues) or carboxypeptidase B (cleaving basic amino residues). The protein encoded by this gene is activated by trypsin and acts on carboxypeptidase B substrates. After thrombin activation, the mature protein downregulates fibrinolysis. Polymorphisms have been described for this gene and its promoter region. Available sequence data analyses indicate splice variants that encode different isoforms.

ITIH1 (also known as inter-alpha (globulin) inhibitor H1; H1P; ITIH; LATIH; and MGC126415) is a serine protease inhibitor family member. It is assembled from two precursor proteins: a light chain and either one or two heavy chains. ITIH1 can increase cell attachment in vitro.

APOH (also known as apolipoprotein H (beta-2-glycoprotein I); BG; and B2G1) has been implicated in a variety of physiologic pathways including lipoprotein metabolism, coagulation, and the production of antiphospholipid autoantibodies. APOH may be a required cofactor for anionic phospholipid binding by the antiphospholipid autoantibodies found in sera of many patients with lupus and primary antiphospholipid syndrome.

AMBP (also known as alpha-1-microglobulin/bikunin precursor; HCP; ITI; UTI; EDC1; HI30; ITIL; IATIL; and ITILC) encodes a complex glycoprotein secreted in plasma. The precursor is proteolytically processed into distinct functioning proteins: alpha-1-microglobulin, which belongs to the superfamily of lipocalin transport proteins and may play a role in the regulation of inflammatory processes, and bikunin, which is a urinary trypsin inhibitor belonging to the superfamily of Kunitz-type protease inhibitors and plays an important role in many physiological and pathological processes. This gene is located on chromosome 9 in a cluster of lipocalin genes.

J42-4-d is also known as t-complex 11 (mouse)-like 2; MGC40368 and TCP11L2.

In one embodiment, biomarker genes are differentially expressed in the first and/or second trimester.

“Differentially expressed,” as applied to nucleotide sequences or polypeptide sequences in a cell or cell nuclei, refers to differences in over/under-expression of that sequence when compared to the level of expression of the same sequence in another sample, a control or a reference sample. In one embodiment, expression differences can be temporal and/or cell-specific. For example, for cell-specific expression of biomarkers, differential expression of one or more biomarkers in the cell(s) of interest can be higher or lower relative to background cell populations. Detection of such a difference in expression of the biomarker can indicate the presence of a rare cell (e.g., fnRBC or a trophoblast) versus other cells in a mixed sample (e.g., background cell populations). In other embodiments, a ratio of two or more such biomarkers that are differentially expressed can be measured and used to detect rare cells.

Threshold of Expression Difference

In one embodiment transcript or protein expression of a gene in a fetal cell can be used as a marker to enrich, enumerate, purify, detect or identify the fetal cell if the expression of the gene is higher or lower in the fetal cell than in a reference sample, e.g., in a maternal cell. In one embodiment, a gene can be a fetal cell marker if the level of its expression (in the form of a transcript or protein) is at least about 10%, 11%, 12%, 13%, 14%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 750%, 1000%, 2000%, 3000%, 4000%, 5000% or 10,000% higher or lower than the level of expression of the gene (in the form of a transcript or protein) in a reference sample (e.g., a maternal cell). In one embodiment a gene has a higher level of protein or transcript expression in comparison to a reference sample (e.g., a maternal cell). In another embodiment a gene can be a marker of a fetal cell if the ratio of the expression of a of protein or transcript of the gene in a fetal cell compared to the expression of the gene in a reference sample (e.g., a maternal cell) is at least about 11:10, 6:5, 13:10, 7:5, 3:2, 8:5, 17:10, 9:5, 2:1, 3:1, 4:1, 5:1, or 10:1, 20:1, 25:1, 30:1, 35:1, 40:1, 45:1, 50:1, 55:1, 60:1, 65:1, 70:1, 75:1, 80:1, 85:1, 90:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, or 1000:1. In another embodiment a gene can be a marker of a fetal cell if the expression of a of protein or transcript of the gene in a fetal cell is at least about 1.1-, 1.2-, 1.3-, 1.4-, 1.5-, 1.6-, 1.7-, 1.8-, 1.9-, 2-, 3-, 4-, 5-, 10-, 15-, 20-, 25-, 30-, 35-, 40-, 45-, 50-, 55-, 60-, 65-, 70-, 75-, 80-, 85-, 90-, 95, or 100-fold higher or lower than expression of the transcript in a reference sample (e.g., a maternal cell). Levels of transcript or protein expression can be normalized to expression levels of other transcripts, or proteins, respectively.

Hemoglobins

In one embodiment, fetal biomarkers comprise differentially expressed hemoglobins. Erythroblasts (nRBCs) are abundant in the early fetal circulation and virtually absent in normal adult blood having a short finite lifespan, there is no risk of obtaining a fnRBC which can persist from a previous pregnancy. Furthermore, unlike trophoblast cells, fetal erythroblasts are not prone to mosaic characteristics.

Yolk sac erythroblasts synthesize ∈-, ζ-, γ- and α-globins, these combine to form the embryonic hemoglobins. Between six and eight weeks, the primary site of erythropoiesis shifts from the yolk sac to the liver, the three embryonic hemoglobins are replaced by fetal hemoglobin (HbF) as the predominant oxygen transport system, and ∈- and ζ-globin production gives way to γ-, α- and β-globin production within definitive erythrocytes (Peschle et al., 1985). HbF remains the principal hemoglobin until birth, when the second globin switch occurs and β-globin production accelerates.

Hemoglobin (Hb) is a heterodimer composed of two identical a globin chains and two copies of a second globin. Due to differential gene expression during fetal development, the composition of the second chain changes from ∈ globin during early embryonic development (1 to 4 weeks of gestation) to γ globin during fetal development (6 to 8 weeks of gestation) to β globin in neonates and adults as illustrated in (Table 1).

TABLE 1
Relative expression of ε, γ and β in maternal and fetal RBCs.

| | | ε | γ | β |
|---|---|---|---|---|
| 1^{st} trimester | Fetal | ++ | ++ | − |
| | Maternal | − | +/− | ++ |
| 2^{nd} trimester | Fetal | − | ++ | +/− |
| | Maternal | − | +/− | ++ |

In the late-first trimester, the earliest time that a fetal cell can be sampled by CVS, a fnRBC contains, in addition to α globin, primarily ∈ and γ globin. In the early to mid second trimester, when amniocentesis is typically performed, a fnRBC contains primarily γ globin with some adult β globin. Maternal cells contain almost exclusively α and β globin, with traces of γ detectable in some samples. Therefore, by measuring the relative expression of the ∈, γ and β genes in one or more RBCs purified from maternal blood samples, the presence of one or more fetal cells in the sample can be determined. Furthermore, positive controls can be utilized to assess the FISH analysis.

In one embodiment, a fetal cell is distinguished from a maternal cell based on the differential expression of hemoglobins β, γ or ∈. Expression levels or RNA levels can be determined in the cytoplasm or in the nucleus of a cell. Thus in one embodiment, the methods herein involve determining levels of messenger RNA (mRNA), ribosomal RNA (rRNA), or nuclear RNA (nRNA).

In one embodiment, identification of an fnRBC can be achieved by measuring the levels of at least two hemoglobins in the cytoplasm or nucleus of a cell. In various embodiments, identification and assay is from 1, 2, 3, 4, 5, 6, 7, 8, 9, 10, 15, or 20 fetal nuclei. Furthermore, total nuclei arrayed on one or more slides can number from about 100, 200, 300, 400, 500, 700, 800, 5000, 10,000, 100,000, 1,000,000, 2,000,000 to about 3,000,000. In one embodiment, a ratio for γ/β or ∈/β is used to determine the presence of one or more fetal cells, where a number less than one indicates that a fnRBC(s) is not present. In one embodiment, the relative expression of γ/β or ∈/β provides an fnRBC index (“FNI”), as measured by γ or ∈ relative to β. In one embodiment, a FNI for γ/β greater than 5, 10, 15, 20, 25, 30, 35, 40, 45, 90, 180, 360, 720, 975, 1020, 1024, 1250 to about 1250, indicate that an fnRBC(s) is present. In yet another embodiment, an FNI for γ/β of less than about 1 indicates that an fnRBC(s) is not present. The above FNI can be determined from a sample obtained during a first trimester. However, similar ratios can be used during second trimester and third trimester.

Detecting Expression of a Marker

Expression of gene expression can be determined by, for example, detecting transcripts or protein expressed from a gene. Expression of a transcript from a gene can be detected by, for example, RNA chromogenic in situ hybridization (CISH), RNA FISH, RNA-FISH using a molecular beacon probe, Q-PCR, RT-PCR, Taqman RT-PCR, Northern blotting, ribonuclease protection assay, or RNA expression profiling using microarrays.

Protein expression can be detected by, e.g., immunohistochemistry, immunocytochemistry, Western blotting, mass spectrometry, ELISA, gel electrophoresis followed by Coomassie staining or silver staining, flow cytometry, FACS, or microfluidic fluorescent cell sorting. The expressed protein can be a cell surface or an internal expressed protein. The cell surface protein can be recognized by a binding moiety, e.g., an antibody based moiety. The binding moieties used in detection can be an antibody, Fab fragment, Fc fragment, scFv fragment, peptidomimetic, or peptoid.

In one embodiment, the expression levels are determined by measuring nuclear RNA transcripts including, nascent or unprocessed transcripts. In another embodiment, expression levels are determined by measuring mRNA, including ribosomal RNA. There are many methods known in the art for imaging (e.g., measuring) nucleic acids or RNA including, but not limited to, using expression arrays from Affymetrix, Inc. or Illumina, Inc.

Primers and Probes

RT-PCR primers can be designed by targeting the globin variable regions, selecting the amplicon size, and adjusting the primers annealing temperature to achieve equal PCR amplification efficiency. Thus TaqMan probes can be designed for each of the amplicons with well-separated fluorescent dyes, Alexa Fluor®-355 for ∈, Alexa Fluor®-488 for γ, and Alexa Fluor-555 for β. The specificity of these primers can be first verified using ∈, γ, and β cDNA as templates. The primer sets that give the best specificity can be selected for further assay development. As an alternative, the primers can be selected from two exons spanning an intron sequence to amplify only the mRNA to eliminate the genomic DNA contamination.

The primers selected can be tested first in a duplex format to verify their specificity, limit of detection, and amplification efficiency using target cDNA templates. The best combinations of primers can be further tested in a triplex format for its amplification efficiency, detection dynamic range, and limit of detection.

Various commercially available reagents are available for RT-PCR, such as One-step RT-PCR reagents, including Qiagen One-Step RT-PCR Kit and Applied Biosystems TaqMan One-Step RT-PCR Master Mix Reagents kit. Such reagents can be used to establish the expression ratio of ∈, γ, and β using purified RNA from enriched samples. Forward primers can be labeled for each of the targets, using Alexa fluor-355 for ∈, Alexa fluor-488 for γ, and Alexa fluor-555 for β. Enriched cells can be deposited by cytospinning onto glass slides. Additionally, cytospinning the enriched cells can be performed after in situ RT-PCR. Thereafter, the presence of the fluorescent-labeled amplicons can be visualized by fluorescence microscopy. The reverse transcription time and PCR cycles can be optimized to maximize the amplicon signal:background ratio to have maximal separation of fetal over maternal signature. In one embodiment, signal:background ratio is greater than 5, 10, 50, or 100 and the overall cell loss during the process is less than 50, 10 or 5%.

Examples of other fluorescent molecules or dyes that can be used with the nucleic acid, antibody or antibody-based fragment probes of the present invention include Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800.

Primers and probes that can be used in the methods and compositions of the provided invention include those listed in Table 2.

TABLE 2
Primers and probes for detecting gene expression
Gene

| Symbol | Type | Name | Sequence |
|---|---|---|---|
| AFP | Forward Primer | AFP_317/318_F | CCCACTGGAGATGAACAGTCTTC |
| | Reverse Primer | AFP_317/318_R | TGGCAAAGTTCTTCCAGAAAGG |
| | Probe | AFP_317/318_P | TGTTTAGAAAACCAGCTACCT |
| | Forward Primer | AFP_1238/1239_F | CCAAGATAAAGGAGAAGAAGAATTACAGAA |
| | Reverse Primer | AFP_1238/1239_R | AGCAACGAGAAACGCATTTTG |
| | Probe | AFP_1238/1239_P | CATCCAGGAGAGCCAAG |
| | Forward Primer | AFP_1475/1476_F | GAGGGAGCGGCTGACATTATT |
| | Reverse Primer | AFP_1475/1476_R | ACACCAGGGTTTACTGGAGTCATT |
| | Probe | AFP_1475/1476_P | TCGGACACTTATGTATCAGACA |
| HPX | Forward Primer | HPX_83/84_F | CCCCTCTTCCTCCGACTAGTG |
| | Reverse Primer | HPX_83/84_R | CGTCTGGGTCTGGCTTGGT |
| | Probe | HPX_83/84_P | CATGGGAATGTTGCTGAA |
| | Forward Primer | HPX_142/143_F | TGACTGAACGCTGCTCAGATG |
| | Reverse Primer | HPX_142/143_R | CCCCTTTAAAAAACAGCATGGT |
| | Probe | HPX_142/143_P | CTGGAGCTTTGATGCTA |
| | Forward Primer | HPX_490/491_F | CACCGTGGAGAATGTCAAGCT |
| | Reverse Primer | HPX_490/491_R | CCGTAGCCAAGTCCCAGAAC |
| | Probe | HPX_490/491_P | CTCTTCTTCCAAGGTGACC |
| AMBP | Forward Primer | AMBP_600/601_F | CACAAATCCAAATGGAACATAACC |
| | Reverse Primer | AMBP_600/601_R | GGTCAGGAAAATGGCATACTCAT |
| | Probe | AMBP_600/601_P | TGGAGTCCTATGTGGTCC |
| | Forward Primer | AMBP_819/820_F | TCCCTGAGGACTCCATCTTTA |
| | Reverse Primer | AMBP_819/820_R | GGGATTAAGATGGGCTCTGGTT |
| | Probe | AMBP_819/820_P | CTGACCGAGGTGAATGT |
| GC | Forward Primer | GC_211/212_F | TGTGGCATTTGGACATGCTT |
| | Reverse Primer | GC_211/212_R | ATGGGAGAATTCCTTGCAGACTT |
| | Probe | GC_211/212_P | AGAGAGGCCGGGATT |
| | Forward Primer | GC_1548/1549_F | TGTTCCATAAACTCACCTCCTCTTT |
| | Reverse Primer | GC_1548/1549_R | TGCTTCAGGACTACAGGATATTCTTC |
| | Probe | GC_1548/1549_P | TGTGATTCAGAGATTGATGC |
| AHSG | Forward Primer | AHSG 654/655_F | TCCAATTTTAGCTGGAGGAA |
| | Reverse Primer | AHSG 654/655_R | CAGACACTGTAAACTCCACATAGGTAGA |
| | Probe | AHSG 654/655_P | TCAGCTTGTGCCCCTC |
| | Forward Primer | AHSG 756/757_F | GCTGGCAGAAAAGCAATATGG |
| | Reverse Primer | AHSG 756/757_R | CAACCTCTGCCCCACCAA |
| | Probe | AHSG 756/757_P | AGGCAACACTCAGTGAGA |
| ITIH1 | Forward Primer | ITIH1_141/142_F | GGCTACAGGCAGGTCCAAGA |
| | Reverse Primer | ITIH1_141/142_R | CGAGAGGTGACTTTGCAGTTGA |
| | Probe | ITIH1_141/142_P | CAGCGAGAAGCGAC |
| | Forward Primer | ITIH1_141/142_F | AGGATTCTCCGCCTTTGGA |
| | Reverse Primer | ITIH1_1948/1949_R | TGGAGCTGGAATGAGTAGGAGAAG |
| | Probe | ITIH1_1948/1949_P | CCAGAAGGACGTTCGTG |
| CPB2 | Forward Primer | CPB2 342/343_F | CGGAATTCCATGCAGTGTCTT |
| | Reverse Primer | CPB2 342/343_R | TGTCGTTGGAAATCTGCTGTTG |
| | Probe | CPB2 342/343_P | CAGATGTGGAAGATCT |
| | Forward Primer | CPB2 553/554_F | GATATGCTTACAAAAATCCACATTGG |
| | Reverse Primer | CPB2 553/554_R | GCATTTTTGGCTGCTTGTTCT |
| | Probe | CPB2 553/554_P | CACTCTATGTTTTAAAGGTTTCT |
| APOH | Forward Primer | APOH_397/398_F | TGAATATCCCAACACGATCAGTTT |
| | Reverse Primer | APOH_397/398_R | GGCAGAATCAGCGCCATT |
| | Probe | APOH_397/398_P | TCTTGTAACACTGGGTTTTA |
| | Forward Primer | APOH 1041/1042_F | GGCACTATCGAAGTCCCCAAA |
| | Reverse Primer | APOH 1041/1042_R | GATGCATCAGTTTTCCAAAAAGC |
| | Probe | APOH 1041/1042_P | CTTCAAGGAACACAGTTC |
| APOC3 | Forward Primer | APOC_3101/102_F | CAGCCCCGGGTACTCCTT |
| | Reverse Primer | APOC_3101/102_R | TTGGTGGCGTGCTTCATGTA |
| | Probe | APOC_3101/102_P | CTCTGCCCGAGCTT |
| APOB | Forward Primer | APOB 249/250_F | AGAGGAAATGCTGGAAAATGTCA |
| | Reverse Primer | APOB 249/250_R | CCGGAGGTGCTTGAATCG |
| | Probe | APOB 249/250_P | TCTGTCCAAAAGATGCG |
| | Forward Primer | APOB 3636/3637_F | TCCACAGTTTCCAAGAGGGTG |
| | Reverse Primer | APOB 3636/3637_R | GCCTGTGTTCCATTCAAATTCA |
| | Probe | APOB 3636/3637_P | GGCATTATGATGAAGAGAA |
| SER | Forward Primer | SERF1 | CCAAGCTGGGTGCCTGTAA |
| | Reverse Primer | SERR1 | GTTTGGCAAAGAAGAAGTGGATCT |
| | Probe | SERPl | TGATGGAGGTATTTAAGTTT |
| HBE | Forward Primer | HBE(GHC) | TGGAAGAGGCTGGAGGTGAA |
| | Reverse Primer | HBE(GHC) | AGACGACAGGTTTCCAAAGCTG |
| | Probe | HBE(GHC) | CAGACTCCTCGTTGTTT |
| | Forward Primer | HBE-1(AHI) | GCTGCATGTGGATCCTGAGA |
| | Reverse Primer | HBE-1(AHI) | TGAGTAGCCAGAATAATCACCATCA |
| | Probe | HBE-1(AHI) | CTTCAAGCTCCTGGGTAA |
| | Forward Primer | HBE-3(AHD | CTAGCCTGTGGAGCAAGATGAA |
| | Reverse Primer | HBE-3(AHI) | GACAGGTTTCCAAAGCTGTCAA |
| | Probe | HBE-3(AHI) | AGGCTGGAGGTGAAGC |
| J42-4d | Forward Primer | J42_4d_68_F | CAAGGCCTGGCCAACTATGT |
| | Reverse Primer | J42_4d_685_R | CGCACGGGAGCACACA |
| | Probe | J42_4d_685_P | ATCAGTACGATGGGAAAG |
| | Forward Primer | J42_4d_139_F | GACCCGGTGCTACCTTTTTACC |
| | Reverse Primer | J42_4d_139_R | CACTGCTTCTCGCCATTGAA |
| | Probe | J42_4d_139_P | TTAAGTGACGCAAAATG |
| | Forward Primer | J42_4d_809_F | GGTGCTGAGACAAATATTCCATGT |
| | Reverse Primer | J42_4d_809_R | TGCGGTCTGAGACTCATAATTGTAA |
| | Probe | J42_4d_809_P | TGCAAATGGACATGGC |
| | Forward Primer | J42_4d_1316_F | GAAGGCATGAACAAAGAGACCTTT |
| | Reverse Primer | J42_4d_1316_R | CCTCAACACAAGTCTGAATACCAATAG |
| | Probe | J42_4d_1316_P | CTTGAAGGAAGTCCTGAAT |
| hPL | Forward Primer | hPL | GCACCAGCTGGCCATTG |
| | Reverse Primer | | TGAATACTTCTGGTCCTTTGGGATA |
| | Probe | | AGGAGTTTGAAGAAACCT |
| | Forward Primer | CGB | CACCATCTGTGCCGGCTACT |
| CGB | Reverse Primer | | GCGCACATCGCGGTAGTT |
| | Probe | | CCCACCATGACCCG |
| KISS1 | Forward Primer | KISS1 | TCTGTGCCACCCACTTTGG |
| | Reverse Primer | | AGGAGGCCCAGGGATTCTAG |
| | Probe | | ACCCACAGGCCAGCA |
| CRH | Forward Primer | CRH | CCGGCTCACCTGCGAA |
| | Reverse Primer | | CGGCAGCCGCATGTTAG |
| | Probe | | CTGGGAAGCGAGTGC |
| LOC90625 | Forward Primer | LOC90625 | TGCACATCGGTCACTGATCTC |
| | Reverse Primer | | GGGTCAGTTTGGCCGATAAA |
| | Probe | | CCTACTGGCACAGACG |
| FN1 | Forward Primer | FN1 | GAAGACATACCACGTAGGAGAACA |
| | Reverse Primer | | AGGTCTGCGGCAGTTGTC |
| | Probe | | Roche Universal Probe #29 |
| PSG9 | Forward Primer | PSG9_163_164_F | GCTCACAGCATCACTTTTAAACTTCT |
| | Reverse Primer | PSG9_163_164_R | CTGGGCTTCAATCGTGACTTC |
| | Probe | PSG9_163_164_P | CCCGCCCACCACT |
| | Forward Primer | PSG9_1087/1088_F | TGGTGGCCTCCGCAGTAA |
| | Reverse Primer | PSG9_1087/1088_R | GGTAATAGGTGAATGAAGGGTAAATTCT |
| | Probe | PSG9_1087/1088_P | CTAAATGTCCTCTATGGTCCAG |

Fetal Cell Detection

In one embodiment the presence of or transcript expression of one or more genes in a fetal cell can be detected using one or more primer/probe sets. For example, at least 1, 2, 3, 4, 5, 6 or more primer/probe sets can be used to detect expression of one or more genes in a fetal cell (e.g., a fnRBC or trophoblast). In one embodiment a primer/probe set comprises two primers and one probe and optionally a quencher. In one embodiment a multiplex primer/probe combination comprises one or more primer/probe sets. In one embodiment a primer/probe set or a multiplex primer/probe combination is combined with a sample for q-PCR. In one embodiment a primer/probe set or a multiplex primer/probe combination is combined with a sample for Real Time-PCR. In one embodiment a multiplex primer/probe combination can be designed so as to balance the amounts of the primers and probes for each set so that a detectable signal is produced for each primer/probe set, if a target sequence is present in a sample. In one embodiment optimum annealing temperatures and thermocycling profiles can be designed so that multiple primer/probe combination can function in the same reaction chamber to detect the presence of a target sequence in sample. In one embodiment the probes are labeled with different fluorescent dyes. The dye labeled probes can be optimized so that each probe from a particular primer/probe set in a multiplex reaction, is labeled with a different dye that fluoresces at a peak wavelength sufficiently different from the other dye labeled probes so as to allow identification of the fluorescence from each sets probe. In one embodiment a probe is labeled with Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800. In one embodiment, a multiplex primer/probe combination comprises one or more primer/probe sets that anneal to a genomic DNA, of the hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d genes. In another embodiment, a multiplex primer/probe combination comprises one or more primer/probe sets that anneal to a RNA expressed by, or a cDNA of an RNA expressed by the hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d genes. In one embodiment, a fnRBC is enriched, enumerated, purified, detected or identified using a multiplex primer/probe combination comprising one or more primer/probe sets that anneal to a genomic DNA, a RNA expressed by, or a cDNA of an RNA expressed by the HBE, AFP, APOC3, SERPINC1, AMBP, CPB2, ITIH1, APOH, HPX, beta-hCG, AHSG, APOB, or J42-4-d genes. In another embodiment, a trophoblast is enriched, enumerated, purified, detected or identified using a multiplex primer/probe combination comprising one or more primer/probe sets that anneal to a genomic DNA, a RNA expressed by, or a cDNA of an RNA expressed by the hPL, CHS2, KISS1, GDF15, CRH, TFP12, CGB, LOC90625, FN1, COL1A2, PSG9, or PSG1 genes. In one embodiment a multiplex primer/probe combination comprises at least three primer/probe sets that anneal to a genomic DNA, a RNA expressed by, or a cDNA of an RNA expressed by the HBE, hPL, or AFP genes. In another embodiment a multiplex primer/probe combination comprises at least three primer/probe sets that anneal to a genomic DNA, a RNA expressed by, or a cDNA of an RNA expressed by the FN1, beta-hCG, or AHSG genes.

In another embodiment at least 1, 2, 3, 4, 5, 6 or more sets of primers can be used to detect the presence of or transcript expression of one or more genes in a fetal cell (e.g., a fnRBC or trophoblast). In one embodiment a primer set comprises two primers. In one embodiment two or more primer sets are included in a multiplex reaction with a sample, comprising a target sequence. In one embodiment a multiplex primer combination can be designed so as to balance the amounts of the primers for each set so that a detectable amplified product is produced for each primer set, if a target sequence is present in a sample. In one embodiment optimum annealing temperatures and thermocycling profiles can be designed so that multiple primer sets can be combined to function in the same reaction chamber to amplify the presence of a target sequence in sample. In one embodiment, a primer set anneals to a genomic DNA, of the hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCGbeta-hCG, AHSG, APOB, or J42-4-d genes. In another embodiment, a primer set anneals to an RNA expressed by, or a cDNA of an RNA expressed by the hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d genes. In one embodiment, a fnRBC is enriched, enumerated, purified, detected or identified using a primer set that anneals to a genomic DNA, a RNA expressed by, or a cDNA of an RNA expressed by the HBE, AFP, APOC3, SERPINC1, AMBP, CPB2, ITIH1, APOH, HPX, beta-hCG, AHSG, APOB, or J42-4-d genes. In another embodiment, a trophoblast is enriched, enumerated, purified, detected or identified using a primer set that anneals to a genomic DNA, a RNA expressed by, or a cDNA of an RNA expressed by the hPL, CHS2, KISS1, GDF15, CRH, TFP12, CGB, LOC90625, FN1, COL1A2, PSG9, or PSG1 genes.

In another embodiment at least 1, 2, 3, 4, 5, 6 or more probes can be used to detect transcript expression of one or more genes by a fetal cell (e.g., a fnRBC or trophoblast). In one embodiment two or more probes are detectably labeled and can bind to an RNA sequence. In one embodiment two or more probes are used to detect more than one RNA sequence expressed by a fetal cell. In one embodiment the two or more probes are used in a method of fluorescent in-situ hybridization. In one embodiment the method of fluorescent in-situ hybridization is RNA-FISH. In one embodiment the probes are nucleic acid probes. In another embodiment the probe is a peptide nucleic acid (PNA). In another embodiment a probe comprises one or more modified nucleic acids, such as an amide modified nucleic acid, a phosphoramidate modified nucleic acid, a boranophosphate modified nucleic acid, a methylphosophonate modified nucleic acid, a deoxyribonucleic guanidine (DNG) modified nucleic acid or a morpholino modified nucleic acid.

In one embodiment two or more probes are labeled with a detectable tag, such as biotin or streptavidin, which can bind to a labeled conjugate. In another embodiment the probe is labeled with an enzyme (such as alkaline phosphatase) that can convert a substrate (such as Fast Red) into a detectable label. In one embodiment the enzyme is alkaline phosphatase, horseradish peroxidase, beta-galactosidase, or glucose oxidase.

Alkaline phosphatase substrates include, but are not limited to, AP-Blue substrate (blue precipitate, Zymed); AP-Orange substrate (orange, precipitate, Zymed), AP-Red substrate (red, red precipitate, Zymed), 5-bromo, 4-chloro, 3-indolyphosphate (BCIP substrate, turquoise precipitate), 5-bromo, 4-chloro, 3-indolyl phosphate/nitroblue tetrazolium/iodonitrotetrazolium (BCIP/INT substrate, yellow-brown precipitate, Biomeda), 5-bromo, 4-chloro, 3-indolyphosphate/nitroblue tetrazolium (BCIP/NBT substrate, blue/purple), 5-bromo, 4-chloro, 3-indolyl phosphate/nitroblue tetrazolium/iodonitrotetrazolium (BCIP/NBT/INT, brown precipitate, DAKO, Fast Red (Red), Magenta-phos (magenta), Naphthol AS-BI-phosphate (NABP)/Fast Red, TR (Red), Naphthol AS-BI-phosphate (NABP)/New Fuchsin (Red), Naphthol AS-MX-phosphate (NAMP)/New Fuchsin (Red), New Fuchsin AP substrate (red), p-Nitrophenyl phosphate (PNPP, Yellow, water soluble), VECTOR.Black (black), VECTOR. Blue (blue), VECTOR. Red (red), or Vega Red (raspberry red color).

Horseradish Peroxidase (HRP, sometimes abbreviated PO) substrates include, but are not limited to, 2,2′ Azino-di-3-ethylbenz-thiazoline sulfonate (ABTS, green, water soluble), aminoethyl carbazole, 3-amino, 9-ethylcarbazole AEC (3A9EC, red). Alpha-naphthol pyronin (red), 4-chloro-1-naphthol (4C1N, blue, blue-black), 3,3′-diaminobenzidine tetrahydrochloride (DAB, brown), ortho-dianisidine (green), o-phenylene diamine (OPD, brown, water soluble), TACS Blue (blue), TACS Red (red), 3,3′,5,5′ Tetramethylbenzidine (TMB, green or green/blue), TRUE BLUE. (blue), VECTOR.VIP (purple), VECTOR. SG (smoky blue-gray), or Zymed Blue HRP substrate (vivid blue).

Glucose Oxidase (GO) substrates, include, but are not limited to, nitroblue tetrazolium (NBT, purple precipitate), tetranitroblue tetrazolium (TNBT, black precipitate), 2-(4-iodophenyl)-5-(4-nitorphenyl)-3-phenyltetrazolium chloride (INT, red or orange precipitate), Tetrazolium blue (blue), Nitrotetrazolium violet (violet), or 3-(4,5-dimethylthiazol-2-yl)-2,5-diphenyltetrazolium bromide (MTT, purple). Tetrazolium substrates generally require glucose as a co-substrate. The glucose is oxidized and the tetrazolium salt are reduced and form an insoluble formazan which forms the color precipitate.

Beta-Galactosidase substrates, include, but are not limited to, 5-bromo-4-chloro-3-indoyl beta-D-galactopyranoside (X-gal, blue precipitate).

In one embodiment the conjugate is labeled with a fluorescent dye. In another embodiment, two or more probes are detectably labeled by fluorescent labeling. In one embodiment two or more probes are labeled with the same fluorescent label. In one embodiment two or more probes are labeled with different fluorescent labels. The fluorescently labeled probes can be optimized so that each probe from is labeled with a different label that fluoresces at a peak wavelength sufficiently different from the other fluorescently labeled probe so as to allow identification of the fluorescence from each probe. In one embodiment a probe is directly labeled with, or can bind to a conjugate labeled with: Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800.

In one embodiment, one or more detectably labeled probes anneal to an RNA sequence expressed by an hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d gene. In one embodiment, an fnRBC is enriched, enumerated, purified, detected or identified using one or more detectably labeled probes that anneal to an RNA sequence expressed by one or more of the HBE, AFP, APOC3, SERPINC1, AMBP, CPB2, ITIH1, APOH, HPX, beta-hCG, AHSG, APOB, or J42-4-d genes. In another embodiment, a trophoblast is enriched, enumerated, purified, detected or identified using one or more detectably labeled probes that anneal to an RNA sequence expressed by two or more of the hPL, CHS2, KISS1, GDF15, CRH, TFP12, CGB, LOC90625, FN1, COL1A2, PSG9, or PSG1 genes. In one embodiment a fetal cell is enriched, enumerated, purified, detected or identified using detectably labeled probes that anneal to an RNA sequence expressed by the HBE, hPL, or AFP genes. In another embodiment a fetal cell is enriched, enumerated, purified, detected or identified using detectably labeled probes that anneal to an RNA sequence expressed by the FN1, beta-hCG, or AHSG genes. In another embodiment a fetal cell is enriched, enumerated, purified, detected or identified using detectably labeled probes that anneal to an RNA sequence expressed by the HBE, AFP, hPL, or FN1 genes. In another embodiment a fetal cell is enriched, enumerated, purified, detected or identified using detectably labeled probes that anneal to an RNA sequence expressed by the HBE, AFP, hPL, or beta-hCG genes. In another embodiment a fetal cell is enriched, enumerated, purified, detected or identified using detectably labeled probes that anneal to an RNA sequence expressed by the HBE, AHSG, AFP, hPL, or beta-hCG genes. In another embodiment a fetal cell is enriched, enumerated, purified, detected or identified using detectably labeled probes that anneal to an RNA sequence expressed by one of the HBE, AHSG, AFP, hPL, or FN1 genes. In another embodiment a fetal cell is enriched, enumerated, purified, detected or identified detectably labeled probes that anneal to an RNA sequence expressed by the HBE, AHSG, AFP, hPL, beta-hCG, or FN1 genes.

In another embodiment at least 1, 2, 3, 4, 5, 6 or more antibodies or antibody-based fragments can be used to detect expression of one or more proteins in a fetal cell (e.g., a fnRBC or trophoblast).

In one embodiment an antibody or antibody-based fragment is labeled with a detectable tag, such as biotin or streptavidin, which can bind to a labeled conjugate. In another embodiment an antibody or antibody-based fragment is labeled with an enzyme (such as alkaline phosphatase) that can convert a substrate (such as Fast Red) into a detectable label. In one embodiment the enzyme is alkaline phosphatase, horseradish peroxidase, beta.-galactosidase, or glucose oxidase.

Alkaline phosphatase substrates include, but are not limited to, AP-Blue substrate (blue precipitate, Zymed); AP-Orange substrate (orange, precipitate, Zymed), AP-Red substrate (red, red precipitate, Zymed), 5-bromo, 4-chloro, 3-indolyphosphate (BCIP substrate, turquoise precipitate), 5-bromo, 4-chloro, 3-indolyl phosphate/nitroblue tetrazolium/iodonitrotetrazolium (BCIP/INT substrate, yellow-brown precipitate, Biomeda), 5-bromo, 4-chloro, 3-indolyphosphate/nitroblue tetrazolium (BCIP/NBT substrate, blue/purple), 5-bromo, 4-chloro, 3-indolyl phosphate/nitroblue tetrazolium/iodonitrotetrazolium (BCIP/NBT/INT, brown precipitate, DAKO, Fast Red (Red), Magenta-phos (magenta), Naphthol AS-BI-phosphate (NABP)/Fast Red, TR (Red), Naphthol AS-BI-phosphate (NABP)/New Fuchsin (Red), Naphthol AS-MX-phosphate (NAMP)/New Fuchsin (Red), New Fuchsin AP substrate (red), p-Nitrophenyl phosphate (PNPP, Yellow, water soluble), VECTOR.Black (black), VECTOR. Blue (blue), VECTOR. Red (red), or Vega Red (raspberry red color).

Horseradish Peroxidase (HRP, sometimes abbreviated PO) substrates include, but are not limited to, 2,2′ Azino-di-3-ethylbenz-thiazoline sulfonate (ABTS, green, water soluble), aminoethyl carbazole, 3-amino, 9-ethylcarbazole AEC (3A9EC, red). Alpha-naphthol pyronin (red), 4-chloro-1-naphthol (4C1N, blue, blue-black), 3,3′-diaminobenzidine tetrahydrochloride (DAB, brown), ortho-dianisidine (green), o-phenylene diamine (OPD, brown, water soluble), TACS Blue (blue), TACS Red (red), 3,3′,5,5′ Tetramethylbenzidine (TMB, green or green/blue), TRUE BLUE. (blue), VECTOR.VIP (purple), VECTOR. SG (smoky blue-gray), or Zymed Blue HRP substrate (vivid blue).

Glucose Oxidase (GO) substrates, include, but are not limited to, nitroblue tetrazolium (NBT, purple precipitate), tetranitroblue tetrazolium (TNBT, black precipitate), 2-(4-iodophenyl)-5-(4-nitorphenyl)-3-phenyltetrazolium chloride (INT, red or orange precipitate), Tetrazolium blue (blue), Nitrotetrazolium violet (violet), or 3-(4,5-dimethylthiazol-2-yl)-2,5-diphenyltetrazolium bromide (MTT, purple). Tetrazolium substrates generally require glucose as a co-substrate. The glucose is oxidized and the tetrazolium salt are reduced and form an insoluble formazan which forms the color precipitate.

Beta-Galactosidase substrates, include, but are not limited to, 5-bromo-4-chloro-3-indoyl beta-D-galactopyranoside (X-gal, blue precipitate).

In one embodiment an antibody or antibody fragment binds to a fetal cell marker protein. In one embodiment an antibody or antibody fragment is labeled with a fluorescent dye. In another embodiment an antibody or antibody fragment binds to an antibody or antibody fragment labeled with a fluorescent dye. In one embodiment more than one antibody or antibody fragments is labeled with the same fluorescent dye. In one embodiment each antibody or antibody fragment is labeled with a different fluorescent dye. The dye labeled antibody or antibody-based fragment can be optimized so that each antibody or antibody-based fragment is labeled with a different dye that fluoresces at a peak wavelength sufficiently different from another dye labeled antibody or antibody-based fragment so as to allow identification of the fluorescence from each antibody or antibody-based fragment. In one embodiment two or more dye labeled antibodies are bound to a fetal cell for detection by FACS or microfluidic fluorescent cell sorting. In one embodiment an antibody that binds to a fetal marker is labeled with Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800.

In one embodiment at least 1, 2, 3, 4, 5, 6 or more anti-hPL, anti-CHS2, anti-KISS1, anti-GDF15, anti-CRH, anti-TFP12, anti-CGB, anti-LOC90625, anti-FN1, anti-COL1A2, anti-PSG9, anti-PSG1, anti-HBE, anti-AFP, anti-APOC3, anti-SERPINC1, anti-AMBP, anti-CPB2, anti-ITIH1, anti-APOH, anti-HPX, anti-beta-hCG, anti-AHSG, anti-APOB, or anti-J42-4-d antibodies or antibody-based fragments are used to detect expression of one or more proteins by a fetal cell. In one embodiment an antibody or antibody-based fragment binds to a protein within a fetal cell. In another embodiment an antibody, or antibody-based fragment binds to a protein expressed on the surface of a fetal cell. In one embodiment, an fnRBC is enriched, enumerated, purified, detected or identified using one or more antibodies or antibody fragments that can bind proteins expressed from the HBE, AFP, APOC3, SERPINC1, AMBP, CPB2, ITIH1, APOH, HPX, beta-hCG, AHSG, APOB, or J42-4-d genes. In another embodiment, a trophoblast is enriched, enumerated, purified, detected or identified using one or more antibodies or antibody fragments that can bind proteins expressed from the hPL, CHS2, KISS1, GDF15, CRH, TFP12, CGB, LOC90625, FN1, COL1A2, PSG9, or PSG1 genes. In one embodiment a fetal cell is enriched, enumerated, purified, detected or identified using antibodies or antibody fragments that bind to proteins expressed by the HBE, hPL, or AFP genes. In another embodiment a fetal cell is enriched, enumerated, purified, detected or identified using antibodies or antibody fragments that bind to proteins expressed by the FN1, beta-hCG, or AHSG genes. In another embodiment a fetal cell is enriched, enumerated, purified, detected or identified using antibodies or antibody fragments that bind to proteins expressed by the HBE, AFP, hPL, or FN1 genes. In another embodiment a fetal cell is enriched, enumerated, purified, detected or identified using antibodies or antibody fragments that bind to proteins expressed by the HBE, AFP, hPL, or beta-hCG genes. In another embodiment a fetal cell is enriched, enumerated, purified, detected or identified using antibodies or antibody fragments that bind to proteins expressed by the HBE, AHSG, AFP, hPL, or FN1 genes. In another embodiment a fetal cell is enriched, enumerated, purified, detected or identified using antibodies or antibody fragments that bind to proteins expressed by the HBE, AHSG, AFP, hPL, beta-hCG, or FN1 genes.

Applications of Fetal Cell Markers

The detection of protein or transcript expression by specific genes can be used to distinguish a fetal cell from a reference cell, e.g., a maternal cell, distinguish between fetal cell types, identify a fetal cell, purify or enrich one or more fetal cells, or for enumeration of one or more fetal cells.

In one embodiment, cell type specific FCMs can be used to identify the fetal cell types by an RT-PCR approach.

In one embodiment, a fetal cell can be labeled by RNA FISH. In one embodiment a fetal cell can be labeled with a molecular beacon. In one embodiment a fetal cell labeled with a molecular beacon can be identified, purified, enriched or enumerated by FACS or microfluidic fluorescent cell sorting.

In one embodiment, by combining RT-PCR and digital PCR, fetal cell types can be identified and the fetal cell numbers counted.

In one embodiment, a fetal cell can be labeled by an antibody or antibody-based fragment that binds to a protein expressed by a FCM gene. In one embodiment a fetal cell labeled with an antibody or antibody-based fragment can be identified, purified, enriched or enumerated by FACS or microfluidic fluorescent cell sorting.

IV. Fetal Cell Analysis

Fetal conditions that can be determined based on the methods and systems herein include the presence of a fetus and/or a condition of the fetus such as fetal aneuploidy e.g., trisomy 13, trisomy 18, trisomy 21 (Down Syndrome), Klinefelter Syndrome (XXY) and other irregular number of sex or autosomal chromosomes, including monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g., XXXX, XXYY, XXXY, XYYY, XXXXX, XXXXY, XXXYY, XYYYY and XXYYY), monoploidy, triploidy (three of every chromosome, e.g., 69 chromosomes in humans), tetraploidy (four of every chromosome, e.g., 92 chromosomes in humans), pentaploidy and multiploidy. Other fetal conditions that can be detected using the methods herein include segmental aneuploidy, such as 1p36 duplication, dup(17)(p11.2p11.2) syndrome, Down syndrome, Pre-eclampsia, Pre-term labor, Edometriosis, Pelizaeus-Merzbacher disease, dup(22)(q11.2q11.2) syndrome, Cat eye syndrome. In one embodiment, the fetal abnormality to be detected is due to one or more deletions in sex or autosomal chromosomes, including Cri-du-chat syndrome, Wolf-Hirschhorn syndrome, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, steroid sulfatase deficiency, Kallmann syndrome, Microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c) and 1p36 deletion. In one embodiment, the fetal abnormality is an abnormal decrease in chromosomal number, such as XO syndrome.

In one embodiment, sample analysis involves performing one or more genetic analyses or detection steps on nucleic acids from the enriched product (e.g., enriched cells or nuclei). Nucleic acids from enriched cells or enriched nuclei that can be analyzed by the methods herein include: double-stranded DNA, single-stranded DNA, single-stranded DNA hairpins, DNA/RNA hybrids, RNA (e.g. mRNA) and RNA hairpins. Examples of genetic analyses that can be performed on enriched cells or nucleic acids include, e.g., SNP detection, STR detection, and RNA expression analysis.

In one embodiment, less than 1 μg, 500 ng, 200 ng, 100 ng, 50 ng, 40 ng, 30 ng, 20 ng, 10 ng, 5 ng, 1 ng, 500 pg, 200 pg, 100 pg, 50 pg, 40 pg, 30 pg, 20 pg, 10 pg, 5 pg, or 1 pg of nucleic acids are obtained from a sample or an enriched sample for further genetic analysis. In one embodiment, about 1-5 μg, 5-10 or 10-100 μg of nucleic acids are obtained from the enriched sample for further genetic analysis.

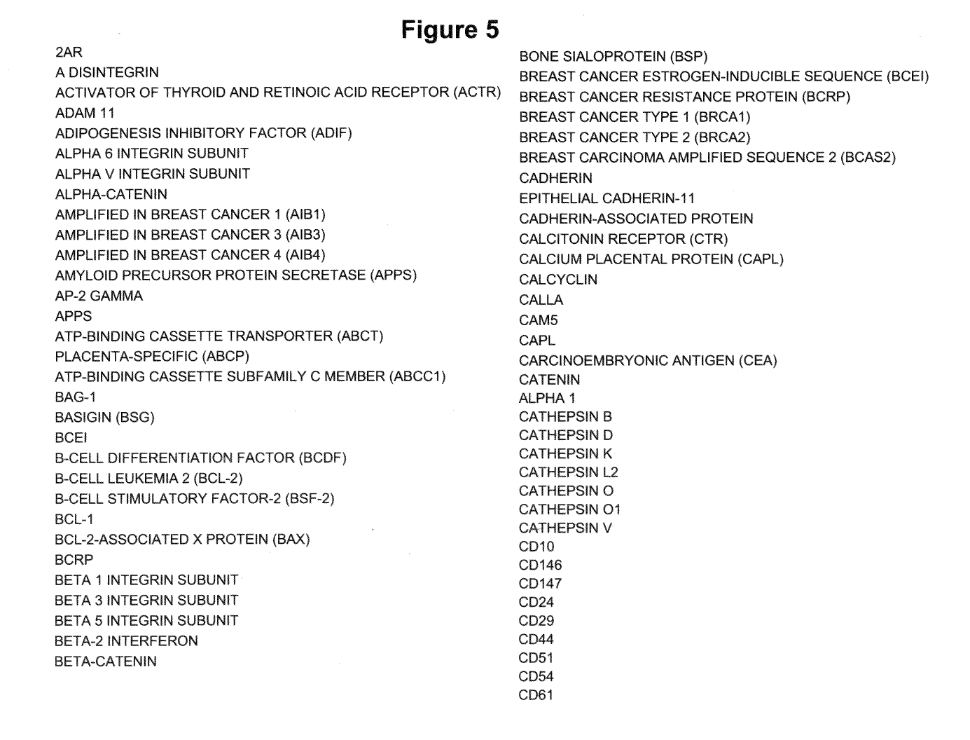

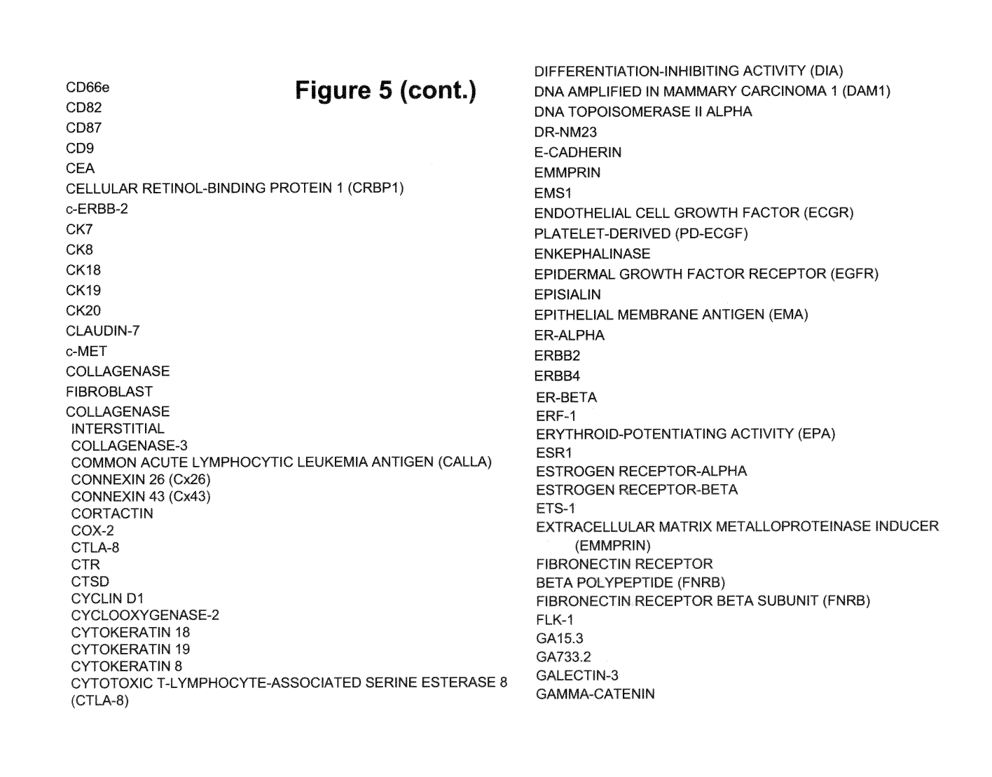

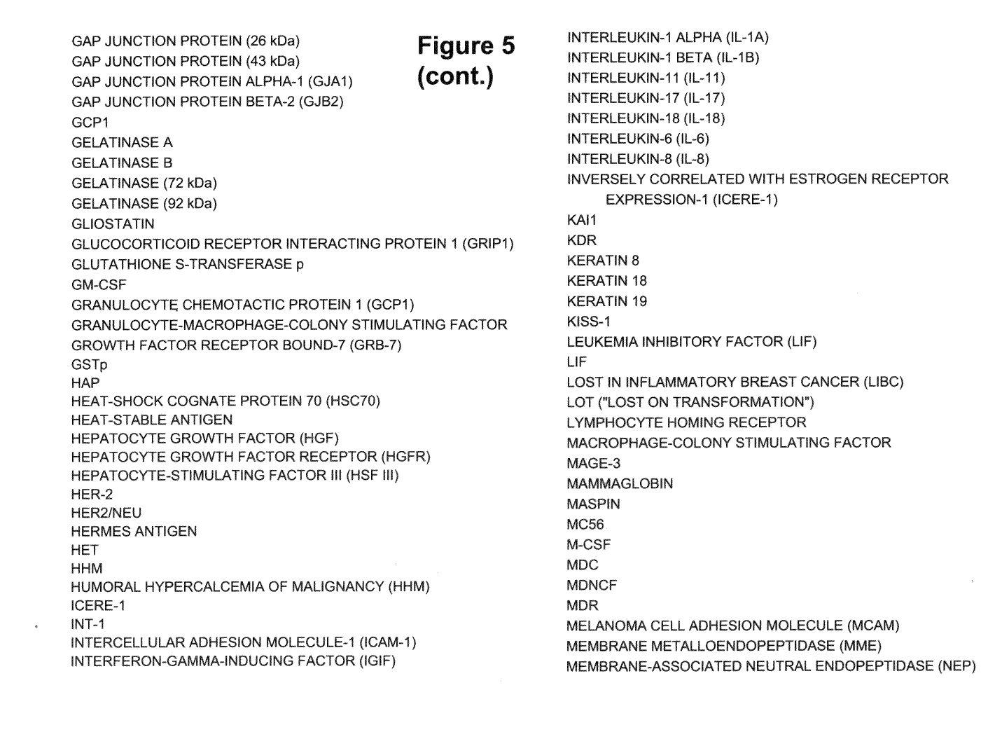

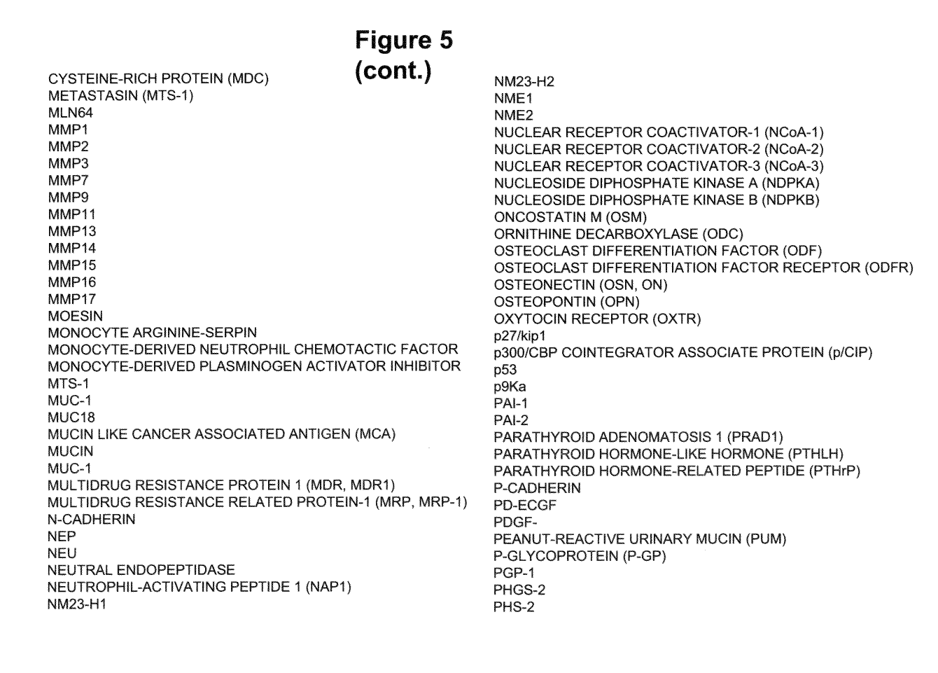

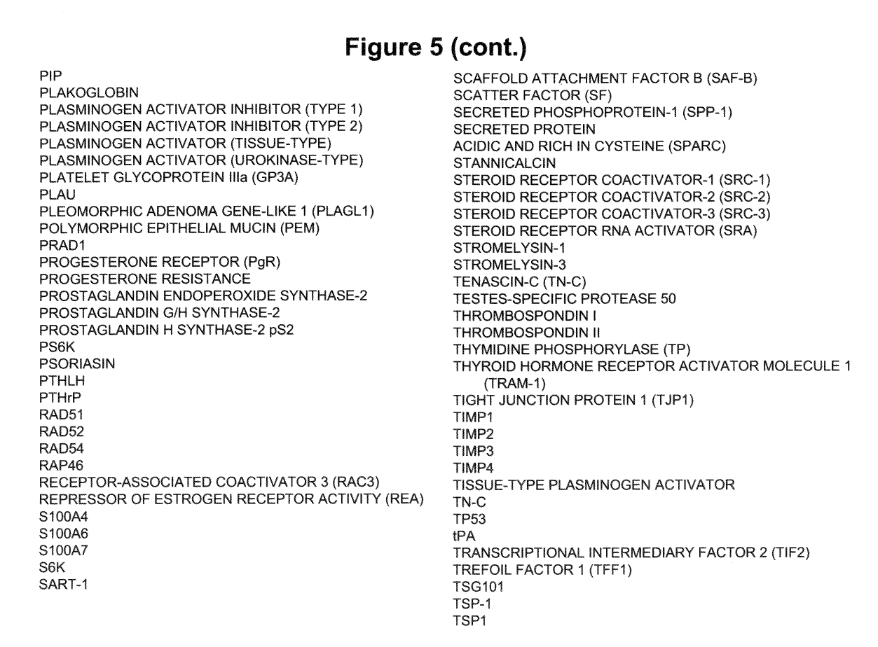

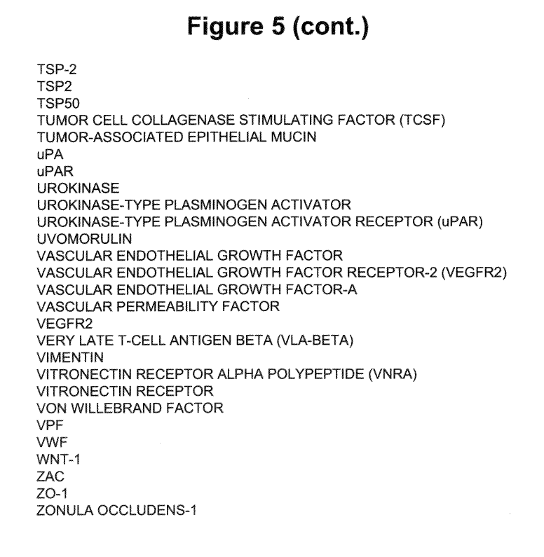

When analyzing, for example, a sample such as a blood sample from a patient to diagnose a condition such as cancer, the genetic analyses can be performed on one or more genes encoding or regulating a polypeptide listed in FIG. 5. In one embodiment, a diagnosis is made by comparing results from such genetic analyses with results from similar analyses from a reference sample (one without one or more fetal cells). For example, a maternal blood sample enriched for one or more fetal cells can be analyzed to determine the presence of one or more fetal cells and/or a condition in such cells by comparing the ratio of maternal to paternal genomic DNA (or alleles) in control and test samples.

In one embodiment, target nucleic acids from a test sample are amplified and optionally results are compared with amplification of similar target nucleic acids from a non-rare cell population (reference sample). Amplification of target nucleic acids can be performed by any means known in the art. In one embodiment, target nucleic acids are amplified by polymerase chain reaction (PCR). Examples of PCR techniques that can be used include, but are not limited to, digital PCR, reverse transcription PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), bridge PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938, which are herein incorporated by reference in their entirety

In any of the embodiments, amplification of target nucleic acids can occur on a bead. In any of the embodiments herein, target nucleic acids can be obtained from a single cell.

In any of the embodiments herein, the nucleic acid(s) of interest can be pre-amplified prior to the amplification step (e.g., PCR). In one embodiment, a nucleic acid sample can be pre-amplified to increase the overall abundance of genetic material to be analyzed (e.g., DNA). Pre-amplification can therefore include whole genome amplification such as multiple displacement amplification (MDA) or amplifications with outer primers in a nested PCR approach.

In one embodiment amplified nucleic acid(s) are quantified. Methods for quantifying nucleic acids are known in the art and include, but are not limited to, gas chromatography, supercritical fluid chromatography, liquid chromatography (including partition, chromatography, adsorption chromatography, ion exchange chromatography, size-exclusion chromatography, thin-layer chromatography, and affinity chromatography), electrophoresis (including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis), comparative genomic hybridization (CGH), microarrays, bead arrays, and high-throughput genotyping such as with the use of molecular inversion probe (MIP).

Quantification of amplified target nucleic acid can be used to determine gene/or allele copy number, gene or exon-level expression, methylation-state analysis, or detect a novel transcript in order to diagnose a condition, e.g., fetal abnormality.

In one embodiment, analysis involves detecting one or more mutations or SNPs in DNA from e.g., enriched rare cells or enriched rare DNA. Such detection can be performed using, for example, DNA microarrays. Examples of DNA microarrays include those commercially available from Affymetrix, Inc. (Santa Clara, Calif.), including the GeneChip™ Mapping Arrays including Mapping 100K Set, Mapping 10K 2.0 Array, Mapping 10K Array, Mapping 500K Array Set, and GeneChip™ Human Mitochondrial Resequencing Array 2.0. The Mapping 10K array, Mapping 100K array set, and Mapping 500K array set analyze more than 10,000, 100,000 and 500,000 different human SNPs, respectively. SNP detection and analysis using GeneChip™ Mapping Arrays is described in part in Kennedy, G. C., et al., Nature Biotechnology 21, 1233-1237, 2003; Liu, W. M., Bioinformatics 19, 2397-2403, 2003; Matsuzaki, H., Genome Research 3, 414-25, 2004; and Matsuzaki, H., Nature Methods, 1, 109-111, 2004 as well as in U.S. Pat. Nos. 5,445,934; 5,744,305; 6,261,776; 6,291,183; 5,799,637; 5,945,334; 6,346,413; 6,399,365; and 6,610,482, and EP 619 321; 373 203, which are herein incorporated by reference in their entirety. In one embodiment, a microarray is used to detect at least 5, 10, 20, 50, 100, 200, 500, 1,000, 2,000, 5,000 10,000, 20,000, 50,000, 100,000, 200,000, or 500,000 different nucleic acid target(s) (e.g., SNPs, mutations or STRs) in a sample.

Methods for analyzing chromosomal copy number using mapping arrays are disclosed, for example, in Bignell et al., Genome Res. 14:287-95 (2004), Lieberfarb, et al., Cancer Res. 63:4781-4785 (2003), Zhao et al., Cancer Res. 64:3060-71 (2004), Nannya et al., Cancer Res. 65:6071-6079 (2005) and Ishikawa et al., Biochem. and Biophys. Res. Comm., 333:1309-1314 (2005), which are herein incorporated by reference in their entirety. Computer implemented methods for estimation of copy number based on hybridization intensity are disclosed in U.S. Publication Application Nos. 20040157243; 20050064476; and 20050130217, which are herein incorporated by reference in their entirety.

In another aspect, mapping analysis using fixed content arrays, for example, 10K, 100K or 500K arrays, identifies one or more regions that show linkage or association with the phenotype of interest. These linked regions can then be analyzed to identify and genotype polymorphisms within the identified region or regions, for example, by designing a panel of MIPs targeting polymorphisms or mutations in the identified region. The targeted regions can be amplified by hybridization of a target specific primer and extension of the primer by a highly processive strand displacing polymerase, such as phi29 and then analyzed, for example, by genotyping.

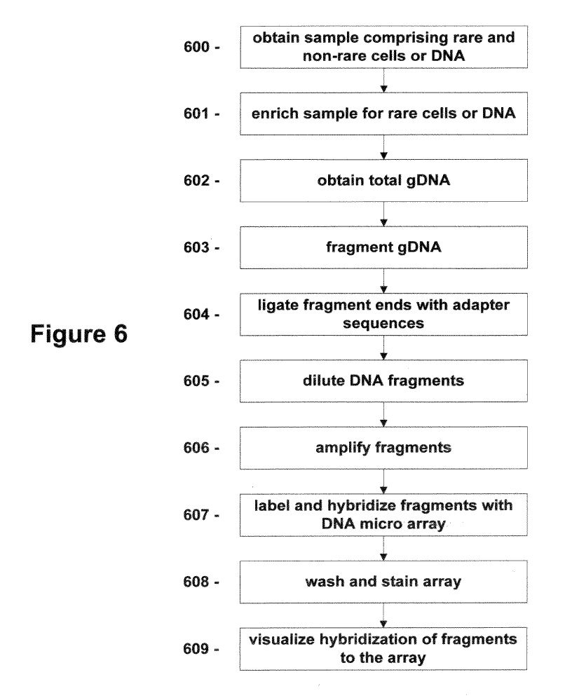

An overview for the process of using a SNP detection microarray (such as the Mapping 100K Set) is illustrated in FIG. 6. First, in step 600 a sample comprising one or more rare cells (e.g., fetal cells) and non-rare cells (e.g., RBC's) is obtained from an animal such as a human. In step 601, rare cells or rare DNA (e.g., rare nuclei) are enriched using one or more methods disclosed herein or known in the art. In one embodiment, rare cells are enriched by flowing the sample through an array of obstacles that selectively directs particles or cells of different hydrodynamic sizes into different outlets. In one embodiment, cDNA is obtained from both rare and non-rare cells enriched by the methods herein.

In step 602, genomic DNA is obtained from the rare cell(s) or nuclei and optionally one or more non-rare cells remaining in the enriched mixture. In step 603, the genomic DNA obtained from the enriched sample is digested with a restriction enzyme, such as XbaI or Hind III. Other DNA microarrays can be designed for use with other restriction enzymes, e.g., Sty I or NspI. In step 604 all fragments resulting from the digestion are ligated on both ends with an adapter sequence that recognizes the overhangs from the restriction digest. In step 605, the DNA fragments are diluted. Subsequently, in step 606 fragments having the adapter sequence at both ends are amplified using a generic primer that recognizes the adapter sequence. The PCR conditions used for amplification preferentially amplify fragments that have a unique length, e.g., between 250 and 2,000 base pairs in length. In step 607, amplified DNA sequences are fragmented, labeled and hybridized with the DNA microarray (e.g., 100K Set Array or other array). Hybridization is followed by a step 608 of washing and staining.

In step 609 results are visualized using a scanner that enables the viewing of intensity of data collected and a software “calls” the bases present at each of the SNP positions analyzed. Computer implemented methods for determining genotype using data from mapping arrays are disclosed, for example, in Liu, et al., Bioinformatics 19:2397-2403, 2003; and Di et al., Bioinformatics 21:1958-63, 2005. Computer implemented methods for linkage analysis using mapping array data are disclosed, for example, in Ruschendorf and Nurnberg, Bioinformatics 21:2123-5, 2005; and Leykin et al., BMC Genet. 6:7, 2005; and in U.S. Pat. No. 5,733,729, which are herein incorporated by reference in their entirety.

In one embodiment, genotyping microarrays that are used to detect SNPs can be used in combination with molecular inversion probes (MIPs) as described in Hardenbol et al., Genome Res. 15(2):269-275, 2005, Hardenbol, P. et al. Nature Biotechnology 21(6), 673-8, 2003; Faham M, et al. Hum Mol. Genet. August 1; 10(16):1657-64, 2001; Maneesh Jain, Ph.D., et all. Genetic Engineering News V24: No. 18, 2004; and Fakhrai-Rad H, et al. Genome Res. July; 14(7):1404-12, 2004; and in U.S. Pat. No. 6,858,412, which are herein incorporated by reference in their entireties. Universal tag arrays and reagent kits for performing such locus specific genotyping using panels of custom MIPs are available from Affymetrix and ParAllele. MIP technology involves the use enzymological reactions that can score up to 10,000; 20,000, 50,000; 100,000; 200,000; 500,000; 1,000,000; 2,000,000 or 5,000,000 SNPs (target nucleic acids) in a single assay. The enzymological reactions are insensitive to cross-reactivity among multiple probe molecules and there is no need for pre-amplification prior to hybridization of the probe with the genomic DNA. In any of the embodiments, the target nucleic acid(s) or SNPs are obtained from a single cell.

Thus, the present invention contemplates obtaining a sample enriched for one or more fetal cells (such as a fnRBC or a placental cell), and analyzing such enriched sample using the MIP technology or oligonucleotide probes that are precircle probes i.e., probes that form a substantially complete circle when they hybridize to a SNP. The precircle probes comprise a first targeting domain that hybridizes upstream to a SNP position, a second targeting domain that hybridizes downstream of a SNP position, at least a first universal priming site, and a cleavage site. Once the probes are allowed to contact genomic DNA regions of interest (comprising SNPs to be assayed), a hybridization complex forms with a precircle probe and a gap at a SNP position region. Subsequently, ligase is used to “fill in” the gap or complete the circle. The enzymatic “gap fill” process occurs in an allele-specific manner. The nucleotide added to the probe to fill the gap is complementary to the nucleotide base at the SNP position. Once the probe is circular, it can be separated from cross-reacted or unreacted probes by a simple exonuclease reaction. The circular probe is then cleaved at the cleavage site such that it becomes linear again. The cleavage site can be any site in the probe other than the SNP site. Linearization of the circular probe results in the placement of universal primer region at one end of the probe. The universal primer region can be coupled to a tag region. The tag can be detected using amplification techniques known in the art. The SNP analyzed can subsequently be detected by amplifying the cleaved (linearized) probe to detect the presence of the target sequence in said sample or the presence of the tag.

Another method contemplated by the present invention to detect SNPs involves the use of bead arrays (e.g., such as one commercially available by Illumina, Inc.) as described in U.S. Pat. Nos. 7,040,959; 7,035,740; 7033,754; 7,025,935, 6,998,274; 6,942,968; 6,913,884; 6,890,764; 6,890,741; 6,858,394; 6,846,460; 6,812,005; 6,770,441; 6,663,832; 6,620,584; 6,544,732; 6,429,027; 6,396,995; 6,355,431 and US Publication Application Nos. 20060019258; 20050266432; 20050244870; 20050216207; 20050181394; 20050164246; 20040224353; 20040185482; 20030198573; 20030175773; 20030003490; 20020187515; and 20020177141; as well as Shen, R., et al. Mutation Research 573: 70-82 (2005), which are herein incorporated by reference in their entirety.

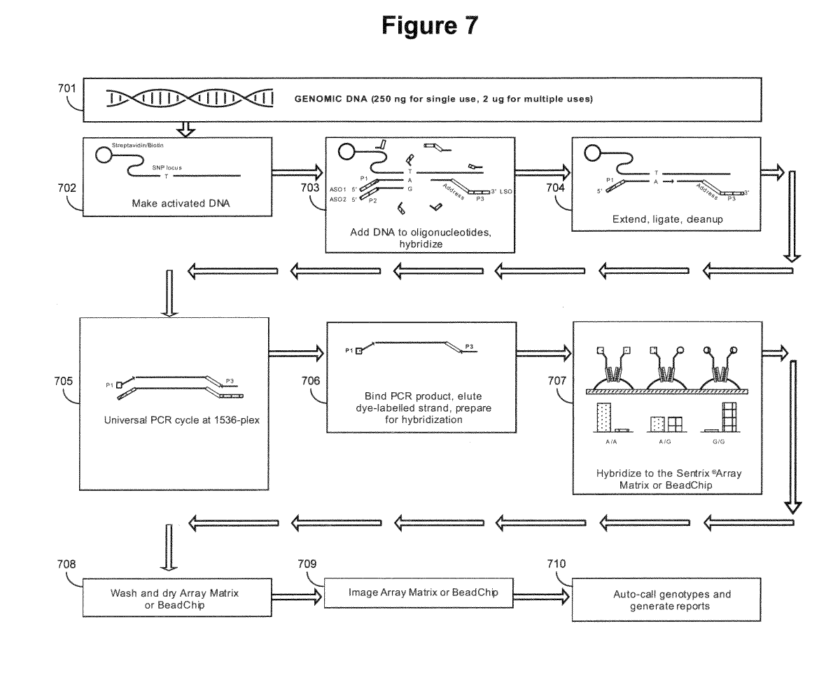

FIG. 7 illustrates an overview of one embodiment of detecting mutations or SNPs using bead arrays. In this embodiment, a sample comprising one or more rare cells (e.g., fnRBC cell or placental cell) and non-rare cells (e.g., RBC's) is obtained from an animal such as a human. Rare cells or rare DNA (e.g., rare nuclei) are enriched using one or more methods disclosed herein or known in the art. In one embodiment, rare cells are enriched by flowing the sample through an array of obstacles that selectively directs particles or cells of different hydrodynamic sizes into different outlets.

In step 701, genomic DNA is obtained from the rare cell(s) or nuclei and, optionally, from the one or more non-rare cells remaining in the enriched mixture. The assays in this embodiment require very little genomic DNA starting material, e.g., between 250 ng-2 μg. Depending on the multiplex level, the activation step can require only 160 pg of DNA per SNP genotype call. In step 702, the genomic DNA is activated such that it can bind paramagnetic particles. In step 703 assay oligonucleotides, hybridization buffer, and paramagnetic particles are combined with the activated DNA and allowed to hybridize (hybridization step). In one embodiment, three oligonucleotides are added for each SNP to be detected. Two of the three oligos are specific for each of the two alleles at a SNP position and are referred to as Allele-Specific Oligos (ASOs). A third oligo hybridizes several bases downstream from the SNP site and is referred to as the Locus-Specific Oligo (LSO). All three oligos contain regions of genomic complementarity (C1, C2, and C3) and universal PCR primer sites (P1, P2 and P3). The LSO also contains a unique address sequence (Address) that targets a particular bead type. (Up to 1,536 SNPs can be assayed in this manner using GoldenGate™ Assay available by Illumina, Inc. (San Diego, Calif.).) During the primer hybridization process, the assay oligonucleotides hybridize to the genomic DNA sample bound to paramagnetic particles. Because hybridization occurs prior to any amplification steps, no amplification bias is introduced into the assay.

In step 704, following the hybridization step, several wash steps are performed reducing noise by removing excess and mis-hybridized oligonucleotides. Extension of the appropriate ASO and ligation of the extended product to the LSO joins information about the genotype present at the SNP site to the address sequence on the LSO. In step 705, the joined, full-length products provide a template for performing PCR reactions using universal PCR primers P1, P2, and P3. Universal primers P1 and P2 are labeled with two different labels (e.g., Cy3 and Cy5). Other labels that can be used include, chromophores, fluorescent moieties, enzymes, antigens, heavy metal, magnetic probes, dyes, phosphorescent groups, radioactive materials, chemiluminescent moieties, scattering or fluorescent nanoparticles, Raman signal generating moieties, or electrochemical detection moieties.

In step 706, the single-stranded, labeled DNAs are eluted and prepared for hybridization. In step 707, the single-stranded, labeled DNAs are hybridized to their complement bead type through their unique address sequence. Hybridization of the GoldenGate Assay™ products onto the Array Matrix™ of Beadchip™ allows for separation of the assay products in solution, onto a solid surface for individual SNP genotype readout.

In step 708, the array is washed and dried. In step 709, a reader such as the BeadArray Reader™ is used to analyze signals from the label. For example, when the labels are dye labels such as Cy3 and Cy5, the reader can analyze the fluorescence signal on the Sentrix Array Matrix or BeadChip.

In step 710, a computer program comprising a computer readable medium having a computer executable logic is used to automate genotyping clusters and callings.

In any of the embodiments herein, more than 1000, 5,000, 10,000, 50,000, 100,000, 500,000, or 1,000,000 SNPs can be assayed in parallel.

In one embodiment, analysis involves detecting levels of expression of one or more genes or exons in e.g., enriched rare cells or enriched rare mRNA. Such detection can be performed using, for example, expression microarrays. Thus, the present invention contemplates a method comprising the steps of: enriching rare cells from a sample as described herein, isolating nucleic acids from the rare cells, contacting a microarray under conditions such that the nucleic acids specifically hybridize to the genetic probes on the microarray, and determining the binding specificity (and amount of binding) of the nucleic acid from the enriched sample to the probes. The results from these steps can be used to obtain a binding pattern that would reflect the nucleic acid abundance and establish a gene expression profile. In one embodiment, the gene expression or copy number results from the enriched cell population is compared with gene expression or copy number of a non-rare cell population to diagnose a disease or a condition.

Examples of expression microarrays include those commercially available from Affymetrix, Inc. (Santa Clara, Calif.), such as the exon arrays (e.g., Human Exon ST Array); tiling arrays (e.g., Chromosome 21/22 1.0 Array Set, ENCODE01 1.0 Array, or Human Genome Arrays +); and 3′ eukaryotic gene expression arrays (e.g., Human Genome Array +, etc.). Examples of human genome arrays include HuGene FL Genome Array, Human Cancer G110 ARray, Human Exon 1.0 ST, Human Genome Focus Array, Human Genome U133 Plus 2.0, Human Genome U133 Set, Human Genome U133A 2.0, Human Promoter U95 SetX, Human Tiling 1.0R Array Set, Human Tiling 2.0R Array Set, and Human X3P Array.

Expression detection and analysis using microarrays is described in part in Valk, P. J. et al. New England Journal of Medicine 350(16), 1617-28, 2004; Modlich, O. et al. Clinical Cancer Research 10(10), 3410-21, 2004; Onken, Michael D. et al. Cancer Res. 64(20), 7205-7209, 2004; Gardian, et al. J. Biol. Chem. 280(1), 556-563, 2005; Becker, M. et al. Mol. Cancer. Ther. 4(1), 151-170, 2005; and Flechner, S M et al. Am J Transplant 4(9), 1475-89, 2004; as well as in U.S. Pat. Nos. 5,445,934; 5,700,637; 5,744,305; 5,945,334; 6,054,270; 6,140,044; 6,261,776; 6,291,183; 6,346,413; 6,399,365; 6,420,169; 6,551,817; 6,610,482; 6,733,977; and EP 619321; 323 203, which are herein incorporated by reference in their entirety.

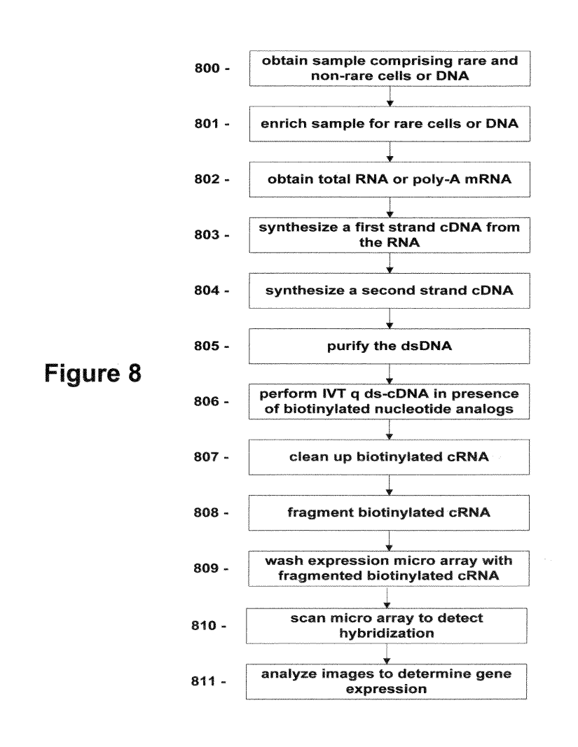

An overview of a protocol that can be used to detect RNA expression (e.g., using Human Genome U133A Set) is illustrated in FIG. 8. In step 800 a sample comprising one or more rare cells (e.g., a fnRBC cell or a placental cell) and non-rare cells (e.g., RBC's) is obtained from an animal, such as a human. In step 801, rare cells or rare DNA (e.g., rare nuclei) are enriched using one or more methods disclosed herein or known in the art. In one embodiment, rare cells are enriched by flowing the sample through an array of obstacles that selectively directs particles or cells of different hydrodynamic sizes into different outlets such that rare cells and cells larger than rare cells are directed into a first outlet and one or more cells or particles smaller than the rare cells are directed into a second outlet.

In step 802 total RNA or poly-A mRNA is obtained from enriched cell(s) (e.g., a fnRBC cell or a placental cell) using purification techniques known in the art. Generally, about 1 μg-2 μg of total RNA is sufficient. In step 803, a first-strand complementary DNA (cDNA) is synthesized using reverse transcriptase and a single T7-oligo(dT) primer. In step 804, a second-strand cDNA is synthesized using DNA ligase, DNA polymerase, and RNase enzyme. In step 805, the double stranded cDNA (ds-cDNA) is purified. In step 806, the ds-cDNA serves as a template for in vitro transcription reaction. The in vitro transcription reaction is carried out in the presence of T7 RNA polymerase and a biotinylated nucleotide analog/ribonucleotide mix. This generates roughly ten times as many complementary RNA (cRNA) transcripts.

In step 807, biotinylated cRNAs are cleaned up, and subsequently in step 808, they are fragmented randomly. Finally, in step 809 the expression microarray (e.g., Human Genome U133 Set) is washed with the fragmented, biotin-labeled cRNAs and subsequently stained with streptavidin phycoerythrin (SAPE). And in step 810, after final washing, the microarray is scanned to detect hybridization of cRNA to probe pairs.

In step 811 a computer program product comprising a computer executable logic analyzes images generated from the scanner to determine gene expression. Such methods are disclosed in part in U.S. Pat. No. 6,505,125, which is herein incorporated by reference in its entirety.

Another method contemplated by the present invention to detect and quantify gene expression involves the use of bead as is commercially available by Illumina, Inc. (San Diego) and as described in U.S. Pat. Nos. 7,035,740; 7033,754; 7,025,935, 6,998,274; 6, 942,968; 6,913,884; 6,890,764; 6,890,741; 6,858,394; 6,812,005; 6,770,441; 6,620,584; 6,544,732; 6,429,027; 6,396,995; 6,355,431 and U.S. Publication Application Nos. 20060019258; 20050266432; 20050244870; 20050216207; 20050181394; 20050164246; 20040224353; 20040185482; 20030198573; 20030175773; 20030003490; 20020187515; and 20020177141; and in B. E. Stranger, et al., Public Library of Science—Genetics, 1 (6), December 2005; Jingli Cai, et al., Stem Cells, published online Nov. 17, 2005; C. M. Schwartz, et al., Stem Cells and Development, 14, 517-534, 2005; Barnes, M., J. et al., Nucleic Acids Research, 33 (18), 5914-5923, October 2005; and Bibikova M, et al. Clinical Chemistry, Volume 50, No. 12, 2384-2386, December 2004, which are herein incorporated by reference in their entirety.

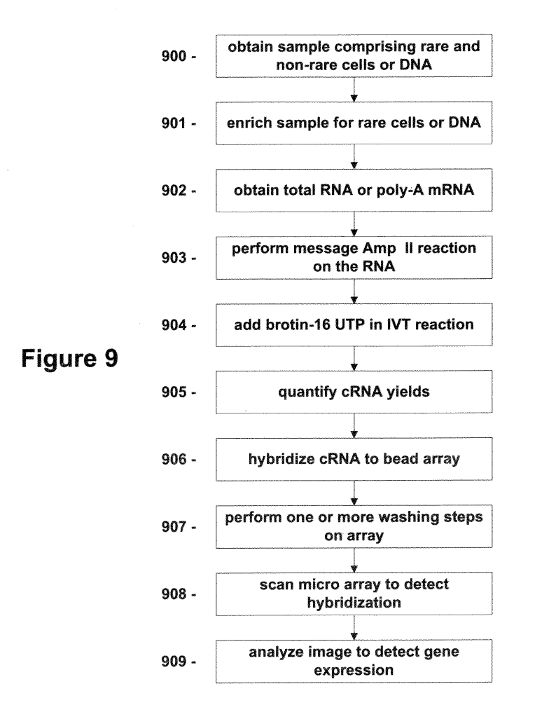

FIG. 9 illustrates an overview of one embodiment of detecting mutations or SNPs using bead arrays. In step 900 a sample comprising one or more rare cells (e.g., a fnRBC cell or a placental cell) and non-rare cells (e.g., RBC's) is obtained from an animal, such as a human. In step 901, rare cells or rare DNA (e.g., rare nuclei) are enriched using one or more methods disclosed herein or known in the art. In one embodiment, rare cells are enriched by flowing the sample through an array of obstacles that selectively directs particles or cells of different hydrodynamic sizes into different outlets such that rare cells and cells larger than rare cells are directed into a first outlet and one or more cells or particles smaller than the rare cells are directed into a second outlet.

In step 902, total RNA is extracted from one or more enriched cells (e.g., one or more fnRBC cells or placental cells). In step 903, two one-quarter scale Message Amp II reactions (Ambion, Austin, Tex.) are performed for each RNA extraction using 200 ng of total RNA. MessageAmp is a procedure based on antisense RNA (aRNA) amplification, and involves a series of enzymatic reactions resulting in linear amplification of exceedingly small amounts of RNA for use in array analysis Unlike exponential RNA amplification methods, such as NASBA and RT-PCR, aRNA amplification maintains representation of the starting mRNA population. The procedure begins with total or poly(A) RNA that is reverse transcribed using a primer containing both oligo(dT) and a T7 RNA polymerase promoter sequence. After first-strand synthesis, the reaction is treated with RNase H to cleave the mRNA into small fragments. These small RNA fragments serve as primers during a second-strand synthesis reaction that produces a double-stranded cDNA template for transcription. Contaminating rRNA, mRNA fragments and primers are removed and the cDNA template is then used in a large scale in vitro transcription reaction to produce linearly amplified aRNA. The aRNA can be labeled with biotin rNTPS or amino allyl-UTP during transcription.

In step 904, biotin-16-UTP (Perkin Elmer, Wellesley, Calif.) is added such that half of the UTP is used in the in vitro transcription reaction. In step 905, cRNA yields are quantified using RiboGreen (Invitrogen, Carlsbad, Calif.). In step 906, 1 μg of cRNA is hybridized to a bead array (e.g.; Illumina Bead Array). In step 907, one or more washing steps is performed on the array. In step 908, after final washing, the microarray is scanned to detect hybridization of cRNA. In step 908, a computer program product comprising an executable program analyzes images generated from the scanner to determine gene expression.

Additional description for preparing RNA for bead arrays is described in Kacharmina J E, et al., Methods Enzymol 303: 3-18, 1999; Pabon C, et al., Biotechniques 31(4): 874-9, 2001; Van Gelder R N, et al., Proc Natl Acad Sci USA 87: 1663-7 (1990); and Murray, S S. BMC Genetics 6(Suppl I):S85 (2005), which are herein incorporated by reference in their entirety.

In one embodiment, more than 1000, 5,000, 10,000, 50,000, 100,000, 500,000, or 1,000,000 transcripts can be assayed in parallel.

In any of the embodiments herein, genotyping (e.g., SNP detection) and/or expression analysis (e.g., RNA transcript quantification) of genetic content from enriched rare cells or enriched rare cell nuclei can be accomplished by sequencing. Sequencing be accomplished through classic Sanger sequencing methods which are well known in the art. Sequence can also be accomplished using high-throughput systems some of which allow detection of a sequenced nucleotide immediately after or upon its incorporation into a growing strand, i.e., detection of sequence in real time or substantially real time. In one embodiment, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read. Sequencing can be preformed using genomic DNA or cDNA derived from RNA transcripts as a template.

In one embodiment, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. This fast sequencing method also allows for detection of a SNP/nucleotide in a sequence in substantially real time or real time. Finally, SMSS is powerful because, like the MIP technology, it does not require a preamplification step prior to hybridization. In fact, SMSS does not require any amplification. SMSS is described in part in U.S. Publication Application Nos. 2006002471.1; 20060024678; 20060012793; 20060012784; and 20050100932, which are herein incorporated by reference in their entirety.

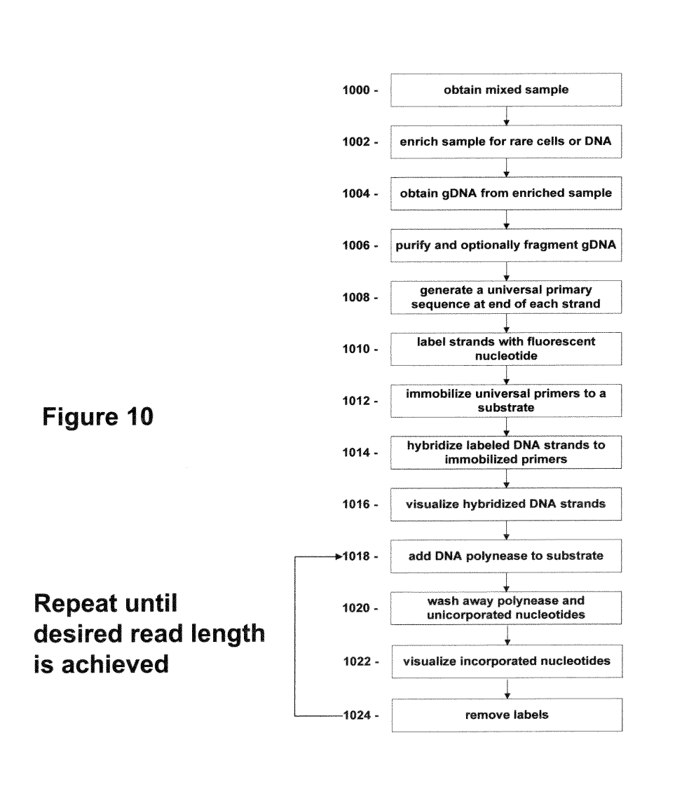

An overview the use of SMSS for analysis of enriched cells/nucleic acids (e.g., one or more fnRBC cells or placental cells) is outlined in FIG. 10.

First, in step 1000 a sample comprising one or more rare cells (e.g., fnRBC cells or placental cells) and one or more non-rare cells (e.g., RBC's) is obtained from an animal, such as a human. In step 1002, rare cells or rare DNA (e.g., rare nuclei) are enriched using one or more methods disclosed herein or known in the art. In one embodiment, rare cells are enriched by flowing the sample through an array of obstacles that selectively directs particles or cells of different hydrodynamic sizes into different outlets. In step 1004, genomic DNA is obtained from the rare cell(s) or nuclei and optionally one or more non-rare cells remaining in the enriched mixture.

In step 1006 the genomic DNA is purified and optionally fragmented. In step 1008, a universal priming sequence is generated at the end of each strand. In step 1010, the strands are labeled with a fluorescent nucleotide. These strands will serve as templates in the sequencing reactions.

In step 1012 universal primers are immobilized on a substrate (e.g., glass surface) inside a flow cell.

In step 1014, the labeled DNA strands are hybridized to the immobilized primers on the substrate.

In step 1016, the hybridized DNA strands are visualized by illuminating the surface of the substrate with a laser and imaging the labeled DNA with a digital TV camera connected to a microscope. In this step, the position of all hybridization duplexes on the surface is recorded.

In step 1018, DNA polymerase is flowed into the flow cell. The polymerase catalyzes the addition of the labeled nucleotides to the correct primers.

In step 1020, the polymerase and unincorporated nucleotides are washed away in one or more washing procedures.

In step 1022, the incorporated nucleotides are visualized by illuminating the surface with a laser and imaging the incorporated nucleotides with a camera. In this step, recordation is made of the positions of the incorporated nucleotides.

In step 1024, the fluorescent labels on each nucleotide are removed.

Steps 1018-1024 are repeated with the next nucleotide such that the steps are repeated for A, G, T, and C. This sequence of events is repeated until the desired read length is achieved.

SMSS can be used, e.g., to sequence DNA from one or more enriched fetal cells to identify one or more genetic mutations (e.g., SNPs) in DNA, or to profile gene expression of one or more mRNA transcripts of such one or more cells or other cells (e.g., one or more fetal cells). SMSS can also be used to identify one or more genes in a fetal cell that are methylated (“turned off”) and develop cancer diagnostics based on such methylation. Finally, one or more enriched cells/DNA can be analyzed using SMSS to detect minute levels of DNA from pathogens such as viruses, bacteria or fungi. Such DNA analysis can further be used for serotyping to detect, e.g., drug resistance or susceptibility to disease. Furthermore, one or more enriched stem cells can be analyzed using SMSS to determine if various expression profiles and differentiation pathways are turned “on” or “off”. This allows for a determination to be made of the enriched stem cells are prior to or post differentiation.

In one embodiment, high-throughput sequencing involves the use of technology available by 454 Lifesciences, Inc. (Branford, Conn.) such as the PicoTiterPlate device which includes a fiber optic plate that transmits chemilluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density pricolitre reactors”, Nature, doi:10.1038/nature03959; and well as in U.S. Publication Application Nos. 20020012930; 20030068629; 20030100102; 20030148344; 20040248161; 20050079510, 20050124022; and 20060078909, which are herein incorporated by reference in their entirety.

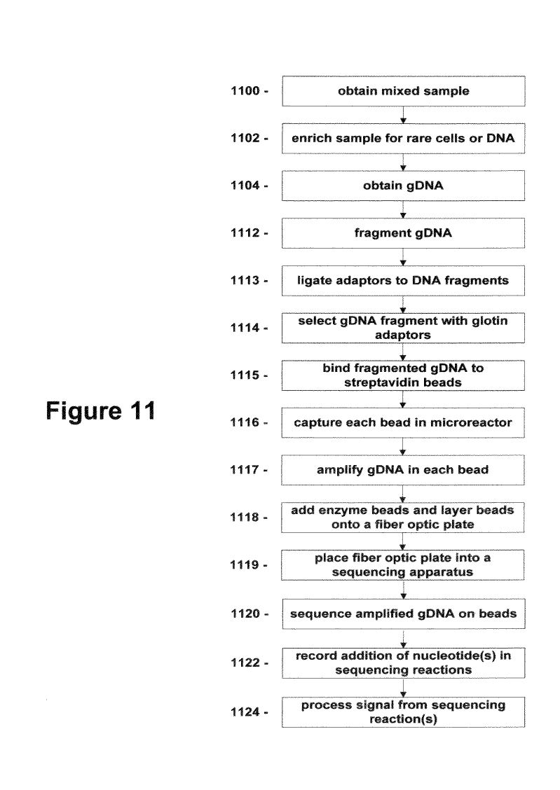

An overview of this embodiment is illustrated in FIG. 11.

First, in step 1100 a sample comprising one or more rare cells (e.g., fnRBC cells or placental cells) and one or more non-rare cells (e.g., RBC's) is obtained from an animal, such as a human. In step 1102, rare cells or rare DNA (e.g., rare nuclei) are enriched using one or more methods disclosed herein or known in the art. In one embodiment, rare cells are enriched by flowing the sample through an array of obstacles that selectively directs particles or cells of different hydrodynamic sizes into different outlets. In step 1104, genomic DNA is obtained from the rare cell(s) or nuclei and optionally one or more non-rare cells remaining in the enriched mixture.

In step 1112, the enriched genomic DNA is fragmented to generate a library of hundreds of DNA fragments for sequencing runs. Genomic DNA (gDNA) is fractionated into smaller fragments (300-500 base pairs) that are subsequently polished (blunted). In step 1113, short adaptors (e.g., A and B) are ligated onto the ends of the fragments. These adaptors provide priming sequences for both amplification and sequencing of the sample-library fragments. One of the adaptors (e.g., Adaptor B) contains a 5′-biotin tag or other tag that enables immobilization of the library onto beads (e.g., streptavidin coated beads). In step 1114, only gDNA fragments that include both Adaptor A and B are selected using avidin-blotting purification. The sstDNA library is assessed for its quality and the optimal amount (DNA copies per bead) needed for subsequent amplification is determined by titration. In step 1115, the sstDNA library is annealed and immobilized onto an excess of capture beads (e.g., streptavidin coated beads). The latter occurs under conditions that favor each bead to carry only a single sstDNA molecule. In step 1116, each bead is captured in its own microreactor, such as a well, which can optionally be addressable, or a picolitre-sized well. In step 1117, the bead-bound library is amplified using, e.g., emPCR. This can be accomplished by capturing each bead within a droplet of a PC-reaction-mixture-in-oil-emulsion. Thus, the bead-bound library can be emulsified with the amplification reagents in a water-in-oil mixture. EmPCR enables the amplification of a DNA fragment immobilized on a bead from a single fragment to 10 million identical copies. This amplification step generates sufficient identical DNA fragments to obtain a strong signal in the subsequent sequencing step. The amplification step results in bead-immobilized, clonally amplified DNA fragments. The amplification on the bead results can result in each bead carrying at least one million, at least 5 million, or at least 10 million copies of the unique target nucleic acid.

The emulsion droplets can then be broken, genomic material on each bead can be denatured, and single-stranded nucleic acids clones can be deposited into wells, such as picolitre-sized wells, for further analysis including, but are not limited to quantifying said amplified nucleic acid, gene and exon-level expression analysis, methylation-state analysis, novel transcript discovery, sequencing, genotyping or resequencing. In step 1118, the sstDNA library beads are added to a DNA bead incubation mix (containing DNA polymerase) and are layered with enzyme beads (containing sulfurylase and luciferase as is described in U.S. Pat. Nos. 6,956,114 and 6,902,921) onto a fiber optic plate such as the PicoTiterPlate device. The fiber optic plate is centrifuged to deposit the beads into wells (˜up to 50 or 45 microns in diameter). The layer of enzyme beads ensures that the DNA beads remain positioned in the wells during the sequencing reaction. The bead-deposition process maximizes the number of wells that contain a single amplified library bead (avoiding more than one sstDNA library bead per well). In one embodiment, each well contains a single amplified library bead. In step 1119, the loaded fiber optic plate (e.g., PicoTiterPlate device) is then placed into a sequencing apparatus (e.g., the Genome Sequencer 20 Instrument). Fluidics subsystems flow sequencing reagents (containing buffers and nucleotides) across the wells of the plate. Nucleotides are flowed sequentially in a fixed order across the fiber optic plate during a sequencing run. In step 1120, each of the hundreds of thousands of beads with millions of copies of DNA is sequenced in parallel during the nucleotide flow. If a nucleotide complementary to the template strand is flowed into a well, the polymerase extends the existing DNA strand by adding nucleotide(s) which transmits a chemilluminescent signal. In step 1122, the addition of one (or more) nucleotide(s) results in a reaction that generates a chemilluminescent signal that is recorded by a digital camera or CCD camera in the instrument. The signal strength of the chemilluminescent signal is proportional to the number of nucleotides added. Finally, in step 1124, a computer program product comprising an executable logic processes the chemilluminescent signal produced by the sequencing reaction. Such logic enables whole genome sequencing for de novo or resequencing projects.

In one embodiment, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 20040106110; 20030064398; 20030022207; and Constans, A., The Scientist 2003, 17(13):36, which are herein incorporated by reference in their entirety.

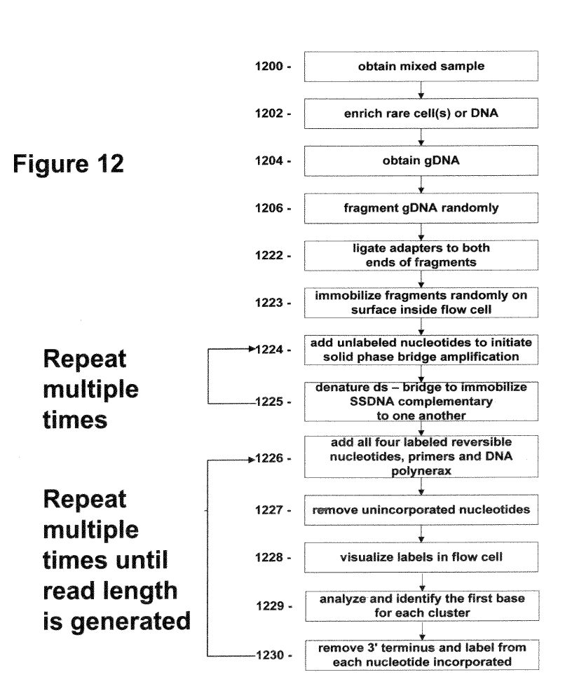

FIG. 12 illustrates a first embodiment using the SBS approach described above.

First, in step 1200 a sample comprising one or more rare cells (e.g., fnRBC cells or placental cells) and one or more non-rare cells (e.g., RBC's) is obtained from an animal, such as a human. In step 1202, rare cells, rare DNA (e.g., rare nuclei), or raremRNA is enriched using one or more methods disclosed herein or known in the art. In one embodiment, rare cells are enriched by flowing the sample through an array of obstacles that selectively directs particles or cells of different hydrodynamic sizes into different outlets.

In step 1204, enriched genetic material e.g., gDNA is obtained using methods known in the art or disclosed herein. In step 1206, the genetic material e.g., gDNA is randomly fragmented. In step 1222, the randomly fragmented gDNA is ligated with adapters on both ends. In step 1223, the genetic material, e.g., ssDNA are bound randomly to inside surface of a flow cell channels. In step 1224, unlabeled nucleotides and enzymes are added to initiate solid phase bridge amplification. The above step results in genetic material fragments becoming double stranded and bound at either end to the substrate. In step 1225, the double stranded bridge is denatured to create to immobilized single stranded genomic DNA (e.g., ssDNA) sequencing complementary to one another. The above bridge amplification and denaturation steps are repeated multiple times (e.g., at least 10, 50, 100, 500, 1,000, 5,000, 10,000, 50,000, 100,000, 500,000, 1,000,000, 5,000,000 times) such that several million dense clusters of dsDNA (or immobilized ssDNA pairs complementary to one another) are generated in each channel of the flow cell. In step 1226, the first sequencing cycle is initiated by adding all four labeled reversible terminators, primers, and DNA polymerase enzyme to the flow cell. This sequencing-by-synthesis (SBS) method utilizes four fluorescently labeled modified nucleotides that are especially created to posses a reversible termination property, which allow each cycle of the sequencing reaction to occur simultaneously in the presence of all four nucleotides (A, C, T, G). In the presence of all four nucleotides, the polymerase is able to select the correct base to incorporate, with the natural competition between all four alternatives leading to higher accuracy than methods where only one nucleotide is present in the reaction mix at a time which require the enzyme to reject an incorrect nucleotide. In step 1227, all unincorporated labeled terminators are then washed off. In step 1228, laser is applied to the flow cell. Laser excitation captures an image of emitted fluorescence from each cluster on the flow cell. In step 1229, a computer program product comprising a computer executable logic records the identity of the first base for each cluster. In step 1230, before initiated the next sequencing step, the 3′ terminus and the fluorescence from each incorporated base are removed.

Subsequently, a second sequencing cycle is initiated, just as the first was by adding all four labeled reversible terminators, primers, and DNA polymerase enzyme to the flow cell. A second sequencing read occurs by applying a laser to the flow cell to capture emitted fluorescence from each cluster on the flow cell which is read and analyzed by a computer program product that comprises a computer executable logic to identify the first base for each cluster. The above sequencing steps are repeated as necessary to sequence the entire gDNA fragment. In one embodiment, the above steps are repeated at least 5, 10, 50, 100, 500, 1,000, 5,000, to 10,000 times.

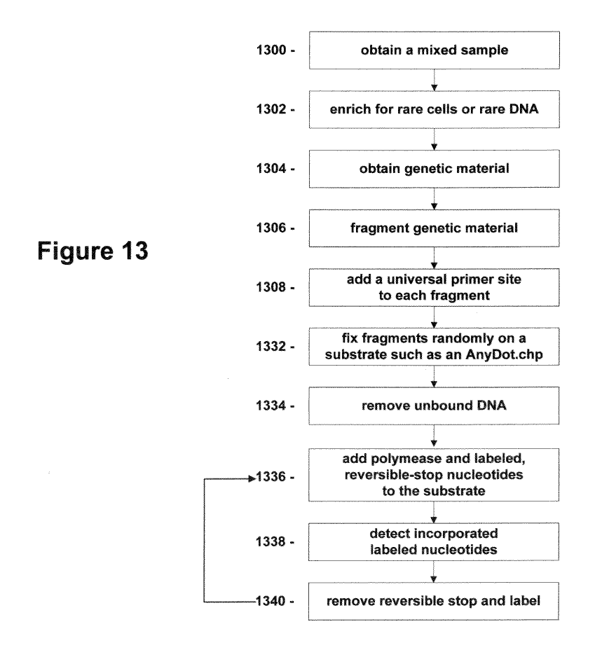

In one embodiment, high-throughput sequencing of mRNA or gDNA can take place using AnyDot.chips (Genovoxx, Germany), which allows for the monitoring of biological processes (e.g., mRNA expression or allele variability (SNP detection). In particular, the AnyDot.chips allow for 10×-50× enhancement of nucleotide fluorescence signal detection. AnyDot.chips and methods for using them are described in part in International Publication Application Nos. WO 02088382, WO 03020968, WO 03031947, WO 2005044836, PCT/EP 05/05657, PCT/EP 05/05655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301, which are herein incorporated by reference in their entirety. An overview of one embodiment of the present invention is illustrated in FIG. 13.

First, in step 1300 a sample comprising one or more rare cells (e.g., fnRBC cells or placental cells) and one or more non-rare cells (e.g., RBC's) is obtained from an animal, such as a human. In step 1302, rare cells or rare genetic material (e.g., gDNA or RNA) is enriched using one or more methods disclosed herein or known in the art. In one embodiment, rare cells are enriched by flowing the sample through an array of obstacles that selectively directs particles or cells of different hydrodynamic sizes into different outlets. In step 1304, genetic material is obtained from the enriched sample. In step 1306, the genetic material (e.g., gDNA) is fragmented into millions of individual nucleic acid molecules and in step 1308, a universal primer binding site is added to each fragment (nucleic acid molecule). In step 1332, the fragments are randomly distributed, fixed and primed on a surface of a substrate, such as an AnyDot.chip. Distance between neighboring molecules averages 0.1-10 μm or about 1 μM. A sample is applied by simple liquid exchange within a microfluidic system. Each mm² contains 1 million single DNA molecules ready for sequencing. In step 1334, unbound DNA fragments are removed from the substrate; and in step 1336, a solution containing polymerase and labeled nucleotide analogs having a reversible terminator that limits extension to a single base, such as AnyBase.nucleotides are applied to the substrate. When incorporated into the primer-DNA hybrid, such nucleotide analogs cause a reversible stop of the primer-extension (terminating property of nucleotides). This step represents a single-base extension. During the stop, incorporated bases, which include a fluorescence label, can be detected on the surface of the substrate.

In step 1338, fluorescent dots are detected by a single-molecule fluorescence detection system (e.g., fluorescent microscope). In one embodiment, a single fluorescence signal (300 nm in diameter) can be properly tracked over the complete sequencing cycles (see below). After detection of the single-base, in step 1340, the terminating property and fluorescent label of the incorporated nucleotide analogs (e.g., AnyBase.nucleotides) are removed. The nucleotides are now extendable similarly to native nucleotides. Thus, steps 1336-1340 are thus repeated, e.g., at least 2, 10, 20, 100, 200, 1,000, 2,000 times. For generating sequence data that can be compared with a reference database (for example human mRNA database of the NCBI), length of the sequence snippets has to exceed 15-20 nucleotides. Therefore, steps 1 to 3 are repeated until the majority of all single molecules reach the required length. This will take, on average, 2 offers of nucleotide incorporations per base.

Other high-throughput sequencing systems include those disclosed in Venter, J., et al. Science 16 Feb. 2001; Adams, M. et al. Science 24 Mar. 2000; and M. J. Levene, et al. Science 299:682-686, January 2003; as well as U.S. Publication Application No. 20030044781 and 2006/0078937, which are herein incorporated by reference in their entirety. Overall such system involve sequencing a target nucleic acid molecule having a plurality of bases by the temporal addition of bases via a polymerization reaction that is measured on a molecule of nucleic acid, i.e. the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishable type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In one embodiment, cDNAs, which are reverse transcribed from mRNAs obtained from fetal or maternal cells, are analyzed (e.g., SNP analysis or sequencing) by the methods disclosed herein. The type and abundance of the cDNAs can be used to determine whether a cell is a fetal cell (such as by the presence of Y chromosome specific transcripts) or whether the fetal cell has a genetic abnormality (such as aneuploidy, abundance or type of alternative transcripts or problems with DNA methylation or imprinting).

In one embodiment, one or more fetal or maternal cells are enriched using one or more methods disclosed herein. In one embodiment, one or more fetal cells are enriched by flowing the sample through an array of obstacles that selectively directs particles or cells of different hydrodynamic sizes into different outlets such that one or more fetal cells and cells larger than a fetal cell are directed into a first outlet and one or more cells or particles smaller than a rare cell (e.g., a fetal cell) are directed into a second outlet.

Total RNA or poly-A mRNA is then obtained from enriched cell(s) (fetal or maternal cells) using purification techniques known in the art. Generally, about 1 μg-2 μg of total RNA is sufficient. Next, a first-strand complementary DNA (cDNA) is synthesized using reverse transcriptase and a single T7-oligo(dT) primer. Next, a second-strand cDNA is synthesized using DNA ligase, DNA polymerase, and RNase enzyme. Next, the double stranded cDNA (ds-cDNA) is purified.

Analyzing one or more rare cells to determine the existence of a condition or disease can also include detecting mitochondrial DNA, telomerase, or a nuclear matrix protein in the enriched rare cell sample; detecting the presence or absence of perinuclear compartments in a cell of the enriched sample; or performing gene expression analysis, determining nucleic acid copy number, in-cell PCR, or fluorescence in-situ hybridization of the enriched sample.

In one embodiment, PCR-amplified single-strand nucleic acid is hybridized to a primer and incubated with a polymerase, ATP sulfurylase, luciferase, apyrase, and the substrates luciferin and adenosine 5′ phosphosulfate. Next, deoxynucleotide triphosphates corresponding to the bases A, C, G, and T (U) are added sequentially. Each base incorporation step is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process repeats until the entire sequence is determined. In one embodiment, pyrosequencing analyzes DNA methylations, mutation and SNPs. In another embodiment, pyrosequencing also maps surrounding sequences as an internal quality control. Pyrosequencing analysis methods are known in the art.

In one embodiment, sequence analysis of the rare cell's genetic material can include a four-color sequencing by ligation scheme (degenerate ligation), which involves hybridizing an anchor primer to one of four positions. Then an enzymatic ligation reaction of the anchor primer to a population of degenerate nonamers that are labeled with fluorescent dyes is performed. At any given cycle, the population of nonamers that is used is structure such that the identity of one of its positions is correlated with the identity of the fluorophore attached to that nonamer. To the extent that the ligase discriminates for complementarity at that queried position, the fluorescent signal allows the inference of the identity of the base. After performing the ligation and four-color imaging, the anchor primer: nonamer complexes are stripped and a new cycle begins. Methods to image sequence information after performing ligation are known in the art.

Another embodiment includes kits for performing some or all of the steps of the invention. The kits can include devices and reagents in any combination to perform any or all of the steps. For example, the kits can include the arrays for the size-based separation or enrichment, the device and reagents for magnetic separation and the reagents needed for the genetic analysis. In one embodiment, the methods herein are used for detecting the presence or conditions of one or more rare cells that are present in a mixed sample at a concentration of less than or equal to 90%, 80%, 70%, 60%, 50%, 40%, 30%, 20%, 10%, 5%, 1%, 1×10⁻¹%, 1×10⁻²%, 1×10⁻³%, 1×10⁻⁴%, 1×10⁻⁵%, 1×10⁻⁶%, 1×10⁻⁷%, 1×10⁻⁸%, or 1×10⁻⁹% of all cells in the mixed sample. In another embodiment, the methods herein are used for detecting the presence or conditions of one or more rare cells that are present in a mixed sample at a concentration of less than or equal to 1:2, 1:4, 1:10, 1:50, 1:100, 1:200, 1:500, 1:1000, 1:2000, 1:5000, 1:10,000, 1:20,000, 1:50,000, 1:100,000, 1:200,000, 1:1,000,000, 1:2,000,000, 1:5,000,000, 1:10,000,000, 1:20,000,000, 1:50,000,000 or 1:100,000,000 of all cells in the sample. In another embodiment, the methods herein are used for detecting the presence or conditions of one or more rare cells that are present in a mixed sample at a concentration of less than 1×10⁻³, 1×10⁻⁴, 1×10⁻⁵, 1×10⁻⁶, or 1×10⁻⁷ cells/μL of a fluid sample. In one embodiments, the mixed sample has a total of less than or equal to 2, 3, 4, 5, 6, 7, 8, 9, 10, 15, 20, 30, 40, 50, or 100 rare cells.

A rare cell can be, for example, a fetal cell derived from a maternal sample (e.g., blood sample).

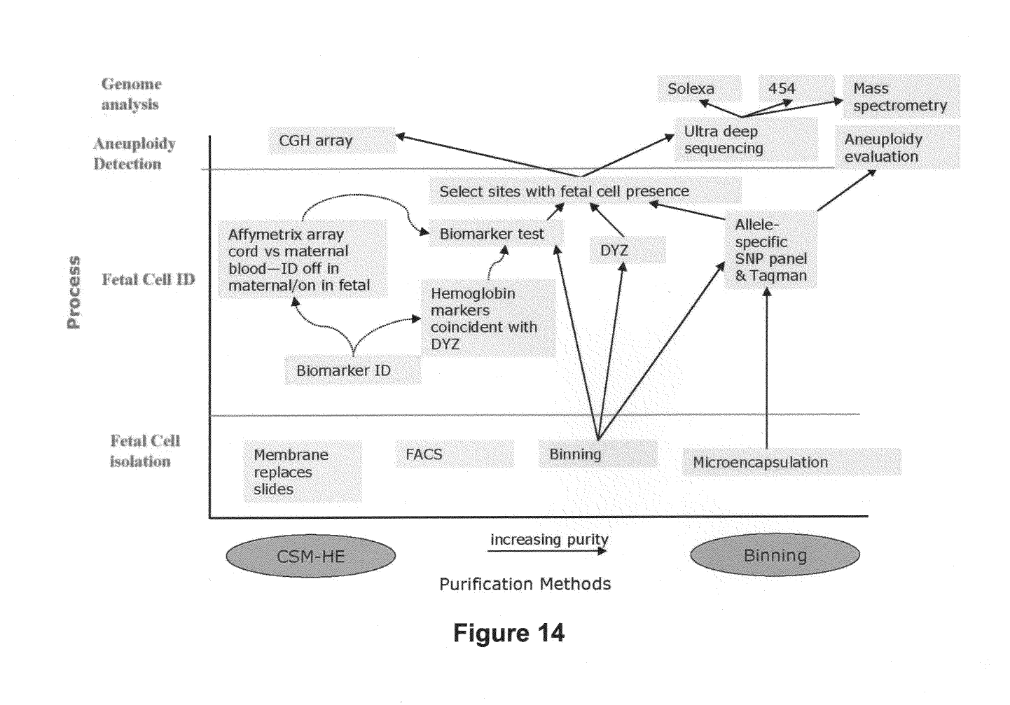

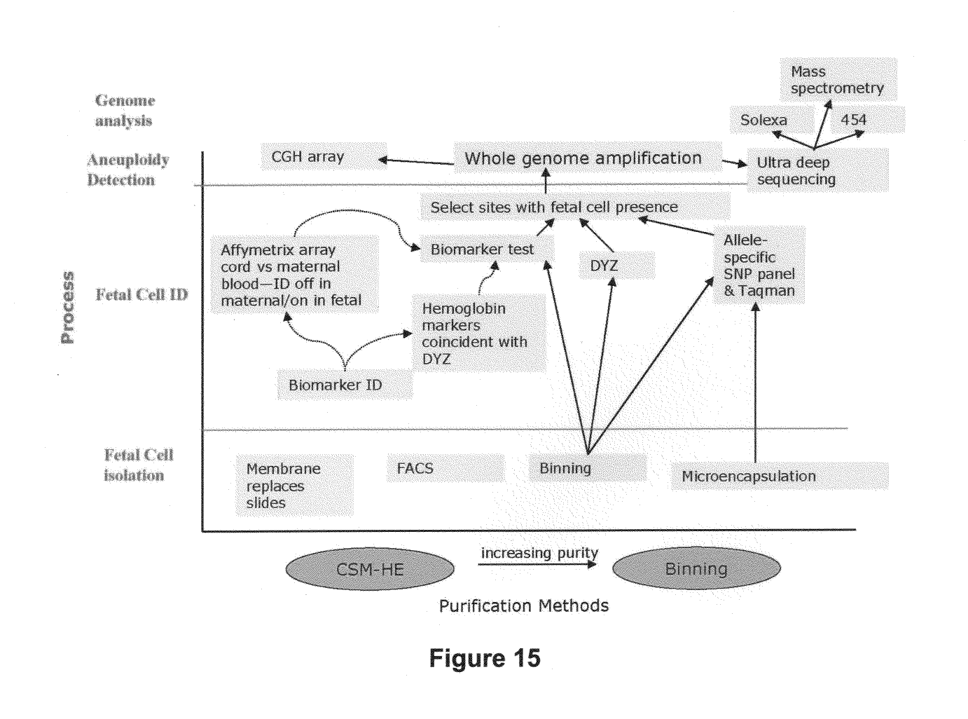

One or more enriched target cells (e.g., fnRBC) can be “binned” prior to analysis of the one or more enriched cells (FIGS. 14 and 15). Binning is any process which results in the reduction of complexity and/or total cell number of the enriched cell output. Binning can be performed by any method known in the art or described herein. One method of binning the enriched cells is by serial dilution. Such dilution can be carried out using any appropriate platform (e.g., PCR wells, microtiter plates). Other methods include nanofluidic systems which separate samples into droplets (e.g., BioTrove, Raindance, Fluidigm). Such nanofluidic systems can result in the presence of a single cell present in a nanodroplet.

Binning can be preceded by positive selection for target cells including, but not limited to affinity binding (e.g., using anti-CD71 antibodies). Alternately, negative selection of non-target cells can precede binning. For example, output from the size-based separation module can be passed through a magnetic hemoglobin enrichment module (MHEM) which selectively removes WBCs from the enriched sample.

For example, the possible cellular content of output from enriched maternal blood which has been passed through a size-based separation module (with or without further enrichment by passing the enriched sample through a MHEM) can consist of: 1) approximately 20 fnRBC; 2) 1,500 nmRBC; 3) 4,000-40,000 WBC; 4) 15×10⁶ RBC. If this sample is separated into 100 bins (PCR wells or other acceptable binning platform), each bin would be expected to contain: 1) 80 negative bins and 20 bins positive for one fnRBC; 2) 150 nmRBC; 3) 400-4,000 WBC; 4) 15×10⁴ RBC. If separated into 10,000 bins, each bin would be expected to contain: 1) 9,980 negative bins and 20 bins positive for one fnRBC; 2) 8,500 negative bins and 1,500 bins positive for one mnRBC; 3) <1-4 WBC; 4) 15×10² RBC. One of skill in the art will recognize that the number of bins can be increased depending on experimental design and/or the platform used for binning. The reduced complexity of the binned cell populations can facilitate further genetic and cellular analysis of the target cells.

Analysis can be performed on individual bins to confirm the presence of target cells (e.g. fnRBC) in the individual bin. Such analysis can consist of any method known in the art, including, but not limited to, FISH, PCR, STR detection, SNP analysis, biomarker detection, and sequence analysis (FIGS. 14 and 15).

### STR Analysis

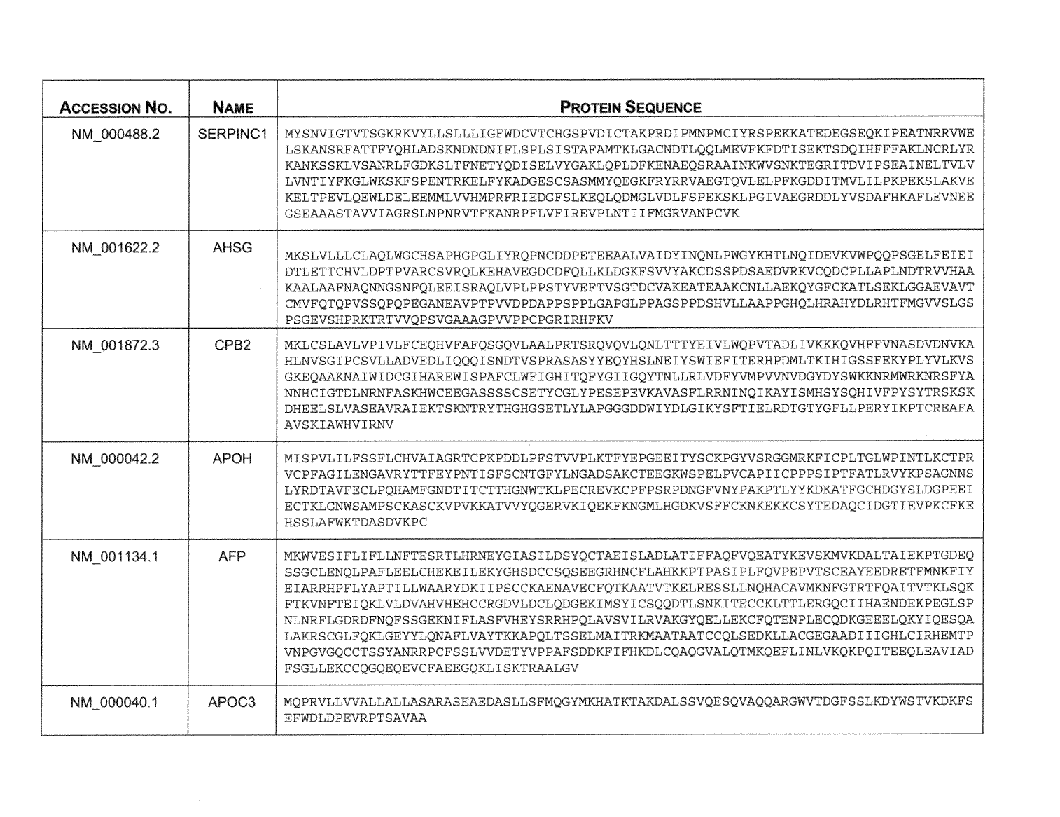

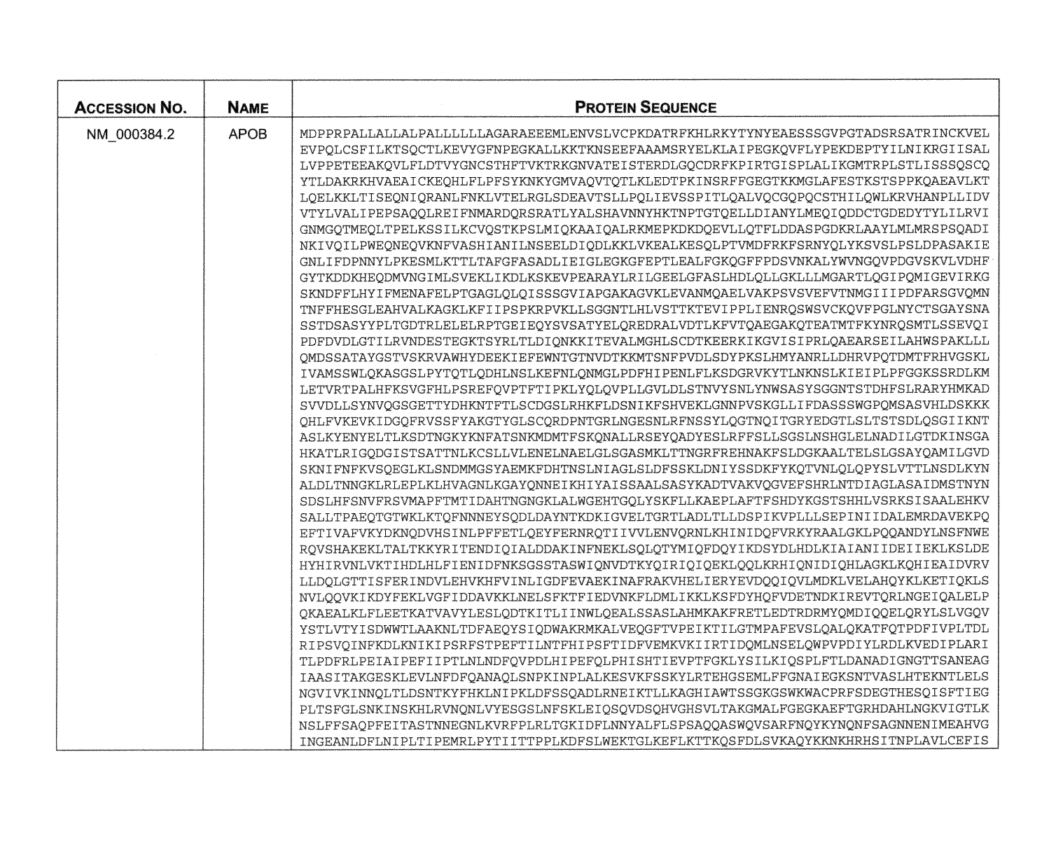

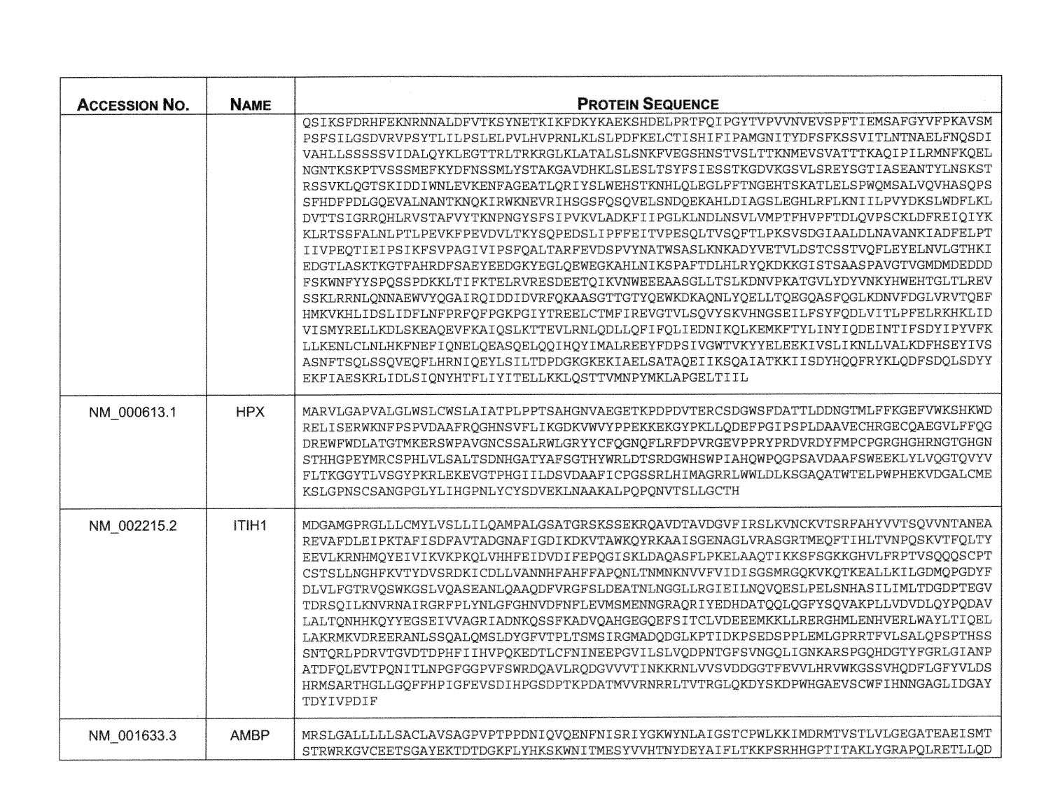

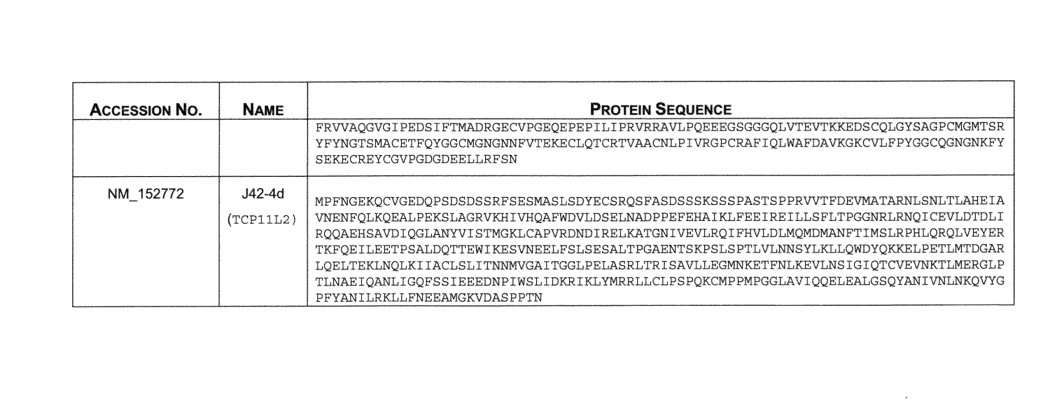

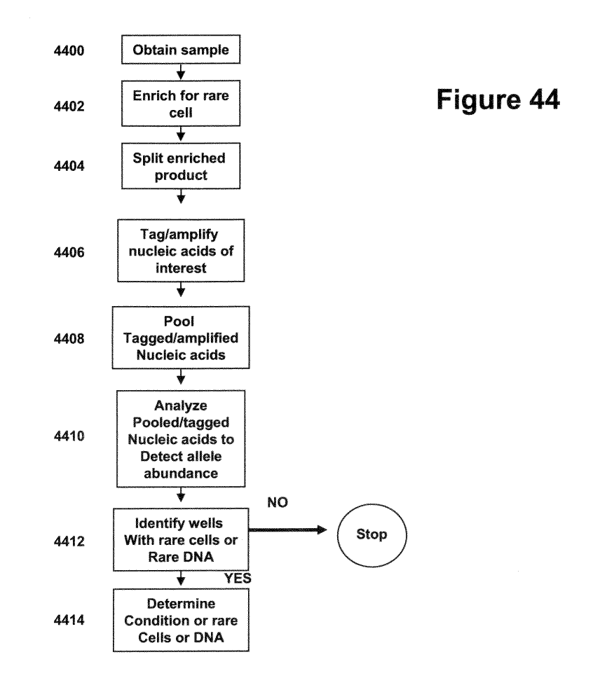

FIG. 44 illustrates an overview of one embodiment of the present invention.

Aneuploidy means the condition of having less than or more than the normal diploid number of chromosomes. In other words, it is any deviation from euploidy. Aneuploidy includes conditions such as monosomy (the presence of only one chromosome of a pair in a cell's nucleus), trisomy (having three chromosomes of a particular type in a cell's nucleus), tetrasomy (having four chromosomes of a particular type in a cell's nucleus), pentasomy (having five chromosomes of a particular type in a cell's nucleus), triploidy (having three of every chromosome in a cell's nucleus), and tetraploidy (having four of every chromosome in a cell's nucleus). Birth of a live triploid is extraordinarily rare and such individuals are quite abnormal, however triploidy occurs in about 2-3% of all human pregnancies and appears to be a factor in about 15% of all miscarriages. Tetraploidy occurs in approximately 8% of all miscarriages. (http://www.emedicine.com/med/topic3241.htm).

In step 4400, a sample is obtained from an animal, such as a human. In one embodiment, an animal or human is pregnant, suspected of being pregnant, or may have been pregnant, and, the systems and methods herein are used to diagnose pregnancy and/or conditions of the fetus (e.g. aneuploidy). In some embodiments, the animal or human is suspected of having a condition, has a condition, or had a condition (e.g., cancer) and, the systems and methods herein are used to diagnose the condition, determine appropriate therapy, and/or monitor for recurrence.

In both scenarios a sample obtained from the animal can be a blood sample e.g., of up to 50, 40, 30, 20, or 15 mL. In some cases multiple samples are obtained from the same animal at different points in time (e.g. before therapy, during therapy, and after therapy, or during 1^{st} trimester, 2^{nd} trimester, and 3^{rd} trimester of pregnancy).

In optional step 4402, one or more rare cells (e.g., one or more fetal cells or epithelial cells) or DNA of such rare cells are enriched using one or more methods known in the art or described herein. For example, to enrich one or more fetal cells from a maternal blood sample, the sample can be applied to a size-base separation module (e.g., two-dimensional array of obstacles) configured to direct cells or particles in the sample greater than 8 microns to a first outlet and cells or particles in the sample smaller than 8 microns to a second outlet. The fetal cells can subsequently be further enriched from maternal white blood cells (which are also greater than 8 microns) based on their potential magnetic property. For example, N₂ or anti-CD71 coated magnetic beads are added to the first enriched product to make the hemoglobin in the red blood cells (maternal and fetal) paramagnetic. The enriched sample is then flowed through a column coupled to an external magnet. This captures both the fnRBC's and mnRBC's creating a second enriched product. The sample can then be subjected to hyperbaric pressure or other stimulus to initiate apoptosis in the fetal cells. Fetal cells/nuclei can then be enriched using microdissection, for example. It should be noted that even an enriched product can be dominated (>50%) by cells not of interest (e.g., maternal red blood cells). In some cases an enriched sample has one or more of the rare cells (or rare genomes) consisting of up to 0.01, 0.02, 0.05, 0.1, 0.2, 0.5, 1, 2, 5, 10, 20, or 50% of all cells (or genomes) in the enriched sample. For example, using the systems herein, a maternal blood sample of 20 mL from a pregnant human can be enriched for one or more fetal cells such that the enriched sample has a total of about 500 cells, 2% of which are fetal and the rest are maternal.

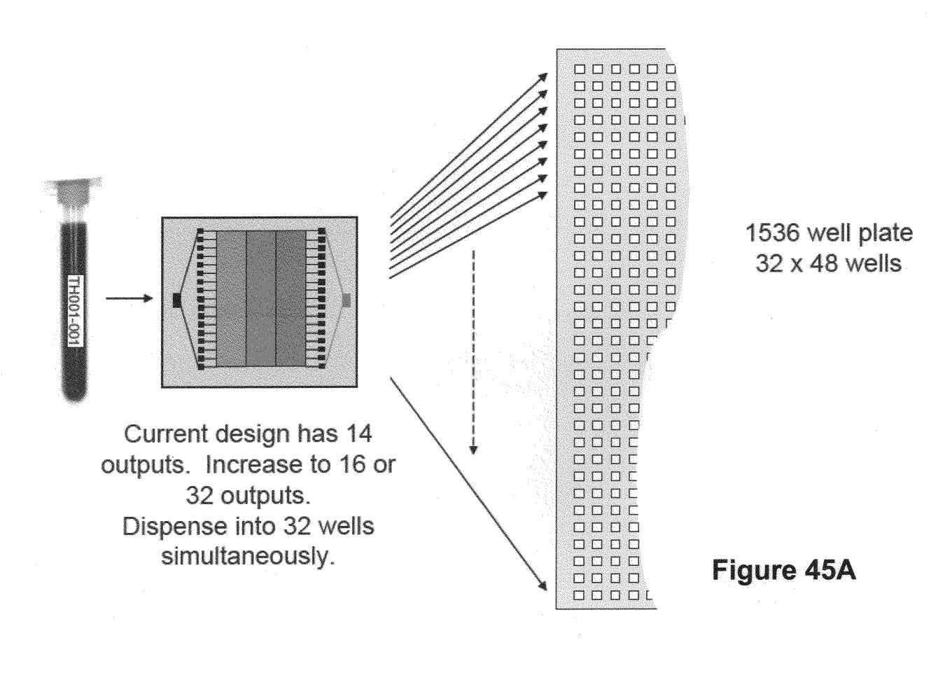

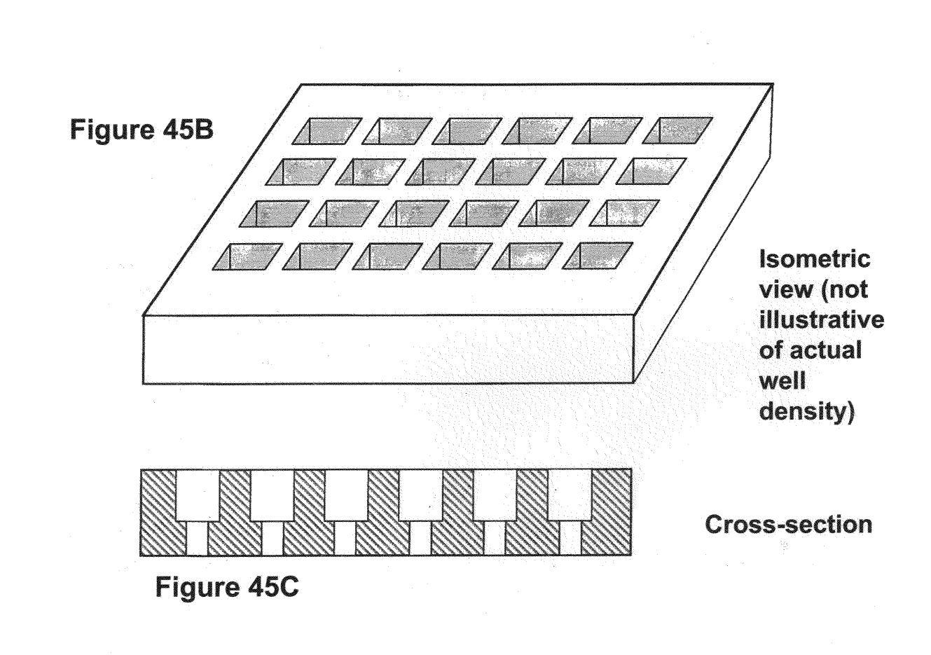

In step 4404, the enriched product is split between two or more discrete locations. In one embodiment, a sample is split into at least 2, 10, 20, 50, 100, 200, 300, 400, 500, 600, 700, 800, 900, 1000, 2000, 3,000, 4,000, 5000, or 10,000 total different discrete sites or about 100, 200, 500, 1000, 1200, 1500 sites. In one embodiment, output from an enrichment module is serially divided into wells of a 1536 microwell plate (FIG. 45A). This can result in one cell or genome per location or 0 or 1 cell or genome per location. In one embodiment, cell splitting results in more than 1, 2, 3, 4, 5, 6, 7, 8, 9, 10, 20, 50, 100, 200, 500, 1000, 2000, 5000, 10,000, 20,000, 50,000, 100,000, 200,000, or 500,000 cells or genomes per location. When splitting a sample enriched for fnRBC cells or placental cells the load at each discrete location (e.g., well) can include several leukocytes, while one only some of the loads includes one or more fnRBC cells or placental cells. When splitting a sample enriched for fetal cells, preferably each site includes 0 or 1 fetal cells.

Examples of discrete locations which could be used as addressable locations include, but are not limited to, wells, bins, sieves, pores, geometric sites, slides, matrixes, membranes, electric traps, gaps, obstacles, or in-situ within a cell or nuclear membrane. In one embodiment, the discrete cells are addressable such that one can correlate a cell or cell sample with a particular location.

Examples of methods for splitting a sample into discrete addressable locations include, but are not limited to, microfluidic fluorescent cell sorting or fluorescent activated cell sorting (FACS) (Sherlock, J V et al. Ann. Hum. Genet. 62 (Pt. 1): 9-23 (1998)), micromanipulation (Samura, 0., et al Hum. Genet. 107(1):28-32 (2000)) and dilution strategies (Findlay, I. et al. Mol. Cell. Endocrinol. 183 Suppl 1: S5-12 (2001)), each of which are herein incorporated by reference in their entireties. Other methods for sample splitting cell sorting and splitting methods known in the art can be used. For example, samples can be split by affinity sorting techniques using affinity agents (e.g., antibodies) bound to any immobilized or mobilized substrate (Samura O., et al., Hum. Genet. 107(1):28-32 (2000), which is herein incorporated by reference in its entirety). Such affinity agents can be specific to a cell type e.g., RBC's fetal cells epithelial cells including those specifically binding EpCAM, antigen-i, or CD-71.

In one embodiment, a sample or enriched sample is transferred to a cell sorting device that includes an array of discrete locations for capturing cells traveling along a fluid flow. The discrete locations can be arranged in a defined pattern across a surface such that the discrete sites are also addressable. In one embodiment, the sorting device is coupled to any of the enrichment devices known in the art or disclosed herein. Examples of cell sorting devices included are described in International Publication No. WO 01/35071, which is herein incorporated by reference in its entirety. Examples of surfaces that can be used for creating arrays of cells in discrete addressable sites include, but are not limited to, cellulose, cellulose acetate, nitrocellulose, glass, quartz or other crystalline substrates such as gallium arsenide, silicones, metals, semiconductors, various plastics and plastic copolymers, cyclo-olefin polymers, various membranes and gels, microspheres, beads and paramagnetic or supramagnetic microparticles.

In one embodiment, a sorting device comprises an array of wells or discrete locations wherein each well or discrete location is configured to hold up to 1 cell. Each well or discrete addressable location can have a capture mechanism adapted for retention of such cell (e.g., gravity, suction, etc.) and optionally a release mechanism for selectively releasing a cell of interest from a specific well or site (e.g., bubble actuation). FIG. 45B illustrates such an embodiment.

In step 4406, nucleic acids of interest from each cell or nuclei arrayed are tagged by amplification. In one embodiment, the amplified/tagged nucleic acids include at least 1, 2, 3, 4, 5, 6, 7, 8, 9, 10, 20, 30, 40, 50, 60, 70, 90, 90 or 100 polymorphic genomic DNA regions such as short tandem repeats (STRs) or variable number of tandem repeats (“VNTR”). When the amplified DNA regions include one or more STR/s/, the STR/s/ are selected for high heterozygosity (variety of alleles) such that the paternal allele of any fetal cell is more likely to be distinct in length from the maternal allele. This results in improved power to detect the presence of fetal cells in a mixed sample and any potential of fetal abnormalities in such cells. In some embodiment, STR(s) amplified are selected for their association with a particular condition. For example, to determine fetal abnormality an STR sequence comprising a mutation associated with fetal abnormality or condition is amplified. Examples of STRs that can be amplified/analyzed by the methods herein include, but are not limited to D21S1414, D21S1411, D21S1412, D21S11 MBP, D13S634, D13S631, D18S535, AmgXY and XHPRT. Additional STRs that can be amplified/analyzed by the methods herein include, but are not limited to, those at locus F13B (1:q31-q32); TPDX (2:p23-2pter); FIBRA (FGA) (4:q28); CSFIPO (5:q33.3-q34); FI3A (6:p24-p25); THOI (11:p15-15.5); VWA (12:p12-pter); CDU (12p12-pter); D14S1434 (14:q32.13); CYAR04 (p450) (15:q21.1) D21 S11 (21:q11-q21) and D22S1045 (22:q12.3). In some cases, STR loci are chosen on a chromosome suspected of trisomy and on a control chromosome. Examples of chromosomes that are often trisomic include chromosomes 21, 18, 13, and X. In some cases, 1 or more than 1, 2, 3, 4, 5, 6, 7, 8, 9, 10, 15, or 20 STRs are amplified per chromosome tested (Samura, O. et al., Clin. Chem. 47(9):1622-6 (2001), which is herein incorporated by reference in its entirety). For example amplification can be used to generate amplicons of up to 20, up to 30, up to 40, up to 50, up to 60, up to 70, up to 80, up to 90, up to 100, up to 150, up to 200, up to 300, up to 400, up to 500 or up to 1000 nucleotides in length. Di-, tri-, tetra-, or penta-nucleotide repeat STR loci can be used in the methods described herein.

To amplify and tag genomic DNA region(s) of interest, PCR primers can include: (i) a primer element, (ii) a sequencing element, and (iii) a locator element.

The primer element is configured to amplify the genomic DNA region of interest (e.g. STR). The primer element includes, when necessary, the upstream and downstream primers for the amplification reactions. Primer elements can be chosen which are multiplexible with other primer pairs from other tags in the same amplification reaction (e.g., fairly uniform melting temperature, absence of cross-priming on the human genome, and absence of primer-primer interaction based on sequence analysis). The primer element can have at least 4, 5, 6, 7, 8, 9, 10, 15, 20, 25, 30, 40 or 50 nucleotide bases, which are designed to specifically hybridize with and amplify the genomic DNA region of interest.

The sequencing element can be located on the 5′ end of each primer element or nucleic acid tag. The sequencing element is adapted to cloning and/or sequencing of the amplicons (Marguiles, M, Nature 437 (7057): 376-80, which is herein incorporated by reference in its entirety). The sequencing element can be about 4, 6, 8, 10, 18, 20, 28, 36, 46 or 50 nucleotide bases in length.

The locator element (also known as a unique tag sequence), which is often incorporated into the middle part of the upstream primer, can include a short DNA or nucleic acid sequence between 4-20 by in length (e.g., about 4, 6, 8, 10, or 20 nucleotide bases). The locator element makes it possible to pool the amplicons from all discrete addressable locations following the amplification step and analyze the amplicons in parallel. In one embodiment each locator element is specific for a single addressable location.

Tags are added to the cells/DNA at each discrete location using an amplification reaction. Amplification can be performed using PCR or by a variety of methods including, but not limited to, singleplex PCR, quantitative PCR, quantitative fluorescent PCR (QF-PCR), multiplex fluorescent PCR (MF-PCR), real time PCR(RT-PCR), single cell PCR, restriction fragment length polymorphism PCR(PCR-RFLP), PCR-RFLP/RT-PCR-RFLP, hot start PCR, nested PCR, in situ polony PCR, in situ rolling circle amplification (RCA), bridge PCR, picotiter PCR, multiple strand displacement amplification (MDA), and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Additional examples of amplification techniques using PCR primers are described in, U.S. Pat. Nos. 5,242,794, 5,494,810, 4,988,617 and 6,582,938, which are herein incorporated by reference in their entirety.

In some embodiments, a further PCR amplification is performed using nested primers for the one or more genomic DNA regions of interest to ensure optimal performance of the multiplex amplification. The nested PCR amplification generates sufficient genomic DNA starting material for further analysis such as in the parallel sequencing procedures below.

In step 4408, genomic DNA regions tagged/amplified are pooled and purified prior to further processing. Methods for pooling and purifying genomic DNA are known in the art.

In step 4410, pooled genomic DNA/amplicons are analyzed to measure, e.g., allele abundance of genomic DNA regions (e.g. STRs amplified). In one embodiment such analysis involves the use of capillary gel electrophoresis (CGE). In another embodiment, such analysis involves sequencing or ultra deep sequencing.

Sequencing can be performed using the classic Sanger sequencing method or any other method known in the art.

For example, sequencing can occur by sequencing-by-synthesis, which involves inferring the sequence of the template by synthesizing a strand complementary to the target nucleic acid sequence. Sequence-by-synthesis can be initiated using sequencing primers complementary to the sequencing element on the nucleic acid tags. The method involves detecting the identity of each nucleotide immediately after (substantially real-time) or upon (real-time) the incorporation of a labeled nucleotide or nucleotide analog into a growing strand of a complementary nucleic acid sequence in a polymerase reaction. After the successful incorporation of a label nucleotide, a signal is measured and then nulled by methods known in the art. Examples of sequence-by-synthesis methods are described in U.S. Application Publication Nos. 2003/0044781, 2006/0024711, 2006/0024678 and 2005/0100932, which are herein incorporated by reference in their entirety. Examples of labels that can be used to label nucleotide or nucleotide analogs for sequencing-by-synthesis include, but are not limited to, chromophores, fluorescent moieties, enzymes, antigens, heavy metal, magnetic probes, dyes, phosphorescent groups, radioactive materials, chemiluminescent moieties, scattering or fluorescent nanoparticles, Raman signal generating moieties, and electrochemical detection moieties. Sequencing-by-synthesis can generate at least 1,000, at least 5,000, at least 10,000, at least 20,000, 30,000, at least 40,000, at least 50,000, at least 100,000 or at least 500,000 reads per hour. Such reads can have at least 50, at least 60, at least 70, at least 80, at least 90, at least 100, at least 120 or at least 150 bases per read.

Another sequencing method involves hybridizing the amplified genomic region of interest to a primer complementary to it. This hybridization complex is incubated with a polymerase, ATP sulfurylase, luciferase, apyrase, and the substrates luciferin and adenosine 5′ phosphosulfate. Next, deoxynucleotide triphosphates corresponding to the bases A, C, G, and T (U) are added sequentially. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

Yet another sequencing method involves a four-color sequencing by ligation scheme (degenerate ligation), which involves hybridizing an anchor primer to one of four positions. Then an enzymatic ligation reaction of the anchor primer to a population of degenerate nonamers that are labeled with fluorescent dyes is performed. At any given cycle, the population of nonamers that is used is structure such that the identity of one of its positions is correlated with the identity of the fluorophore attached to that nonamer. To the extent that the ligase discriminates for complementarily at that queried position, the fluorescent signal allows the inference of the identity of the base. After performing the ligation and four-color imaging, the anchor primer: nonamer complexes are stripped and a new cycle begins. Methods to image sequence information after performing ligation are known in the art.

In one embodiment, analysis involves the use of ultra-deep sequencing, such as described in Marguiles et al., Nature 437 (7057): 376-80 (2005), which is herein incorporated by reference in its entirety. Briefly, the amplicons are diluted and mixed with beads such that each bead captures a single molecule of the amplified material. The DNA molecule on each bead is then amplified to generate millions of copies of the sequence which all remain bound to the bead. Such amplification can occur by PCR. Each bead can be placed in a separate well, which can be a (optionally addressable) picolitre-sized well. In one embodiment, each bead is captured within a droplet of a PCR-reaction-mixture-in-oil-emulsion and PCR amplification occurs within each droplet. The amplification on the bead results in each bead carrying at least one million, at least 5 million, or at least 10 million copies of the original amplicon coupled to it. Finally, the beads are placed into a highly parallel sequencing by synthesis machine which generates over 400,000 reads (˜100 bp per read) in a single 4 hour run.

Other methods for ultra-deep sequencing that can be used are described in Hong, S. et al. Nat. Biotechnol. 22(4):435-9 (2004); Bennett, B. et al. Pharmacogenomics 6(4):373-82 (2005); Shendure, P. et al. Science 309 (5741):1728-32 (2005), which are herein incorporated by reference in their entirety.

The role of the ultra-deep sequencing is to provide an accurate and quantitative way to measure the allele abundances for each of the STRs. The total required number of reads for each of the aliquot wells is determined by the number of STRs, the error rates of the multiplex PCR, and the Poisson sampling statistics associated with the sequencing procedures.

In one example, the enrichment output from step 4402 results in approximately 500 cells of which 98% are maternal cells and 2% are fetal cells. Such enriched cells are subsequently split into 500 discrete locations (e.g., wells) in a microtiter plate such that each well contains 1 cell. PCR is used to amplify STR's (˜3-10 STR loci) on each chromosome of interest. Based on the above example, as the fetal/maternal ratio goes down, the aneuploidy signal becomes diluted and more loci are needed to average out measurement errors associated with variable DNA amplification efficiencies from locus to locus. The sample division into wells containing ˜1 cell proposed in the methods described herein achieves pure or highly enriched fetal/maternal ratios in some wells, alleviating the requirements for averaging of PCR errors over many loci.

In one example, let ‘f’ be the fetal/maternal DNA copy ratio in a particular PCR reaction. Trisomy increases the ratio of maternal to paternal alleles by a factor 1+f/2. PCR efficiencies vary from allele to allele within a locus by a mean square error in the logarithm given by σₐₗₗₑₗₑ², and vary from locus to locus by σ_{locus}², where this second variance is apt to be larger due to differences in primer efficiency. N_{c} is the loci per suspected aneuploid chromosome and N_{c} is the control loci. If the mean of the two maternal allele strengths at any locus is ‘m’ and the paternal allele strength is ‘p,’ then the squared error expected is the mean of the ln(ratio(m/p)), where this mean is taken over N loci is given by 2(σₐₗₗₑₗₑ²)/N. When taking the difference of this mean of ln(ratio(m/p)) between a suspected aneuploidy region and a control region, the error in the difference is given by

σ_{diff}²=2(σₐₗₗₑₗₑ²)/Nₐ+2(σₐₗₗₑₗₑ²)/N_{c} (1)

A robust detection of aneuploidy requires

3σ_{diff}<f/2.

For simplicity, assuming Nₐ=N_{c}=N in Equation 1, this gives the requirement

6σₐₗₗₑₗₑ/N^{1/2}<f/2, (3)

or a minimum N of

N=144(σₐₗₗₑₗₑ/f)² (4)

In the context of trisomy detection, the suspected aneuploidy region is usually the entire chromosome and N denotes the number of loci per chromosome. For reference, Equation 3 is evaluated for N in the following Table 3 for various values of σₐₗₗₑₗₑ and f.

TABLE 3
Required number of loci per chromosome
as a function of σₐₗₗₑₗₑ and f.

| | f | | |
|---|---|---|---|
| σₐₗₗₑₗₑ | 0.1 | 0.3 | 1.0 |
| 0.1 | 144 | 16 | 1 |
| 0.3 | 1296 | 144 | 13 |
| 1.0 | 14400 | 1600 | 144 |

Since sample splitting decreases the number of starting genome copies which increases σₐₗₗₑₗₑ at the same time that it increases the value of fin some wells, the methods herein are based on the assumption that the overall effect of splitting is favorable; i.e., that the PCR errors do not increase too fast with decreasing starting number of genome copies to offset the benefit of having some wells with large f. The required number of loci can be somewhat larger because for many loci the paternal allele is not distinct from the maternal alleles, and this incidence depends on the heterozygosity of the loci. In the case of highly polymorphic STRs, this amounts to an approximate doubling of N.

The role of the sequencing is to measure the allele abundances output from the amplification step. It is desirable to do this without adding significantly more error due to the Poisson statistics of selecting only a finite number of amplicons for sequencing. The rms error in the ln(abundance) due to Poisson statistics is approximately (N_{reads)}^{-1/2}. It is desirable to keep this value less than or equal to the PCR error a σₐₗₗₑₗₑ. Thus, a typical paternal allele needs to be allocated at least (σₐₗₗₑₗₑ)⁻² reads. The maternal alleles, being more abundant, do not add appreciably to this error when forming the ratio estimate for m/p. The mixture input to sequencing contains amplicons from N_{loci} loci of which roughly an abundance fraction f/2 are paternal alleles. Thus, the total required number of reads for each of the aliquot wells is given approximately by 2N_{loci}/(f σₐₗₗₑₗₑ²). Combining this result with Equation 4, it is found a total number of reads over all the wells given approximately by

N_{reads}=288 N_{wells} f³. (5)

When performing sample splitting, a rough approximation is to stipulate that the sample splitting causes f to approach unity in at least a few wells. If the sample splitting is to have advantages, then it must be these wells which dominate the information content in the final result. Therefore, Equation (5) with f=1 is adopted, which suggests a minimum of about 300 reads per well. For 500 wells, this gives a minimum requirement for 150,000 sequence reads. Allowing for the limited heterozygosity of the loci tends to increase the requirements (by a factor of ˜2 in the case of STRs), while the effect of reinforcement of data from multiple wells tends to relax the requirements with respect to this result (in the baseline case examined above it is assumed that ˜10 wells have a pure fetal cell). Thus the required total number of reads per patient is expected to be in the range 100,000-300,000.

In step 4412, wells with rare cells/alleles (e.g., fetal alleles) are identified. The locator elements of each tag can be used to sort the reads (˜200,000 sequence reads) into ‘bins’ which correspond to the individual wells of the microtiter plates (˜500 bins). The sequence reads from each of the bins (˜400 reads per bin) are then separated into the different genomic DNA region groups, (e.g. STR loci,) using standard sequence alignment algorithms. The aligned sequences from each of the bins are used to identify rare (e.g., non-maternal) alleles. It is estimated that on average a 15 ml blood sample from a pregnant human will result in ˜10 bins having a single fetal cell each.

The following are two examples by which rare alleles can be identified. In a first approach, an independent blood sample fraction known to contain only maternal cells can be analyzed as described above in order to obtain maternal alleles. This sample can be a white blood cell fraction or simply a dilution of the original sample before enrichment. In a second approach, the sequences or genotypes for all the wells can be similarity-clustered to identify the dominant pattern associated with maternal cells. In either approach, the detection of non-maternal alleles determines which discrete location (e.g. well) contained fetal cells. Determining the number of bins with non-maternal alleles relative to the total number of bins provides an estimate of the number of fetal cells that were present in the original cell population or enriched sample. Bins containing fetal cells are identified with high levels of confidence because the non-maternal alleles are detected by multiple independent polymorphic DNA regions, e.g. STR loci.

In step 4414, condition of one or more rare cells or DNA is determined. This determination can be accomplished by determining abundance of selected alleles (polymorphic genomic DNA regions) in bin(s) with rare cells/DNA. In one embodiment, allele abundance is used to determine aneuploidy, e.g., chromosomes 13, 18 and 21. Abundance of alleles can be determined by comparing the ratio of maternal to paternal alleles for each genomic region amplified (e.g., ˜12 STR's). For example, if 12 STRs are analyzed, for each bin there are 33 sequence reads for each of the STRs. In a normal fetus, a given STR will have 1:1 ratio of the maternal to paternal alleles with approximately 16 sequence reads corresponding to each allele (normal diallelic). In a trisomic fetus, three doses of an STR marker will be detected either as three alleles with a 1:1:1 ratio (trisomic triallelic) or two alleles with a ratio of 2:1 (trisomic diallelic) (Adinolfi, P. et al., Prenat. Diagn, 17(13):1299-311 (1997), which is herein incorporated by reference in its entirety). In rare instances all three alleles can coincide and the locus will not be informative for that individual patient. In one embodiment, the information from the different DNA regions on each chromosome are combined to increase the confidence of a given aneuploidy call. In one embodiment, the information from the independent bins containing fetal cells can also be combined to further increase the confidence of the call.

In one embodiment allele abundance is used to determine segmental aneuploidy. Normal diploid cells have two copies of each chromosome and thus two alleles of each gene or loci. Changes in the allele abundance for a particular chromosomal region can be indicative of a chromosomal rearrangement, such as a deletion, duplication or translocation event. In some embodiments, the information from the different DNA regions on each chromosome are combined to increase the confidence of a given segmental aneuploidy call. In some embodiments, the information from the independent bins containing fetal cells can also be combined to further increase the confidence of the call.

The determination of fetal trisomy can be used to diagnose conditions such as abnormal fetal genotypes, including, trisomy 13, trisomy 18, trisomy 21 (Down syndrome) and Klinefelter Syndrome (XXY). Other examples of abnormal fetal genotypes include, but are not limited to, aneuploidy such as, monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g. XXXX, XXYY, XXXY, XYYY, XXXXX, XXXXY, XXXYY, XYYYY and XXYYY), triploidy (three of every chromosome, e.g. 69 chromosomes in humans), tetraploidy (four of every chromosome, e.g. 92 chromosomes in humans) and multiploidy. In some embodiments, an abnormal fetal genotype is a segmental aneuploidy. Examples of segmental aneuploidy include, but are not limited to, 1p36 duplication, dup(17)(p11.2p11.2) syndrome, Down syndrome, Pelizaeus-Merzbacher disease, dup(22)(q11.2q11.2) syndrome, and cat-eye syndrome. In some cases, an abnormal fetal genotype is due to one or more deletions of sex or autosomal chromosomes, which can result in a condition such as Cri-du-chat syndrome, Wolf-Hirschhorn, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, Steroid sulfatase deficiency, Kallmann syndrome, Microphthalmia with linear skin defects, Adrenal hypoplasia, Glycerol kinase deficiency, Pelizaeus-Merzbacher disease, Testis-determining factor on Y, Azospermia (factor, a), Azospermia (factor b), Azospermia (factor c), or 1p36 deletion. In some embodiments, a decrease in chromosomal number results in an XO syndrome.

In one embodiment, the methods of the invention allow for the determination of maternal or paternal trisomy. In some embodiments, the methods of the invention allow for the determination of trisomy or other conditions in fetal cells in a mixed maternal sample arising from more than one fetus.

In another aspect of the invention, standard quantitative genotyping technology is used to declare the presence of fetal cells and to determine the copy numbers (ploidies) of the fetal chromosomes. Several groups have demonstrated that quantitative genotyping approaches can be used to detect copy number changes (Wang, Moorhead et al. 2005, which is herein incorporated by reference in its entirety). However, these approaches do not perform well on mixtures of cells and typically require a relatively large number of input cells

### EXAMPLES

### Example 1

### Screening Method for Fetal Cell Markers

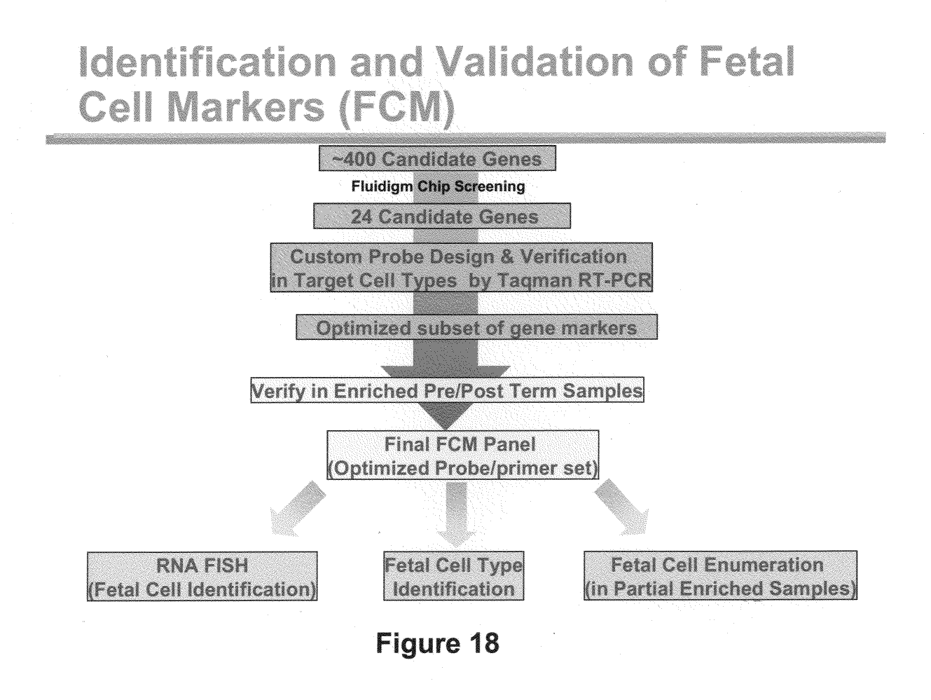

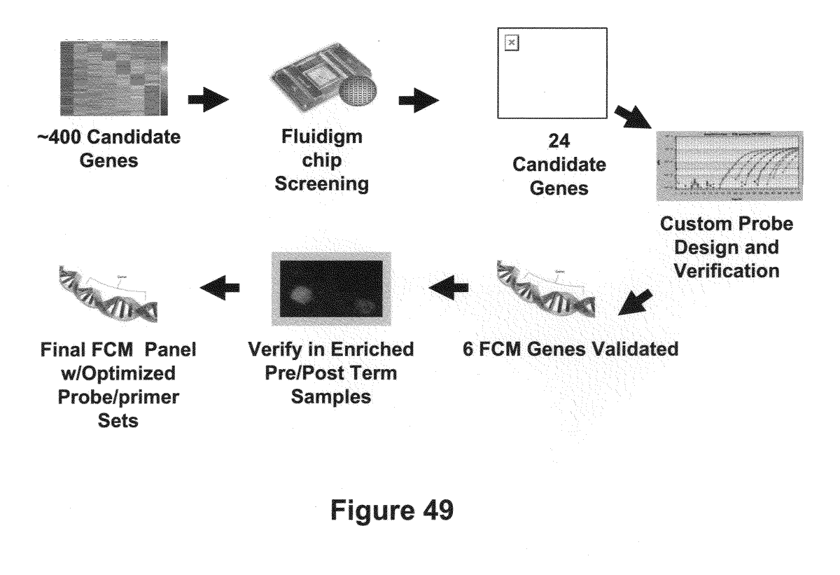

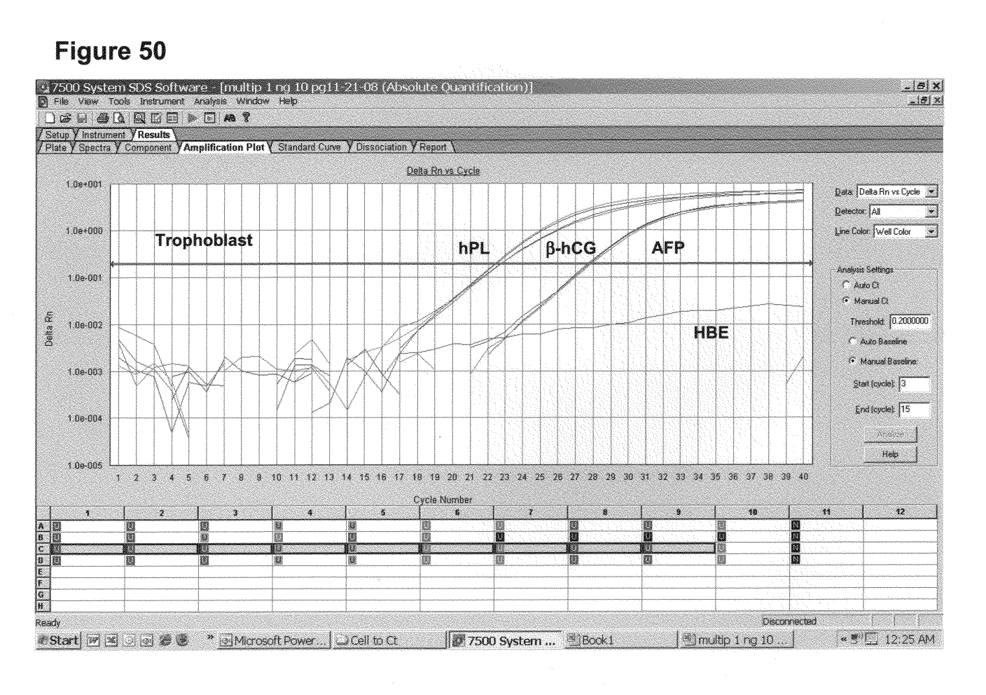

The process of identifying fetal cell markers includes the initial screening of pre-selected gene candidates by a Fluidigm PCR array approach followed by verification by Quantitative RT-PCR and further validation in clinical samples (FIGS. 18 and 49).

Model Tissues/Cell Systems

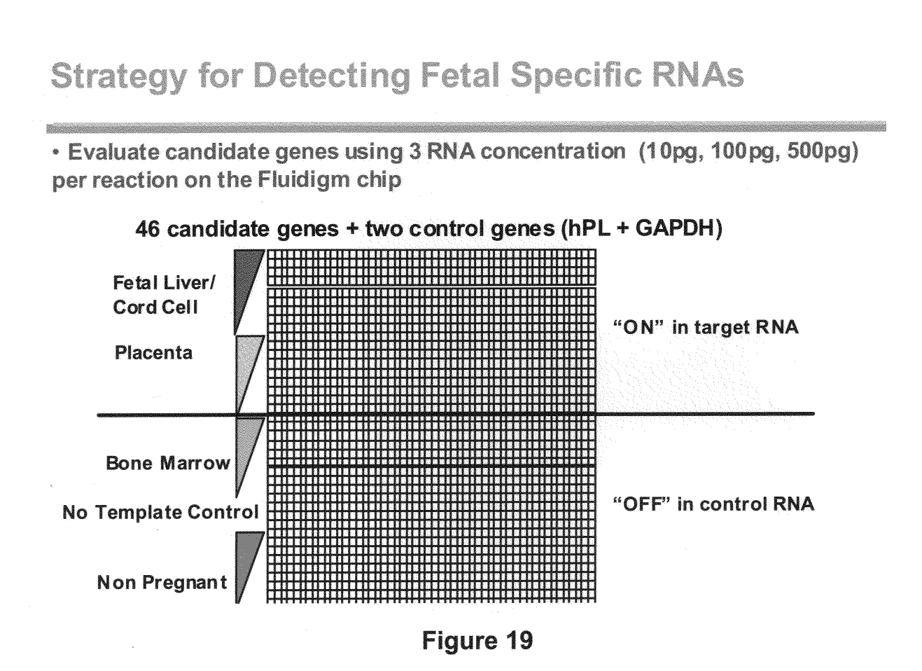

Several types of model tissues/cell systems were used to screen for fetal cell markers (FIG. 19). These include cord blood, which contains fetal blood cells, and non-pregnant peripheral blood cells (NP-PBC), which are normal adult blood cells. A fetal cell marker (FCM) is anticipated to be highly expressed in cord blood cells and at no or low expression level in NP-PBC. Another tissue source was bone marrow, which contains immature blood cells. ABM is used to distinguish the genes expressed in immature cells from ones expressed only in fetus. A FCM is expected to be at a low expression level in ABM. Finally, other cell sources include fetal liver and placenta. Fetal liver is a major organ for the generation of fetal blood cells in early fetal development. RNA from fetal liver contains abundant fetal nucleated red blood cells (fnRBC). Placenta contains different types of trophoblasts (TBC) and other cells of connective tissues.

Screening Process

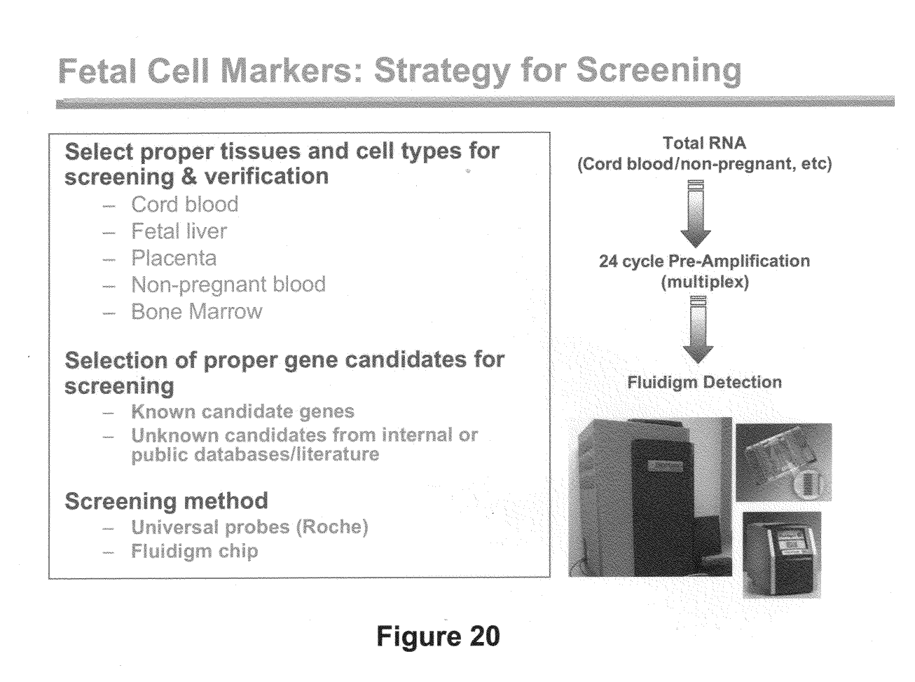

The gene candidates for initial screening were selected from various sources, including data from microarray experiments, public databases, and scientific literature. Approximately 400 genes were selected in the initial screening process. Initial screening was performed using a Fluidigm Chip (FIG. 20). Universal probe/primer sets from commercially available sources for selected genes were used in the initial Fluidigm PCR array screening. Total RNA (10 pg, 50 pg and 100 pg) from each of the selected tissues/cells was first pre-amplified by multiplex primer sets for all genes before being loaded for PCR in the Fluidigm chip. The pre-amplification step selectively amplified target genes. In each chip run, 3-6 repeats were done for each condition.

Verification and Further Selection

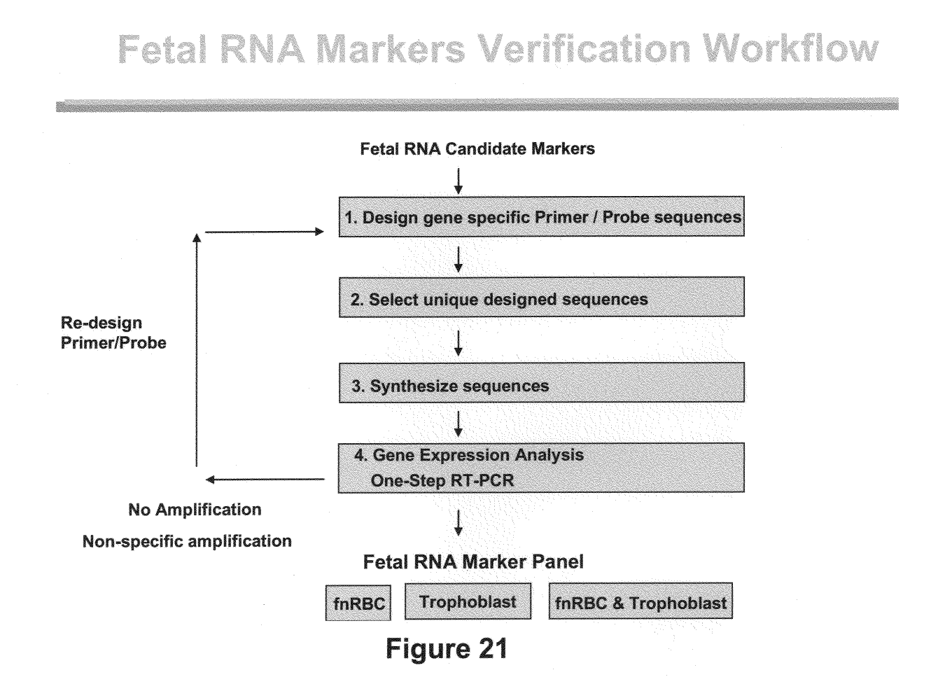

The verification and further selection of the genes markers were done by Taqman RT-PCR using custom probe/primer designs and testing in more defined target tissues and cells (FIG. 21). Probe and primers for each gene were custom designed. Several criteria were used to guide probe and primer design. First, common sequence regions for genes with multiple variants were selected that cover genes with multiple variants. Second, undesirable sequence regions were avoided, including regions with SNPs, the junctions of alternative variants, and areas with low complexity DNA sequences. Third, probe/primer sets that are specific for a target gene by BLAST analysis were selected.

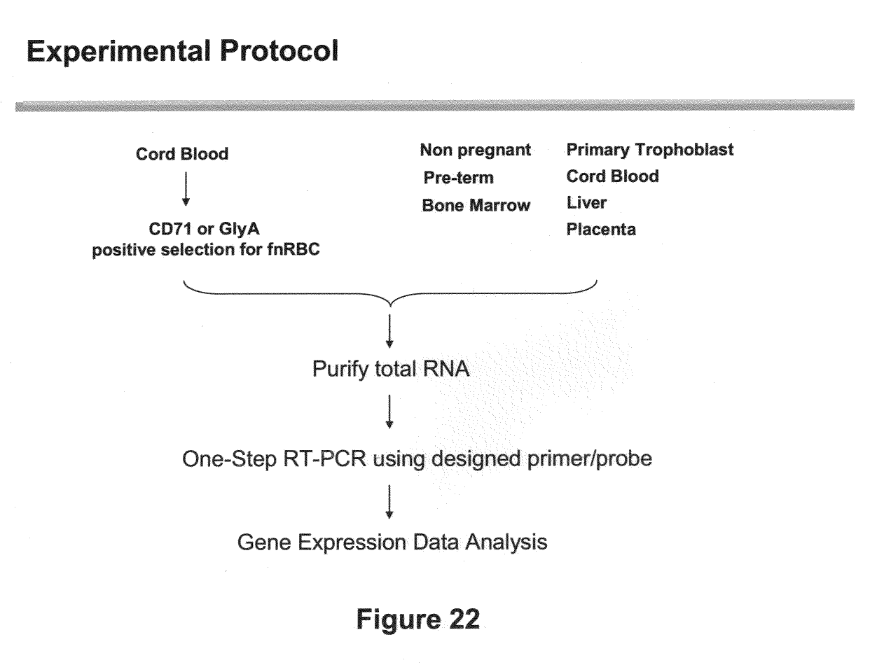

Defined tissues and cells were used to test for cell type specific verification (FIG. 22). Whole tissues included placenta and fetal liver. Additional material included cord blood, bone marrow, and non-pregnant peripheral blood cells. Anti-CD71 and/or anti-GLA purified fetal cells from cord blood and fetal liver were used (CD71 and GLA are relatively specific for fnRBC). CD71 also exists on the surface of trophoblasts. Also used were primary cultures of trophoblasts, which include cytotrophoblasts, the trophoblast type that most likely exists in maternal blood. The gestational ages of the samples from fetal liver, placenta and CD71-purified cells cover both 1^{st} trimester and early 2^{nd} trimester.

Method for Selection of Subset of Candidate Genes

The Ct value represents the gene expression level. In order to compare gene expression levels among different tissues/cells and different experimental sample lots, the Ct value for each gene and experiment was normalized by GAPDH, a constitutively active gene using the formula below:

ΔCt=Ct_{GAPDH}−Ct_{Target Gene}

By this expression, the positive ΔCt indicates a higher expression level of a target gene than GAPDH. A negative ΔCt indicates a lowered expression of a target gene than GAPDH.

The selection of final gene set is based on several criteria. First, expression of the gene should be tissue and cell-type specific. There gene should display a) no expression in non-pregnant samples, b) no expression or extremely low expression in bone marrow, and c) a high level of expression in target cell types and/or tissues, such as cold blood samples. Second, the overall gene panel should cover both 1st and early 2nd trimesters

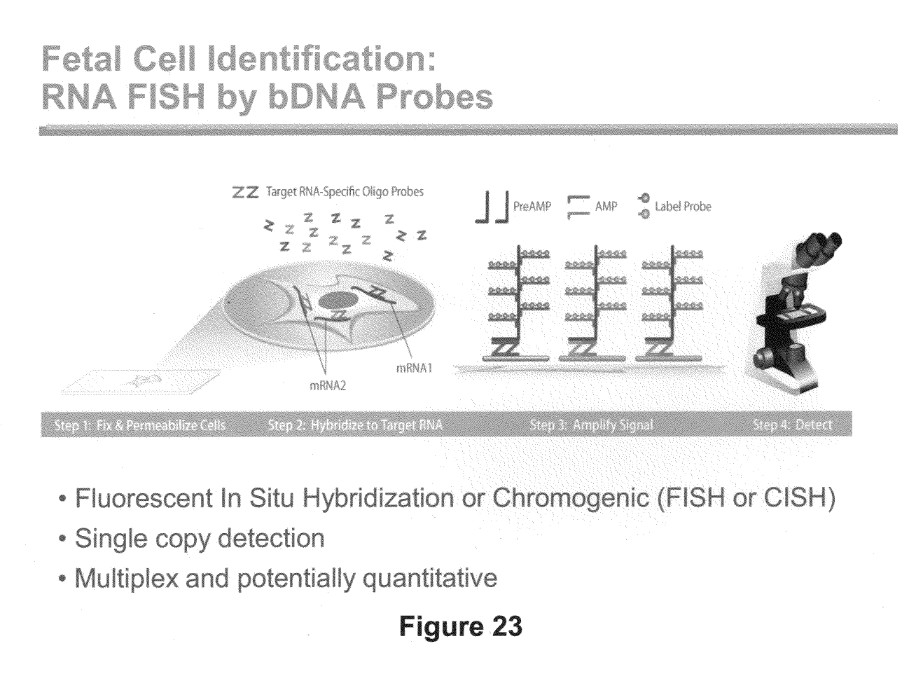

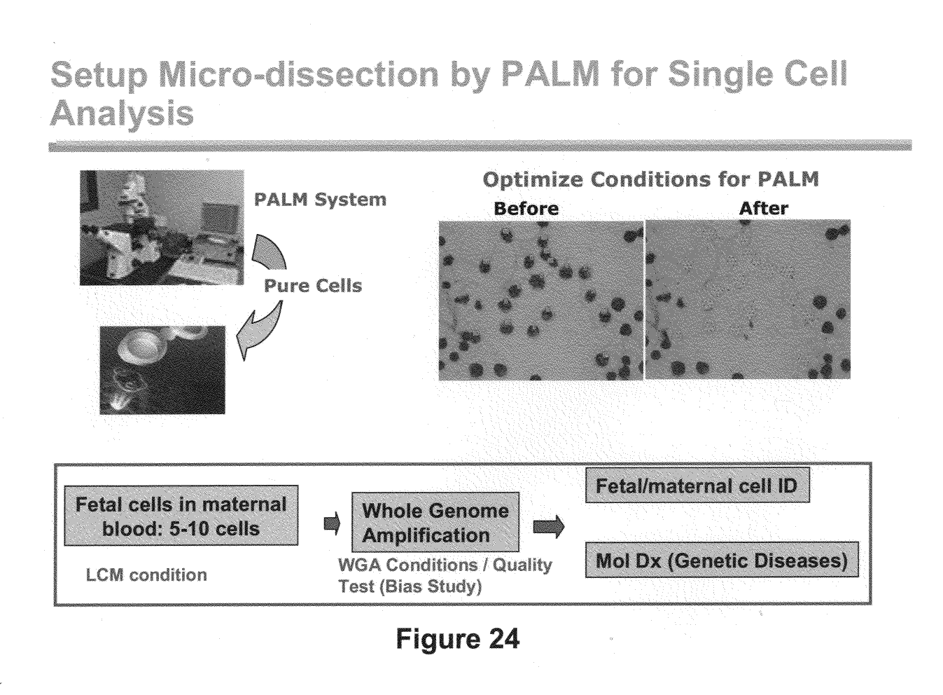

RNA FISH in model cell systems and pregnant blood samples can be used to verify and validate the markers (FIG. 23). In addition, single cell analysis can be used to validated gene labeling specificity (FIG. 24). One of the approaches to verify if expression of the selected genes is specific for a fetal cell is by identifying and micro-dissecting a fetal cell for further molecular analysis. For examples, if AFP or FN1 gene expression is detected in a micro-dissected fnRBC or trophoblast, then the results indicate that AFP and FN1 are expressed for these target cells.

### Example 2

### Summary of Screening Results

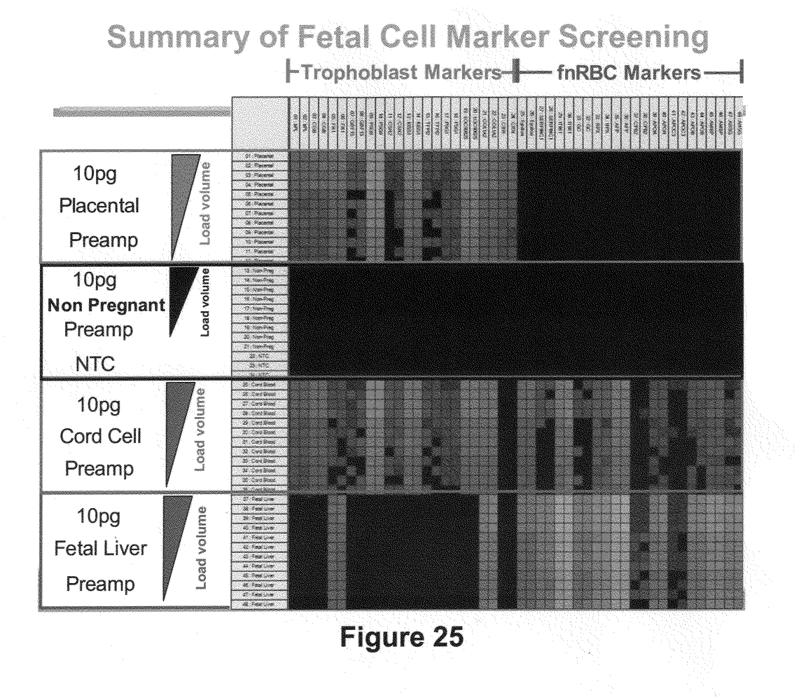

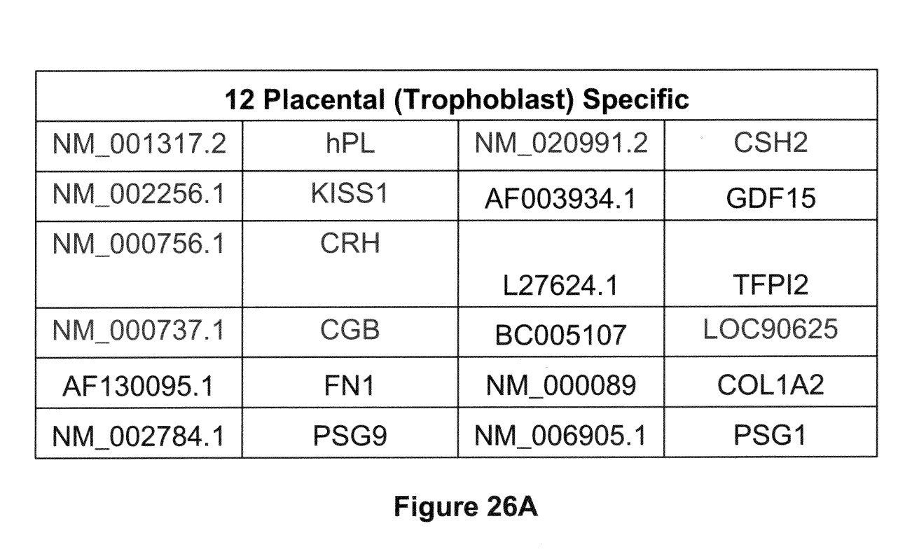

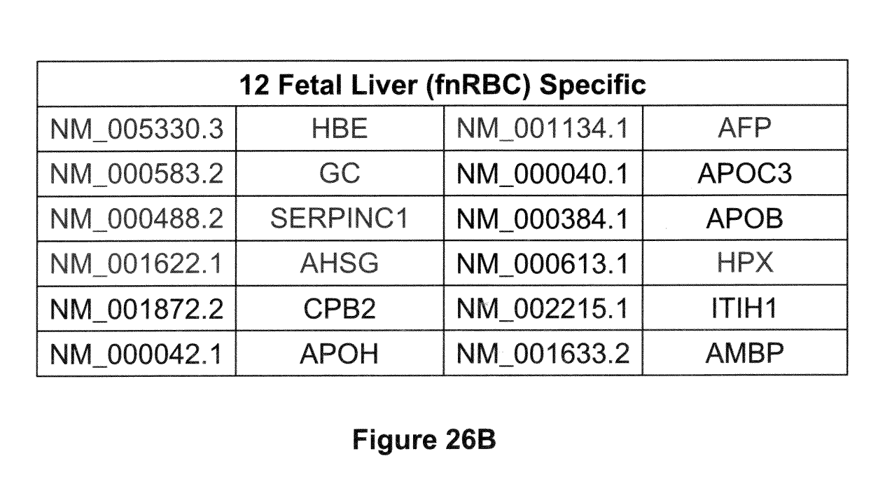

Approximately 400 pre-selected candidate genes were screened by PCR array using the Fluidigm Biomark Genetic Analysis platform. A summary of final screening results is shown in the (FIG. 25). 12 genes displaying specific expression in trophoblast and 12 genes displaying specific expression in fnRBC were identified. All trophoblast marker genes were not detected in non-pregnant samples and ABM (not shown), but strongly expressed in placental tissues and cord blood samples. Two genes were also expressed in fetal liver. All fnRBC marker genes are not detectable in non-pregnant, placenta and ABM (not shown), but are strongly expressed in cord blood and fetal liver. FIGS. 26A and 26B list the selected gene symbols and accession numbers.

Selection of FCM for Validation

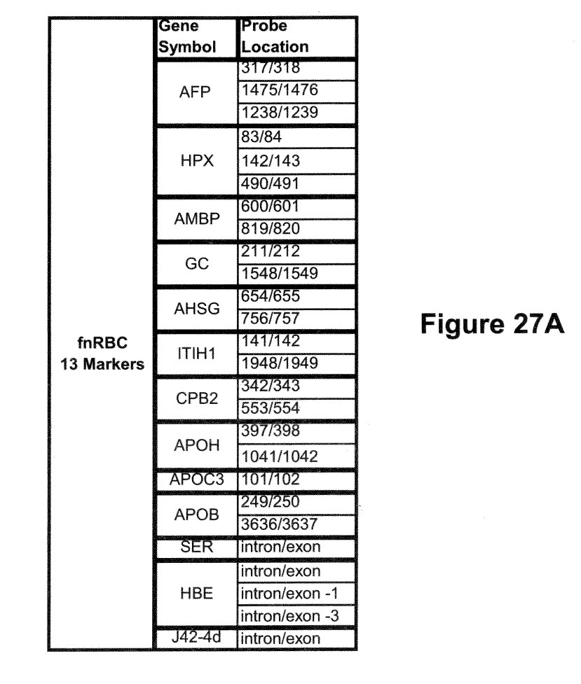

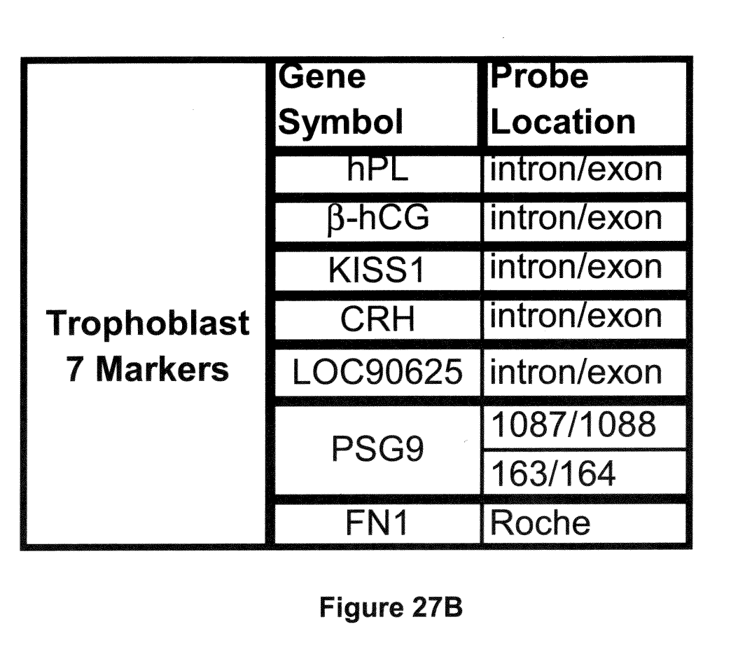

Twelve genes for fnRBC from the screening results and another gene called J42-4-d, a putative candidate gene of fnRBC, were selected for further testing and verification. Seven genes for trophoblast from screening results were selected based on prior experimental information or knowledge from literature. FIGS. 27A and 27B list the gene symbol and probe location for each gene for further verification.

Summary of Verification Results

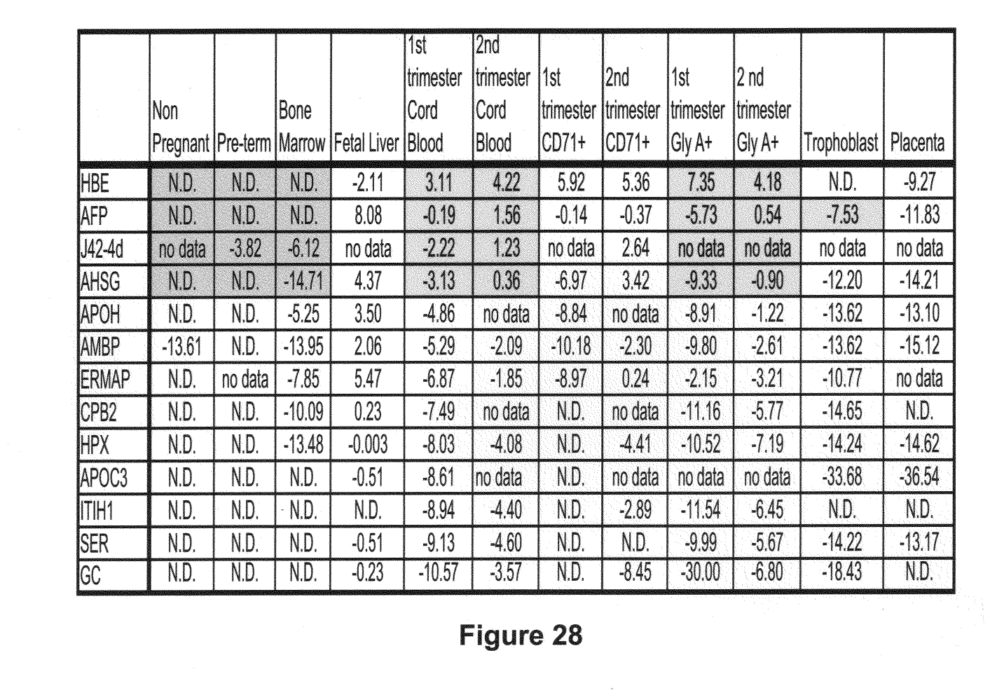

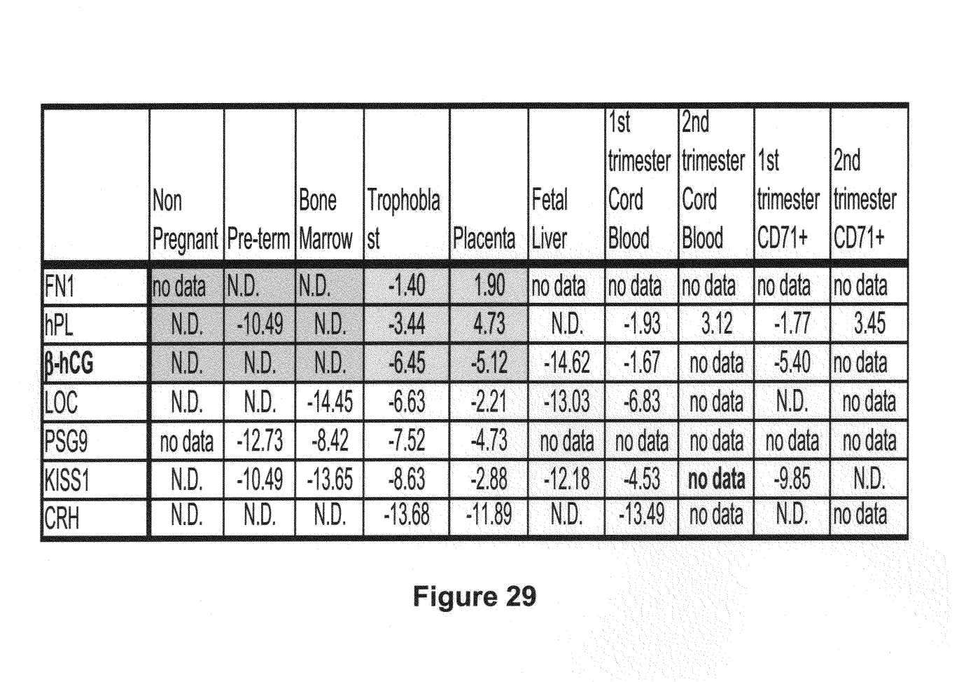

The expression data of putative fetal nRBC markers and trophoblast markers are presented in FIGS. 28 and 29. Gene Markers expressed in the 1^{st} and early 2^{nd} trimester samples indicate that:

HBE is the highest fnRBC expression marker in cord blood and CD71+ and GlyA+ selected cells. HBE expression is not detected in non-pregnant and preterm whole blood and bone marrow.

AFP is an fnRBC expression marker, the most abundant expressed in the fetal liver, and is also moderately expressed in primary trophoblast and placenta samples. The AFP expression level is increased in the early 2^{nd} trimester samples. AFP expression is not detected in non-pregnant and preterm whole blood and bone marrow.

AHSG is an expression marker for early 2^{nd} trimester fnRBC. The expression level is significantly increased in the early 2″ trimester cord blood and CD71+ and GlyA+ selected cells, although AHSG is relative low expressed in the 1^{st} trimester samples. AHSG expression is not detected in non-pregnant and preterm whole blood but is expressed at a low level in the bone marrow.

J42-4-d is a potential expression marker for early 2^{nd} trimester fnRBC, and it shows some expression in the preterm and bone marrow.

For trophoblasts, gene markers expressed in the 1^{st} and early 2^{nd} trimester samples include the following:

hPL displays high expression in the placenta, and is abundantly expressed in primary trophoblasts, cord blood, and CD71+ selected cells. hPL expression is not detected in non-pregnant and bone marrow, and displays very low expression in preterm whole blood.

β-hCG is a good expression trophoblast marker. It is moderately expressed in primary trophoblasts and placenta and relatively abundantly expressed in cord blood. β-hCG expression is not detected in non-pregnant peripheral blood cells and preterm whole blood and bone marrow.

FN1 is the highest expression marker in primary trophoblast. FN1 is abundantly expressed in placenta. FN1 is also expressed in the fetal liver shown in the FIG. 25.

Selection of Fetal Gene Markers

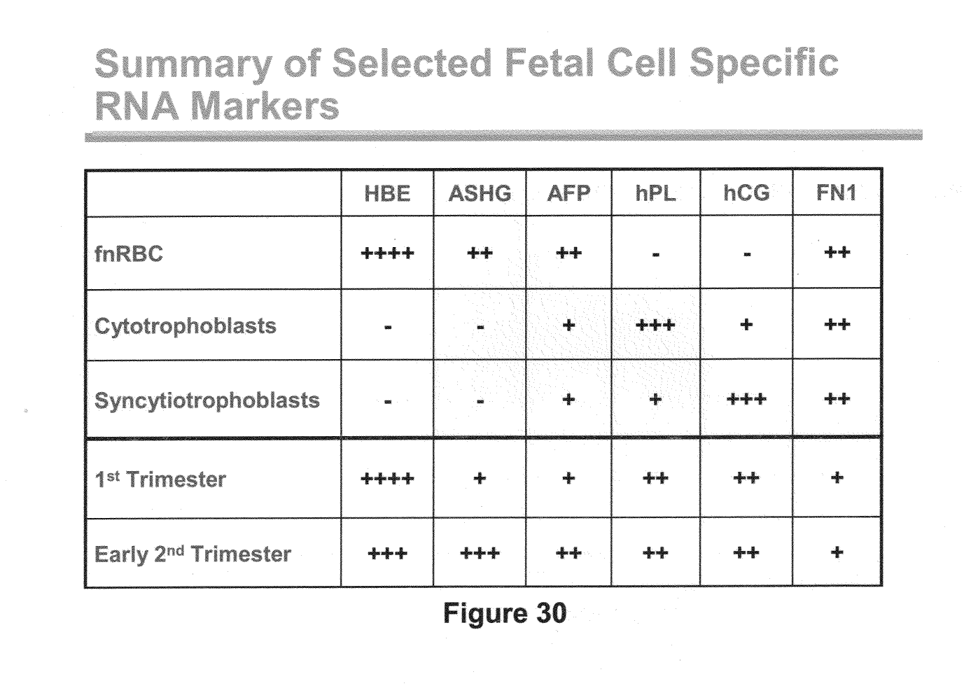

A subset of gene markers from the screening experiments based on the verification by quantitative RT-PCR was selected. The relative gene expression results and cell type selectivity are listed in FIG. 30.

Other candidate gene marker include J42-4-d for fnRBC and KISS1 and LOC90625 for trophoblasts.

Results by RNA FISH

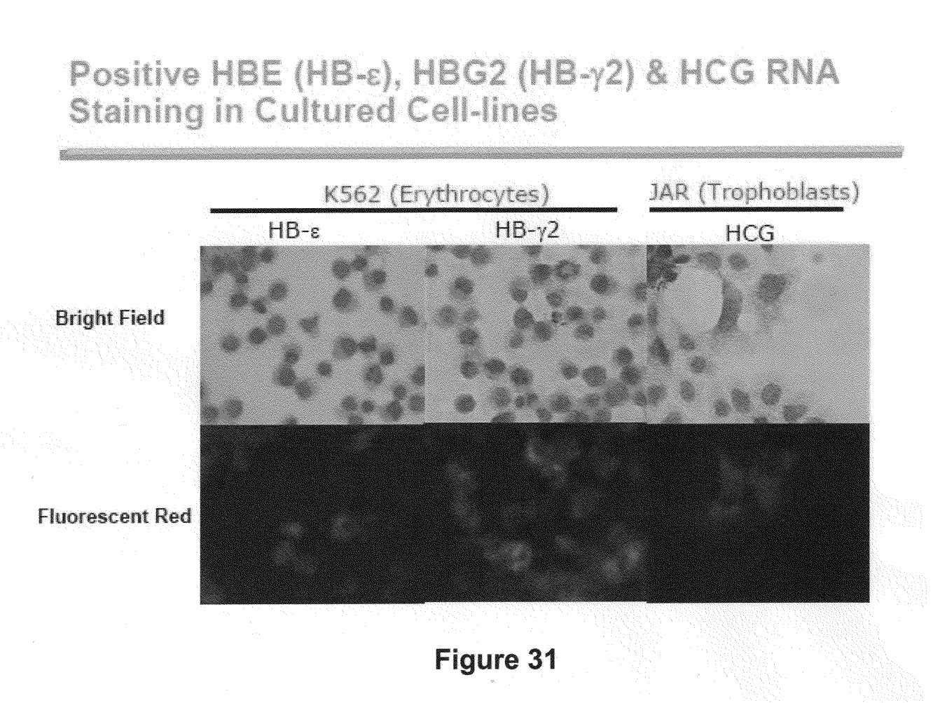

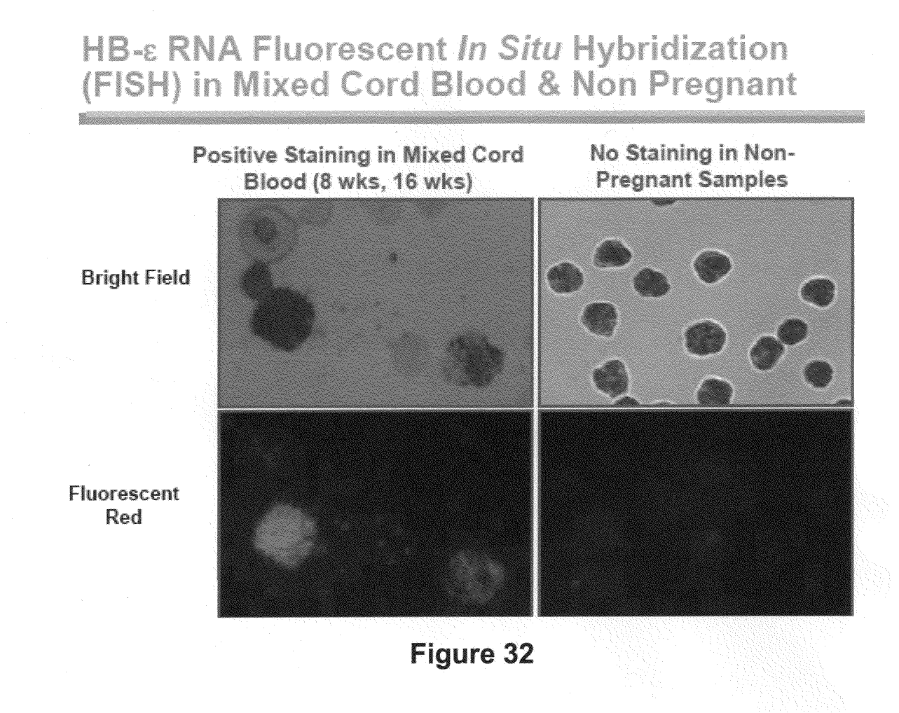

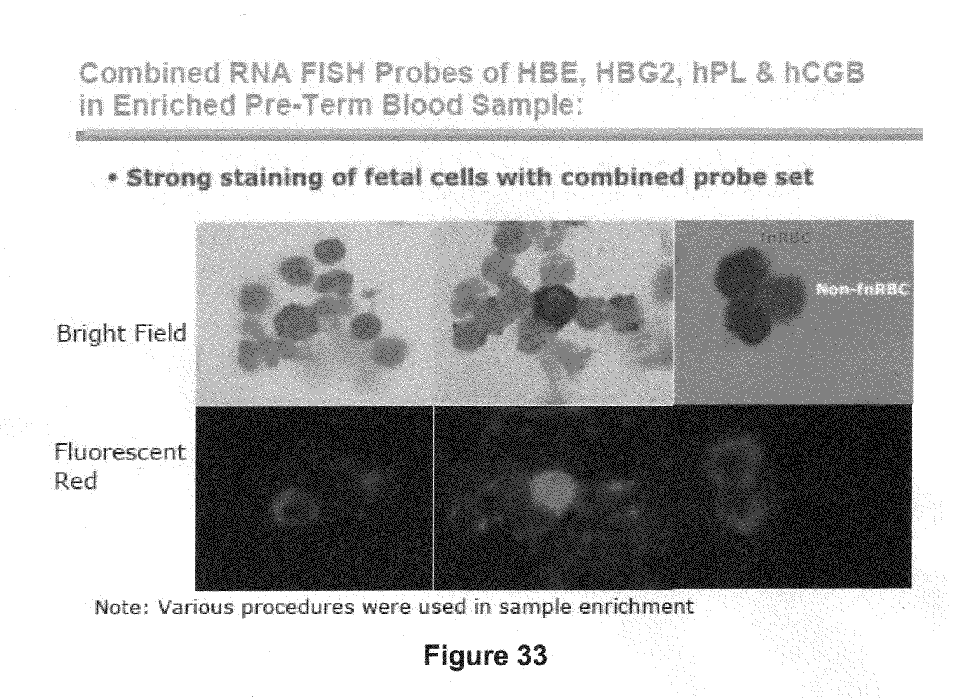

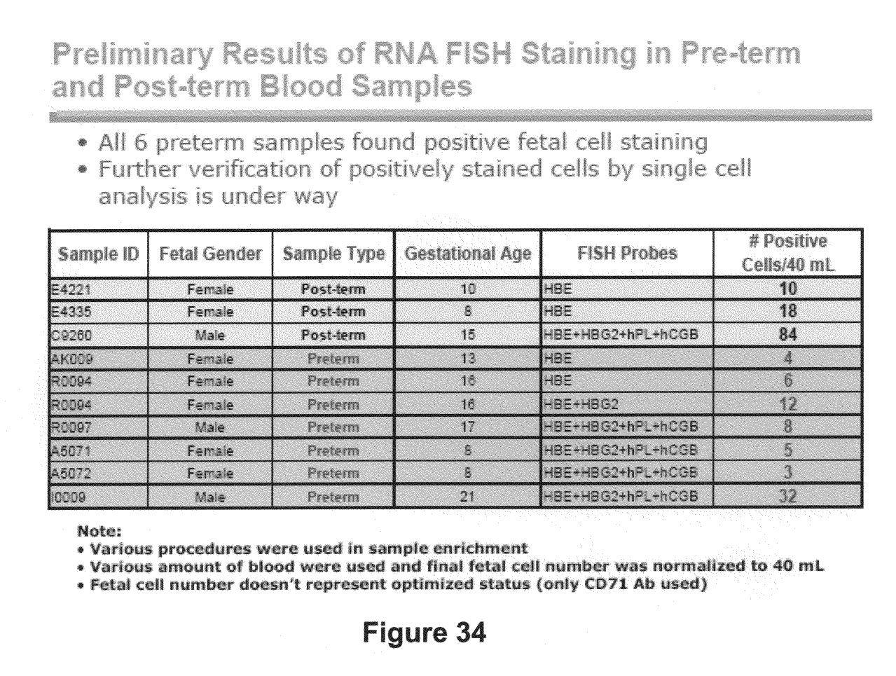

Transcripts from fetal marker genes have been detected by RNA FISH in model cell systems and blood samples (FIGS. 31, 32, 33, and 34). The results show that fnRBC and trophoblast specific gene markers specifically stain fetal nucleated RBC and trophoblasts, respectively, in the tested samples.

Example of Validation by Single Cell Analysis—Preliminary Demonstration

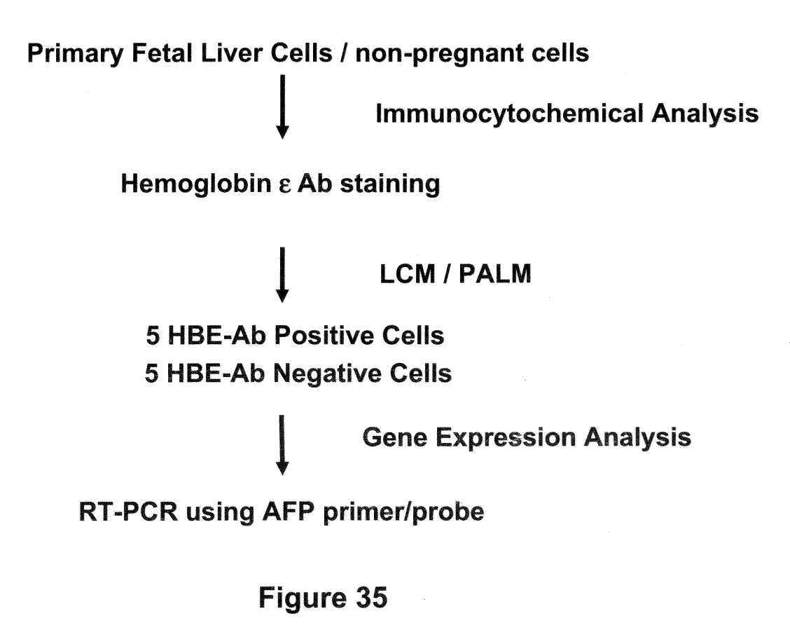

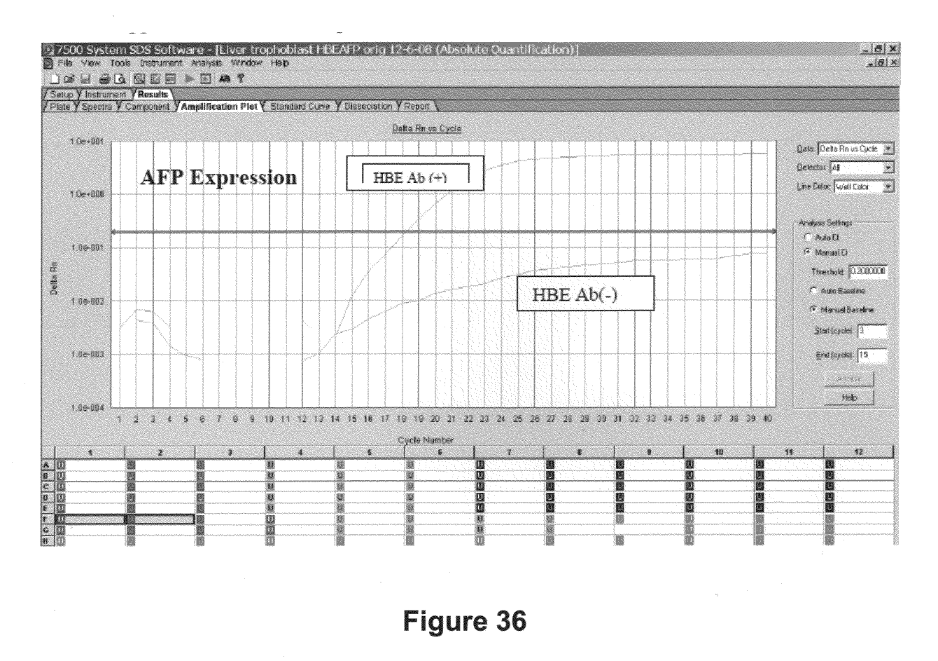

Primary liver cells, isolated from fetal liver, spiked into non-pregnant maternal cells samples were fixed onto the glass slides and used for immunocytochemical staining with hemoglobin ∈ antibody. Groups of 5 positive and 5 negative antibody staining cells were micro-dissected by PALM, separately. AFP gene expression of the group cells was analyzed directly using cell lysate and pre-amplification protocols as outlined in FIG. 35. AFP is expressed in the HBE antibody-stained positive cells, but not in negative cells (FIG. 36). The results suggest that AFP is expressed in fnRBC. The data indicate that the AFP gene was expressed in the HBE-∈ antibody positive, but not the negative, staining cells isolated by LCM/PALM.

### Example 3

### Simultaneous Detection and Enumeration of Fetal Cell Types

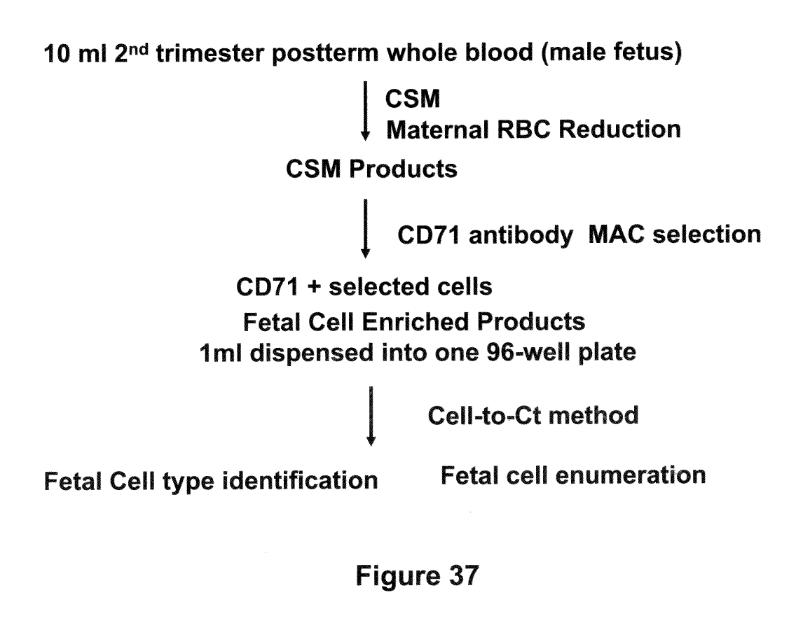

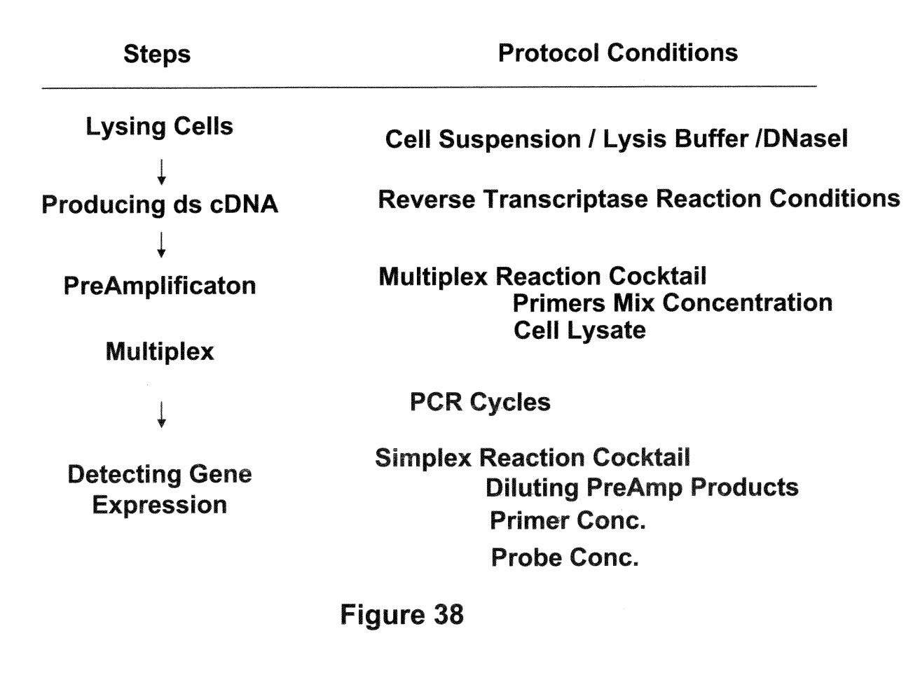

Fetal cells were partially enriched from maternal blood, as illustrated in FIG. 37. Next, direct gene expression profiling was performed on the fetal cell enriched products. Gene expression was analyzed by a Cell-to-Ct protocol using multiplex and pre-amplification steps with HBE (hemoglobin) and hPL gene specific primers and probes, as illustrated in FIG. 38.

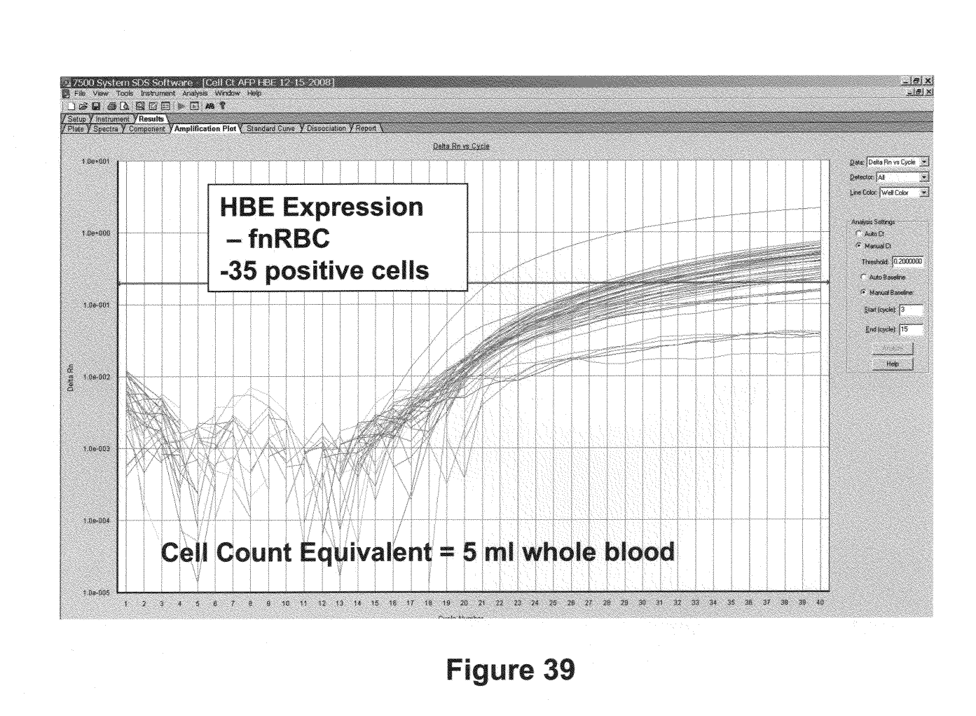

(A) Fetal nRBC cell type and count: As shown in FIG. 39, 35 HBE positive cell counts (one count/well) were detected. A HBE positive cell (well) count is defined as a well with a Ct value less than 37. Total 35 positive counts and 9 negative counts is equivalent to 35 fnRBC counts in 5 ml of whole blood. The data were converted to 70 fnRBC counts in 10 ml whole blood.

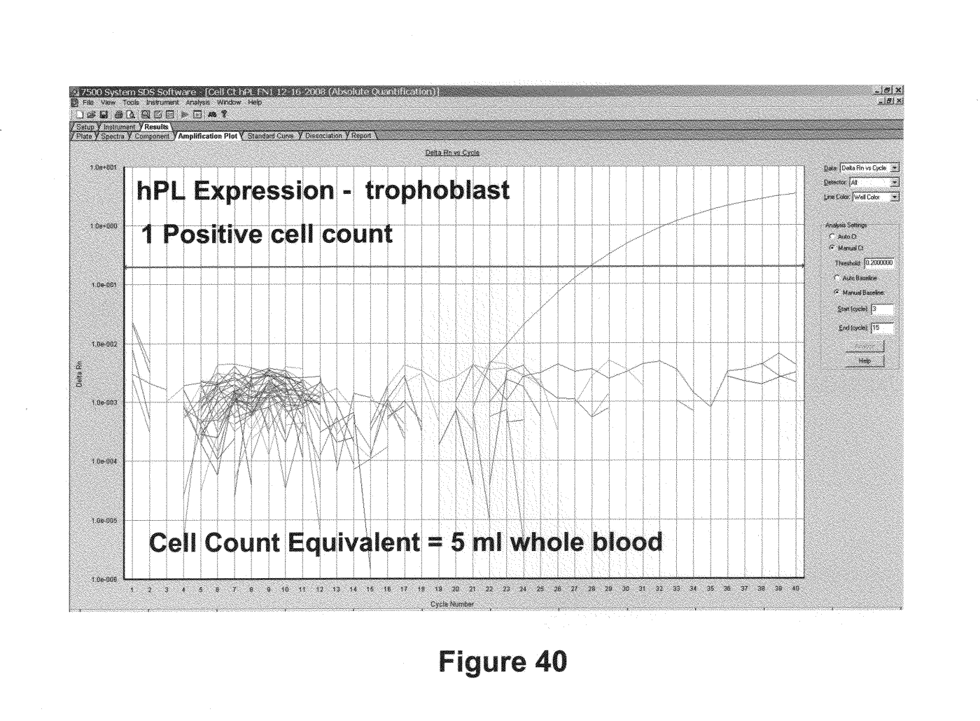

The hPL positive cell (well) count is defined as Ct value less than 37. There was a total of 1 positive count, which is equivalent to one fetal trophoblast in 5 ml whole blood (FIG. 40). Data was converted into 2 fetal trophoblast counts in 10 ml whole blood.

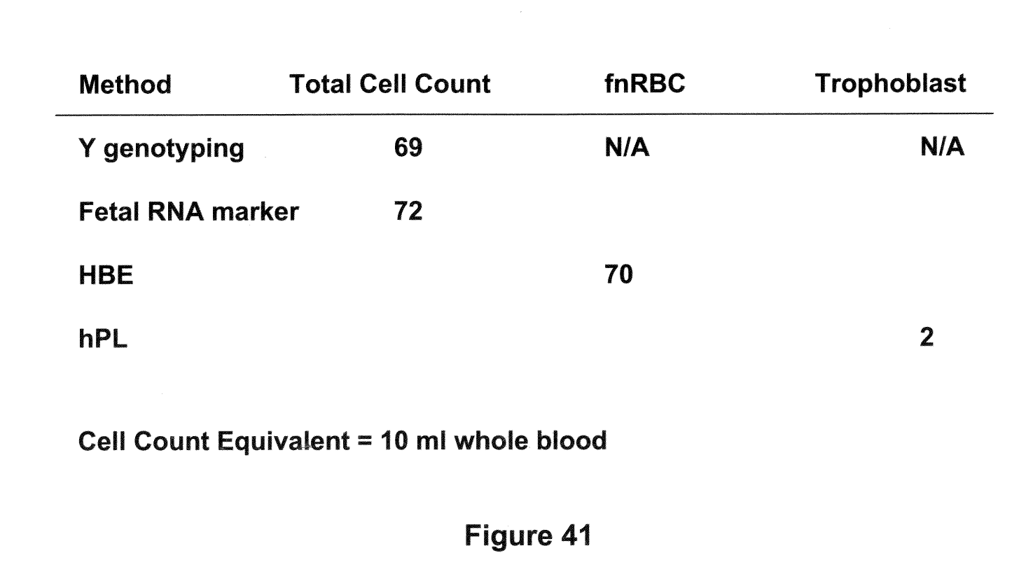

The data indicate that approximately 2 trophoblasts and 70 fetal nRBCs were present in 10 ml whole blood sample, which is similar to the 69 fetal cell counts obtained from Y chromosome genotyping (FIG. 41 and data not shown). Thus, using fetal cell enriched products purified from post-term whole blood, the preliminary data indicated that the identified fetal cell markers can be used to simultaneously detect different fetal cell types and enumerate these cell types.

### Example 4

### Separation of Fetal Cord Blood

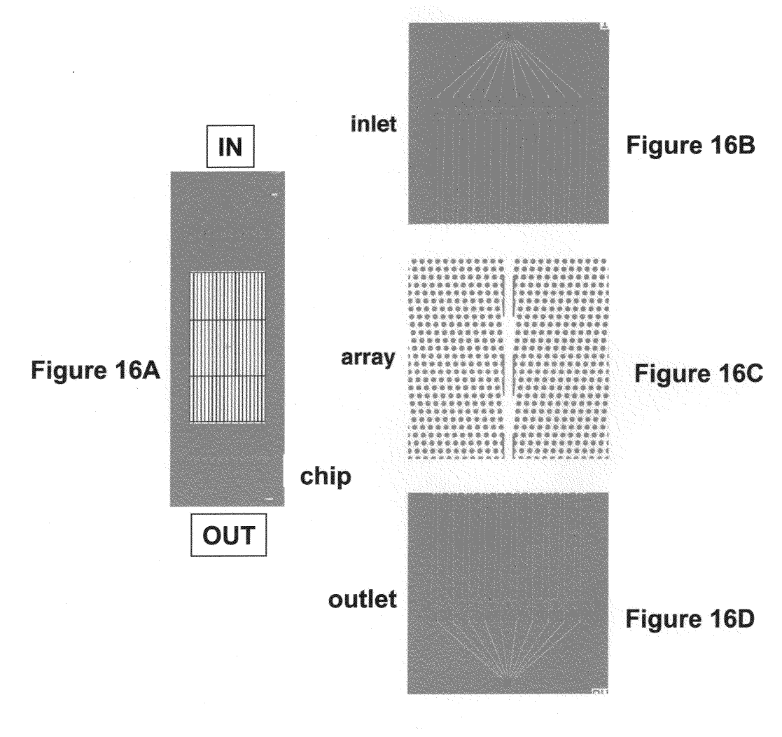

FIGS. 16A-160 shows a schematic of the device used to separate nucleated cells from fetal cord blood.

Dimensions: 100 mm×28 mm×1 mm

Array design: 3 stages, gap size=18, 12 and 8 μm for the first, second and third stage, respectively.

Device fabrication: The arrays and channels were fabricated in silicon using standard photolithography and deep silicon reactive etching techniques. The etch depth is 140 μm. Through holes for fluid access are made using KOH wet etching. The silicon substrate was sealed on the etched face to form enclosed fluidic channels using a blood compatible pressure sensitive adhesive (9795, 3M, St Paul, Minn.).

Device packaging: The device was mechanically mated to a plastic manifold with external fluidic reservoirs to deliver blood and buffer to the device and extract the generated fractions.

Device operation: An external pressure source was used to apply a pressure of 2.0 PSI to the buffer and blood reservoirs to modulate fluidic delivery and extraction from the packaged device.

Experimental conditions: Human fetal cord blood was drawn into phosphate buffered saline containing Acid Citrate Dextrose anticoagulants. 1 mL of blood was processed at 3 mL/hr using the device described above at room temperature and within 48 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100 ML, Sigma-Aldrich, St Louis, Mo.) and 2 mM EDTA (15575-020, Invitrogen, Carlsbad, Calif.).

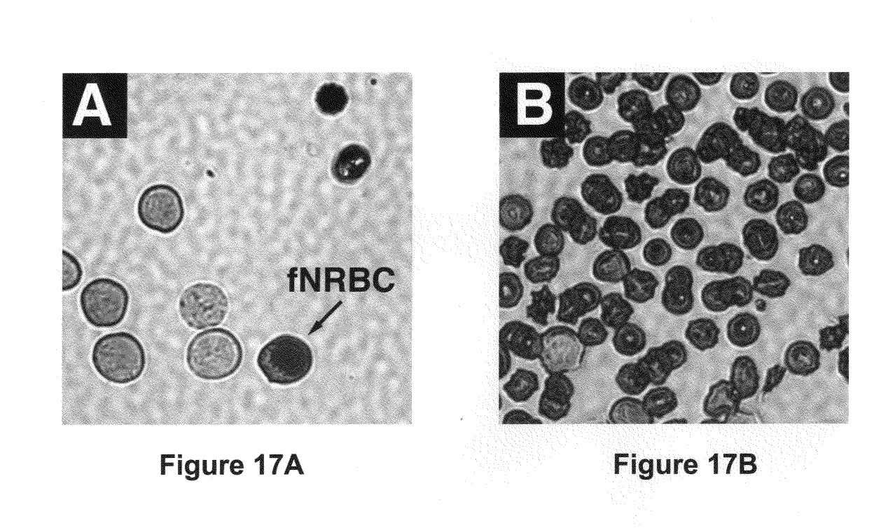

Measurement techniques: Cell smears of the product and waste fractions (FIG. 17A-17B) were prepared and stained with modified Wright-Giemsa (WG16, Sigma Aldrich, St. Louis, Mo.).

Performance: Fetal nucleated red blood cells were observed in the product fraction (FIG. 17A) and absent from the waste fraction (FIG. 17B).

### Example 5

### Isolation of Fetal Cells from Maternal Blood

The device and process described in detail in Example 4 were used in combination with immunomagnetic affinity enrichment techniques to demonstrate the feasibility of isolating fetal cells from maternal blood.

Experimental conditions: blood from consenting maternal donors carrying male fetuses was collected into K₂EDTA vacutainers (366643, Becton Dickinson, Franklin Lakes, N.J.) immediately following elective termination of pregnancy. The undiluted blood was processed using the device described in Example 1 at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100 mL, Sigma-Aldrich, St Louis, Mo.). Subsequently, the nucleated cell fraction was labeled with anti-CD71 microbeads (130-046-201, Miltenyi Biotech Inc., Auburn, Calif.) and enriched using the MiniMACS™ MS column (130-042-201, Miltenyi Biotech Inc., Auburn, Calif.) according to the manufacturer's specifications. Finally, the CD71-positive fraction was spotted onto glass slides.

Measurement techniques: Spotted slides were stained using fluorescence in situ hybridization (FISH) techniques according to the manufacturer's specifications using Vysis probes (Abbott Laboratories, Downer's Grove, Ill.). Samples were stained from the presence of X and Y chromosomes. In one case, a sample prepared from a known Trisomy 21 pregnancy was also stained for chromosome 21.

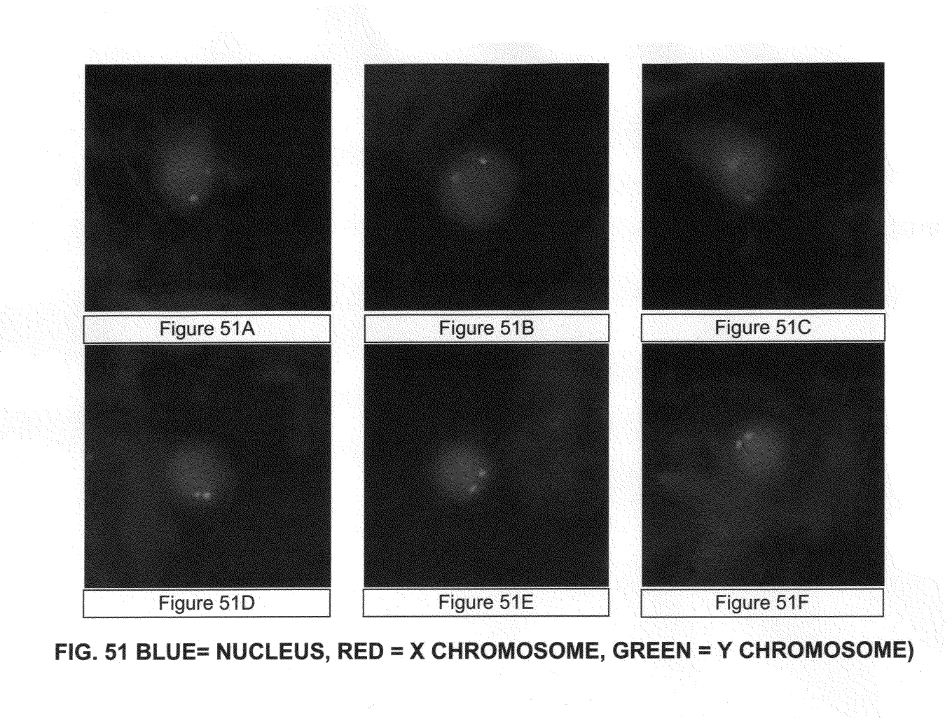

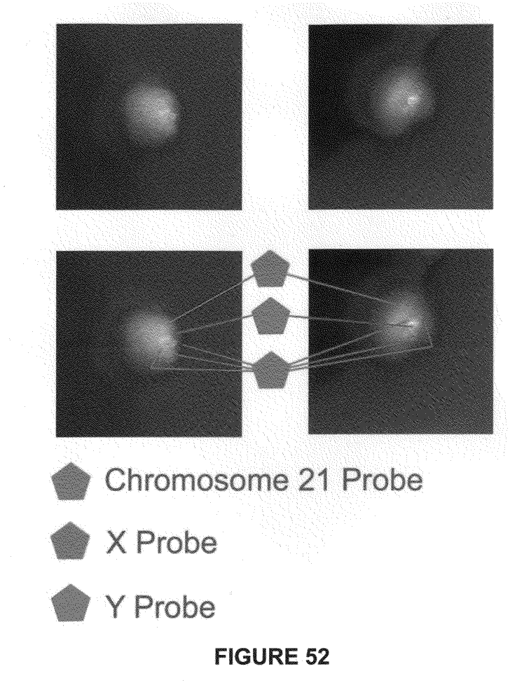

Performance: Isolation of fetal cells was confirmed by the reliable presence of male cells in the CD71-positive population prepared from the nucleated cell fractions (FIG. 51). In the single abnormal case tested, the trisomy 21 pathology was also identified (FIG. 52).

### Example 6

### RT-PCR Protocol

RNA was extracted from blood cells using Qiagen's RNeasy Midi Kit following manufacturer's blood protocol. Briefly, for nucleated cells <3×10⁷, 2 ml of buffer RLT (with β-ME added) to lyse the cells, then 2 ml of 70% ethanol was added to the lysate, and mixed thoroughly by shaking vigorously. Sample was applied to the RNeasy midi column and centrifuged for 5 min at 3000×g. DNA digestion was performed on column at this step to remove genomic DNA. The column was then washed sequentially with RW1 and PRE buffers and eluted with 150 ul RNase-free water. RNA was quantified by NanoDrop™.

Primers and Taqman probe (Applied Biosystems) were specifically designed for each gene of interest. Quantitative RT-PCR was performed using TaqMan One-Step RT-PCR Kit (Applied Biosystems) on ABI 7300 or 7500 Real-time PCR System (Applied Biosystems). Each reaction contained 1× TaqMan One-Step RT-PCR Master Mix without UNG, 1× MultiScribe and RNase Inhibitor Mix, 400 nM of each primer (Integrated DNA Technologies), 250 nM of the corresponding Taqman each probe (Applied Biosystems) and 10 ng of the RNA sample. The RT-PCR was performed at 42° C. for 30 min for RT process, followed by 95° C. for 10 min and 45 cycles of 95° C. for 15 s and 60° C. for 1 min.

### Example 7

### Fetal Diagnosis with CGH

Fetal cells or nuclei can be isolated as described in the enrichment section or as described in Examples 4 or 5. Comparative genomic hybridization (CGH) can be used to determine copy numbers of genes and chromosomes. DNA extracted from the enriched fetal cells will be hybridized to immobilized reference DNA which can be in the form of bacterial artificial chromosome (BAC) clones, or PCR products, or synthesized DNA oligos representing specific genomic sequence tags. Comparing the strength of hybridization fetal cells and maternal control cells to the immobilized DNA segments gives a copy number ratio between the two samples. To perform CGH effectively starting with small numbers of cells, the DNA from the enriched fetal cells can be amplified according to the methods described in the amplification section.

A ratio-preserving amplification of the DNA would be done to minimize these errors; i.e. this amplification method would be chosen to produce as close as possible the same amplification factor for all target regions of the genome. Appropriate methods would include multiple displacement amplification, the two-stage PCR, and linear amplification methods such as in vitro transcription.

To the extent the amplification errors are random their effect can be reduced by averaging the copy number or copy number ratios determined at different loci over a genomic region in which aneuploidy is suspected. For example, a microarray with 1000 oligo probes per chromosome could provide a chromosome copy number with error bars ˜sqrt(1000) times smaller than those from the determination based on a single probe. It is also important to perform the probe averaging over the specific genomic region(s) suspected for aneuploidy. For example, a common known segmental aneuploidy would be tested for by averaging the probe data only over that known chromosome region rather than the entire chromosome. Segmental aneuploidies can be caused by a chromosomal rearrangement, such as a deletion, duplication or translocation event. Random errors could be reduced by a very large factor using DNA microarrays such as Affymetrix arrays that could have a million or more probes per chromosome.

In practice other biases will dominate when the random amplification errors have been averaged down to a certain level, and these biases in the CGH experimental technique must be carefully controlled. For example, when the two biological samples being compared are hybridized to the same array, it is helpful to repeat the experiment with the two different labels reversed and to average the two results—this technique of reducing the dye bias is called a ‘fluor reversed pair’. To some extent the use of long ‘clone’ segments, such as BAC clones, as the immobilized probes provides an analog averaging of these kinds of errors; however, a larger number of shorter oligo probes should be superior because errors associated with the creation of the probe features are better averaged out.

Differences in amplification and hybridization efficiency from sequence region to sequence region can be systematically related to DNA sequence. These differences can be minimized by constraining the choices of probes so that they have similar melting temperatures and avoid sequences that tend to produce secondary structure. Also, although these effects are not truly ‘random’, they will be averaged out by averaging the results from a large number of array probes. However, these effects can result in a systematic tendency for certain regions or chromosomes to have slightly larger signals than others, after probe averaging, which can mimic aneuploidy. When these particular biases are in common between the two samples being compared, they divide out if the results are normalized so that control genomic regions believed to have the same copy number in both samples yield a unity ratio.

After performing CGH analysis trisomy can be diagnosed by comparing the strength of hybridization fetal cells and maternal control cells to the immobilized DNA segments which would give a copy number ratio between the two samples.

In one method, DNA samples will be obtained from the genomic DNA from enriched fetal cells and a maternal control sample. These samples are digested with the Alu I restriction enzyme (Promega, catalog #R6281) in order to introduce nicks into the genomic DNA (e.g. 10 minutes at 55° C. followed by immediately cooling to ˜32° C.). The partially digested sample is then boiled and transferred to ice. This is followed by Terminal Deoxynucleotidyl (TdT) tailing with dTTP at 37° C. for 30 minutes. The sample is boiled again after completion of the tailing reaction, followed by a ligation reaction wherein capture sequences, complementary to the poly T tail and labeled with a fluorescent dye, such as Cy3/green and Cy5/red, are ligated onto the strands. If fetal DNA is labeled with Cy3 then the maternal DNA is labeled with FITC, or vice versa. The ligation reaction is allowed to proceed for 30 minutes at room temperature before it is stopped by the addition of 0.5M EDTA. Labeled DNAs are then purified from the reaction components using a cleanup kit, such as the Zymo DNA Clean and Concentration kit. Purified tagged DNAs are resuspended in a mixture containing 2× hybridization buffer, which contains LNA dT blocker, calf thymus DNA, and nuclease free water. The mixture is vortexed at 14,000 RPM for one minute after the tagged DNA is added, then it is incubated at 95° C.-100° C. for 10 minutes. The tagged DNA hybridization mixture containing both labeled DNAs is then incubated on a glass hybridization slide, which has been prepared with human bacterial artificial chromosomes (BAC), such as the 32K array set. BAC clones covering at least 98% of the human genome are available from BACPAC Resources, Oakland Calif.

The slide is then incubated overnight (˜16 hours) in a dark humidified chamber at 52° C. The slide is then washed using multiple post hybridization washed. The BAC microarray is then imaged using an epifluorescence microscope and a CCD camera interfaced to a computer. Analysis of the microarray images is performed using the GenePix Pro 4.0 software (Axon Instruments, Foster City Calif.). For each spot the median pixel intensity minus the median local background for both dyes is used to obtain a test over reference gene copy number ratio. Data normalization is performed per array sub-grid using lowest curve fitting with a smoothing factor of 0.33. To identify imbalances the MATLAB toolbox CGH plotter is applied, using moving mean average over three clones and limits of log 2>o.2. Classification as gain or loss is based on (1) identification as such by the CGH plotter and (2) visual inspection of the log 2 ratios. In general, log 2 ratios >0.5 in at least four adjacent clones will be considered to be deviating. Ratios of 0.5-1.0 will be classified as duplications/hemizygous deletions; whereas, ratios >1 will be classified as amplifications/homozygous deletions. All normalizations and analyses are carried out using analysis software, such as the BioArray Software Environment database. Regions of the genome that are either gained or lost in the fetal cells are indicated by the fluorescence intensity ratio profiles. Thus, in a single hybridization it is possible to screen the vast majority of chromosomal sites that can contain genes that are either deleted or amplified in the fetal cells

The sensitivity of CGH in detecting gains and losses of DNA sequences is approximately 0.2-20 Mb. For example, a loss of a 200 kb region should be detectable under optimal hybridization conditions. Prior to CGH hybridization, DNA can be universally amplified using degenerate oligonucleotide-primed PCR (DOP-PCR), which allows the analysis of, for example, rare fetal cell samples. The latter technique requires a PCR pre-amplification step.

Primers used for DOP-PCR have defined sequences at the 5′ end and at the 3′ end, but have a random hexamer sequence between the two defined ends. The random hexamer sequence displays all possible combinations of the natural nucleotides A, G, C, and T. DOP-PCR primers are annealed at low stringency to the denatured template DNA and hybridize statistically to primer binding sites. The distance between primer binding sites can be controlled by the length of the defined sequence at the 3′ end and the stringency of the annealing conditions. The first five cycles of the DOP-PCR thermal cycle consist of low stringency annealing, followed by a slow temperature increase to the elongation temperature, and primer elongation. The next thirty-five cycles use a more stringent (higher) annealing temperature. Under the more stringent conditions the material which was generated in the first five cycles is amplified preferentially, since the complete primer sequence created at the amplicon termini is required for annealing. DOP-PCR amplification ideally results in a smear of DNA fragments that are visible on an agarose gel stained with ethidium bromide. These fragments can be directly labelled by ligating capture sequences, complementary to the primer sequences and labeled with a fluorescent dye, such as Cy3/green and Cy5/red. Alternatively the primers can be labelled with a florescent dye, in a manner that minimizes steric hindrance, prior to the amplification step.

### Example 8

### Confirmation of the Presence of Male Fetal Cells in Enriched Samples

Confirmation of the presence of a male fetal cell in an enriched sample is performed using qPCR with primers specific for DYZ, a marker repeated in high copy number on the Y chromosome. After enrichment of fnRBC by any of the methods described herein, the resulting enriched fnRBC are binned by dividing the sample into 100 PCR wells. Prior to binning, enriched samples can be screened by FISH to determine the presence of any fnRBC containing an aneuploidy of interest. Because of the low number of fnRBC in maternal blood, only a portion of the wells will contain a single fnRBC (the other wells are expected to be negative for fnRBC). The cells are fixed in 2% Paraformaldehyde and stored at 4° C. Cells in each bin are pelleted and resuspended in 5 μl PBS plus 1 μA 20 mg/ml Proteinase K (Sigma #P-2308). Cells are lysed by incubation at 65° C. for 60 minutes followed by inactivation of the Proteinase K by incubation for 15 minutes at 95° C. For each reaction, primer sets (DYZ forward primer TCGAGTGCATTCCATTCCG; DYZ reverse primer ATGGAATGGCATCAAACGGAA; and DYZ Taqman Probe 6FAM-TGGCTGTCCATTCCA-MGBNFQ), TaqMan Universal PCR master mix, No AmpErase and water are added. The samples are run and analysis is performed on an ABI 7300: 2 minutes at 50° C., 10 minutes 95° C. followed by 40 cycles of 95° C. (15 seconds) and 60° C. (1 minute). Following confirmation of the presence of male fetal cells, further analysis of bins containing fnRBC is performed. Positive bins can be pooled prior to further analysis.

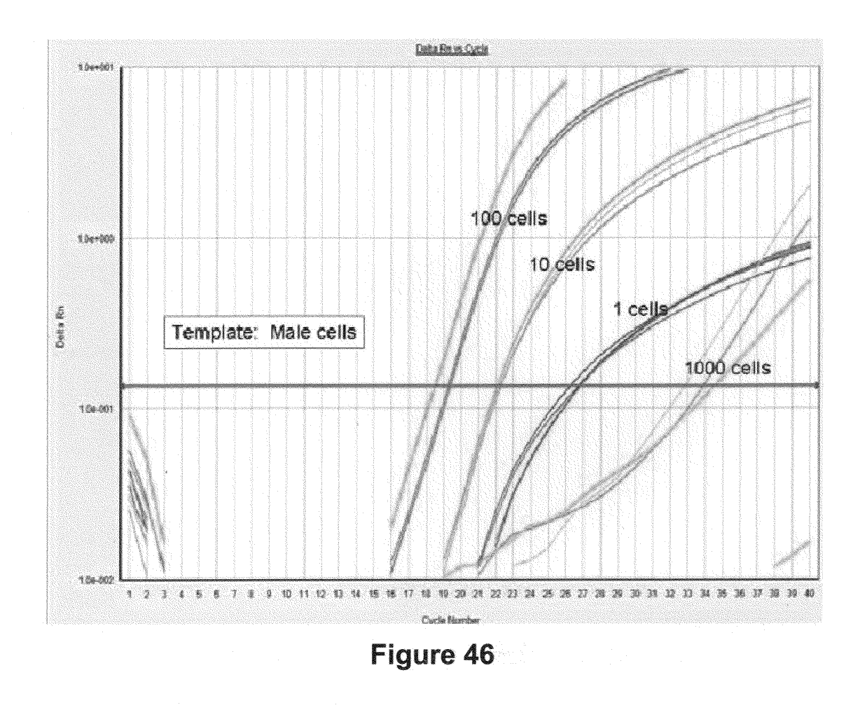

FIG. 46 shows the results expected from such an experiment. The data in FIG. 46 was collected by the following protocol. Nucleated red blood cells were enriched from cord cell blood of a male fetus by Sucrose gradient two Heme Extractions (HE). The cells were fixed in 2% paraformaldehyde and stored at 4° C. Approximately 10×1000 cells were pelleted and resuspended each in 5 μl PBS plus 1 μl 20 mg/ml Proteinase K (Sigma #P-2308). Cells were lysed by incubation at 65° C. for 60 minutes followed by a inactivation of the Proteinase K by 15 minute at 95° C. Cells were combined and serially diluted 10-fold in PBS for 100, 10 and 1 cell per 6 μl final concentration were obtained. Six μl of each dilution was assayed in quadruplicate in 96 well format. For each reaction, primer sets (DYZ forward primer TCGAGTGCATTCCATTCCG; 0.9 uM DYZ reverse primer ATGGAATGGCATCAAACGGAA; and 0.5 uM DYZ TaqMan Probe 6FAM-TGGCTGTCCATTCCA-MGBNFQ), TaqMan Universal PCR master mix, No AmpErase and water were added to a final volume of 25 μl per reaction. Plates were run and analyzed on an ABI 7300: 2 minutes at 50° C., 10 minutes 95° C. followed by 40 cycles of 95° C. (15 seconds) and 60° C. (1 minute). These results show that detection of a single fnRBC in a bin is possible using this method.

### Example 9

### Confirmation of the Presence of Fetal Cells in Enriched Samples by STR Analysis

Maternal blood is processed through a size-based separation module, with or without subsequent MHEM enhancement of fnRBCs. The enhanced sample is then subjected to FISH analysis using probes specific to the aneuploidy of interest (e.g., triploidy 13, triploidy 18, and XYY). Individual positive cells are isolated by “plucking” individual positive cells from the enhanced sample using standard micromanipulation techniques. Using a nested PCR protocol, STR marker sets are amplified and analyzed to confirm that the FISH-positive aneuploid cell(s) are of fetal origin. For this analysis, comparison to the maternal genotype is typical. An example of a potential resulting data set is shown in Table 4. Non-maternal alleles can be proven to be paternal alleles by paternal genotyping or genotyping of known fetal tissue samples. As can be seen, the presence of paternal alleles in the resulting cells, demonstrates that the cell is of fetal origin (cells # 1, 2, 9, and 10). Positive cells can be pooled for further analysis to diagnose aneuploidy of the fetus, or can be further analyzed individually.

TABLE 4
STR locus alleles in maternal and fetal cells

| | STR | STR | STR | STR | STR |
|---|---|---|---|---|---|
| | locus | locus | locus | locus | locus |
| DNA Source | D14S | D16S | D8S | F13B | vWA |
| Maternal alleles | 14, 17 | 11, 12 | 12, 14 | 9, 9 | 16, 17 |
| Cell #1 alleles | | 8 | | | 19 |
| Cell #2 alleles | 17 | | 15 | | |
| Cell #3 alleles | | | 14 | | |
| Cell #4 alleles | | | | | |
| Cell #5 alleles | 17 | 12 | | 9 | |
| Cell #6 alleles | | | | | |
| Cell #7 alleles | | | | | 19 |
| Cell #8 alleles | | | | | |
| Cell #9 alleles | 17 | | 14 | 7, 9 | 17, 19 |
| Cell #10 alleles | | | 15 | | |

### Example 10

### Confirmation of the Presence of Fetal Cells in Enriched Samples by SNP Analysis

Maternal blood is processed through a size-based separation module, with or without subsequent MHEM enhancement of fnRBCs. The enhanced sample is then subjected to FISH analysis using probes specific to the aneuploidy of interest (e.g., triploidy 13, triploidy 18, and XYY). Samples testing positive with FISH analysis are then binned into 96 microtiter wells, each well containing 15 μl of the enhanced sample. Of the 96 wells, 5-10 are expected to contain a single fnRBC and each well should contain approximately. 1000 nucleated maternal cells (both WBC and mnRBC). Cells are pelleted and resuspended in 50 PBS plus 1 μl 20 mg/ml Proteinase K (Sigma #P-2308). Cells are lysed by incubation at 65° C. for 60 minutes followed by a inactivation of the Proteinase K by 15 minute at 95° C.

In this example, the maternal genotype (BB) and fetal genotype (AB) for a particular set of SNPs is known. The genotypes A and B encompass all three SNPs and differ from each other at all three SNPs. The following sequence from chromosome 7 contains these three SNPs (rs7795605, rs7795611 and rs7795233 indicated in brackets, respectively) ATGCAGCAAGGCACAGACTAA[G/A]CAAGGAGA[G/C]GCAAAATTTTC[A/G]TAGGGGAGAGAAA TGGGTCATT).

In the first round of PCR, genomic DNA from binned enriched cells is amplified using primers specific to the outer portion of the fetal-specific allele A and which flank the interior SNP (forward primer ATGCAGCAAGGCACAGACTACG; reverse primer AGAGGGGAGAGAAATGGGTCATT). In the second round of PCR, amplification using real time SYBR Green PCR is performed with primers specific to the inner portion of allele A and which encompass the interior SNP (forward primer CAAGGCACAGACTAAGCAAGGAGAG; reverse primer GGCAAAATTTTCATAGGGGAGAGAAATGGGTCATT).

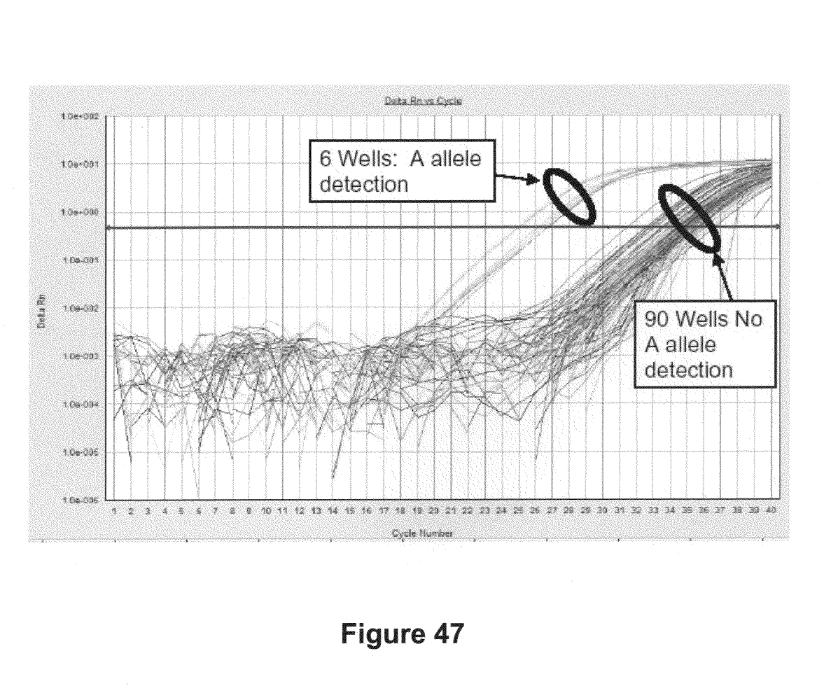

Expected results are shown in FIG. 47. Here, six of the 96 wells test positive for allele A, confirming the presence of cells of fetal origin, because the maternal genotype (BB) is known and cannot be positive for allele A. DNA from positive wells can be pooled for further analysis or analyzed individually.

### Example 11

### Amplification and Sequencing of STRs for Fetal Diagnosis

Fetal cells or nuclei can be isolated as describe in the enrichment section or as described in Examples 4 or 5. DNA from the fetal cells or isolated nuclei from fetal cells can be obtained using any methods known in the art. STR loci can be chosen on the suspected trisomic chromosomes (X, 13, 18, or 21) and on other control chromosomes. These would be selected for high heterozygosity (variety of alleles) so that the paternal allele of the fetal cells is more likely to be distinct in length from the maternal alleles, with resulting improved power to detect. Di-, tri-, or tetra-nucleotide repeat loci can be used. The STR loci can then be amplified according the methods described in the amplification section.

For instance, the genomic DNA from the enriched fetal cells and a maternal control sample can be fragmented, and separated into single strands. The single strands of the target nucleic acids would be bound to beads under conditions that favor each single strand molecule of DNA to bind a different bead. Each bead would then be captured within a droplet of a PCR-reaction-mixture-in-oil-emulsion and PCR amplification occurs within each droplet. The amplification on the bead could results in each bead carrying at least one 10 million copies of the unique single stranded target nucleic acid. The emulsion would be broken, the DNA is denatured and the beads carrying single-stranded nucleic acids clones would be deposited into a picolitre-sized well for further analysis.

The beads can then be placed into a highly parallel sequencing by synthesis machine which can generate over 400,000 reads (˜100 bp per read) in a single 4 hour run. Sequence by synthesis involves inferring the sequence of the template by synthesizing a strand complementary to the target nucleic acid sequence. The identity of each nucleotide would be detected after the incorporation of a labeled nucleotide or nucleotide analog into a growing strand of a complementary nucleic acid sequence in a polymerase reaction. After the successful incorporation of a label nucleotide, a signal would be measured and then nulled and the incorporation process would be repeated until the sequence of the target nucleic acid is identified. The allele abundances for each of the STRs loci can then be determined. The presence of trisomy would be determined by comparing abundance for each of the STR loci in the fetal cells with the abundance for each of the SRTs loci in a maternal control sample. The enrichment, amplification and sequencing methods described in this example allow for the analysis of rare alleles from fetal cells, even in circumstances where fetal cells are in a mixed sample comprising other maternal cells, and even in circumstances where other maternal cells dominate the mixture.

### Example 12

### Analysis of STR's Using Quantitative Fluorescence

Genomic DNA from enriched fetal cells and a maternal control sample will be genotyped for specific STR loci in order to assess the presence of chromosomal abnormalities, such as trisomy. Due to the small number of fetal cells typically isolated from maternal blood it is advantageous to perform a pre-amplification step prior to analysis, using a protocol such as improved primer extension pre-amplification (IPEP) PCR. Cell lysis is carried out in 10 ul High Fidelity buffer (50 mM Tris-HCL, 22 mM (NH.sub.4).sub.2 SO.sub.4 2.5 mM MgCl.sub.2, pH 8.9) which also contained 4 mg/ml proteinase K and 0.5 vol % Tween 20 (Merck) for 12 hours at 48° C. The enzyme is then inactivated for 15 minutes at 94° C. Lysis is performed in parallel batches in 5 ul, 200 mM KOH, 50 mM dithiothreitol for 10 minutes at 65.degree. The batches are then neutralized with 5 ul 900 mM TrisHCl pH 8.3, 300 mM KCl. Preamplication is then carried out for each sample using completely randomized 15-mer primers (16 uM) and dNTP (100 uM) with 5 units of a mixture of Taq polymerase (Boehringer Mannheim) and Pwo polymerase (Boehringer Mannheim) in a ratio of 10:1 under standard PCR buffer conditions (50 mM Tris-HCL, 22 mM (NH.₄)₂ SO₄, 2.5 mM Mg₂, pH 8.9, also containing 5% by vol. of DMSO) in a total volume of 60 ul with the following 50 thermal cycles: Step Temperature Time (1) 92° C. 1 Min 30 Sec; (2) 92° C. 40 Min (3) 37° C. 2 Min; (4) ramp: 0.1° C./sec to 55° C. (5) 55° C. 4 Min (6)68° C. 30 Sec (7) go to step 2, 49 times (8) 8° C. 15 Min.

Dye labeled primers are then chosen from Table 5 based on STR loci on chromosomes of interest, such as 13, 18, 21 or X. The primers are designed so that one primer of each pair contains a fluorescent dye, such as ROX, HEX, JOE, NED, FAM, TAMARA or LIZ. The primers are placed into multiplex mixes based on expected product size, fluorescent tag compatibility and melting temperature. This allows multiple STR loci to be assayed at once and yet still conserves the amount of initial starting material required. All primers are initially diluted to a working dilution of 10 μM. The primers are then combined in a cocktail that has a final volume of 40 ul. Final primer concentration is determined by reaction optimization. Additional PCR grade water is added if the primer mix is below 40 ul. A reaction mix containing 6 ul of Sigma PCR grade water, 1.25 ul of Perkin Elmer Goldamp PCR buffer, 0.5 ul of dNTPs, 8 ul of the primer cocktail, 0.12 ul of Perkin Elmer Taq Gold Polymerase and 1.25 ul of Mg (25 mM) is mixed for each sample. To this a 1 ul sample containing preamplified DNA from enriched fetal cells or maternal control genomic DNA is added.

The reaction mix is amplified in a DNA thermocycler, (PTC-200; MJ Research) using an amplification cycle optimized for the melting temperature of the primers and the amount of sample DNA.

The amplification product will then analyzed using an automated DNA sequencer system, such as the ABI 310, 377, 3100, 3130, 3700 or 3730, or the L1-Cor 4000, 4100, 4200 or 4300. For example when the amplification products are prepared for analysis on a ABI 377 sequencer, 6 ul of products will be removed and combined with 1.6 ul of loading buffer mix. The master loading buffer mix contains 90 ul deionized formamide combined with 25 ul Perkin Elmer loading dye and 10 ul of a size standard, such as the ROX 350 size standard. Various other standards can be used interchangeably depending on the sizes of the labeled PCR products. The loading buffer and sample are then heat denatured at 95° C. for 3 minutes followed by flash cooling on ice. 2 ul of the product/buffer mix is then electrophoresed on a 12 inch 6% (19:1) polyacrylamide gel on an ABI 377 sequencer.

The results are then analyzed using ABI Genotyper software. The incorporation of a fluorochrome during amplification allows product quantification for each chromosome specific STR, with 2 fluorescent peaks observed in a normal heterozygous individual with an approximate ratio of 1:1. By comparison in trisomic samples, either 3 fluorescent peaks with a ratio of 1:1:1 (trialleleic) or 2 peaks with a ratio of around 2:1(diallelic) are observed. Using this method, screening can be carried out for common trisomies and sex chromosome aneuploidy in a single reaction.

TABLE 5
Primer Sets for STRs on Chromosomes 13, 18, 21 and X

| Ch. | STR Marker | Primer 1 | Primer 2 |
|---|---|---|---|
| 13 | D135317 | 5ACAGAAGTCTGGGATGTGGA | GCCCAAAAAGACAGACAGAA |
| | D1351493 | ACCTGTTGTATGGCAGCAGT | AGTTGACTCTTTCCCCAACTA |
| | D1351807 | TTTGGTAAGAAAAACATCTCCC | GGCTGCAGTTAGCTGTCATT |
| | D135256 | CCTGGGCAACAAGAGCAAA | AGCAGAGAGACATAATTGTG |
| | D135258 | ACCTGCCAAATTTTACCAGG | GACAGAGAGAGGGAATAAACC |
| | D135285 | ATATATGCACATCCATCCATG | GGCCAAAGATAGATAGCAAGGTA |
| | D135303 | ACATCGCTCCTTACCCCATC | TGTACCCATTAACCATCCCCA |
| | D135317 | ACAGAAGTCTGGGATGTGGA | GCCCAAAAAGACAGACAGAA |
| | D135779 | AGAGTGAGATTCTGTCTCAATTAA | GGCCCTGTGTAGAAGCTGTA |
| | D135787 | ATCAGGATTCCAGGAGGAAA | ACCTGGGAGGCGGAGCTC |
| | D135793 | GGCATAAAAATAGTACAGCAAGC | ATTTGAACAGAGGCATGTAC |
| | D135796 | CATGGATGCAGAATTCACAG | TCATCTCCCTGTTTGGTAGC |
| | D135800 | AGGGATCTTCAGAGAAACAGG | TGACACTATCAGCTCTCTGGC |
| | D135894 | GGTGCTTGCTGTAAATATAATTG | CACTACAGCAGATTGCACCA |
| 18 | D18551 | CAAACCCGACTACCAGCAAC | GAGCCATGTTCATGCCACTG |
| | D1851002 | CAAAGAGTGAATGCTGTACAAACAGC | CAAGATGTGAGTGTGCTTTTCAGGAG |
| | D18S1357 | ATCCCACAGGATGCCTATTT | ACGGGAGCTTTTGAGAAGTT |
| | D18S1364 | TCAAATTTTTAAGTCTCACCAGG | GCCTGTAGAAAGCAACAACC |
| | D18S1370 | GGTGACAGAGCAAGACCTTG | GCCTCTTGTCATCCCAAGTA |
| | D18S1371 | CTCTCTTCATCCACCATTGG | GCTGTAAGAGACCTGTGTTG |
| | D18S1376 | TGGAACCACTTCATTCTTGG | ATTTCAGACCAAGATAGGC |
| | D18S1390 | CCTATTTAAGTTTCTGTAAGG | ATGGTGTAGACCCTGTGGAA |
| | D18S499 | CTGCACAACATAGTGAGACCTG | AGATTACCCAGAAATGAGATCAGC |
| | D18S535 | TCATGTGACAAAAGCCACAC | AGACAGAAATATAGATGAGAATGCA |
| | D18S535 | TCATGTGACAAAAGCCACAC | AGACAGAAATATAGATGAGAATGCA |
| | D18S542 | TTTCCAGTGGAAACCAAACT | TCCAGCAACAACAAGAGACA |
| | D18S843 | GTCCTCATCCTGTAAAACGGG | CCACTAACTAGTTTGTGACTTTGG |
| | D18S851 | CTGTCCTCTAGGCTCATTTAGC | TTATGAAGCAGTGATGCCAA |
| | D18S858 | AGCTGGAGAGGGATAGCATT | TGCATTGCATGAAAGTAGGA |
| | D18S877 | GATGATAGAGATGGCACATGA | TCTTCATACATGCTTTATCATGC |
| 21 | D21S11 | GTGAGTCAATTCCCCAAG | GTTGTATTAGTCAATGTTCTCC |
| | D21S1411 | ATGATGAATGCATAGATGGATG | AATGTGTGTCCTTCCAGGC |
| | D21S1413 | TTGCAGGGAAACCACAGTT | TCCTTGGAATAAATTCCCGG |
| | D21S1432 | CTTAGAGGGACAGAACTAATAGGC | AGCCTATTGTGGGTTTGTGA |
| | D21S1437 | ATGTACATGTGTCTGGGAAGG | TTCTCTACATATTTACTGCCAACA |
| | D21S1440 | GAGTTTGAAAATAAAGTGTTCTGC | CCCCACCCCTTTTAGTTTTA |
| | D21S1446 | ATGTACGATACGTAATACTTGACAA | GTCCCAAAGGACCTGCTC |
| | D21S2052 | GCACCCCTTTATACTTGGGTG | TAGTACTCTACCATCCATCTATCCC |
| | D21S2055 | AACAGAACCAATAGGCTATCTATC | TACAGTAAATCACTTGGTAGGAGA |
| X | SBMA | TCCGCGAAGTGAAGAAC | CTTGGGGAGAACCATCCTCA |
| | DXS1047 | CCGGCTACAAGTGATGTCTA | CCTAGGTAACATAGTGAGACCTTG |
| | DXS1068 | CCTCTAAAGCATAGGGTCCA | CCCATCTGAGAACACGCTG |
| | DXS1283E | AGTTTAGGAGATTATCAAGCTGG | GTTCCCATAATAGATGTATCCAG |
| | DXS6789 | TTGGTACTTAATAAACCCTCTTTT | CTAGAGGGACAGAACCAATAGG |
| | DXS6795 | TGTCTGCTAATGAATGATTTGG | CCATCCCCTAAACCTCTCAT |
| | DXS6800 | GTGGGACCTTGTGATTGTGT | CTGGCTGACACTTAGGGAAA |
| | DXS6810 | ACAGAAAACCTTTTGGGACC | CCCAGCCCTGAATATTATCA |
| | DXS7127 | TGCACTTAATATCTGGTGATGG | ATTTCTTTCCCTCTGCAACC |
| | DXS7132 | AGCCCATTTTCATAATAAATCC | AATCAGTGCTTTCTGTACTATTGG |
| | DXS8377 | CACTTCATGGCTTACCACAG | GACCTTTGGAAAGCTAGTGT |
| | DXS9893 | TGTCACGTTTACCCTGGAAC | TATTCTTCTATCCAACCAACAGC |
| | DXS9895 | TTGGGTGGGGACACAGAG | CCTGGCTCAAGGAATTACAA |
| | DXS9896 | CCAGCCTGGCTGTTAGAGTA | ATATTCTTATATTCCATATGGCACA |
| | DXS9902 | TGGAGTCTCTGGGTGAAGAG | CAGGAGTATGGGATCACCAG |
| | DXS998 | CAGCAATTTTTCAAAGGC | AGATCATTCATATAACCTCAAAAGA |

### Example 13

### Enumeration of Fetal Cells in Maternal Blood

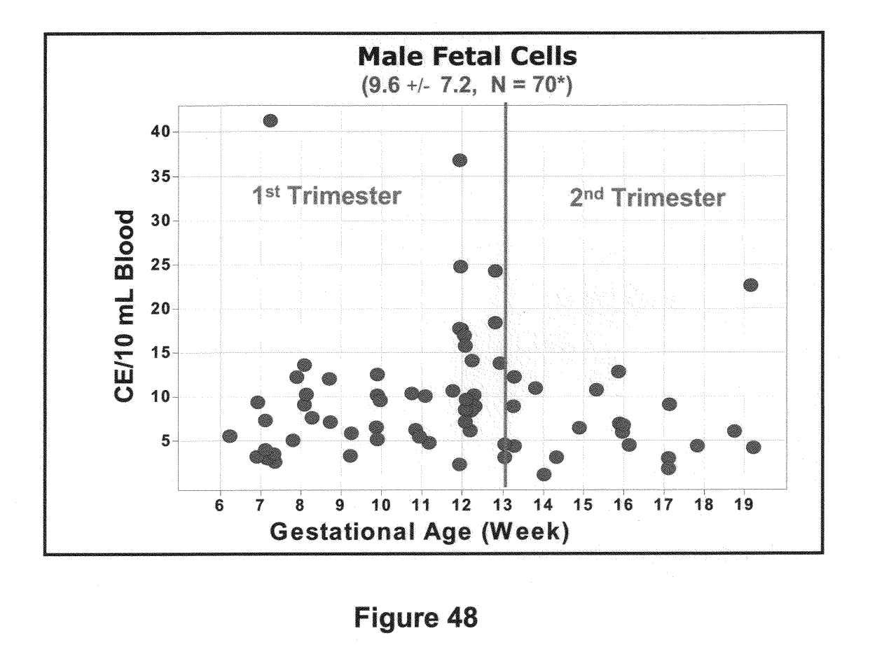

Methods were developed to accurately enumerate circulating fetal cells in early pregnancy, using the Y-chromosome as the fetal cell marker, in order to study the relationship between fetal cell numbers and gestational age (FIG. 48). Fetal DNA markers specific for the Y chromosome were used to count fetal cells in maternal whole blood using PCR analysis PCR analysis has been developed to differentiate between large DNA fragments from intact cells and small fragment from circulating cell-free fetal DNA. Fetal cells are detected in all pregnant samples tested. Fetal cell number is independent of gestational age in 1st & 2nd trimesters.

Blood samples were analyzed from pregnant women with gestational ages ranging from 6 to 19 weeks. The first fetal cell enumeration methods were able to distinguish between DNA from intact fetal cells and that from fragmented cell free fetal nucleic acids. The average number of fetal cells was 9.6±7.2 cells/10 ml whole blood, and ranged from 2-41 cells/10 ml blood.

In the future a method of enumeration will be developed to accurately enumerate circulating fetal cells in early pregnancy, using detection of an hPL, CHS2, KISS1, GDF15, CRH, TFP12, CGB, LOC90625, FN1, COL1A2, PSG9, PSG1, HBE, AFP, APOC3, SERPINC1, AMBP, CPB2, ITIH1, APOH, HPX, beta-hCG, AHSG, APOB, or J42-4-d transcript or protein. The enumeration of fetal cells from a maternal blood sample will be used to study the relationship between fetal cell numbers and a fetal abnormal condition.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A method for identifying a fnRBC comprising detecting transcript or protein expression of a HBE, AFP, AHSG, or J42-4-d gene.

2. The method of claim 1, wherein said detecting comprises using at least two primers and at least one probe that anneals to a cDNA generated from a transcript expressed by said HBE, AFP, AHSG, or J42-4-d gene.

3. A method for identifying a trophoblast comprising detecting transcript or protein expression of a KISS1, LOC90625, AFP, hPL, beta-hCG, or FN1 gene.

4. The method of claim 3, wherein said detecting comprises using at least two primers and at least one probe that anneals to a cDNA generated from a transcript expressed by said KISS1, LOC90625, AFP, hPL, beta-hCG, or FN1 gene.

5. A method for identifying a fetal cell in a maternal sample comprising detecting transcript or protein expression by a cell of one or more of the KISS1, LOC90625, FN1, or AHSG genes to distinguish said fetal cell from a maternal cell.

6. A method for identifying a fetal cell in a maternal sample comprising detecting transcript or protein expression by a cell of three or more of the hPL, KISS1, LOC90625, FN1, PSG9, HBE, AFP, beta-hCG, AHSG or J42-4-d genes to distinguish said fetal cell from a maternal cell.

7. The method of claim 5 or 6, wherein the maternal sample is a maternal blood sample, amniocentesis sample, or cervical swab.

8. The method of claim 5 or 6, wherein said fetal cell is a fetal nucleated RBC or a placental cell.

9. The method of claim 7, wherein said sample is taken in the 1^{st} or early 2^{nd} trimester.

10. The method of claim 7, wherein said sample is taken in the 2^{nd} trimester.

11. The method of claim 5, wherein said fetal cell is a fetal nucleated red blood cell and said gene is AHSG.

12. The method of claim 5, wherein said fetal cell is a trophoblast and said gene is FN1.

13. The method of claim 5 or 6, wherein said detecting comprises RNA FISH, RNA-FISH with a molecule beacon probe, RT-PCR, Q-PCR, digital mRNA profiling, Northern blotting, ribonuclease protection assay, or RNA expression profiling using microarrays.

14. The method of claim 5 or 6, wherein said detecting comprises binding a protein with one or more binding moieties.

15. The method of claim 14, wherein said one or more binding moieties is an antibody, Fab fragment, Fc fragment, scFv fragment, peptidomimetic, or peptoid.

16. A method for identifying a fetal cell in a maternal sample, comprising:
a. enriching a fetal cell, and
b. detecting protein or transcript expression of one or more genes by said fetal cell, wherein said expression of said one or more genes distinguishes said fetal cell from a maternal cell, wherein said one or more genes is hPL, CHS2, KISS1, GDF15, CRH, TFP12, CGB, LOC90625, FN1, COL1A2, PSG9, PSG1, AFP, APOC3, SERPINC1, AMBP, CPB2, ITIH1, APOH, HPX, beta-hCG, AHSG, APOB, or J42-4-d.

17. The method of claim 16, wherein the step of enriching a fetal cell comprises one or more steps of density centrifugation, size based separation, affinity separation, magnetic separation, microfluidic fluorescent cell sorting, dielectrophoretic enrichment, or antibody separation.

18. The method of claim 16, wherein the sample is a maternal blood sample, amniocentesis sample, or cervical swab.

19. The method of claim 16, wherein said cell is a fetal nucleated RBC or a placental cell.

20. The method of claim 16, further comprising enriching a fetal nucleated RBC by magnetic enrichment.

21. The method of claim 16, further comprising enriching one or more fetal nucleated RBCs by anti-CD71 or anti-GLA selection.

22. The method of claim 16, further comprising enriching one or more trophoblasts by anti-HLA-G or anti-EGFR selection.

23. The method of claim 16, wherein said cell is a fetal nucleated RBC and said one or more genes is AFP, AHSG, or J42-4-d.

24. The method of claim 16, wherein said cell is a trophoblast and said one or more genes is KISS1, LOC90625, AFP, hPL, beta-hCG, or FN1.

25. The method of claim 16, wherein said detecting is by RNA FISH, RNA-FISH with a molecule beacon probe, RT-PCR, Q-PCR, digital mRNA profiling, Northern blotting, ribonuclease protection assay, or RNA expression profiling using microarrays.

26. The method of claim 16, wherein said fetal cell is from a maternal sample obtained in the 1^{st} trimester or 2^{nd} trimester of pregnancy.

27. The method of claim 16, wherein said detecting protein expression comprises binding a protein with a binding moiety.

28. The method of claim 27, wherein said binding moiety is an antibody, Fab fragment, Fc fragment, scFv fragment, peptidomimetic, or peptoid.

29. A method for identifying a fetal cell specific transcript comprising
a. isolating a transcript from a sample containing a fetal cell and a transcript from a sample lacking fetal cells;
b. producing cDNAs of said transcripts;
c. performing quantitative PCR on said cDNAs; and
d. comparing results of said quantitative PCR between samples to identify a marker transcript with higher expression in a fetal cell relative to a non-fetal cell.

30. The method of claim 29, wherein said fetal cell is first enriched from a maternal sample by size based separation.

31. The method of claim 29, further comprising a verifying step comprising detecting a marker transcript by quantitative PCR.
